(19) United States
(12) Patent Application Publication
(10) Pub. No.: US 2003/0124536 A1
(43) Pub. Date: Jul. 3, 2003

(54) Diagnosis and treatment of vascular disease

(75) Inventor: Jeanette McCarthy, San Diego, CA (US)

Correspondence Address: LAHIVE & COCKFIELD, 28 STATE STREET, BOSTON, MA, 02109, US

(73) Assignee: Vitivity, Inc., Cambridge, MA

(21) Appl. No.: 10/017,128

(22) Filed: Dec. 14, 2001

Related U.S. Application Data
(60) Provisional application No. 60/306,941, filed on Jul. 20, 2001.
(60) Provisional application No. 60/315,572, filed on Aug. 28, 2001.
(60) Provisional application No. 60/327,488, filed on Oct. 5, 2001.

(51) Int. Cl. C12Q 1/68

(52) U.S. Cl. 435/6

(57) ABSTRACT

The present invention is based at least in part on the discovery of polymorphisms within the phospholipase C gamma 1 (PLCG1) gene and the plasminogen activator inhibitor type 2 (PAI-2) gene. Accordingly, the invention provides nucleic acid molecules having a nucleotide sequence of an allelic variant of a PLCG1 or PAI-2 gene. The invention also provides methods for identifying specific alleles of polymorphic regions of a PLCG1 or PAI-2 gene, methods for determining whether a subject is or is not at risk of developing a disease which is associated with a specific allele of a polymorphic region of a PLCG1 or PAI-2 gene, e.g., a vascular disease, based on detection of polymorphisms within the PLCG1 or PAI-2 gene, and kits for performing such methods. The invention further provides methods for classifying a subject who is or is not at risk for developing, a vascular disease or disorder as a candidate for a particular clinical course of therapy or a particular diagnostic evaluation. The invention further provides methods for selecting a clinical course of therapy or a diagnostic evaluation to treat a subject who is or is not at risk for developing, a vascular disease or disorder.

### BACKGROUND OF THE INVENTION

Cardiovascular disease is a major health risk throughout the industrialized world. Coronary artery disease (CAD), or atherosclerosis, involves the progressional narrowing of the arteries due to a build-up of atherosclerotic plaque. Myocardial infarction (MI), e.g., heart attack, results when the heart is damaged due to reduced blood flow to the heart caused by the build-up of plaque in the coronary arteries.

Coronary artery disease, the most prevalent of cardiovascular diseases, is the principal cause of heart attack, stroke, and gangrene of the extremities, and thereby the principle cause of death in the United States. Coronary artery disease, or atherosclerosis, is a complex disease involving many cell types and molecular factors (described in, for example, Ross, 1993, Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of may conditions that predispose a subject to accelerated development of atherosclerotic cardiovascular disease. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque.

Such plaques occlude the blood vessel concerned and, thus, restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

One of the most important risk factors for coronary artery disease is a familial history. Although family history subsumes both genetic and shared environmental factors, studies suggest that CAD has a very strong genetic component (Marenberg, et al. (1994) NEJM 330:1041). Despite the importance of family history as a risk factor for CAD, it's incomplete genetic basis has not been elucidated. Therefore, the identification of genes which are involved in the development of CAD and MI would be beneficial.

The phospholipase C gamma 1 gene (PLCG1) hydrolyzes phosphatidylinositol 4,5-bisphosphate to generate the second messengers, inositol 1,4,5-trisphosphate (IP3) and diacylglycerol (DAG). IP3 induces a transient increase in intracellular free Ca2+, while DAG directly activates protein kinase C. Upon stimulation of cells with growth factors, PLCG1 is activated upon their association with and phosphorylation by receptor and non-receptor tyrosine kinases as well as interaction with specialized adaptor molecules and, perhaps, other second messenger molecules (Kim, et al. (2000) Exp. Mol. Med. 30;32(3):101-9 and Carpenter, et al. (1999) Exp Cell Res 253(1):15-24). It has been found that the specific binding of phosphatidic acid to PLCG1 is decreased in an experimental animal model of the failing heart (Tappia, et al. (2001) J. Mol. Cell 33(3):431-40).

Plasminogen activator inhibitor type 2 (PAI-2) is an important regulator of plasminogen activation which is involved in the regulation of vascular remodeling, maintenance of intervillous blood flow, and in the regulation of cell migration and proliferation (Irigoyen, et al. (1999) Cell Mol Life 56(1-2):104). PAI-2 has also been identified as playing a role in the inflammatory process (Kruithof, E. (1997) Hematologie 3:7-12).

It would thus be beneficial to identify polymorphic regions within the PLCG1 and PAI-2 genes which are associated with a vascular disease or disorder, such as coronary artery disease or myocardial infarction. It would further be desirable to provide prognostic, diagnostic, pharmacogenomic, and therapeutic methods utilizing the identified polymorphic regions.

### SUMMARY OF THE INVENTION

The present invention is based, at least in part, on the identification of polymorphic regions within the phospholipase C gamma 1 gene (PLCG1) and the plasminogen activator inhibitor type 2 gene (PAI-2), which are associated with specific diseases or disorders, including vascular diseases or disorders. In particular, single nucleotide polymorphisms (SNPs) in these genes which are associated with premature coronary artery disease (CAD) (or coronary heart disease) and myocardial infarction (MI) have been identified.

The present invention is based, also in part, on the discovery that a subject having two copies of the variant allele of the PLCG1 gene (TT) at residue 64001 of the reference sequence GI 11345540 and two copies of the reference allele of the PAI-2 gene (AA) at residue 170871 of the reference sequence GI 6705901, in combination, is at a decreased risk of developing a vascular disease such as CAD or MI compared to a subject having any other possible combination of alleles at these residues. Thus, the invention relates to polymorphic regions and in particular, SNPs identified as described herein, both singly and, preferably, in combination, as well as to the use of these SNPs, and others in these genes, particularly those nearby in linkage disequilibrium with these SNPs, both singly and, preferably, in combination, for predicting the risk of developing a vascular disease or disorder such as CAD and MI in a subject.

The SNPs identified herein may further be used in the development of new treatments for vascular disease based upon comparison of the variant and normal versions of the gene or gene product (e.g., the reference sequence), and development of cell-culture based and animal models for research and treatment of vascular disease. The invention further relates to novel compounds and pharmaceutical compositions for use in the diagnosis and treatment of such disorders. In preferred embodiments, the vascular disease is CAD or MI.

The polymorphisms of the invention may thus be used, both singly, or, preferably, in combination, in prognostic, diagnostic, and therapeutic methods. For example, the polymorphisms of the invention can be used to determine whether a subject is or is not at risk of developing a disease or disorder associated with a specific allelic variant of a PLCG1 or PAI-2 polymorphic region, e.g., a disease or disorder associated with aberrant PLCG1 or PAI-2 activity, e.g., a vascular disease or disorder such as CAD or MI.

The invention thus relates to isolated nucleic acid molecules and methods of using these molecules. The nucleic acid molecules of the invention include specific PLCG1 or PAI-2 allelic variants which differ from the reference PLCG1 or PAI-2 sequences set forth in SEQ ID NO:1 (GI 11345540) or SEQ ID NO:3 (GI 6705901), respectively, or a portion thereof. The preferred nucleic acid molecules of the invention comprise PLCG1 or PAI-2 polymorphic regions or portions thereof having the polymorphisms shown in Table 3 (corresponding to SEQ ID NOs:5 and SEQ ID NO:6), polymorphisms in linkage disequilibrium with the polymorphisms shown in Table 3, and combinations thereof Nucleic acids of the invention can function as probes or primers, e.g., in methods for determining the allelic identity of a PLCG1 or PAI-2 polymorphic region in a nucleic acid of interest.

The nucleic acids of the invention can also be used, singly, or, preferably, in combination, to determine whether a subject is or is not at risk of developing a disease associated with a specific allelic variant of a PLCG1 or PAI-2 polymorphic region, e.g., a disease or disorder associated with aberrant PLCG1 or PAI-2 activity, e.g., a vascular disease or disorder such as CAD or MI. The nucleic acids of the invention can further be used to prepare PLCG1 or PAI-2 polypeptides encoded by specific alleles, such as mutant (variant) alleles. Such polypeptides can be used in therapy. Polypeptides encoded by specific PLCG1 or PAI-2 alleles, such as variant PLCG1 or PAI-2 polypeptides, can also be used as immunogens and selection agents for preparing, isolating or identifying antibodies that specifically bind PLCG1 or PAI-2 proteins encoded by these alleles. Accordingly, such antibodies can be used to detect variant PLCG1 or PAI-2 proteins.

There are two preferred polymorphisms of the invention. One polymorphism found in the population screened is a change from a cytidine (C) to a thymidine (T) in the PLCG1 gene at residue 64001 of the reference sequence GI 11345540 or a change from a C to a T at residue 4363659 of the reference sequence GI 13653753 (polymorphism ID No. G329 ul). This polymorphism results in a change from an isoleucine to a threonine in the amino acid sequence of PLCG1 (SEQ ID NO:2) at amino acid residue 813. A second polymorphism is a change from a thymidine (T) to a cytidine (G) at residue 170871 of the reference sequence GI 6705901 (polymorphism ID No. PAI2 ul). This polymorphism results in a change from an asparagine to an aspartic acid in the amino acid sequence of PAI-2 (SEQ ID NO:4) at amino acid residue 120.

The nucleic acid molecules of the invention can be double- or single-stranded. Accordingly, in one embodiment of the invention, a complement of the nucleotide sequence is provided wherein the polymorphism has been identified. For example, where there has been a single nucleotide change from a thymidine to a cytidine in a single strand, the complement of that strand will contain a change from an adenine to a guanine at the corresponding nucleotide residue. The invention further provides allele-specific oligonucleotides that hybridize to a gene comprising a polymorphism of the present invention or to its complement.

The polymorphisms of the present invention, either singly, in combination with each other, or in combination with previously identified polymorphisms, are shown herein to be associated with specific disorders, e.g., vascular diseases or disorders. Examples of vascular diseases or disorders include, without limitation, atherosclerosis, coronary artery disease (CAD), myocardial infarction (MI), ischemia, stroke, peripheral vascular diseases, venous thromboembolism and pulmonary embolism.

The invention further provides vectors comprising the nucleic acid molecules of the present invention; host cells transfected with said vectors whether prokaryotic or eukaryotic; and transgenic non-human animals which contain a heterologous form of a functional or non-functional PLCG1 or PAI-2 allele described herein. Such a transgenic animal can serve as an animal model for studying the effect of specific PLCG1 or PAI-2 allelic variations, including mutations, as well as for use in drug screening and/or recombinant protein production.

In another preferred embodiment, the method comprises determining the nucleotide content of at least a portion of a PLCG1 or PAI-2 gene, such as by sequence analysis. In yet another embodiment, determining the molecular structure of at least a portion of a PLCG1 or PAI-2 gene is carried out by single-stranded conformation polymorphism (SSCP). In yet another embodiment, the method is an oligonucleotide ligation assay (OLA). Other methods within the scope of the invention for determining the molecular structure of at least a portion of a PLCG1 or PAI-2 gene include hybridization of allele-specific oligonucleotides, sequence specific amplification, primer specific extension, and denaturing high performance liquid chromatography (DHPLC). In at least some of the methods of the invention, the probe or primer is allele specific. Preferred probes or primers are single stranded nucleic acids, which optionally are labeled.

The methods of the invention can be used for determining the identity of a nucleotide or amino acid residue within a polymorphic region of a human PLCG1 or PAI-2 gene present in a subject. For example, the methods of the invention can be useful for determining whether a subject is or is not at risk of developing a disease or condition associated with a specific allelic variant of a polymorphic region in the human PLCG1 or PAI-2 gene, e.g., a vascular disease or disorder.

In one embodiment, the disease or condition is characterized by an aberrant PLCG1 or PAI-2 activity, such as aberrant PLCG1 or PAI-2 protein level, which can result from aberrant expression of a PLCG1 or PAI-2 gene. The disease or condition can be CAD, MI, or another vascular disease. Accordingly, the invention provides methods for predicting a subject's risk for developing a vascular disease associated with aberrant PLCG1 or PAI-2 activity. In a preferred embodiment, a subject having two copies of the variant allele of the PLCG1 gene (TT) at residue 64001 of the reference sequence GI 11345540 and two copies of the reference allele of the PAI-2 gene (TT) at residue 170871 of the reference sequence GI 6705901, in combination, is approximately 3-fold less likely to develop a vascular disease such as CAD or MI compared to a subject having any other possible combination of alleles at these residues (see Example 2).

Additionally, the invention provides a method of identifying a subject who is or is not susceptible to a vascular disorder, which method comprises the steps of i) providing a nucleic acid sample from a subject; and ii) detecting in the nucleic acid sample the presence or absence of a PLCG1 or PAI-2 gene polymorphism, or both in combination, that correlate with the vascular disorder with a P value less than or equal to 0.05.

The invention further provides forensic methods based on detection of polymorphisms within the PLCG1 or PAI-2 gene.

The invention also provides probes and primers comprising oligonucleotides, which correspond to a region of nucleotide sequence which hybridizes to at least 6 consecutive nucleotides of the sequence set forth as SEQ ID NOs:5 and 6 or to the complement of the sequences set forth as SEQ ID NOs:5 and 6, or naturally occurring mutants or variants thereof. In preferred embodiments, the probe/primer further includes a label attached thereto, which is capable of being detected.

A kit of the invention can be used, e.g., for determining whether a subject is or is not at risk of developing a disease associated with a specific allelic variant of a polymorphic region of a PLCG1 or PAI-2 gene, e.g., CAD or MI. In a preferred embodiment, the invention provides a kit for determining whether a subject is or is not at risk of developing a vascular disease such as, for example, atherosclerosis, CAD, MI, ischemia, stroke, peripheral vascular diseases, venous thromboembolism and pulmonary embolism. The kit of the invention can also be used in selecting the appropriate clinical course of clinical treatment to a subject to treat a disease or condition, such as a disease or condition set forth above. Thus, determining the allelic variants of PLCG1 or PAI-2 polymorphic regions of a subject can be useful in predicting how a subject will respond to a specific drug, e.g., a drug for treating a disease or disorder associated with aberrant PLCG1 or PAI-2, e.g., a vascular disease or disorder.

Other features and advantages of the invention will be apparent from the following detailed description and claims.

### BRIEF DESCRIPTION OF THE FIGURES

FIG. 1 depicts the nucleotide sequence corresponding to reference sequence GI 11345540 (SEQ ID NO:1) for the PLCG gene.

FIG. 2 depicts the reference amino acid sequence for the PLCG1 protein (SEQ ID NO:2).

FIG. 3 depicts the nucleotide sequence corresponding to reference sequence GI 6705901 (SEQ ID NO:3) for the PAI-2 gene.

FIG. 4 depicts the reference amino acid sequence for the PAI-2 protein (SEQ ID NO:4).

FIG. 5 is a Table listing the demographic characteristics of cases and controls used in the identification of SNPs associated with vascular disease.

### DETAILED DESCRIPTION OF THE INVENTION

The present invention is based, in part, on the identification of polymorphic regions within the phospholipase C gamma 1 gene (PLCG1) and the plasminogen activator inhibitor type 2 gene (PAI-2). The polymorphic regions of the invention contain polymorphisms which correlate with specific diseases or conditions, including vascular diseases or disorders, including, but not limited to, atherosclerosis, coronary artery disease (CAD), myocardial infarction (MI), ischemia, stroke, peripheral vascular diseases, venous thromboembolism and pulmonary embolism.

The polymorphisms of the present invention are single nucleotide polymorphisms (SNPs) at a specific nucleotide residue within the PLGC1 gene and the PAI-2 gene. The PLGC1 gene and the PAI-2 gene have at least two alleles, referred to herein as the reference allele and the variant allele. The reference alleles (i.e., the consensus sequences) have been designated based on their frequency in a general United States Caucasian population sample. The reference allele is the more common of the two alleles; the variant allele is the more rare of the two alleles. Nucleotide sequences in GenBank may correspond to either allele and correspond to the nucleotide sequence of the nucleotide sequence which has been deposited in GenBank™ and given a specific Accession Number (e.g., GI 11345540, the reference sequence for the PLCG1 gene or GI 6705901, the reference sequence for the PAI-2 gene, corresponding to SEQ ID NO:1 and SEQ ID NO:3, respectively). The reference sequence for the amino acid sequences of PLCG1 and PAI-2 proteins are set forth as SEQ ID NO:2 and SEQ ID NO:4, respectively. The variant allele differs from the reference allele by at least one nucleotide at the site(s) identified in Table 3 (see Example 1, below), and those in linkage disequilibrium therewith. The present invention thus relates to nucleotides comprising variant alleles of the PLCG1 reference sequence, variant alleles of the PAI-2 reference sequence, and/or complements of the variant alleles to be used singly, or, preferably, in combination.

The invention further relates to nucleotides comprising portions of the variant alleles and/or portions of complements of the variant alleles which comprise the site of the polymorphism and are at least 5 nucleotides or basepairs in length. Portions can be, for example, 5-10, 5-15, 10-20, 2-25, 10-30, 10-50 or 10-100 bases or basepairs long. For example, a portion of a variant allele which is 17 nucleotides or basepairs in length includes the polymorphism (i.e., the nucleotide(s) which differ from the reference allele at that site) and twenty additional nucleotides or basepairs which flank the site in the variant allele. These additional nucleotides and basepairs can be on one or both sides of the polymorphism. Polymorphisms which are the subject of this invention are defined in Table 3 with respect to the reference sequences identified in Table 3 (GI 11345540 or GI 6705901), and those polymorphisms in linkage disequilibrium with the polymorphisms of Table 3. For example, the invention relates to nucleotides comprising a portion of the PLCG1 gene having a nucleotide sequence of GI 11345540 (SEQ ID NO:1), or a portion thereof, comprising a polymorphism at a specific nucleotide residue (e.g., a thymidine at residue 64001, or the complement thereof) and nucleotides comprising a portion of the PAI-2 gene having a nucleotide sequence of GI 6705901 (SEQ ID NO:3), or a portion thereof, comprising a polymorphism at a specific nucleotide residue (e.g., a cytidine at residue 170871, or the complement thereof).

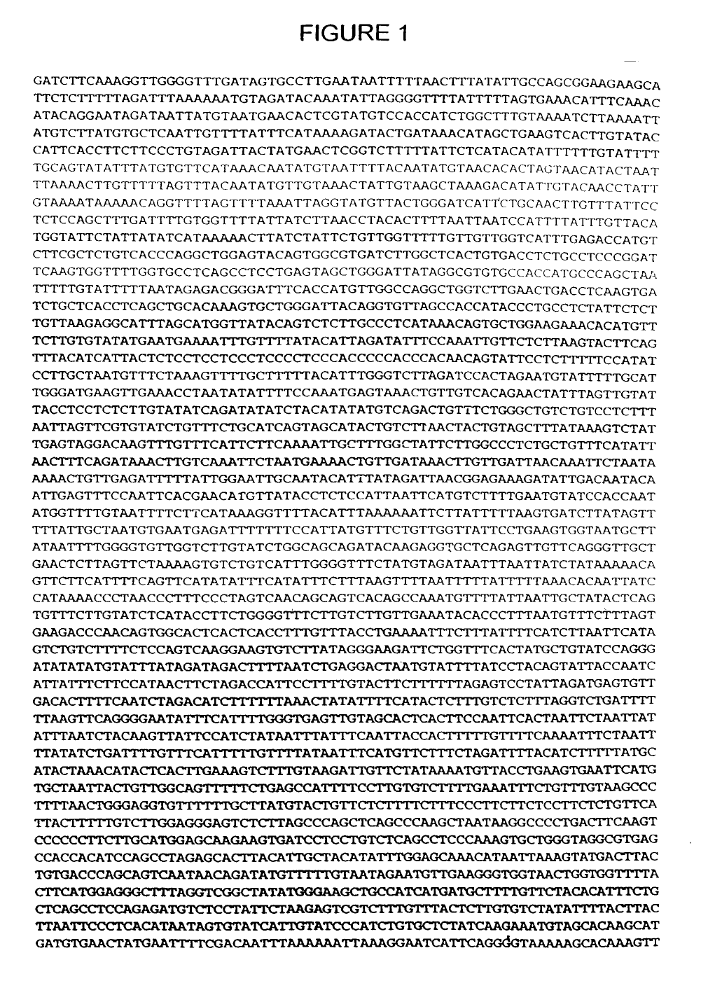

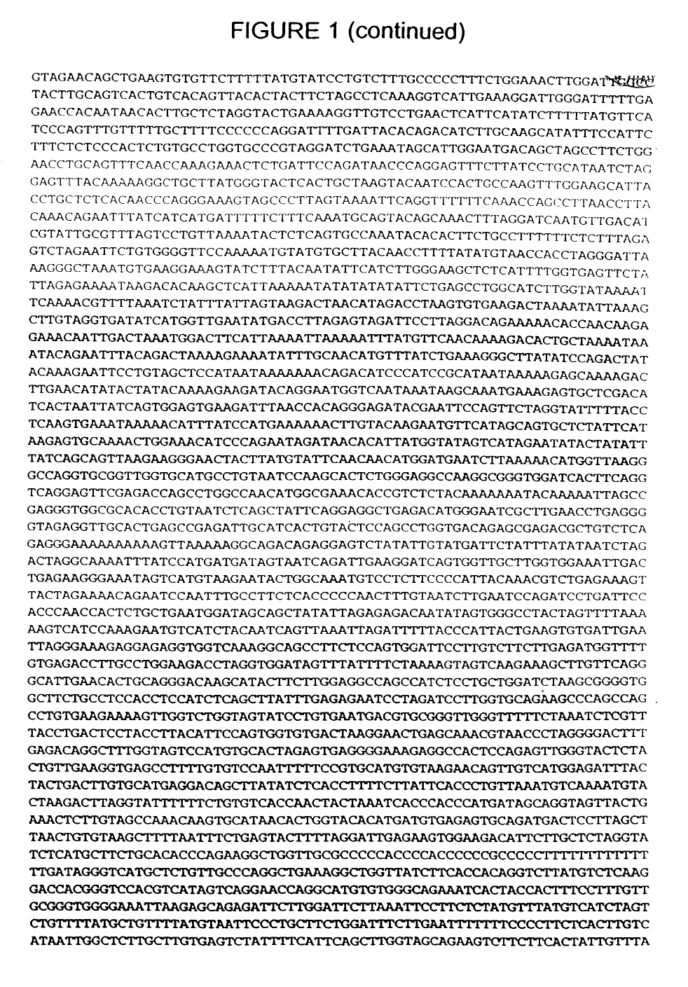

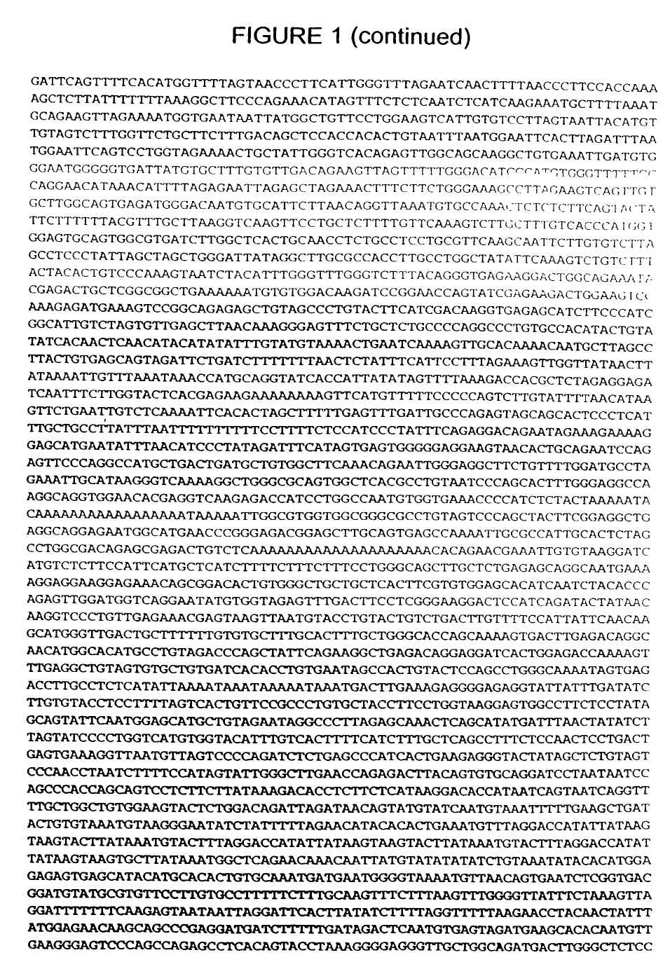

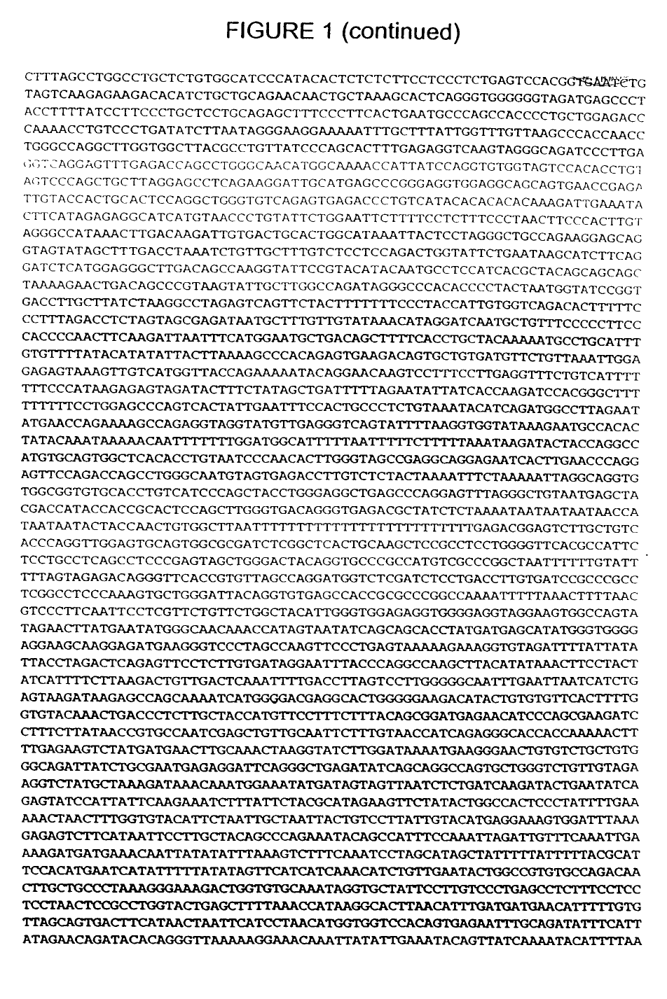

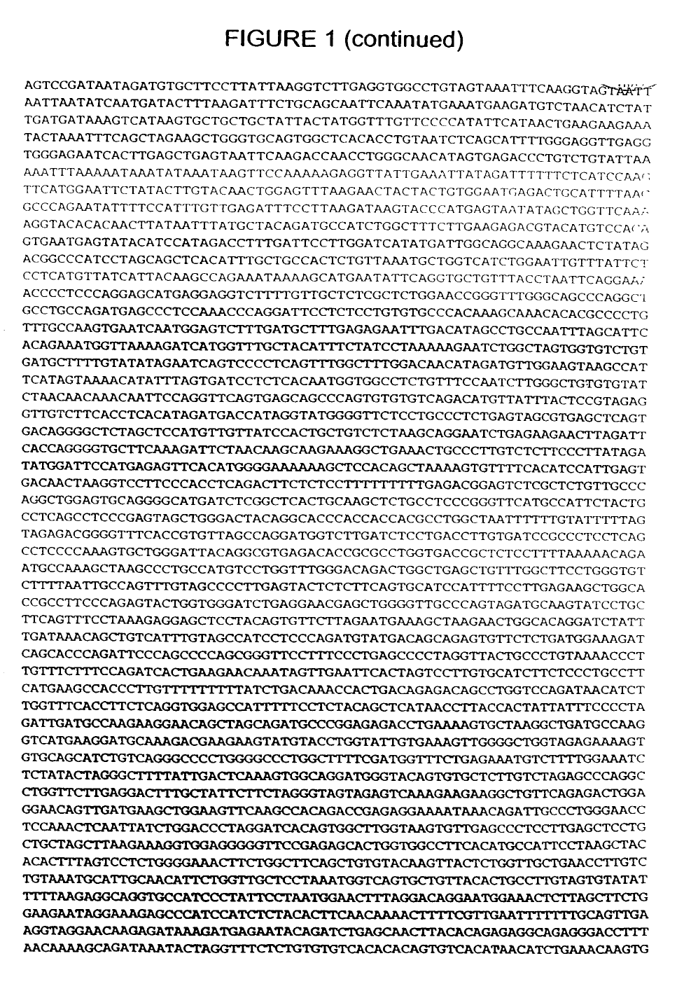

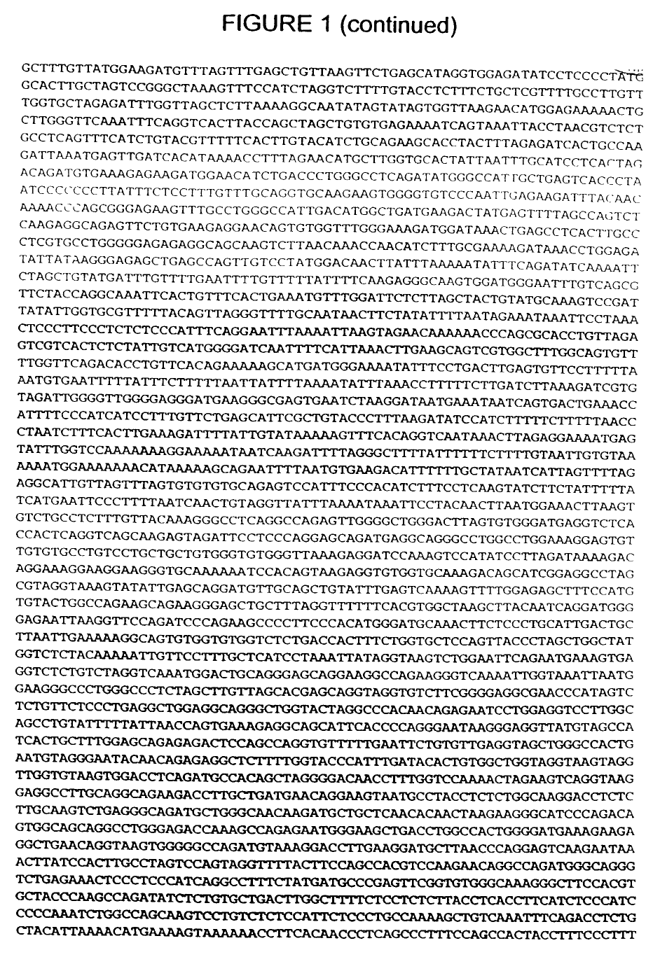

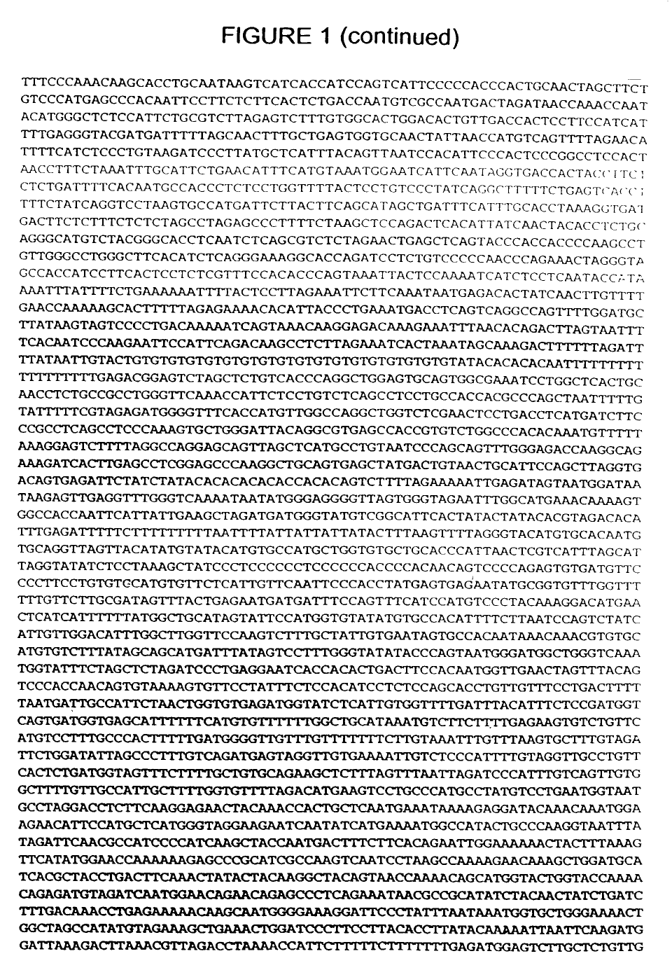

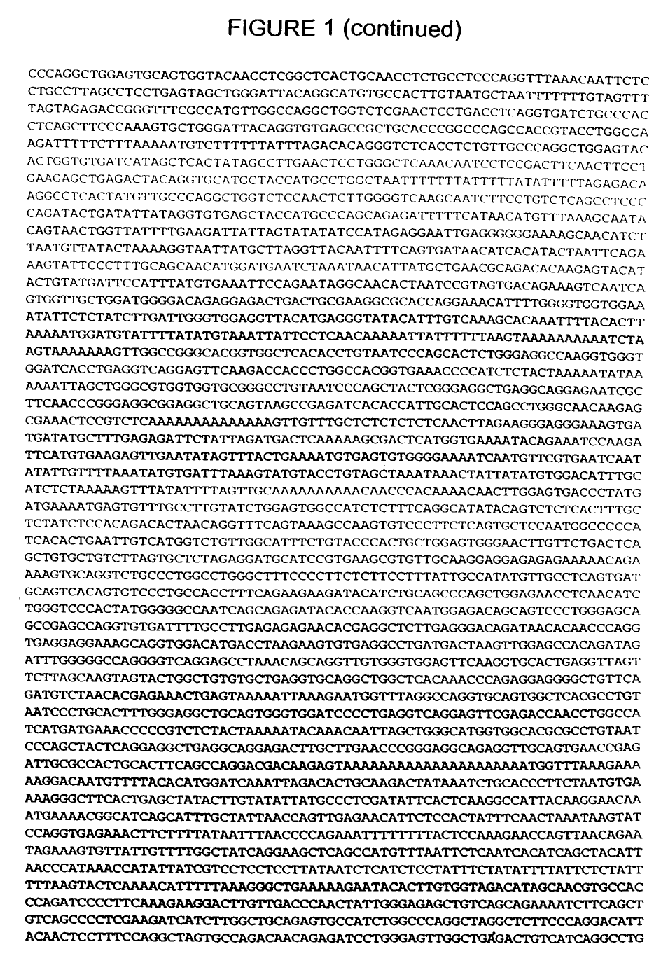

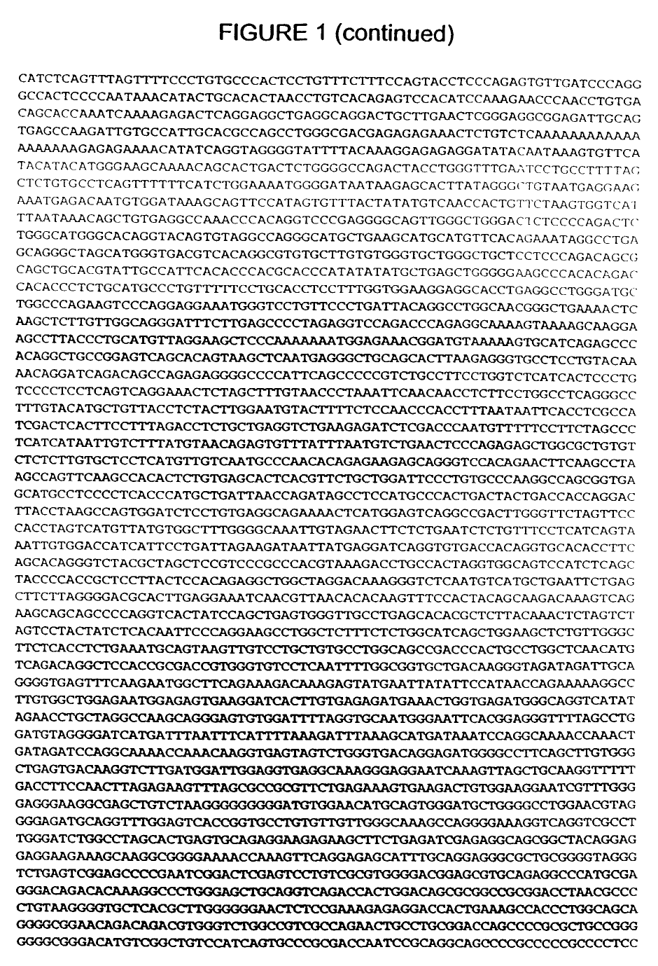

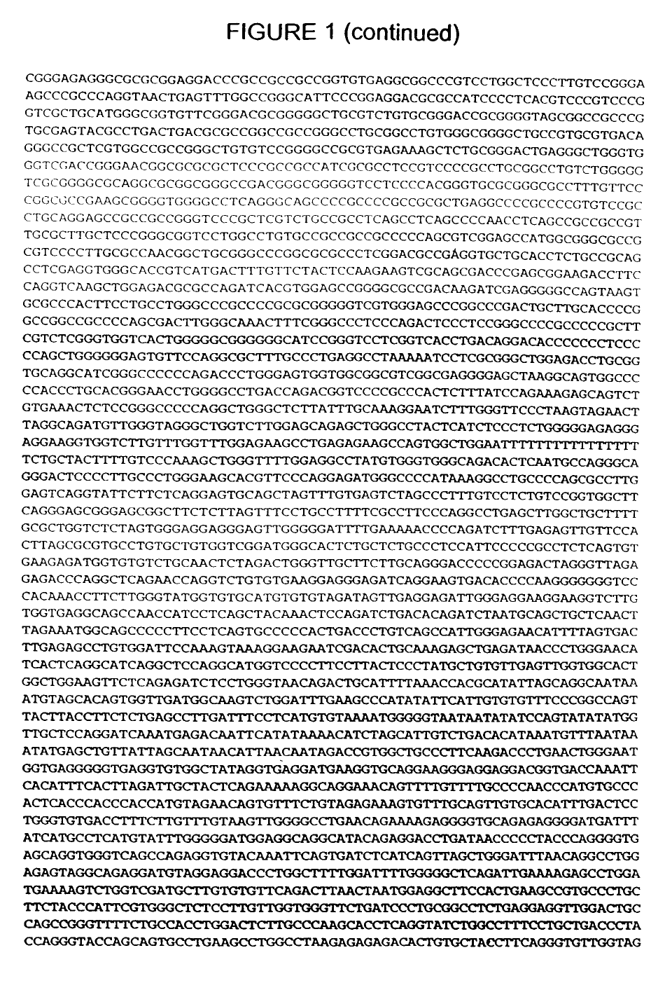

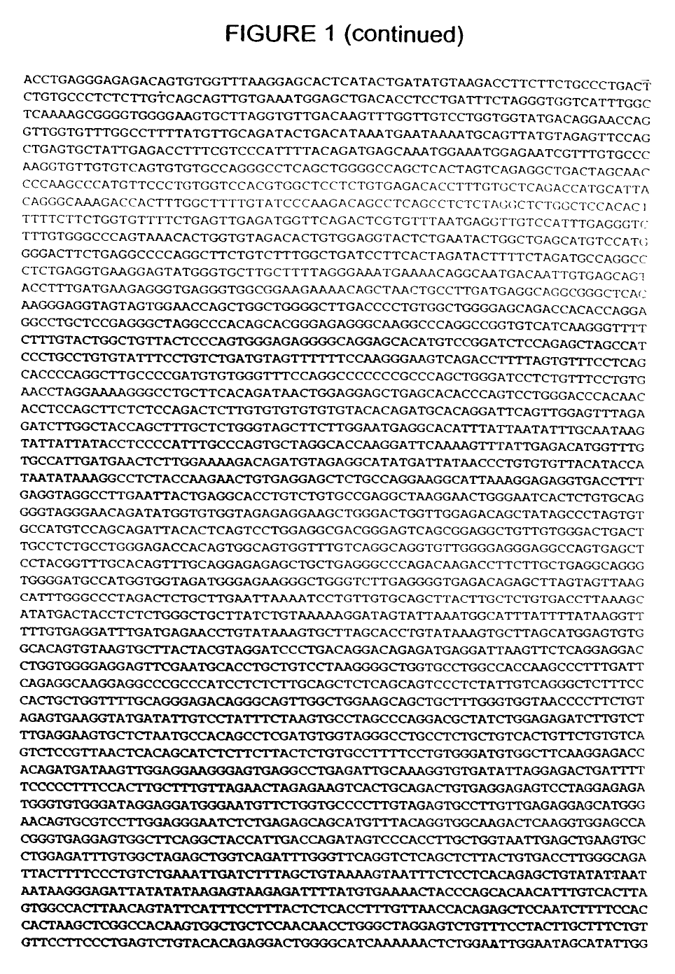

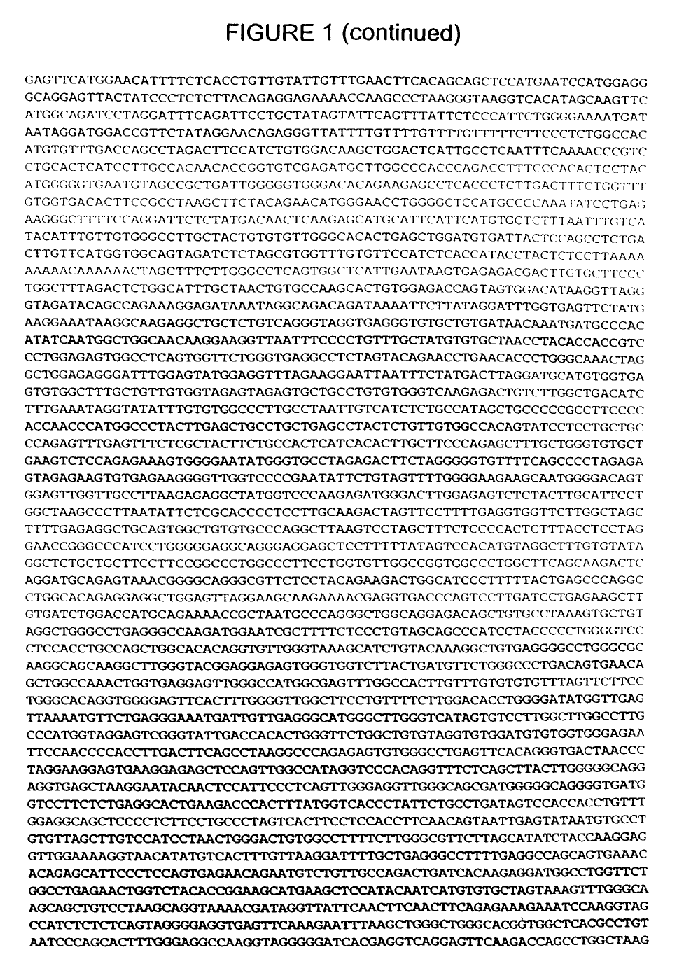

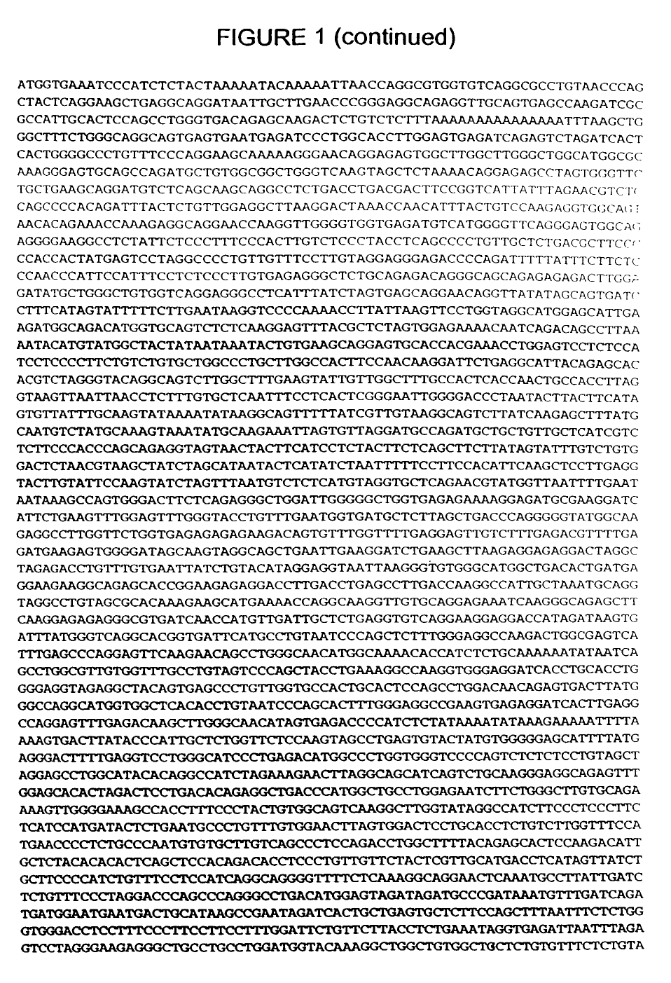

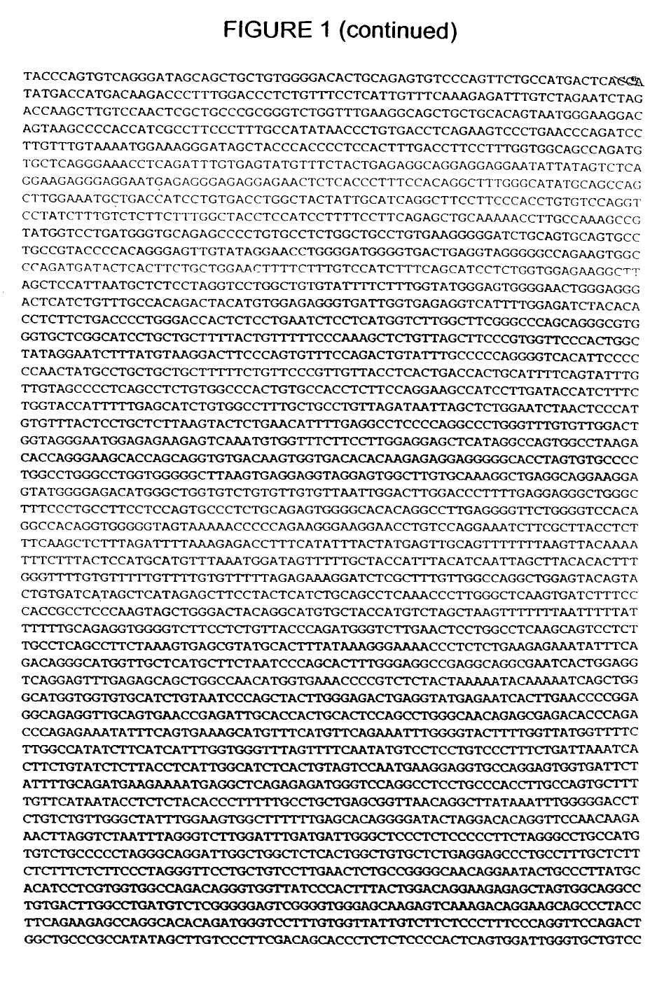

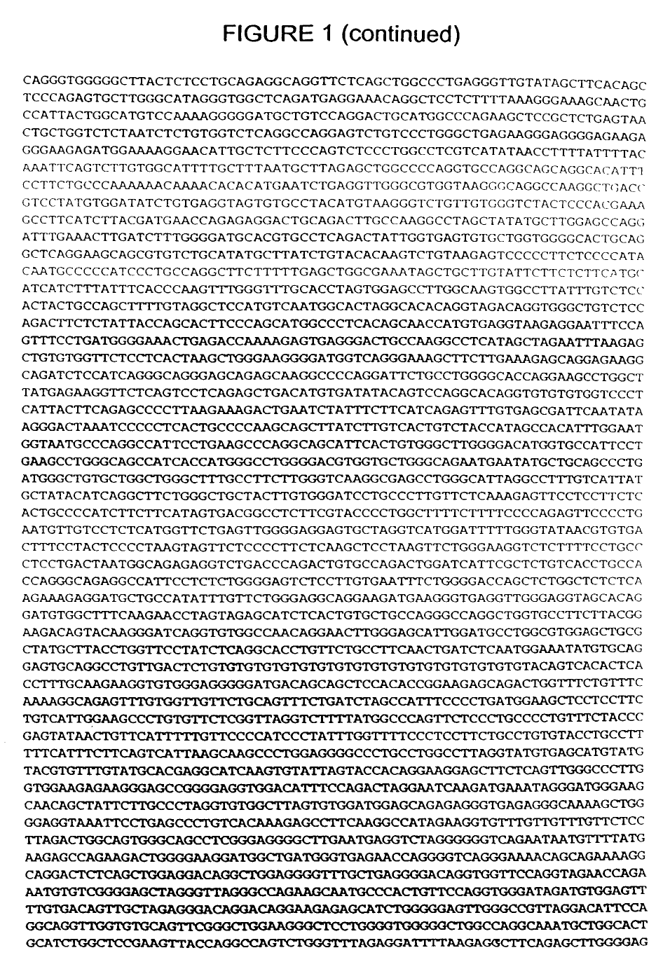

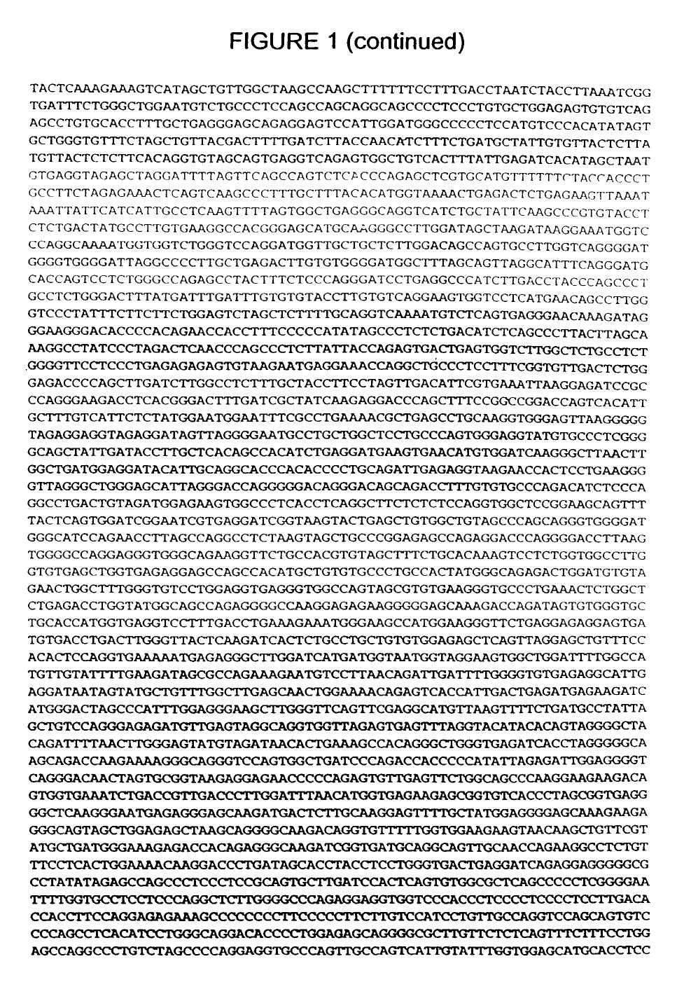

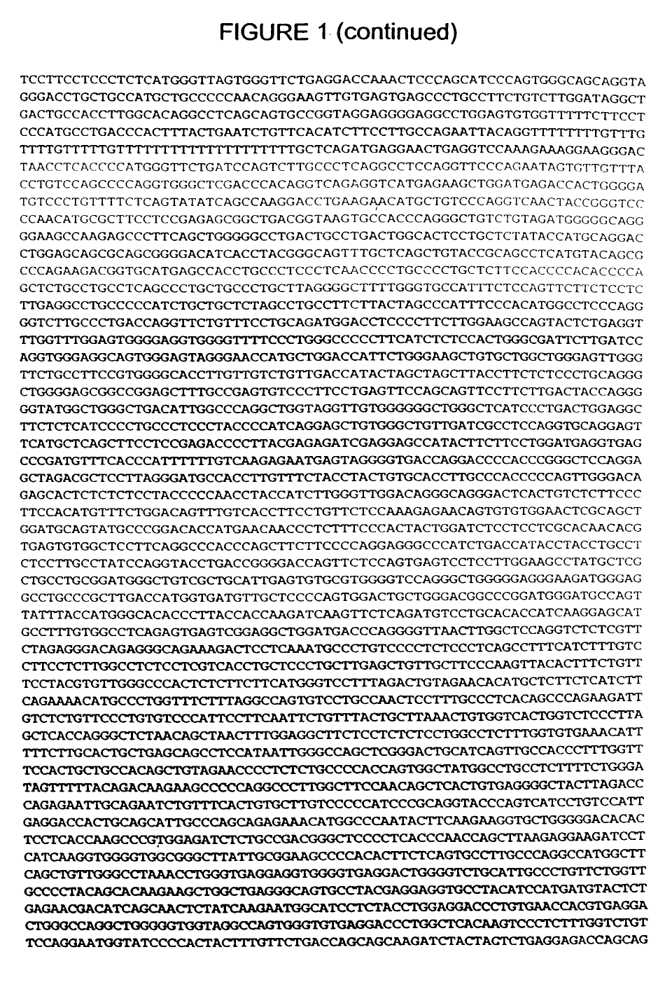

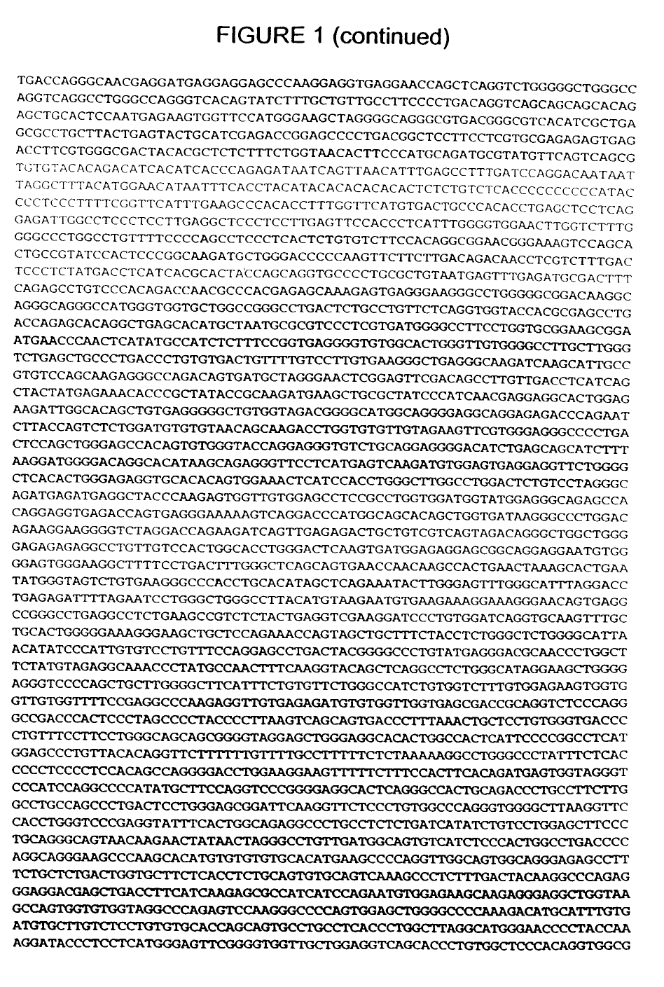

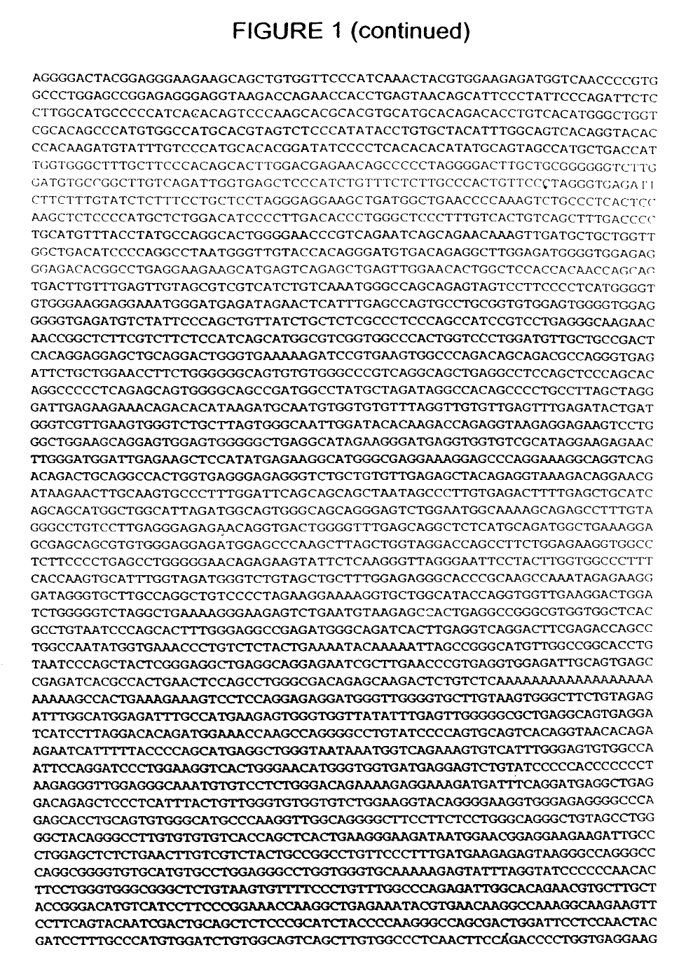

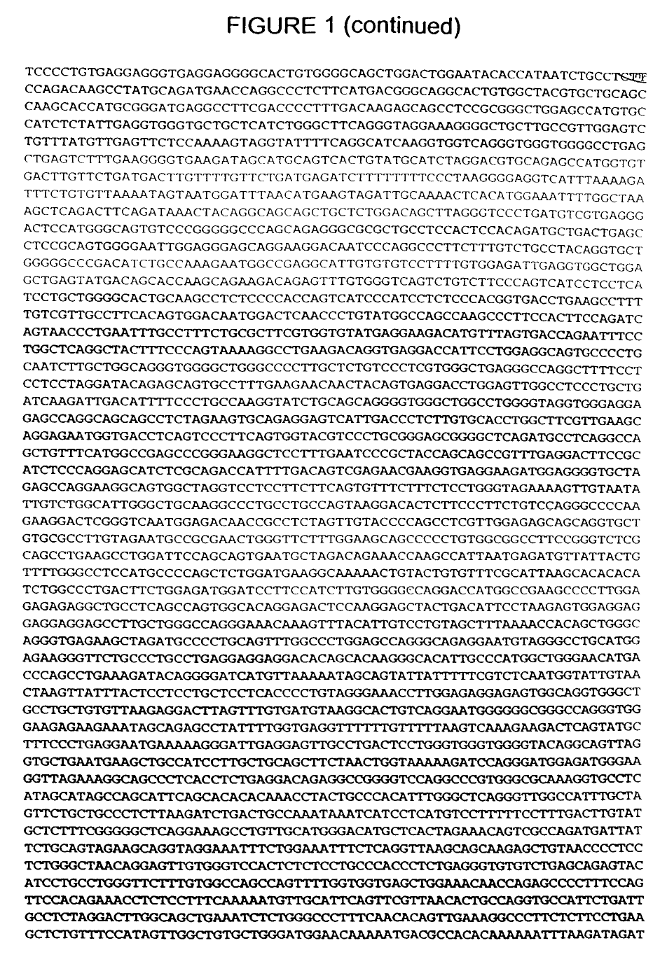

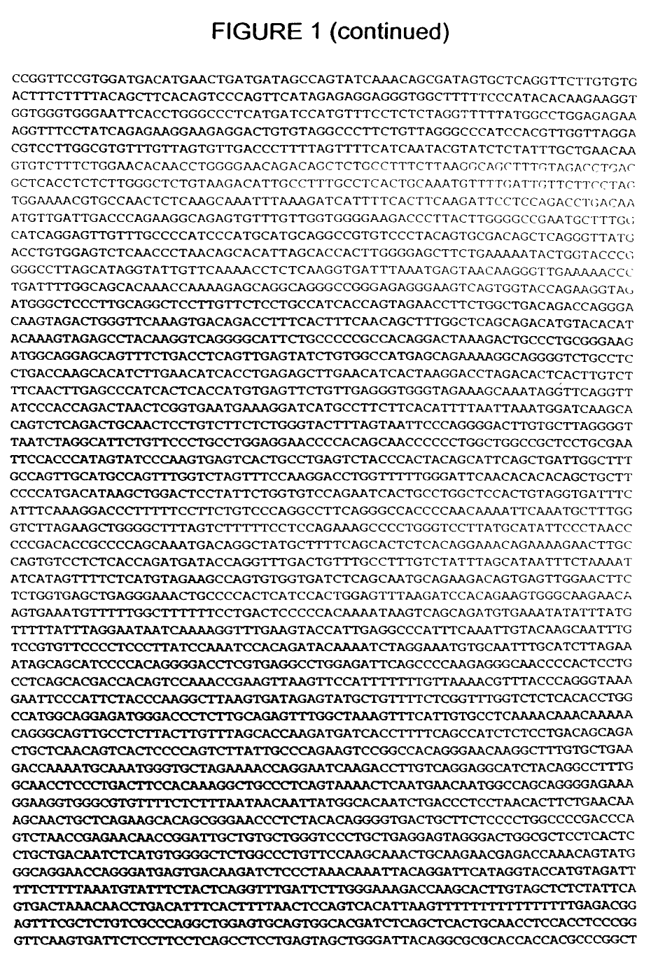

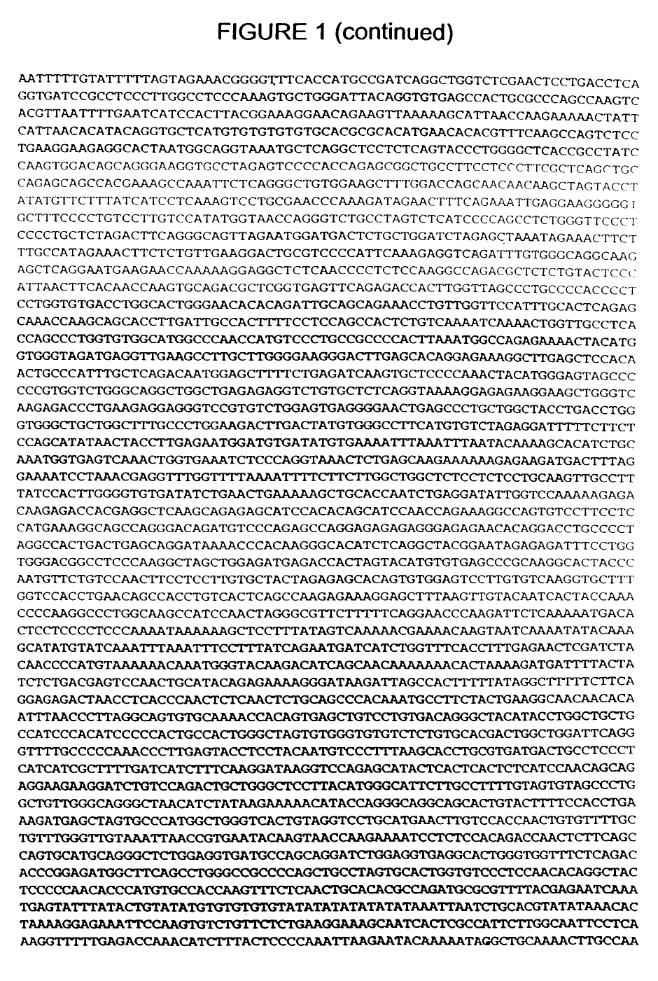

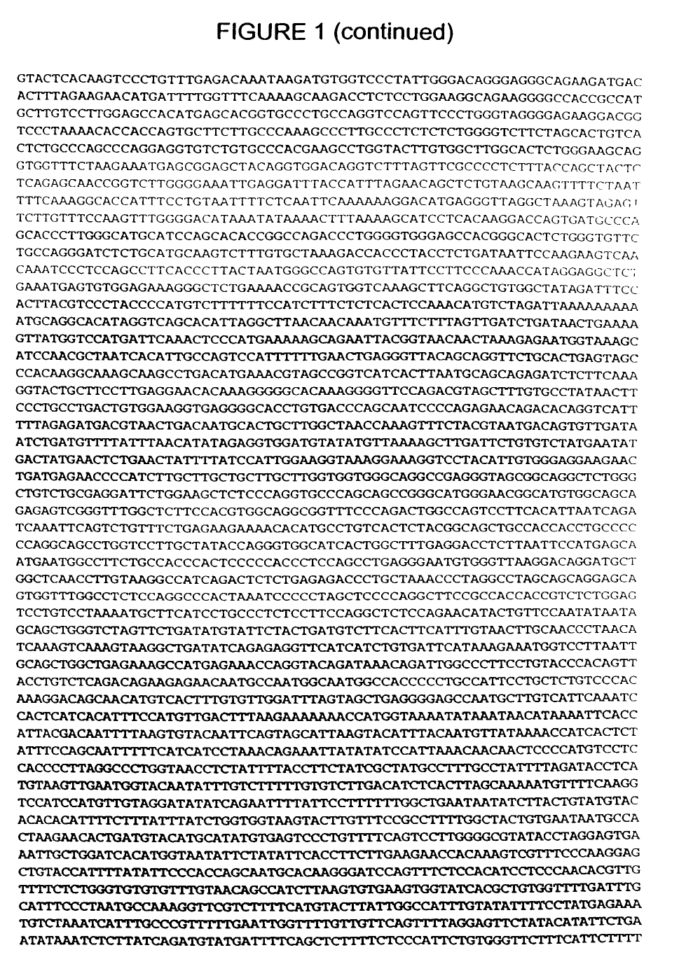

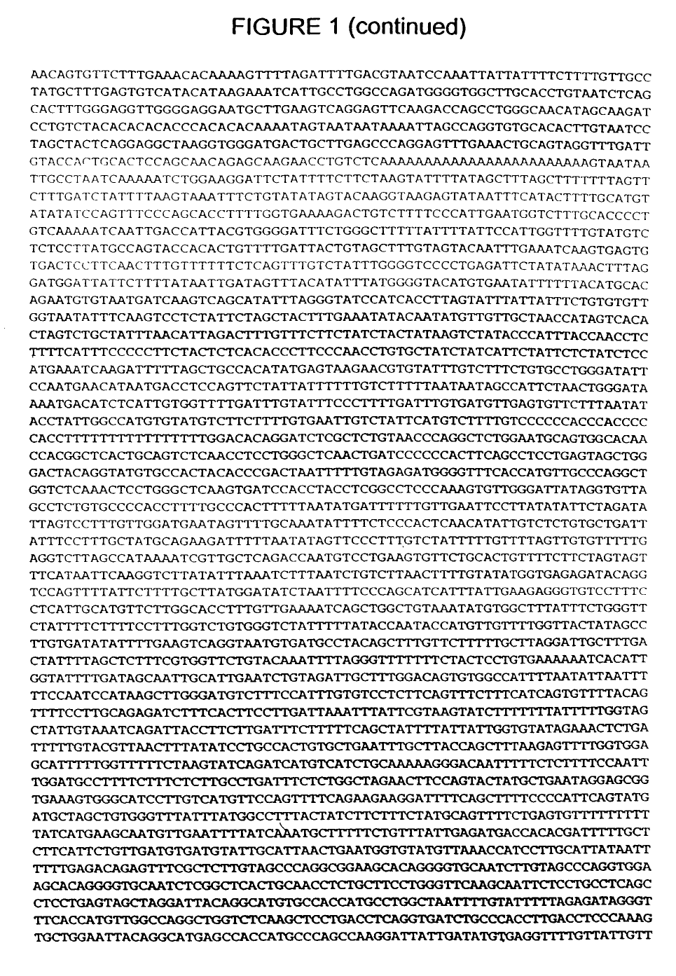

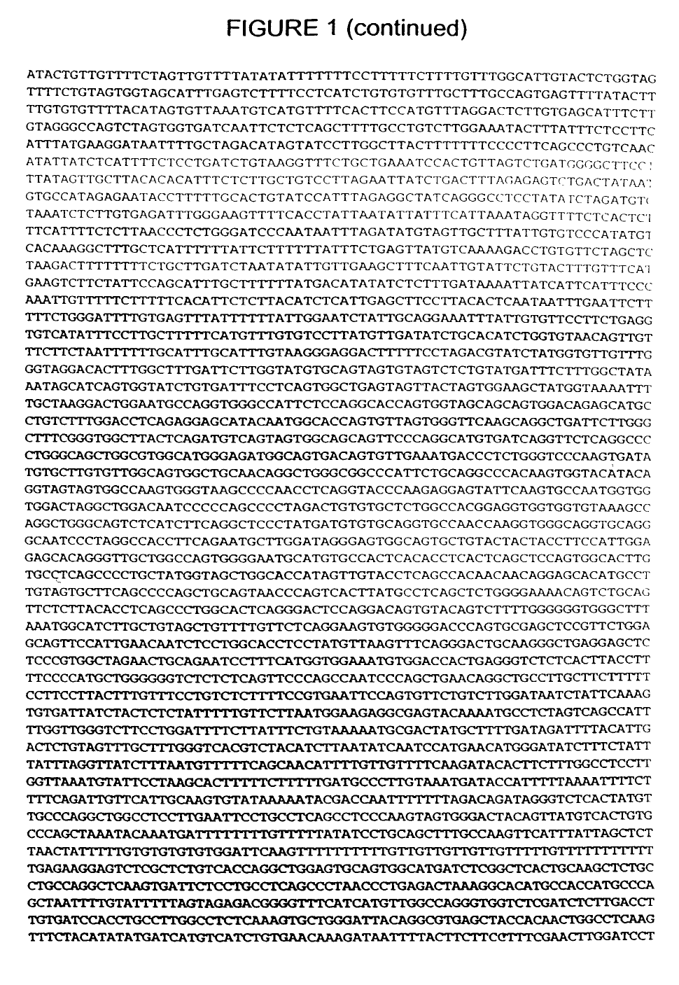

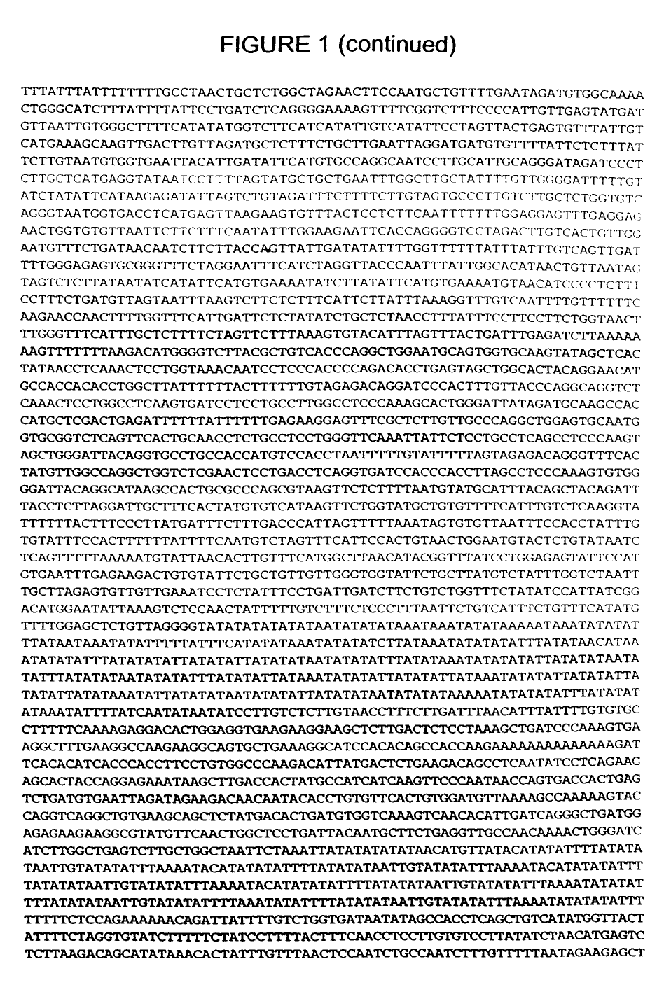

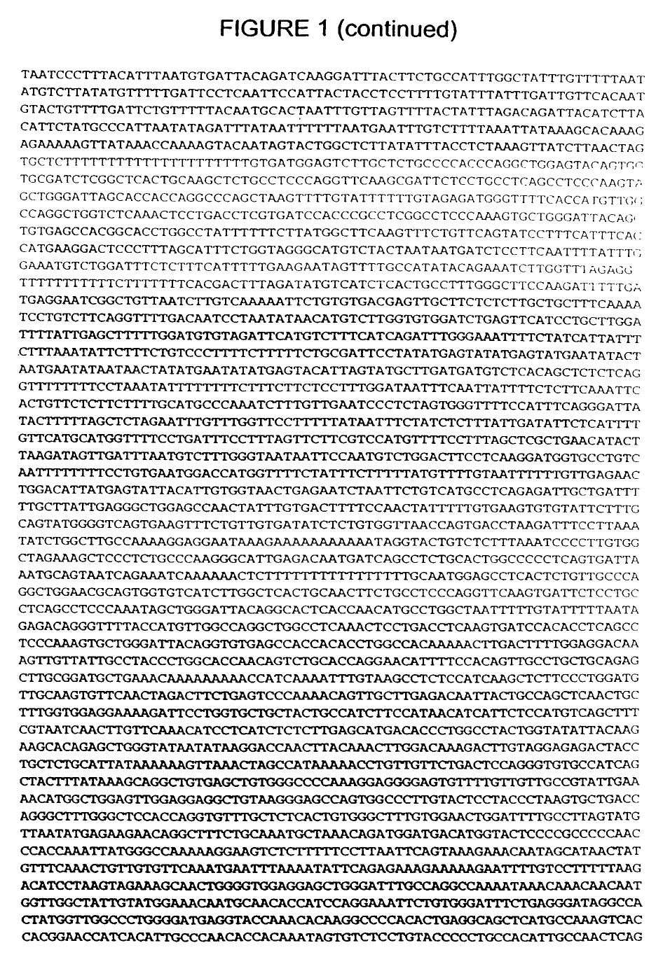

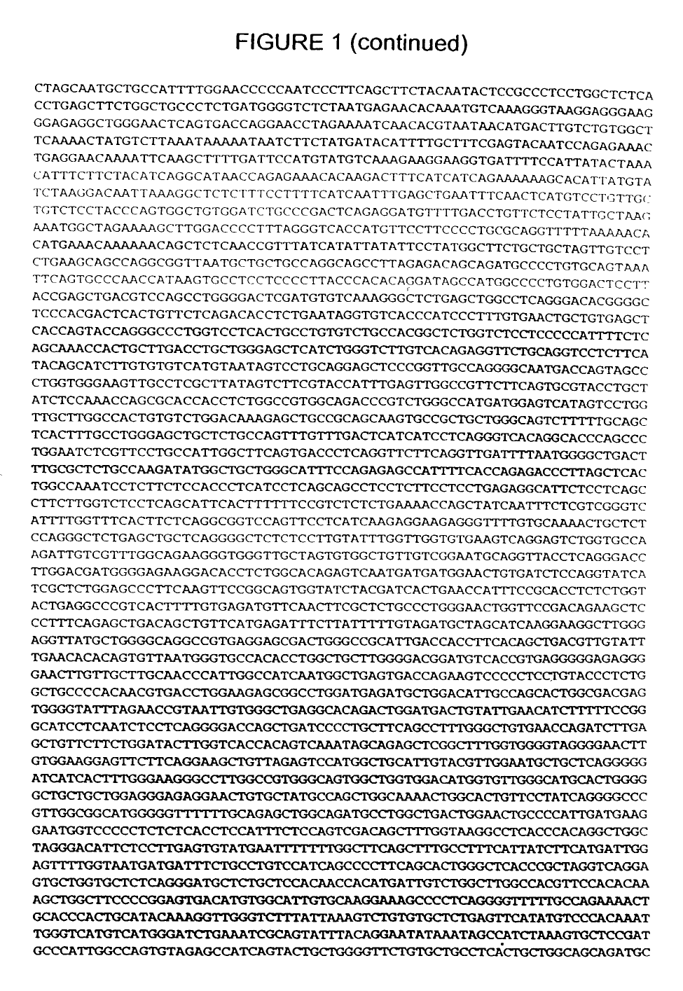

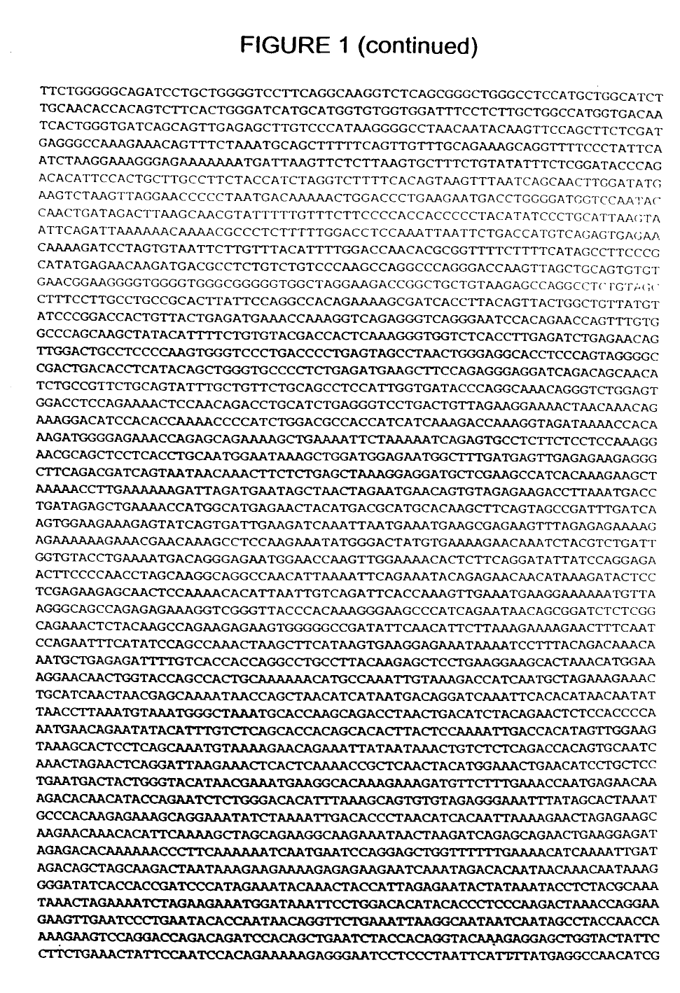

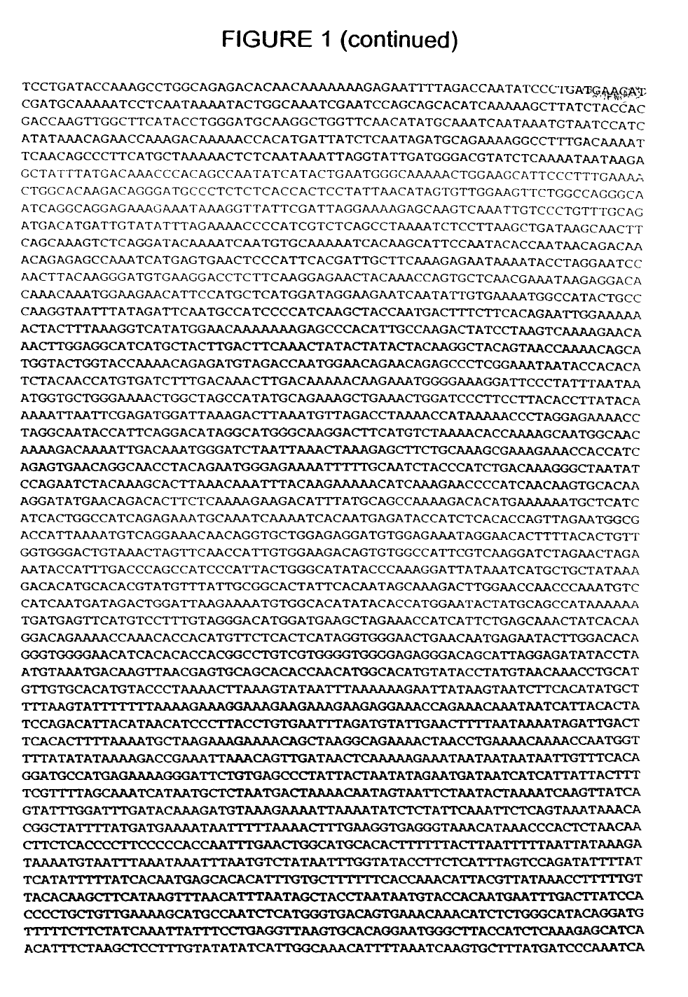

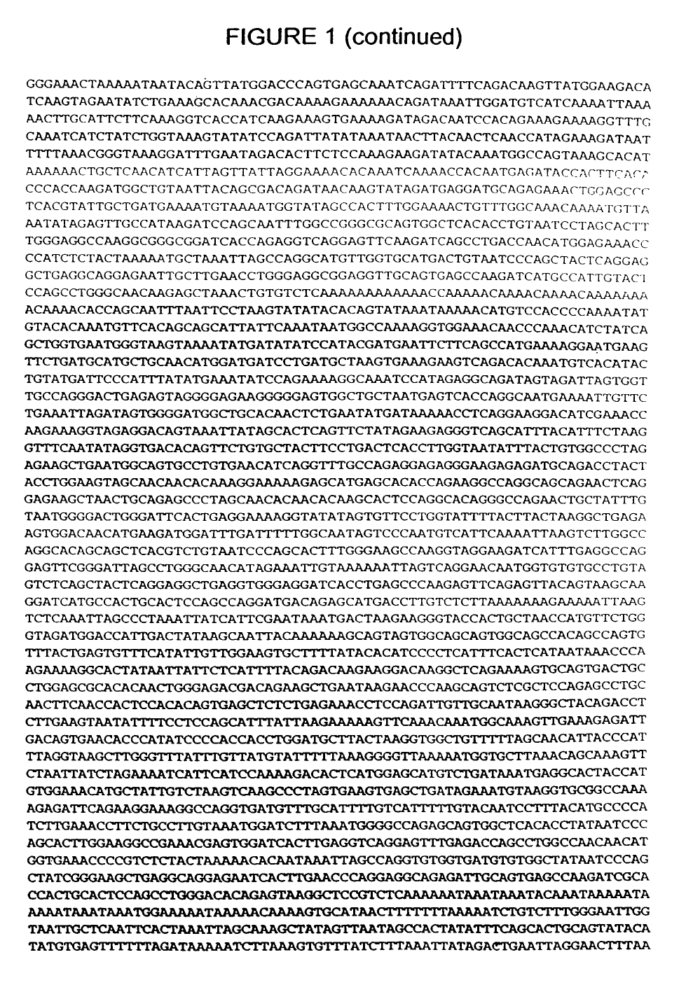

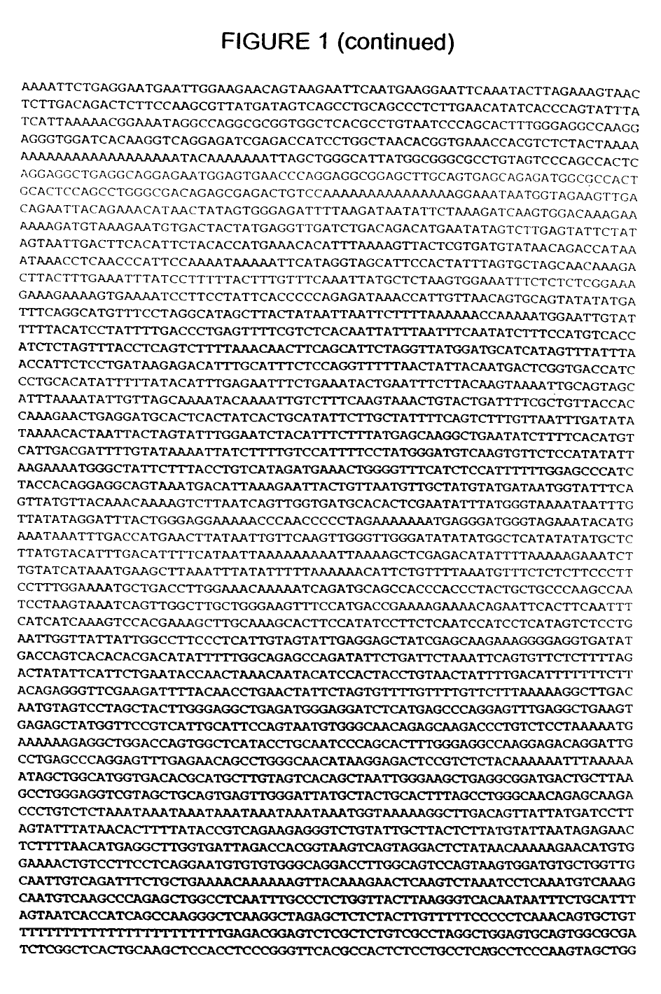

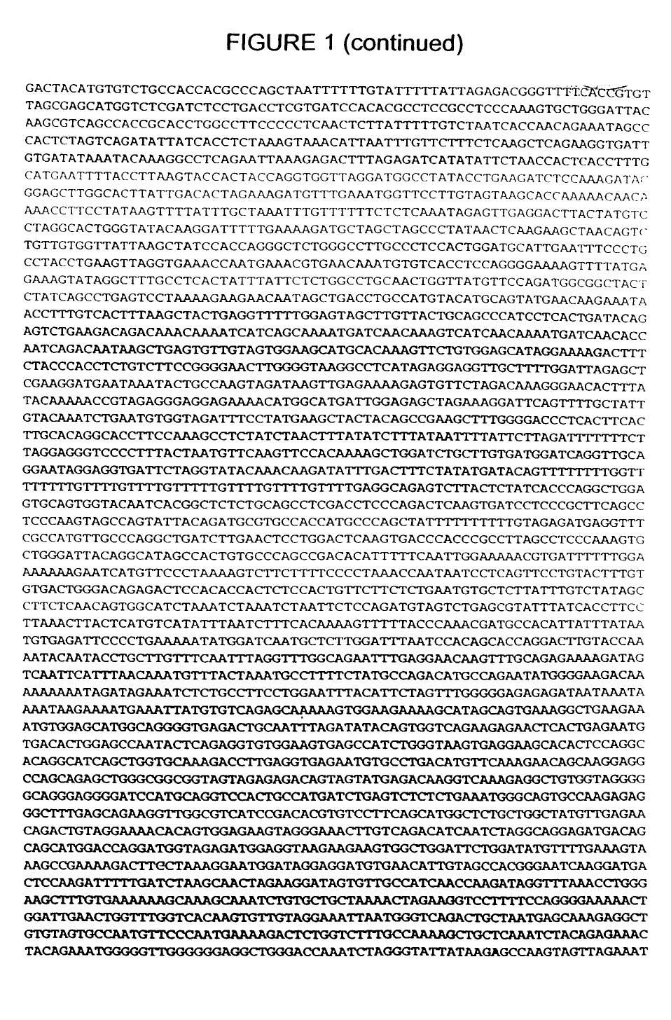

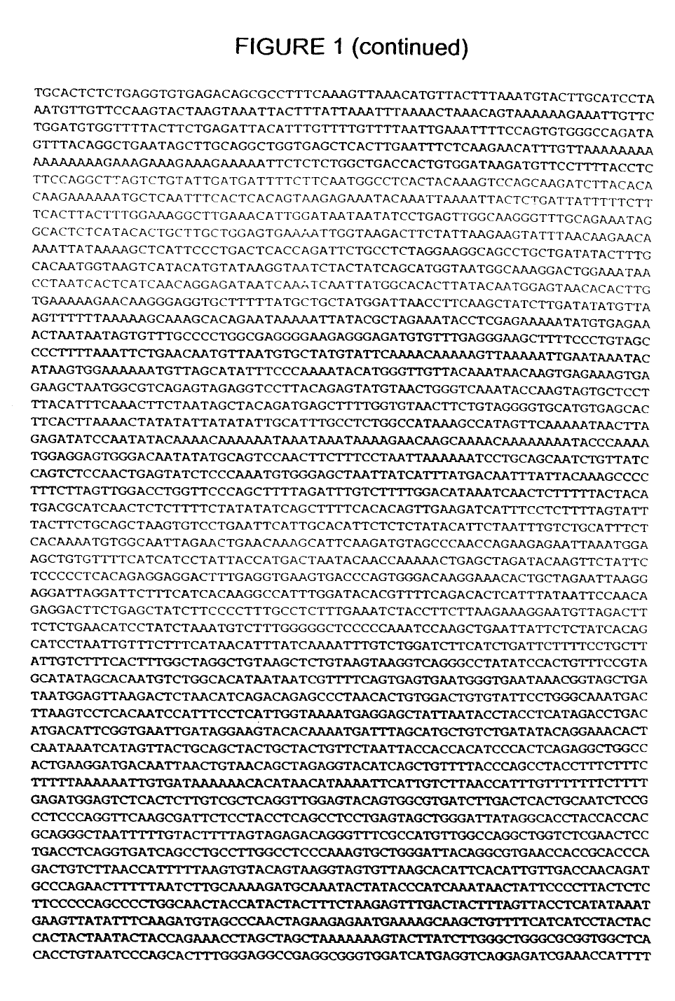

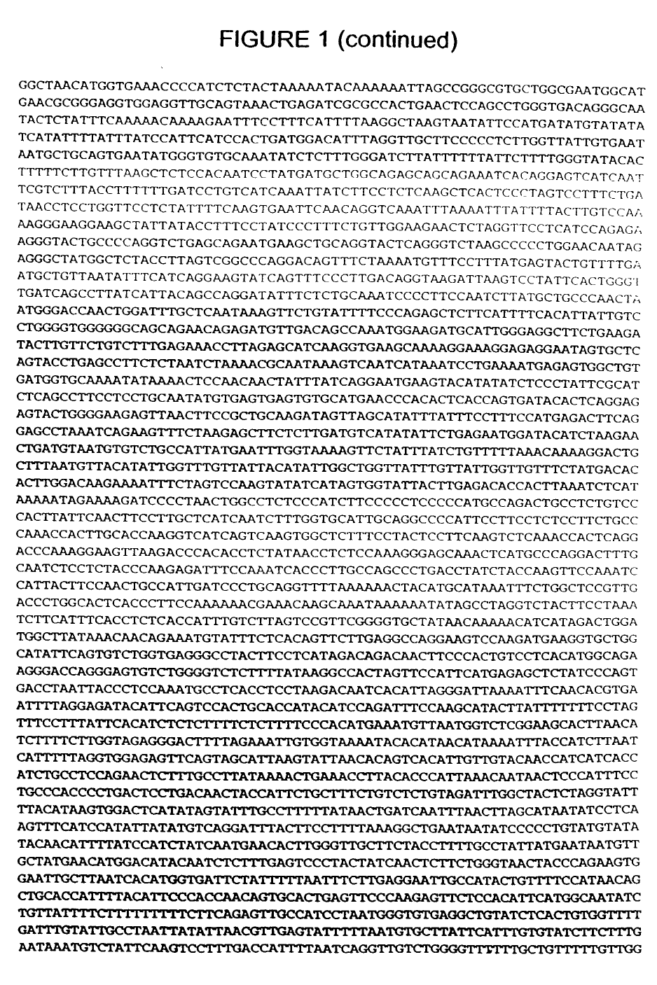

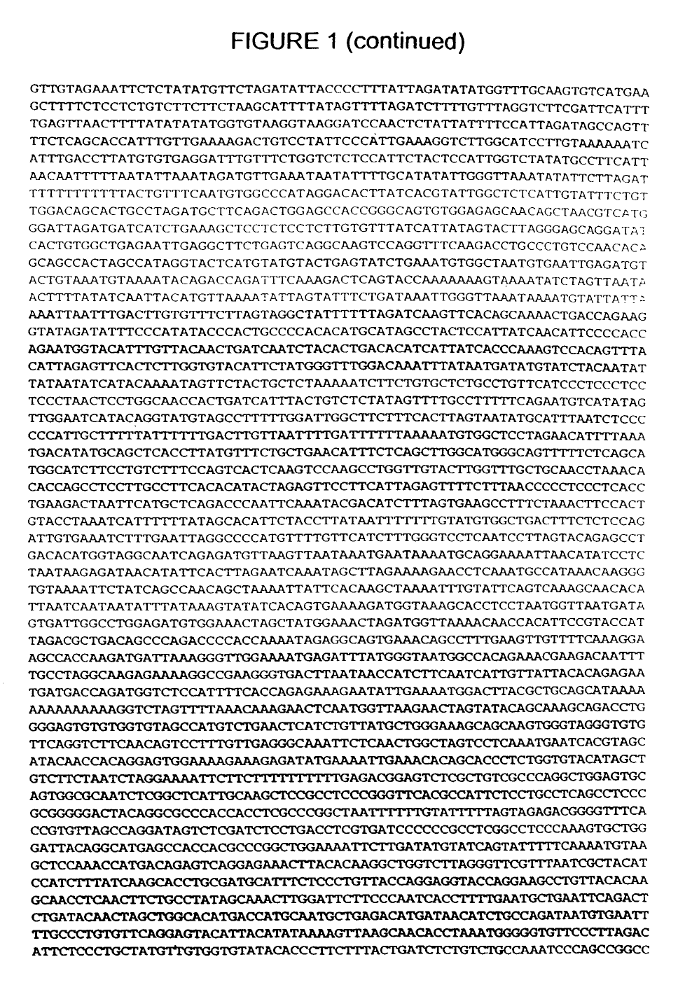

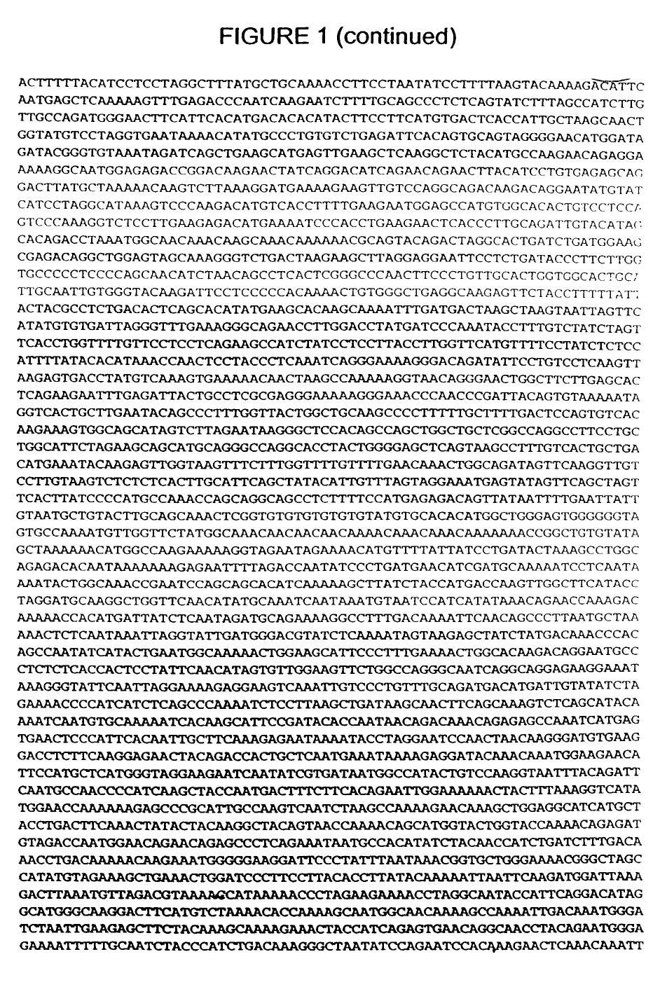

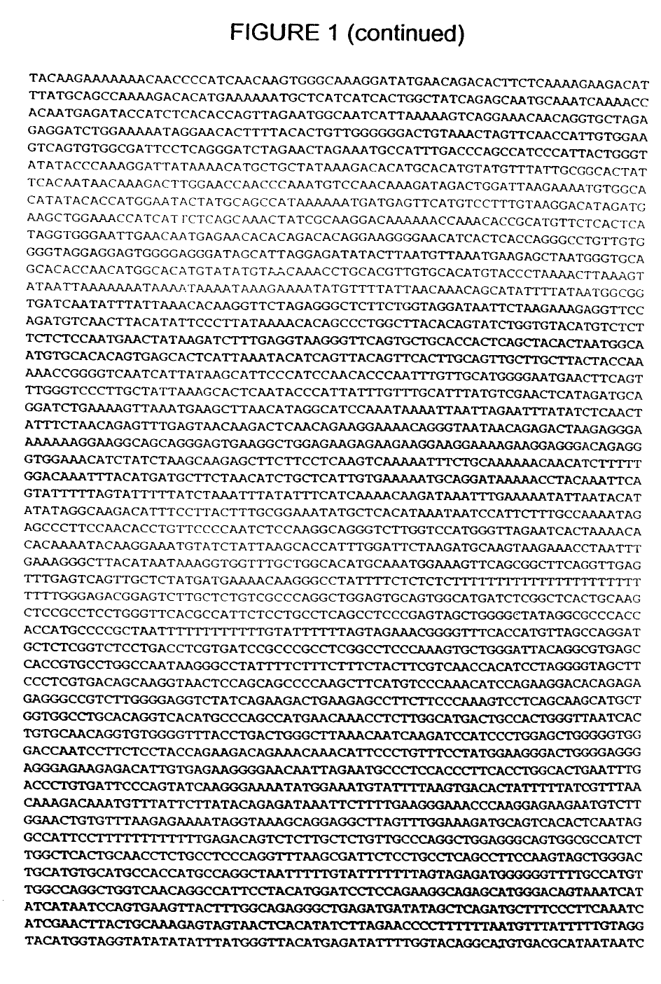

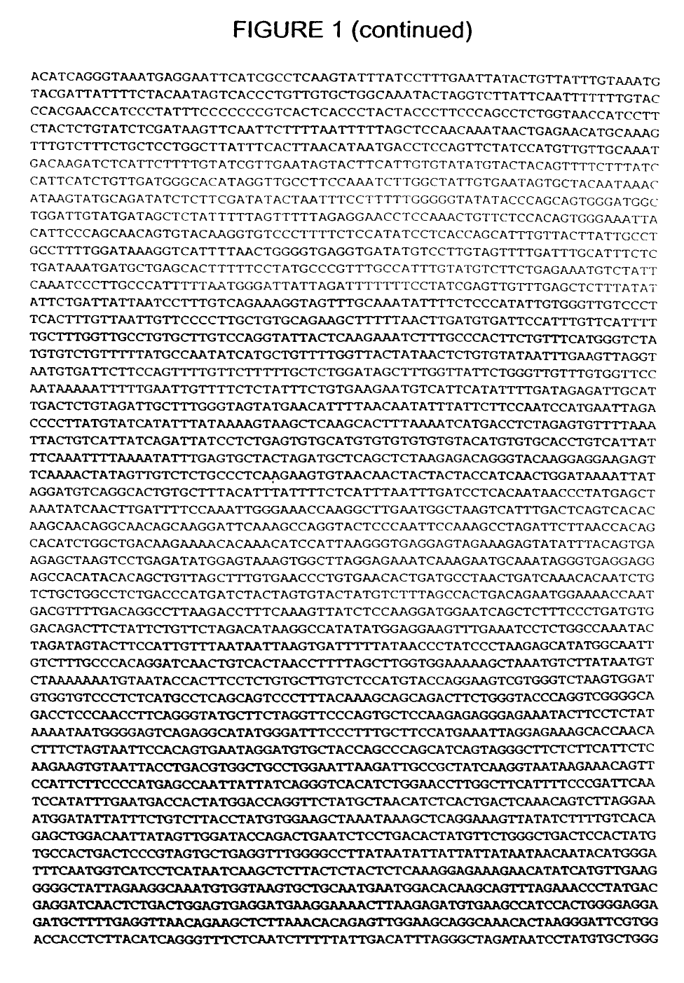

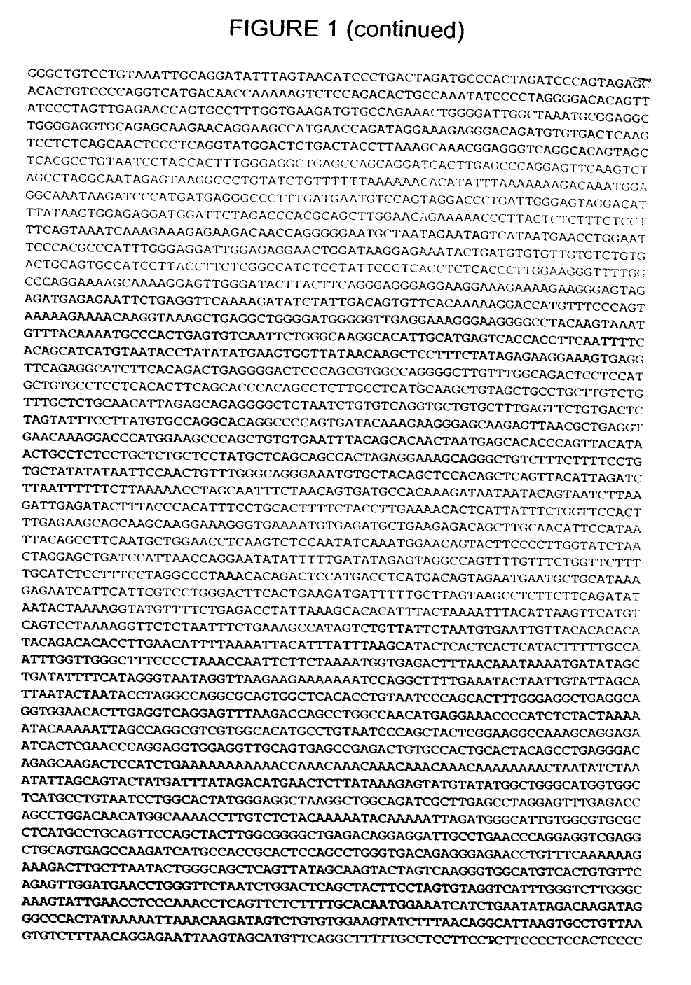

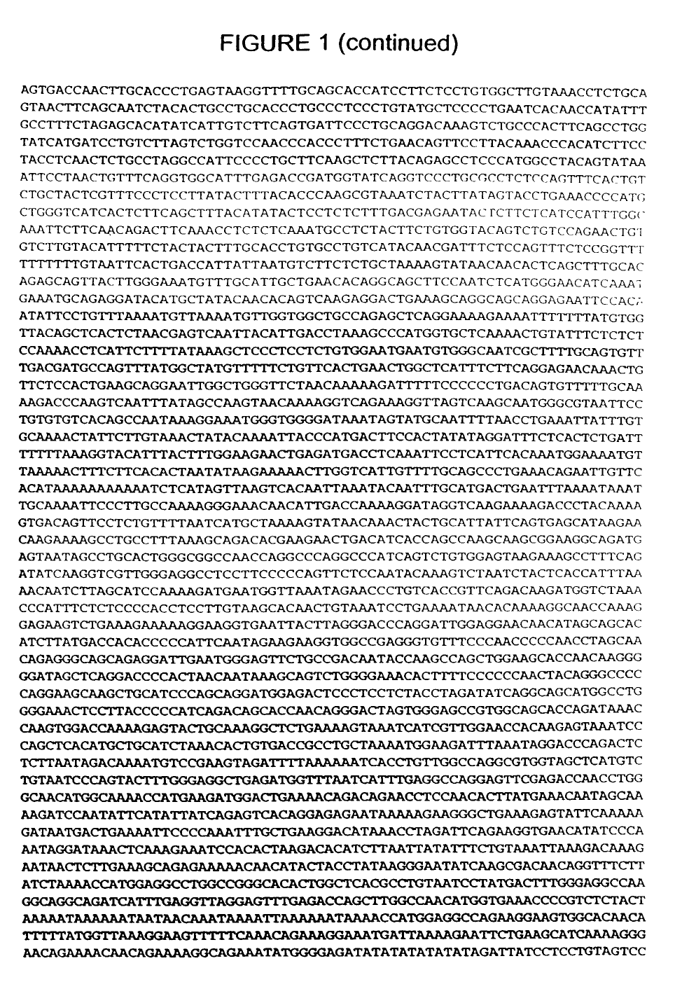

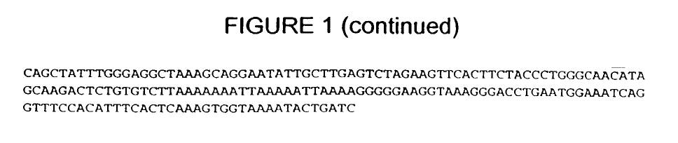

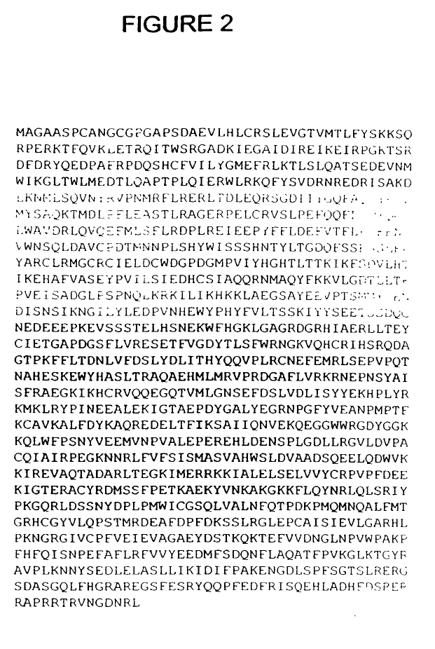

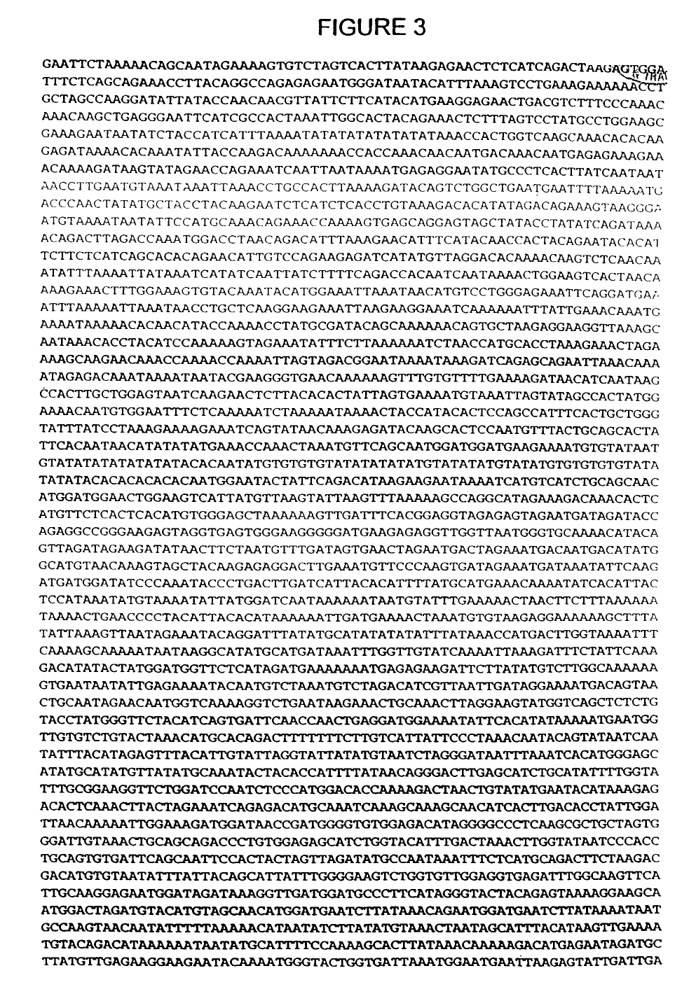

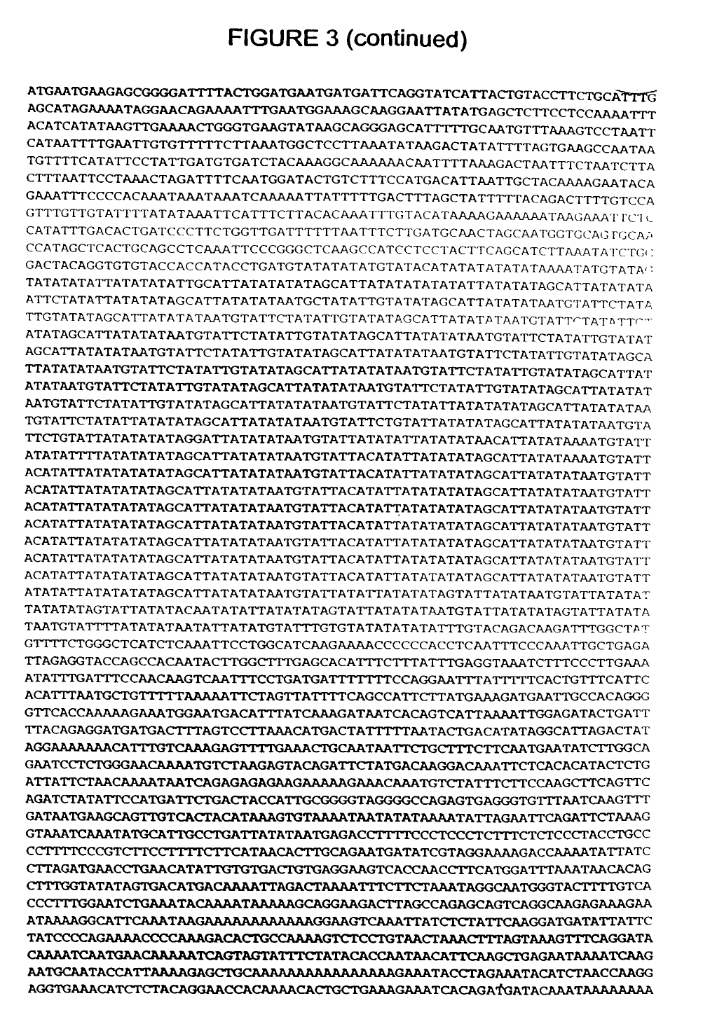

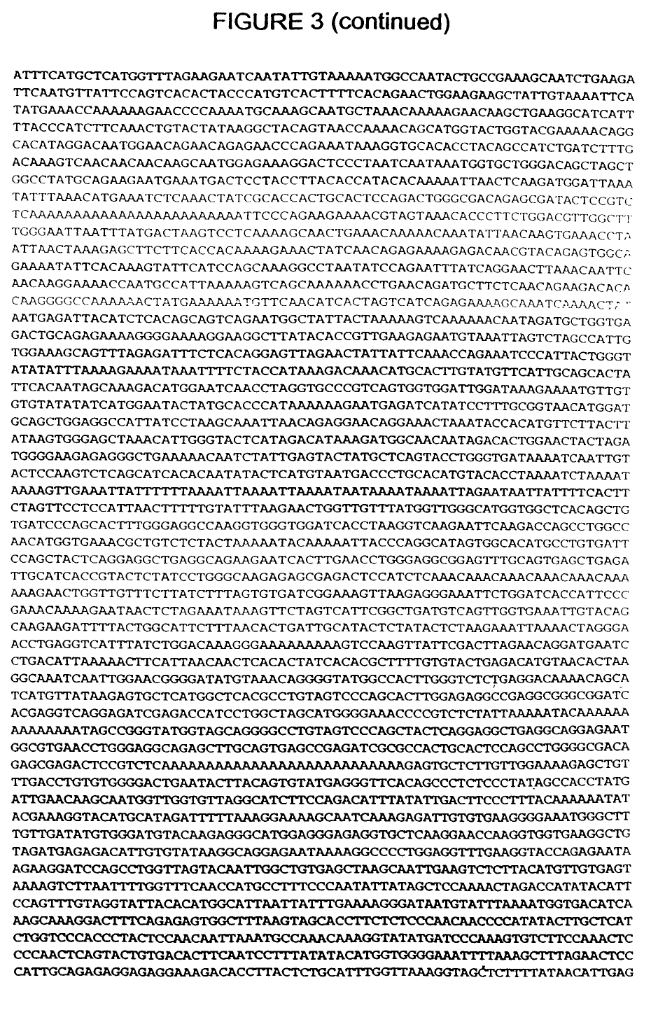

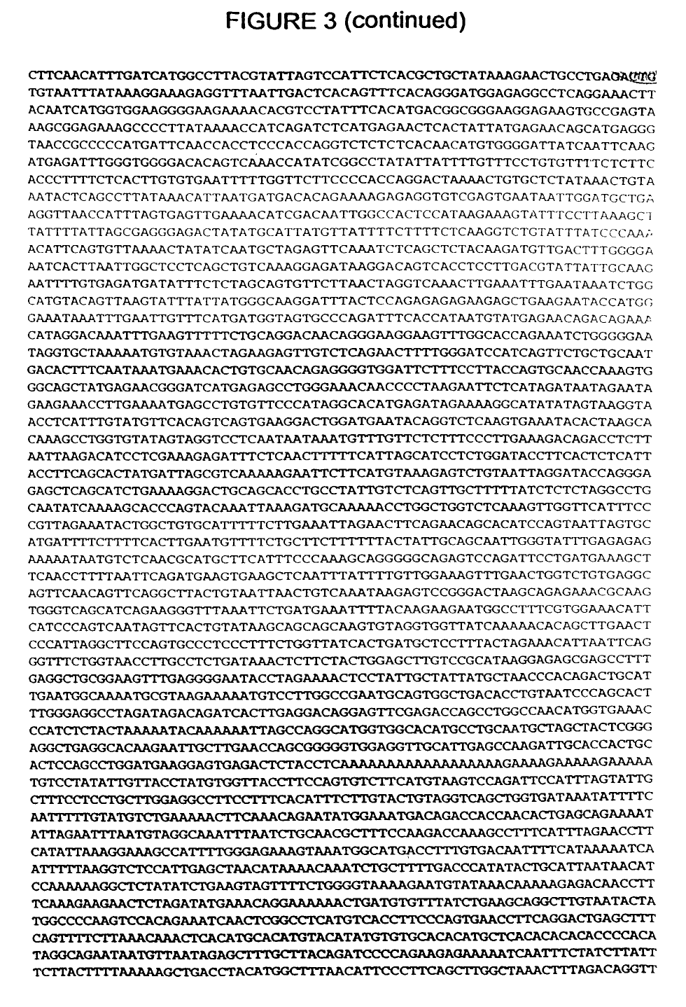

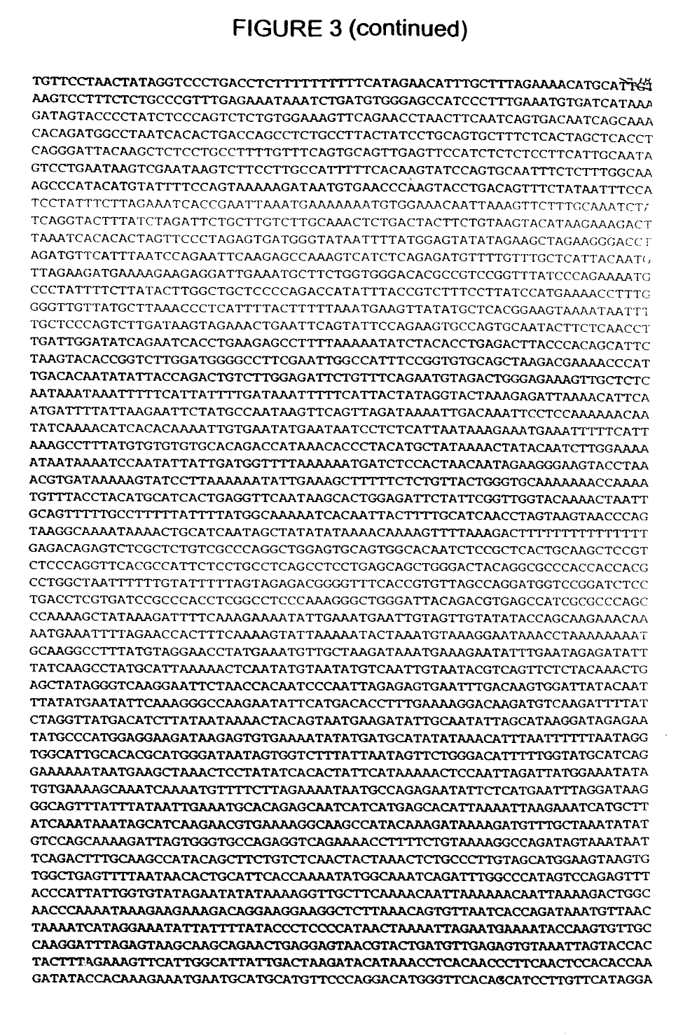

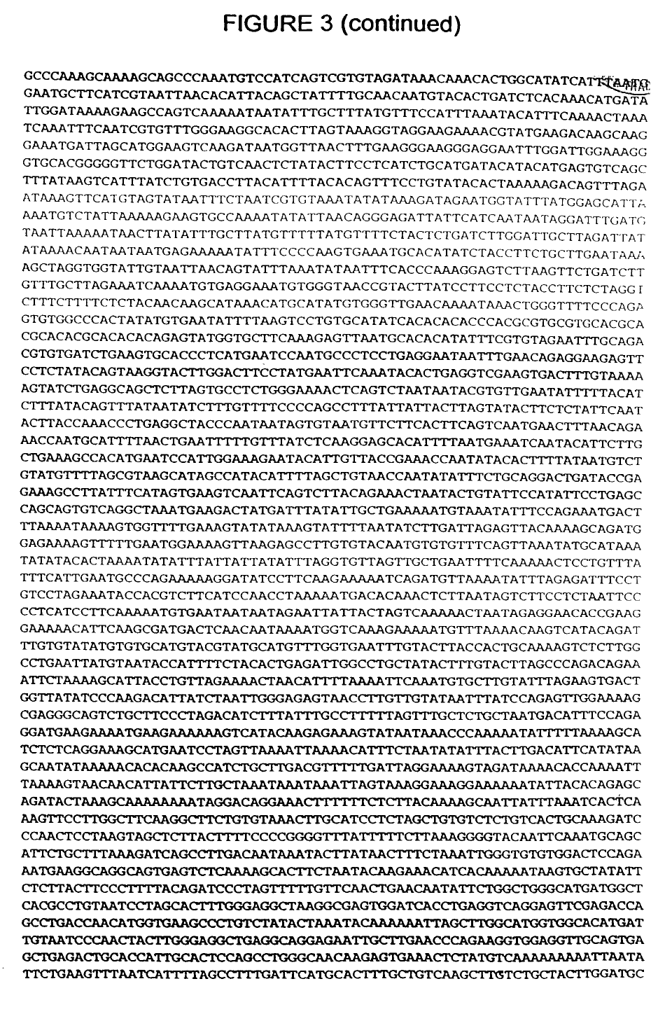

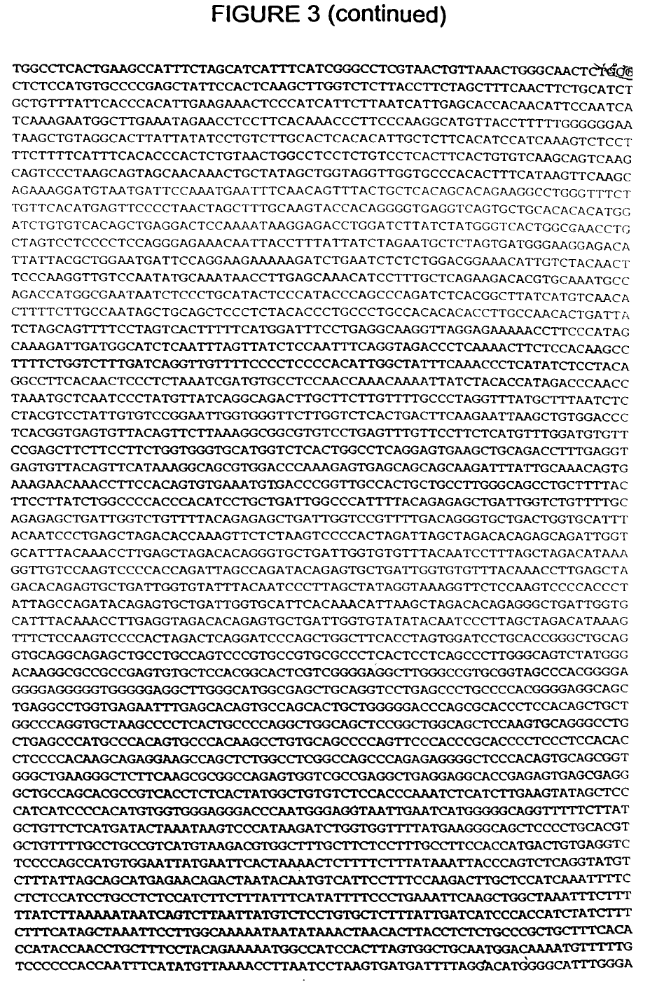

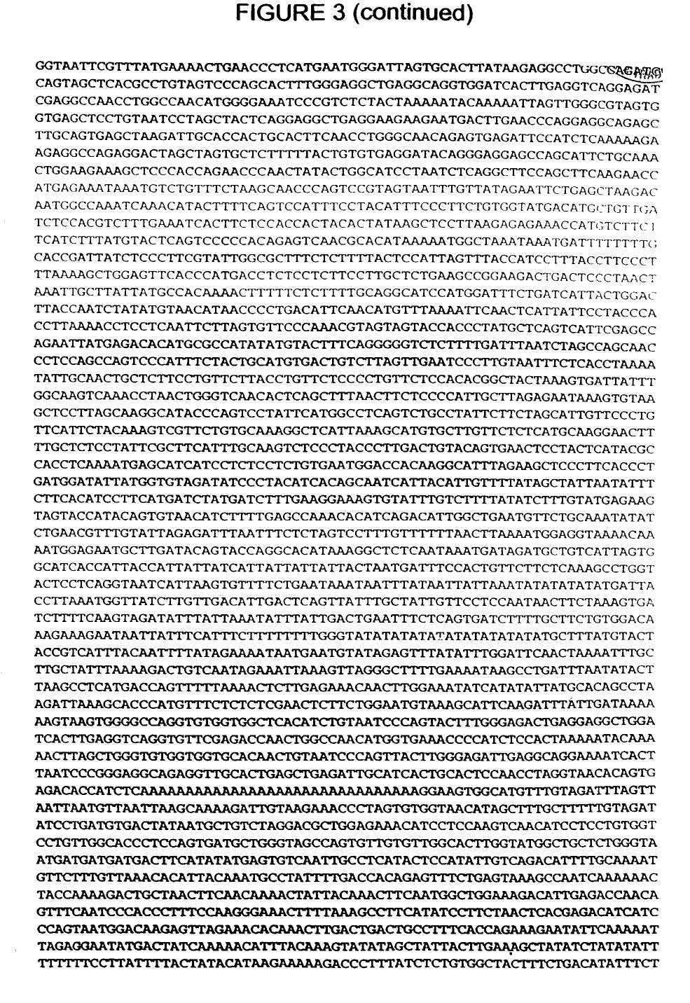

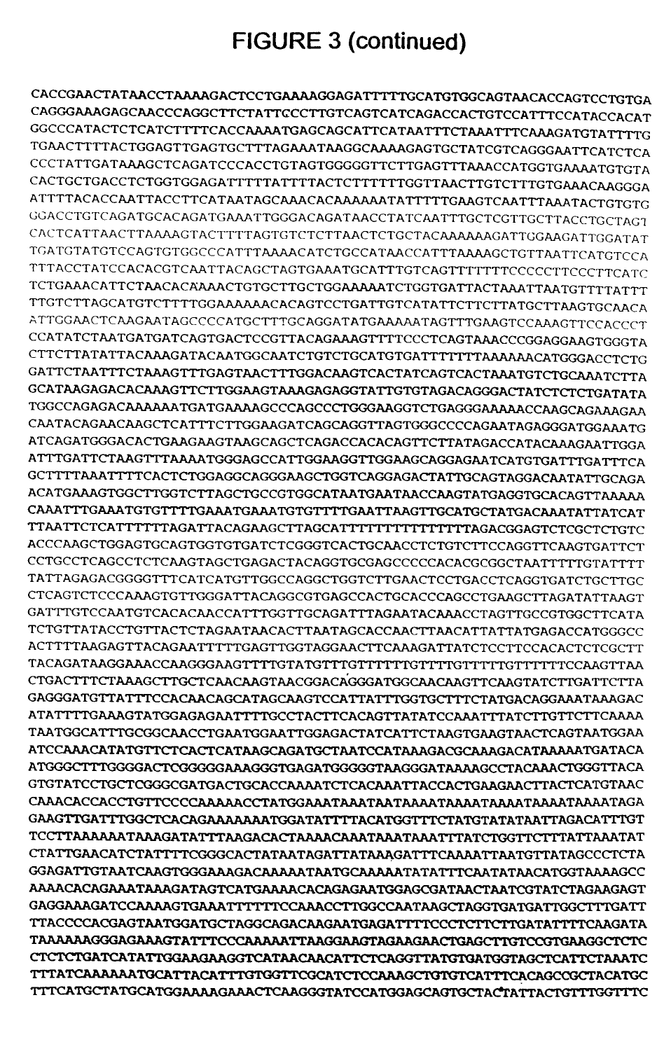

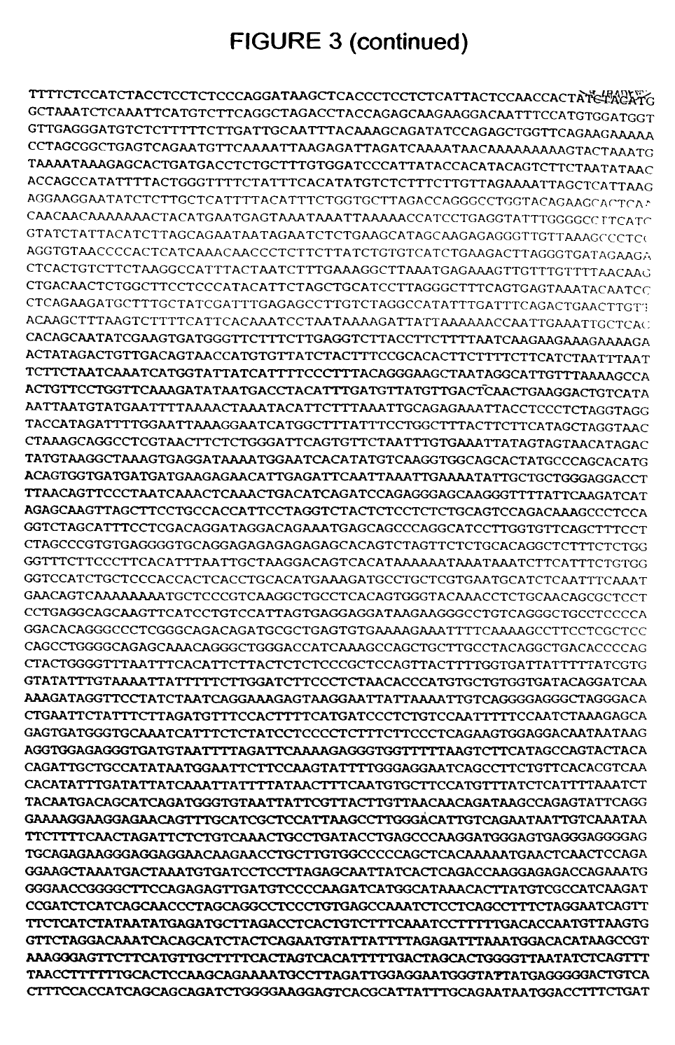

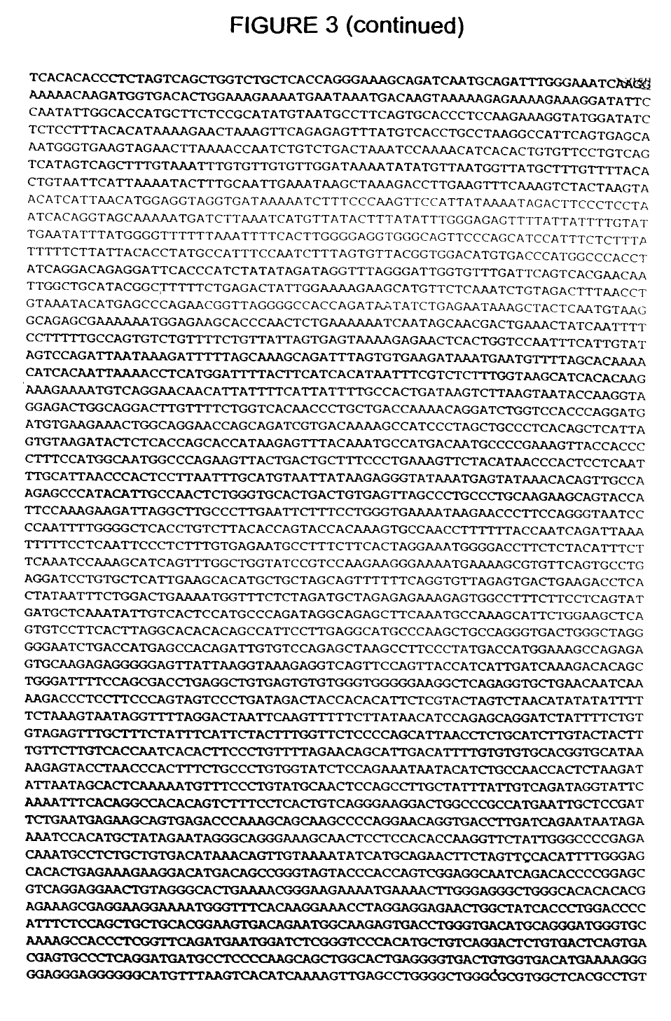

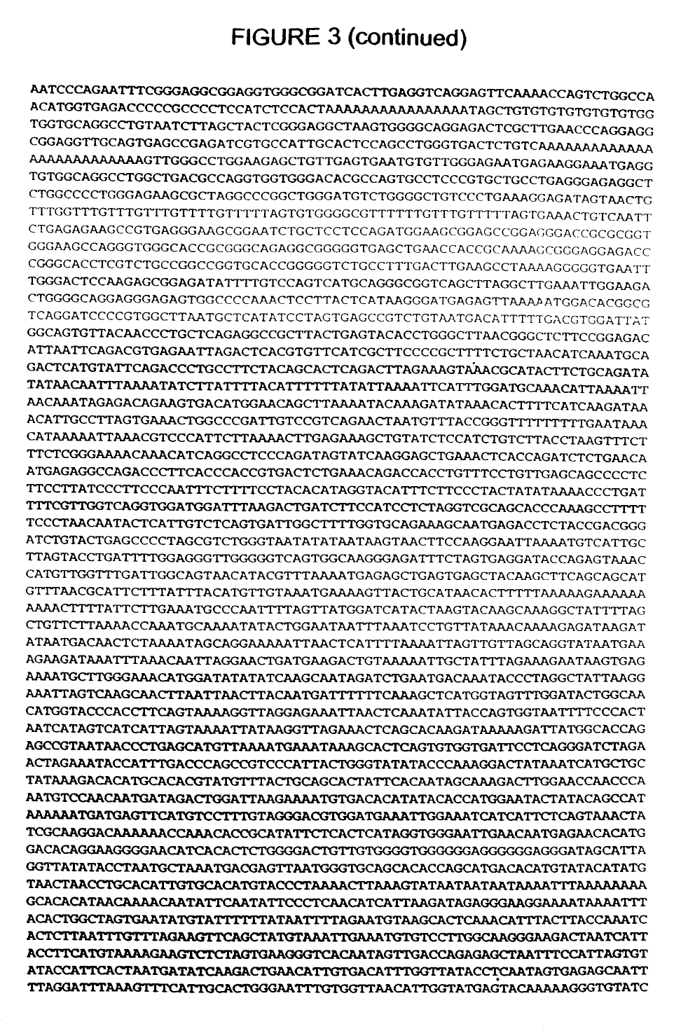

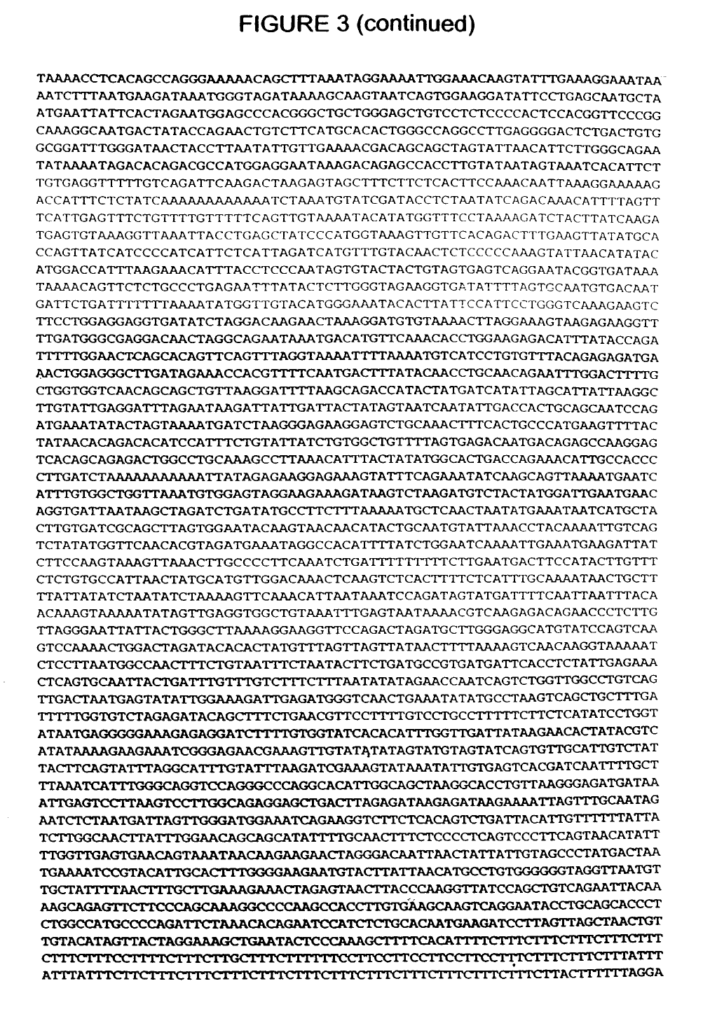

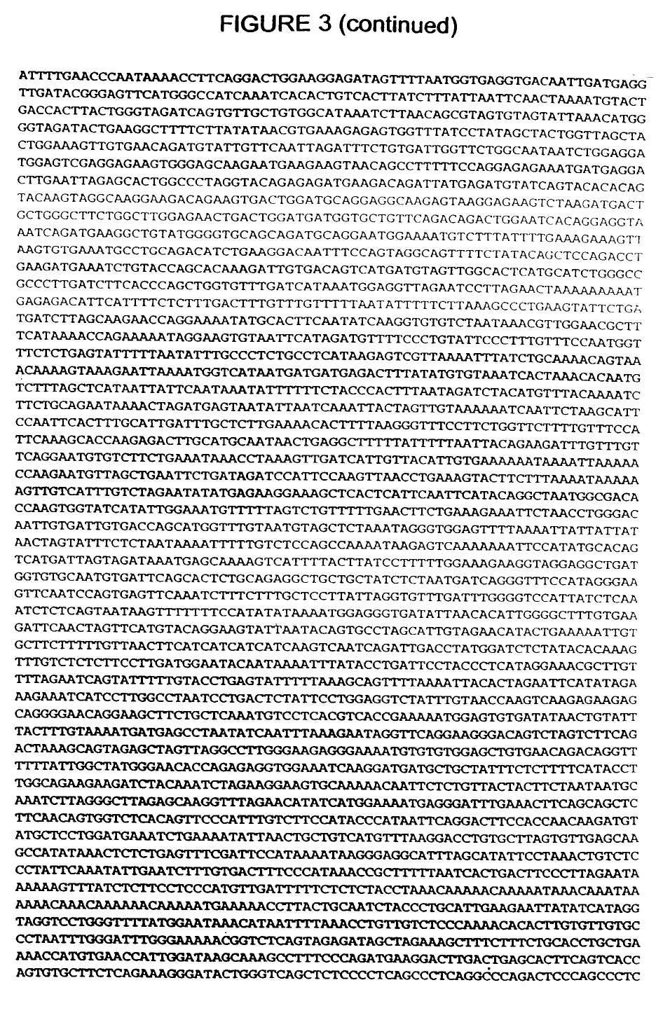

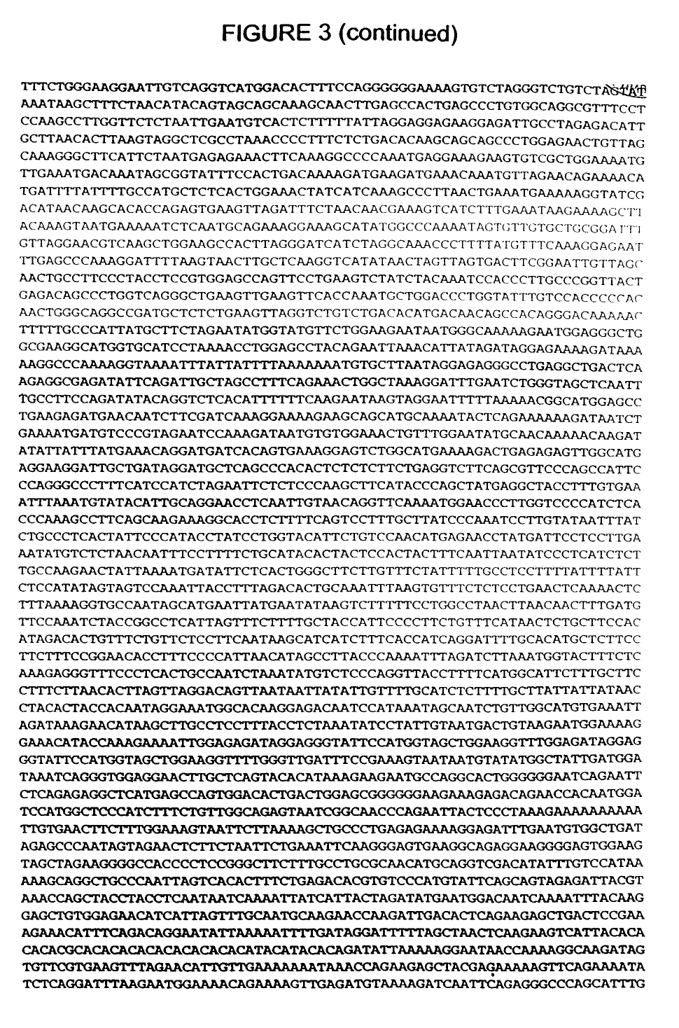

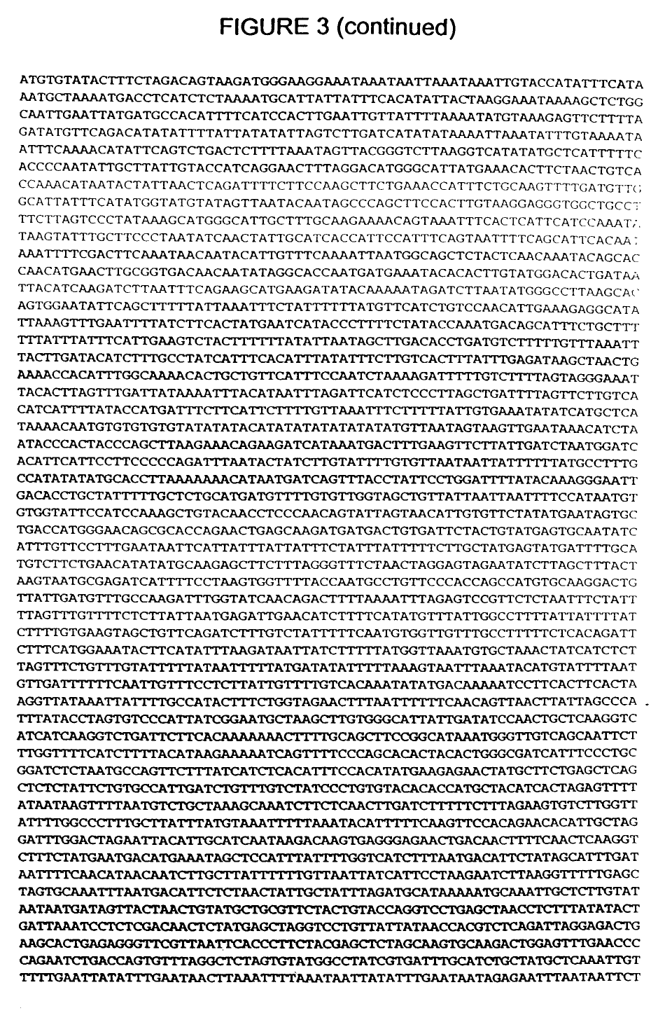

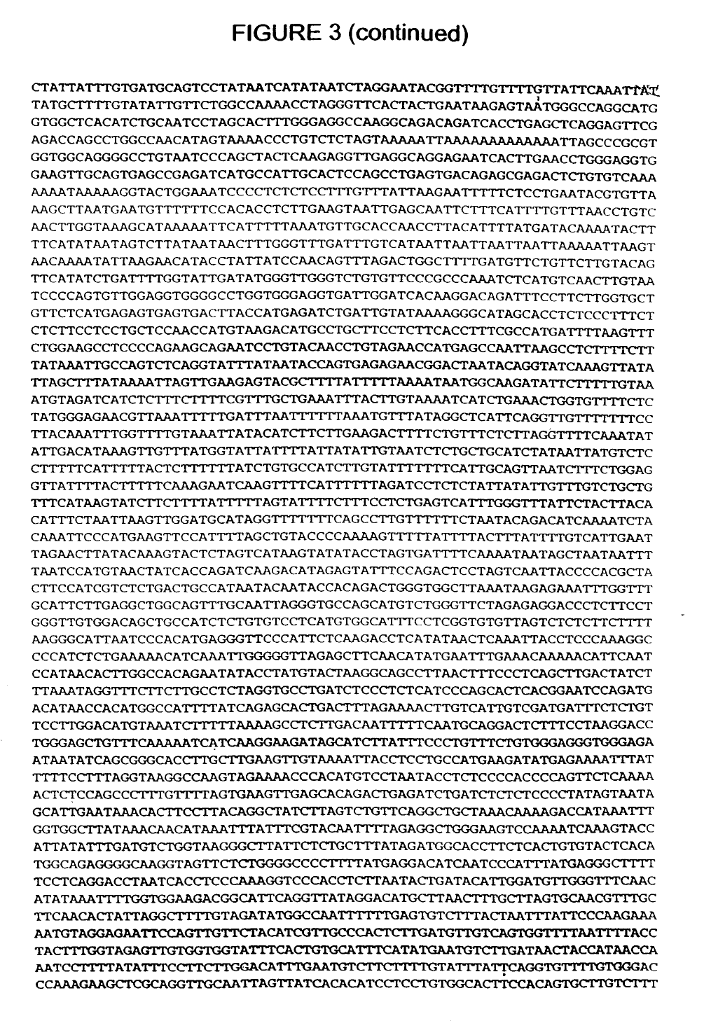

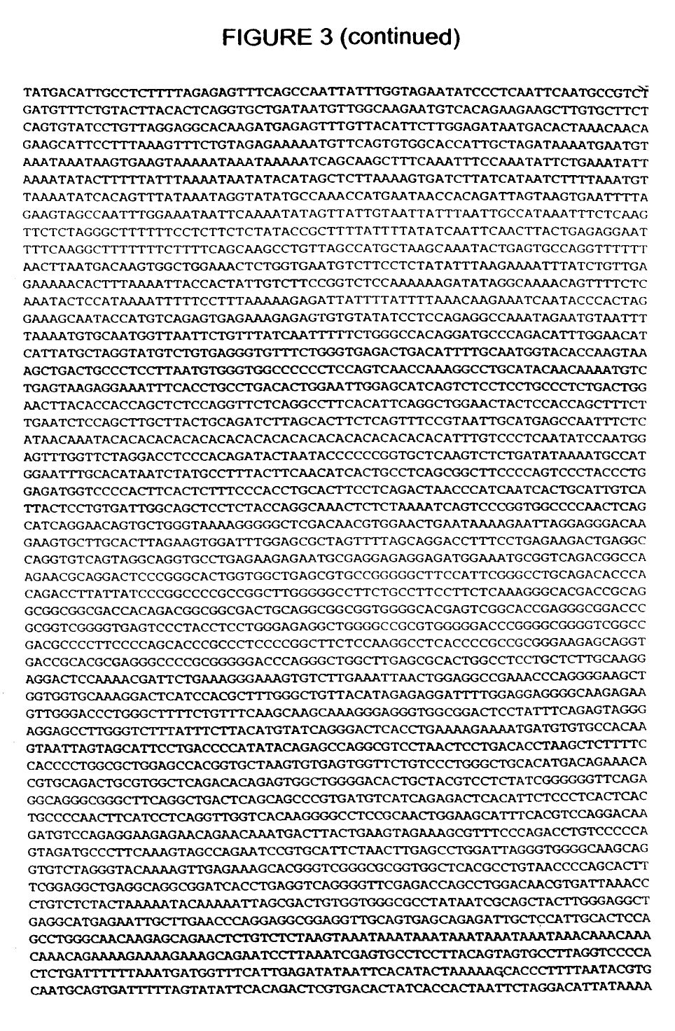

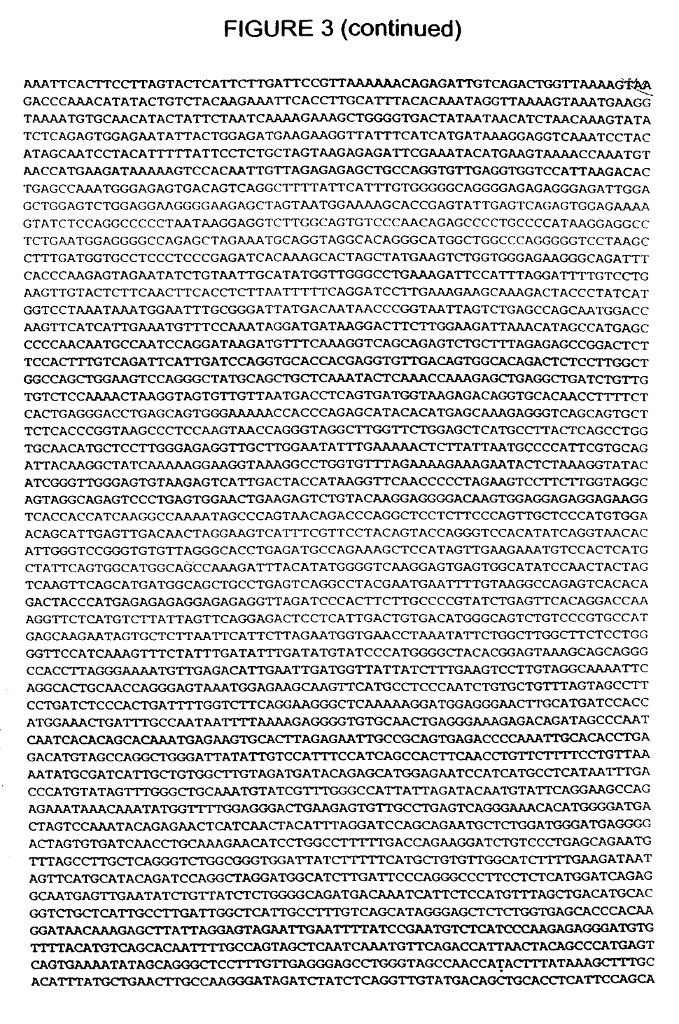

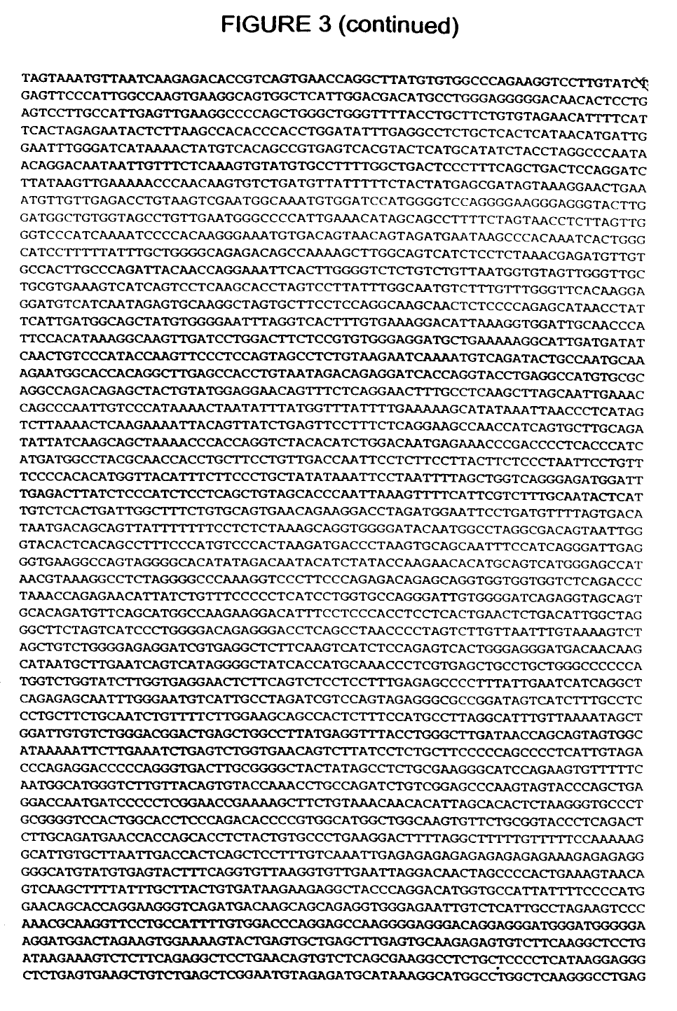

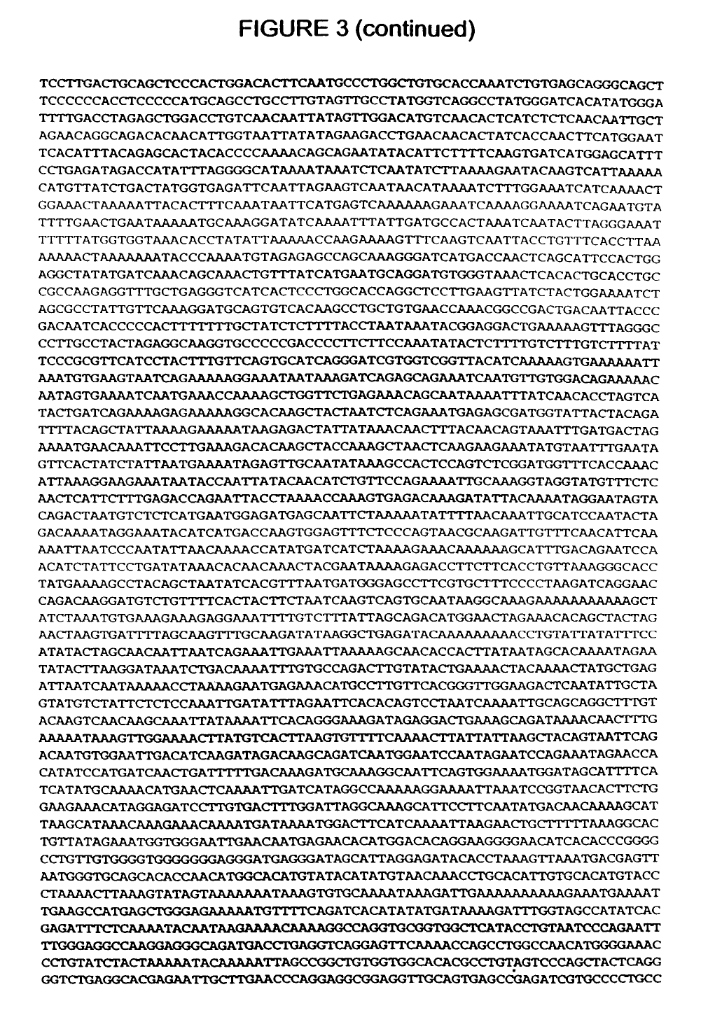

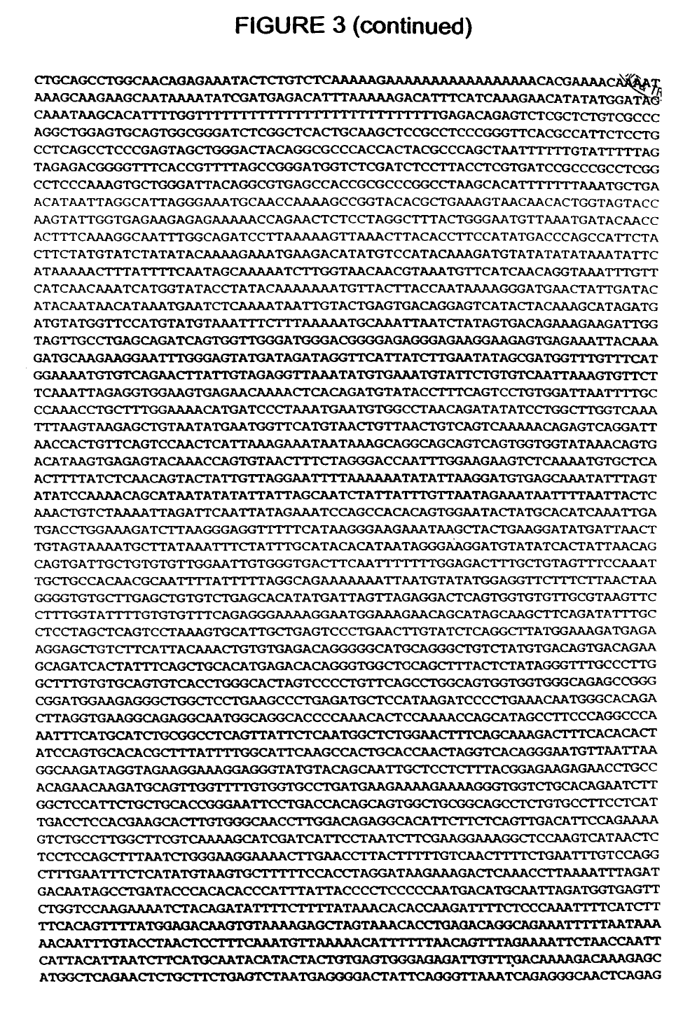

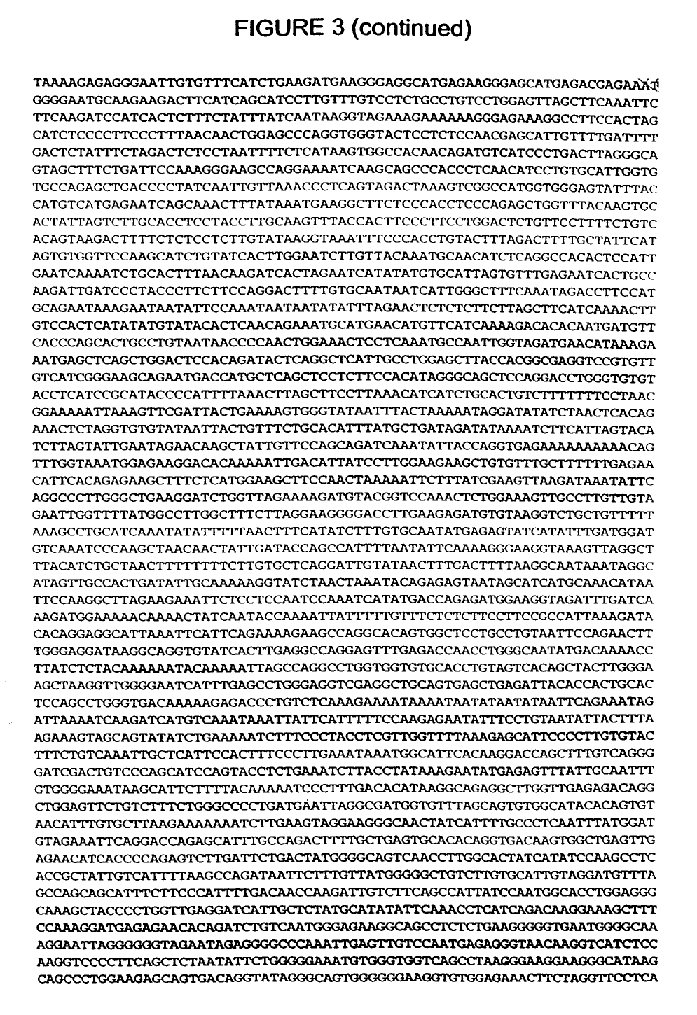

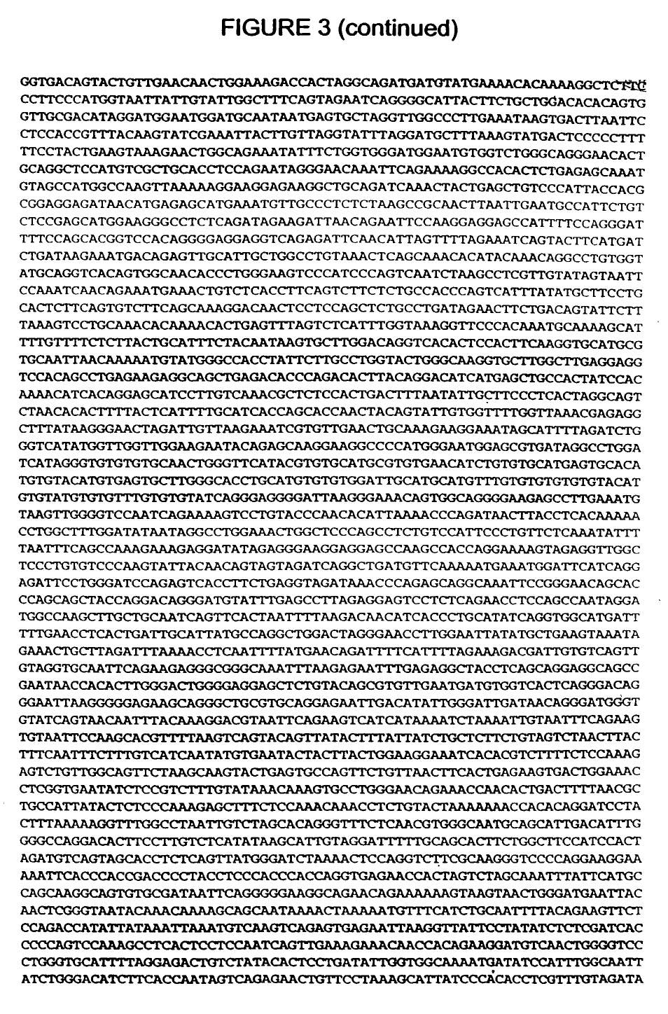

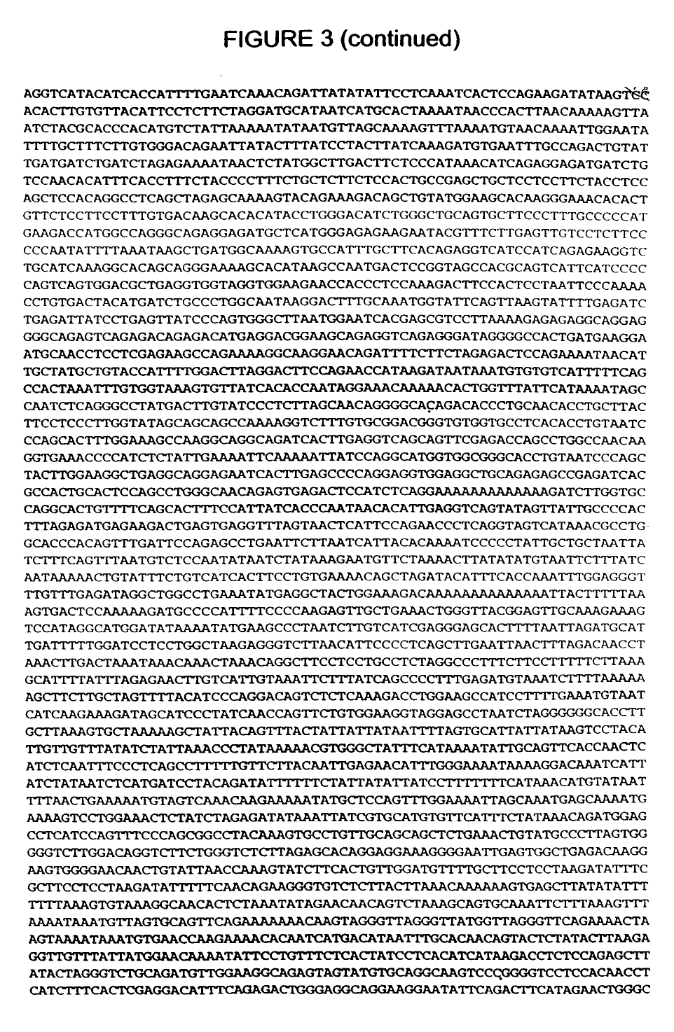

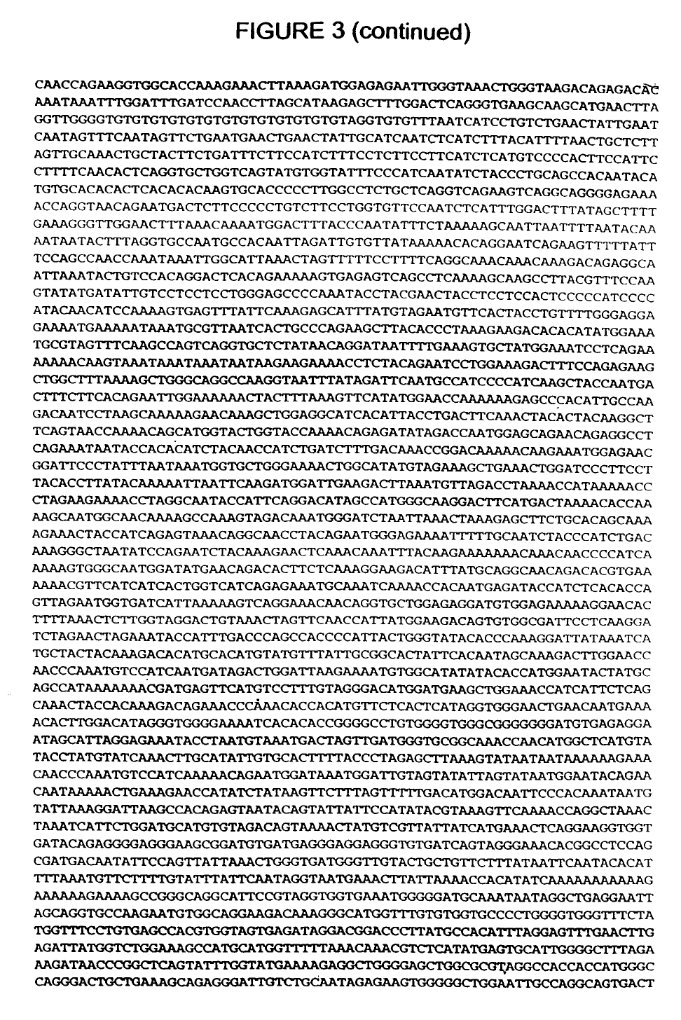

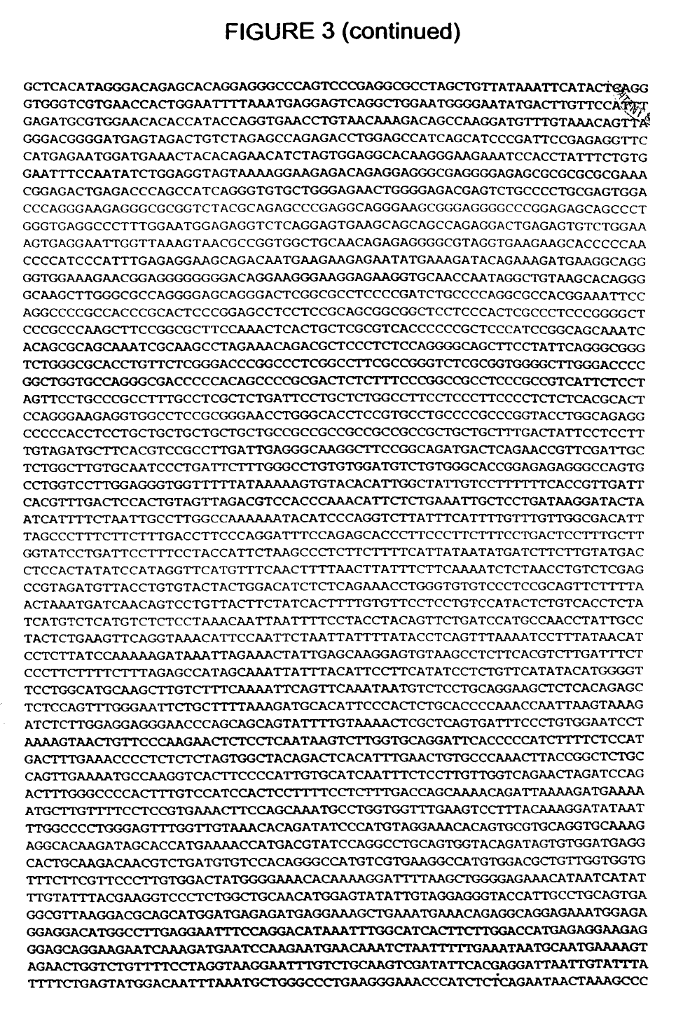

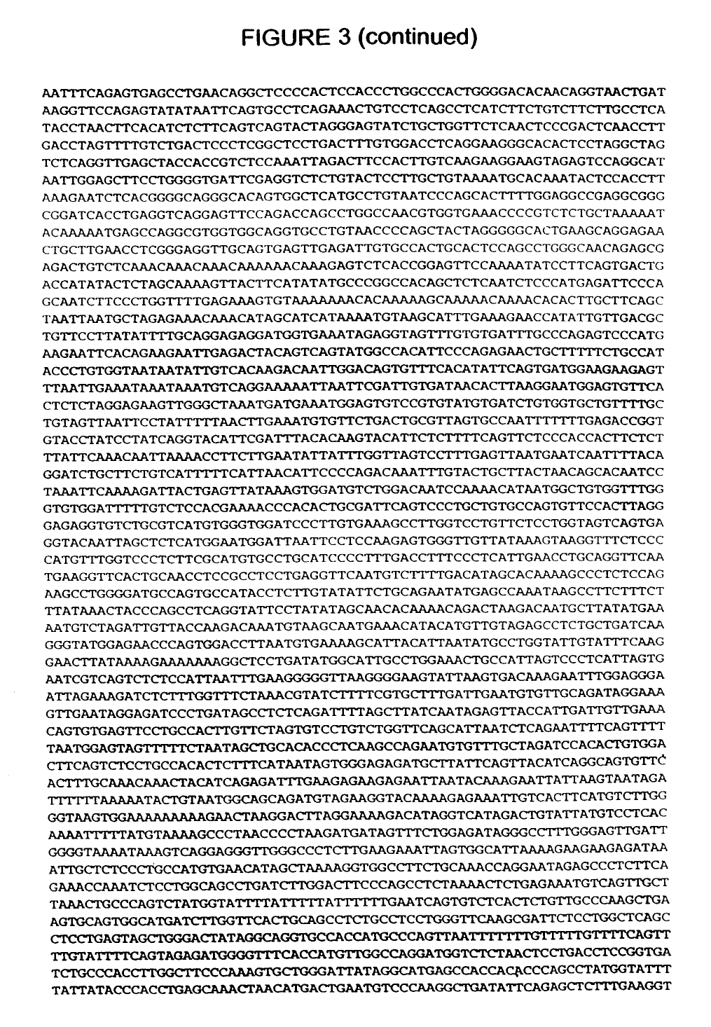

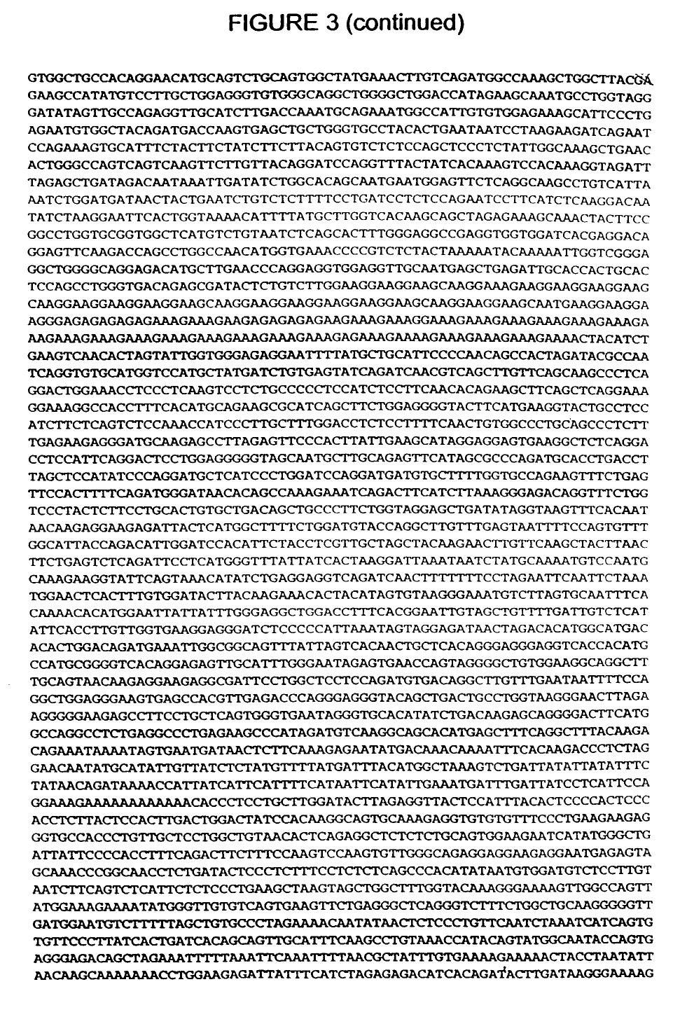

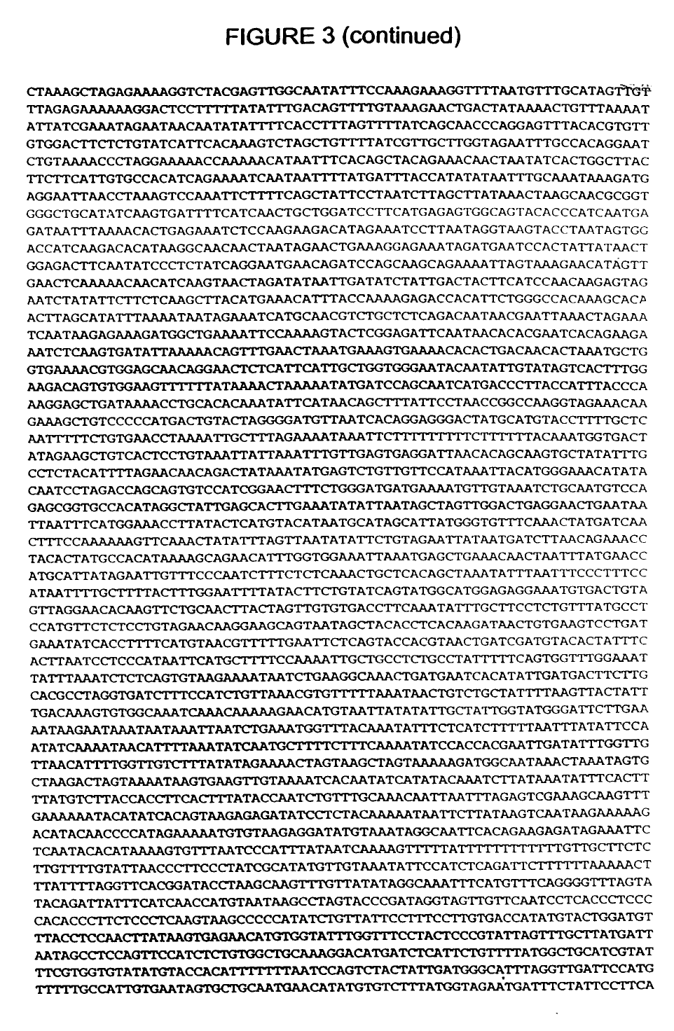

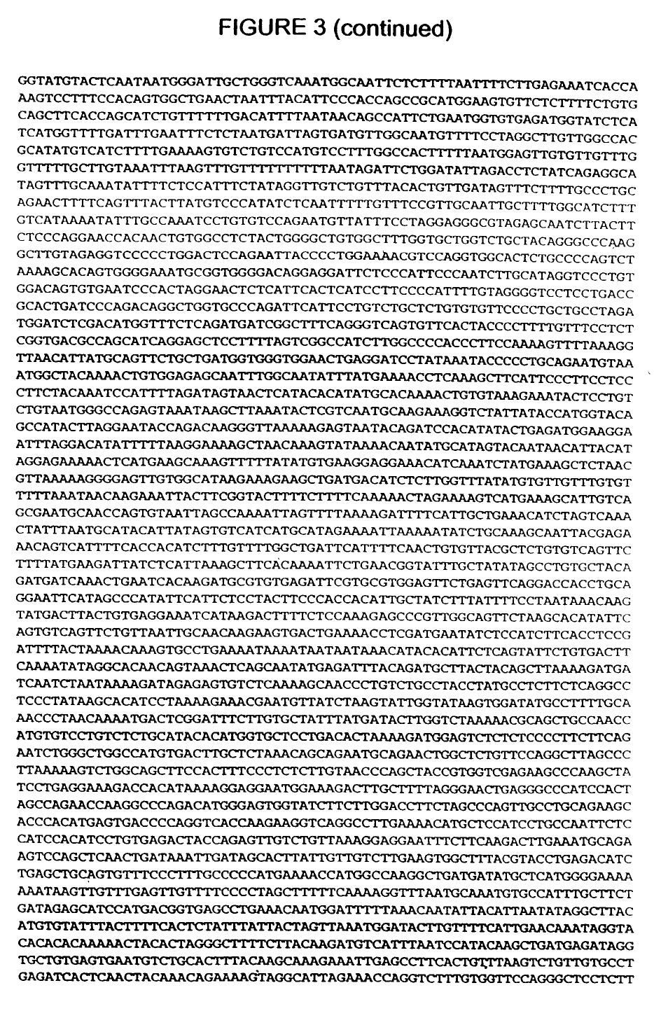

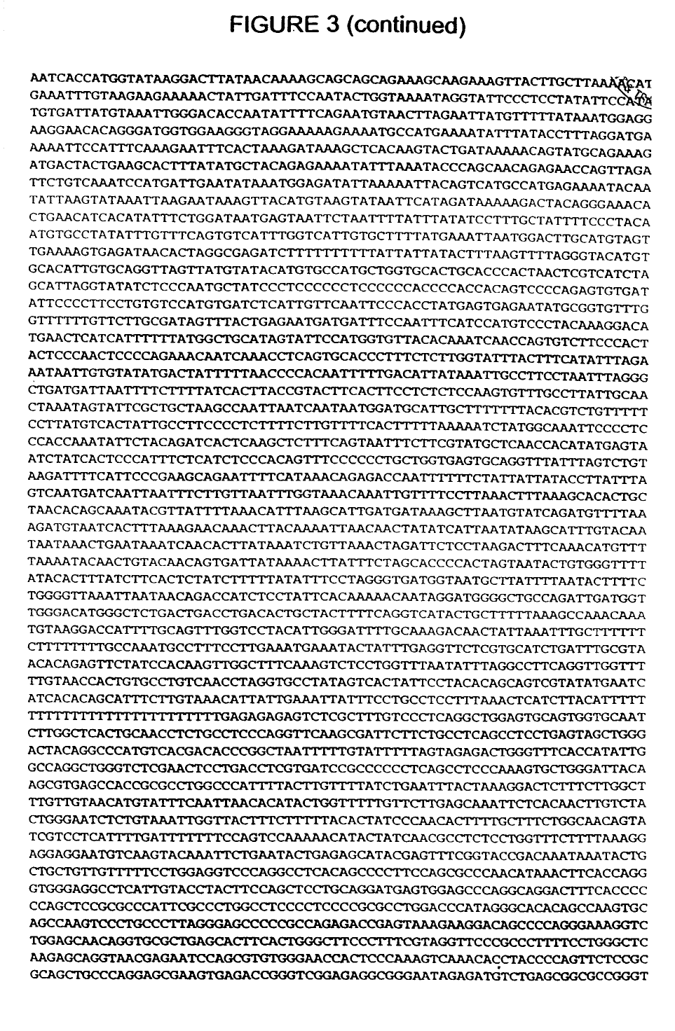

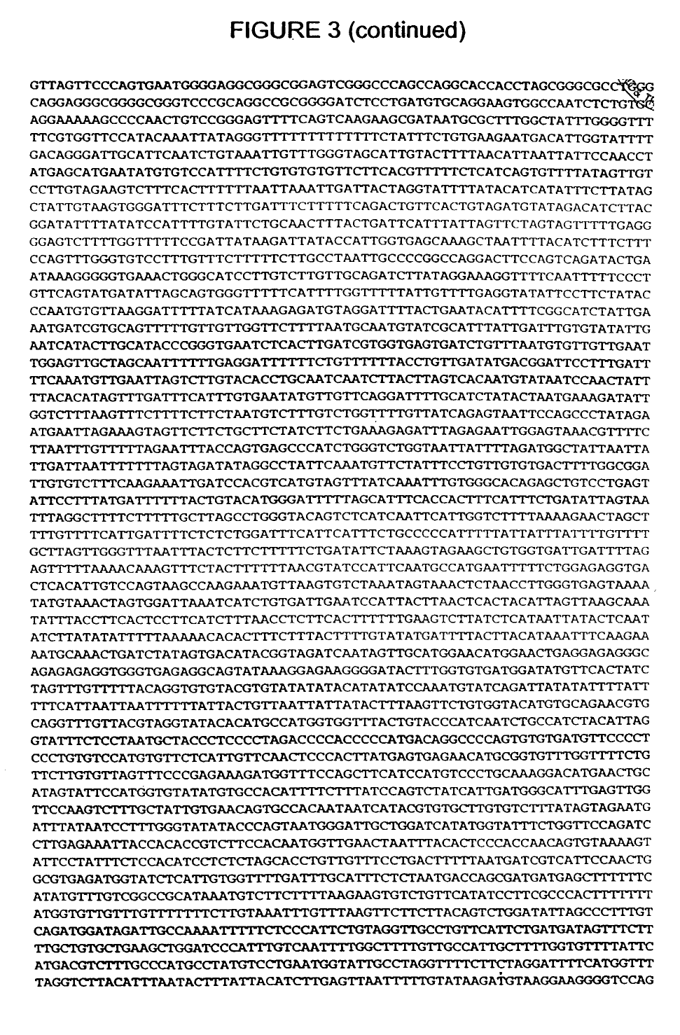

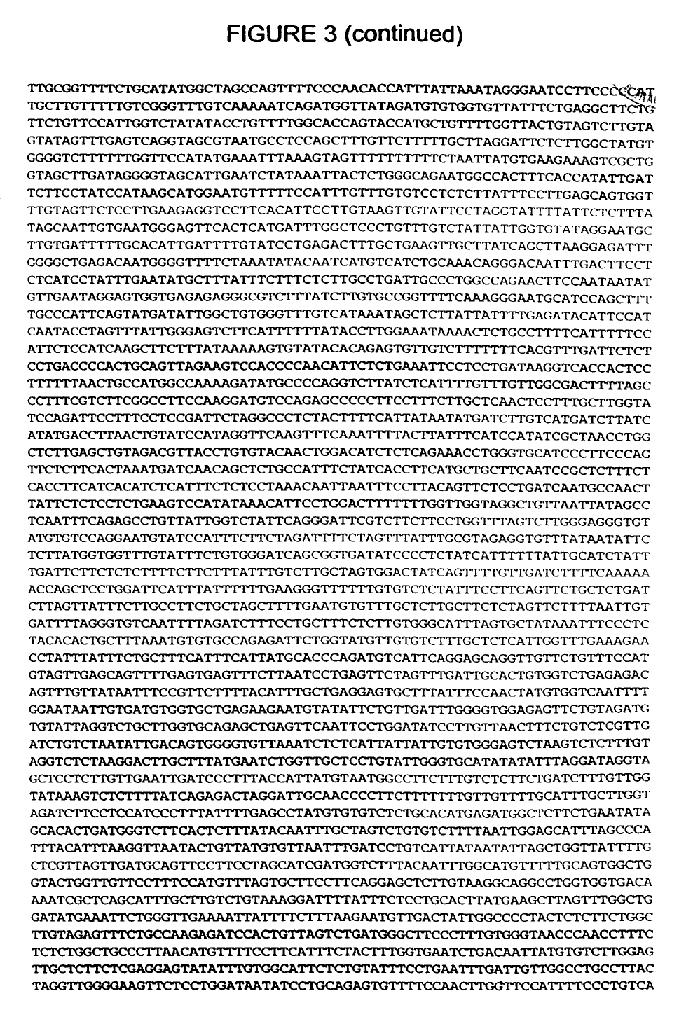

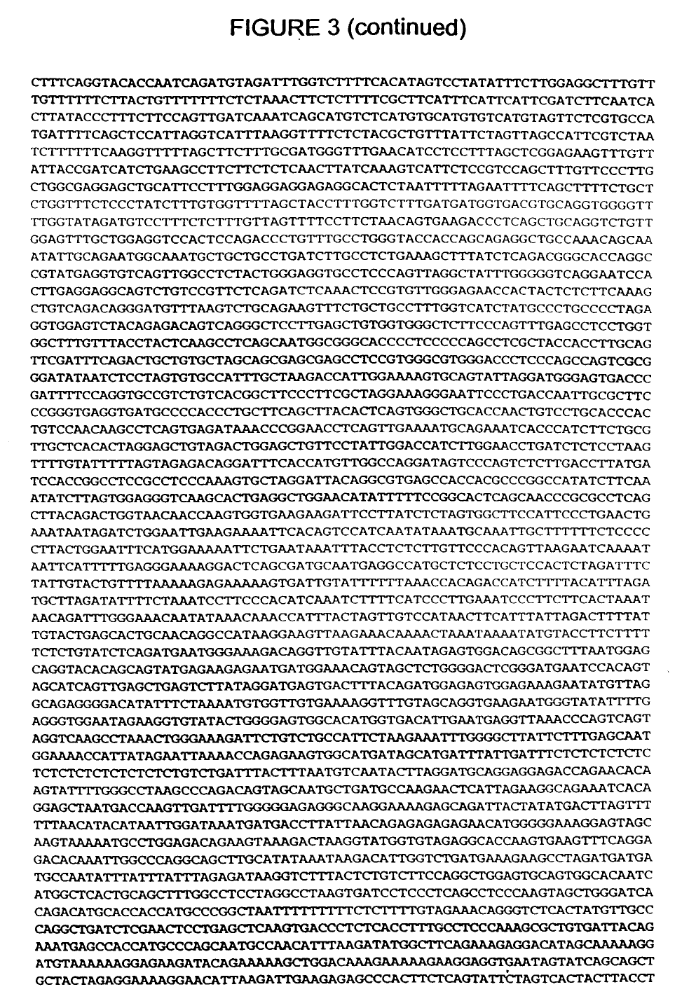

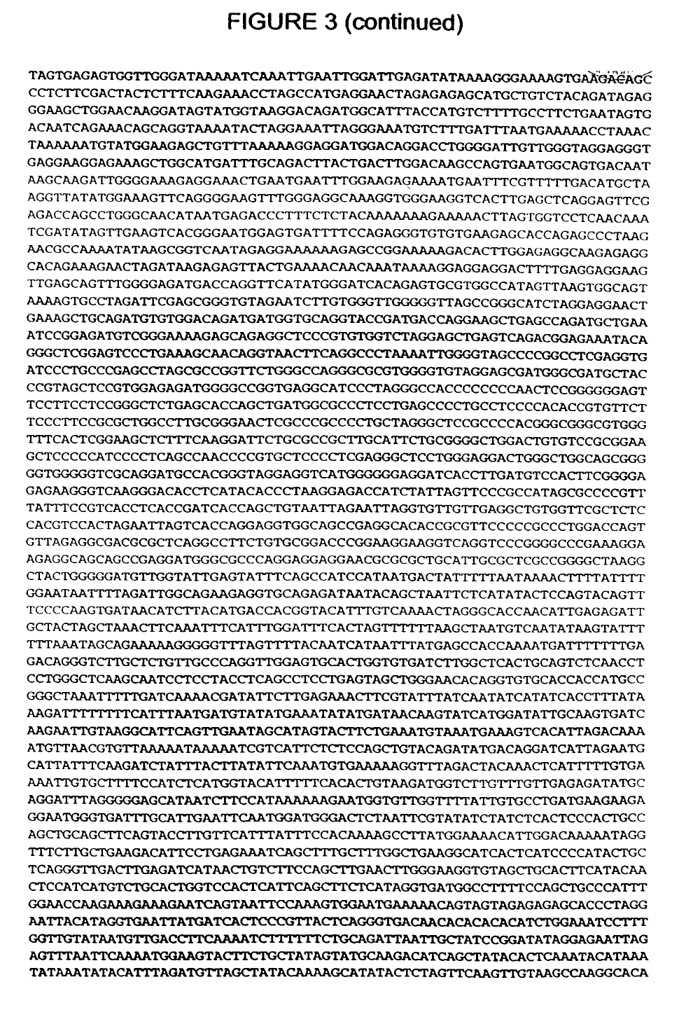

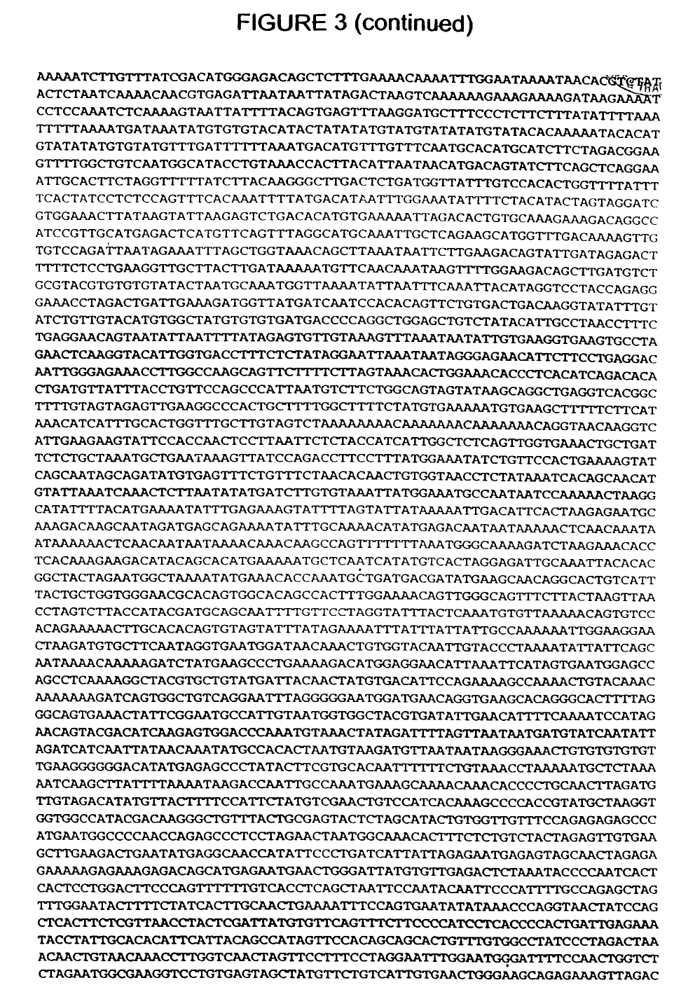

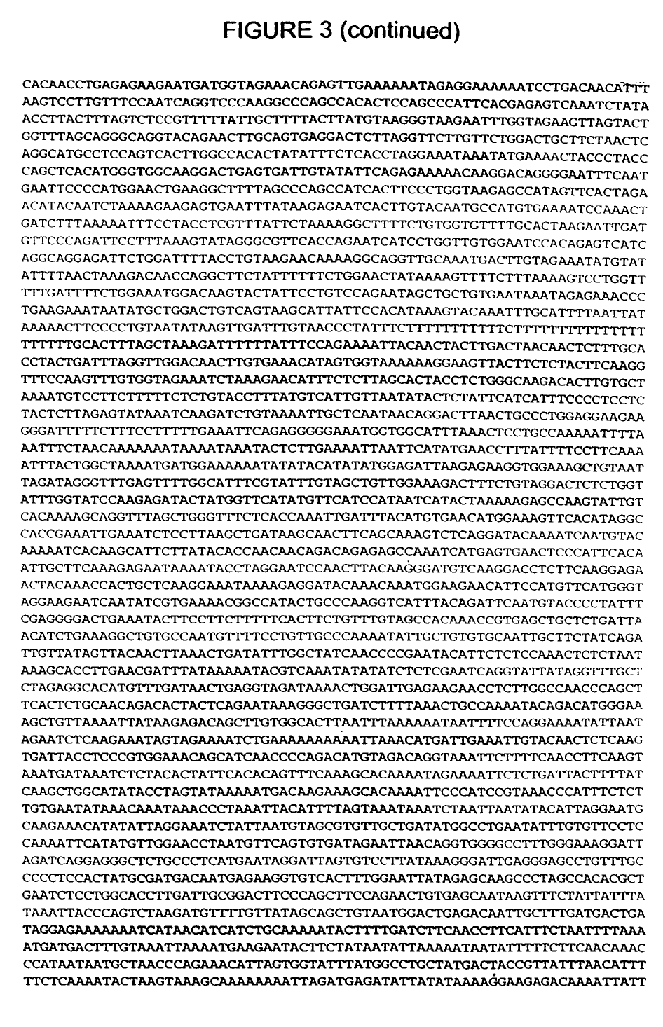

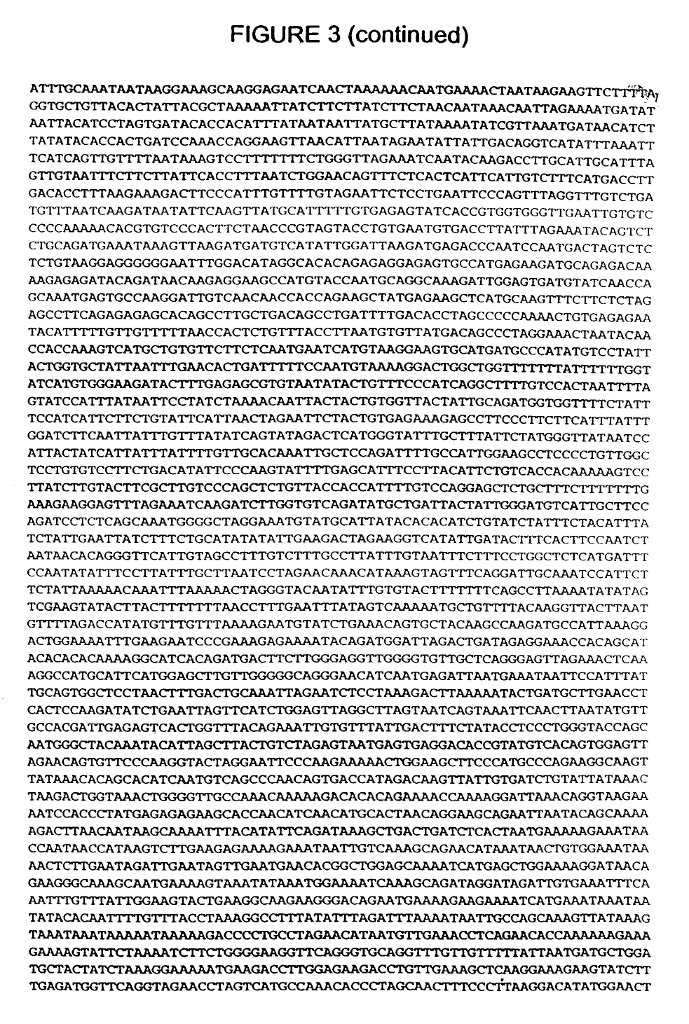

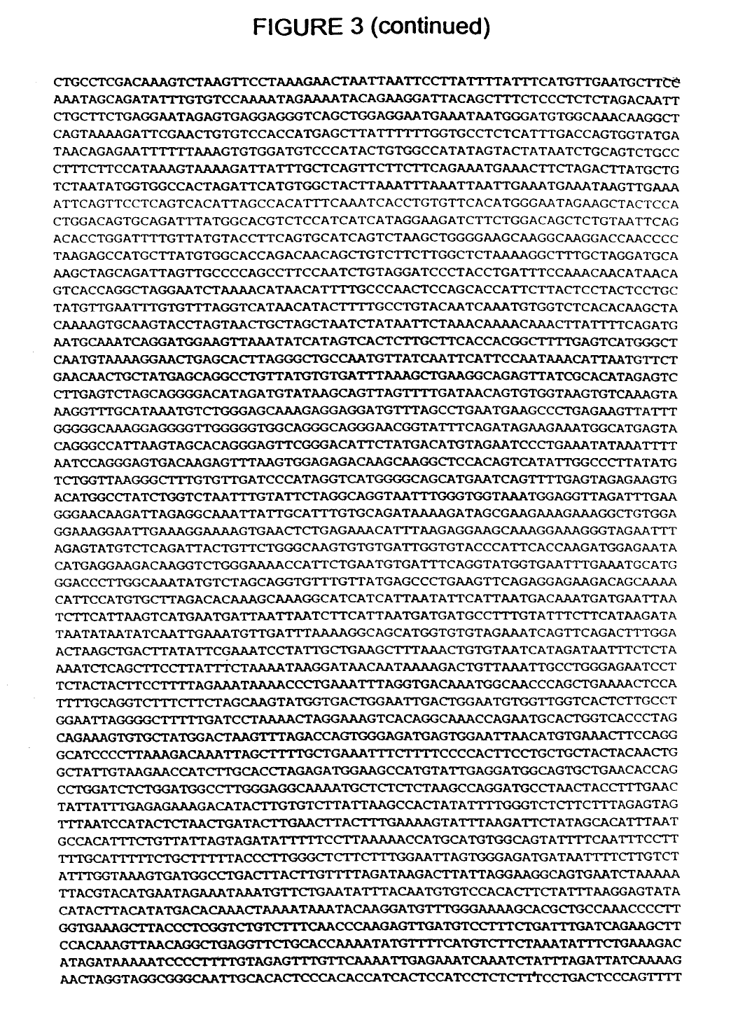

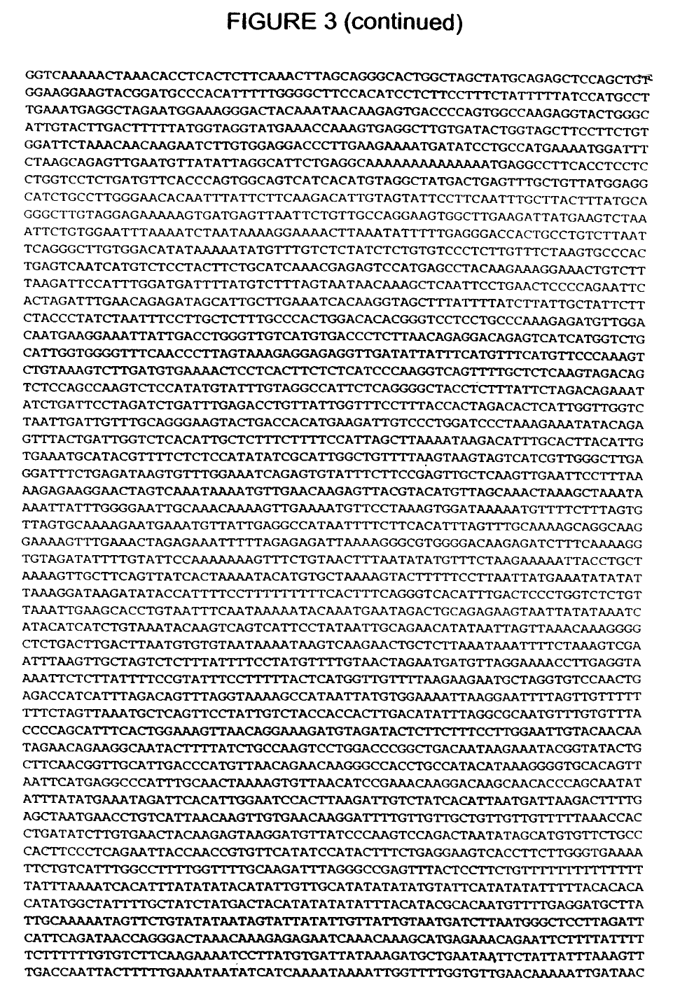

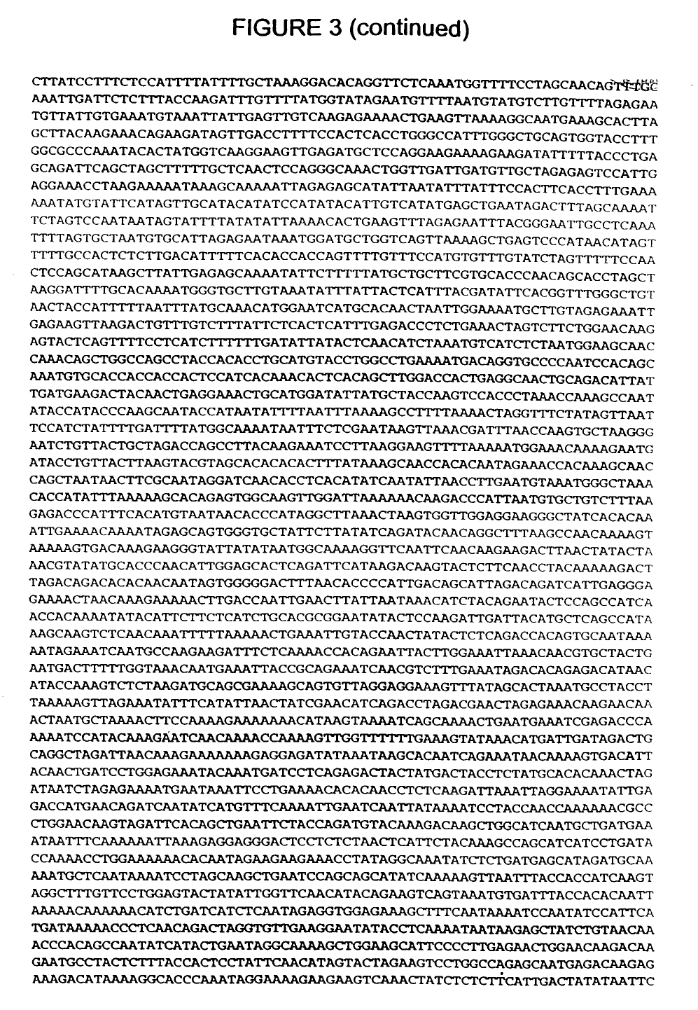

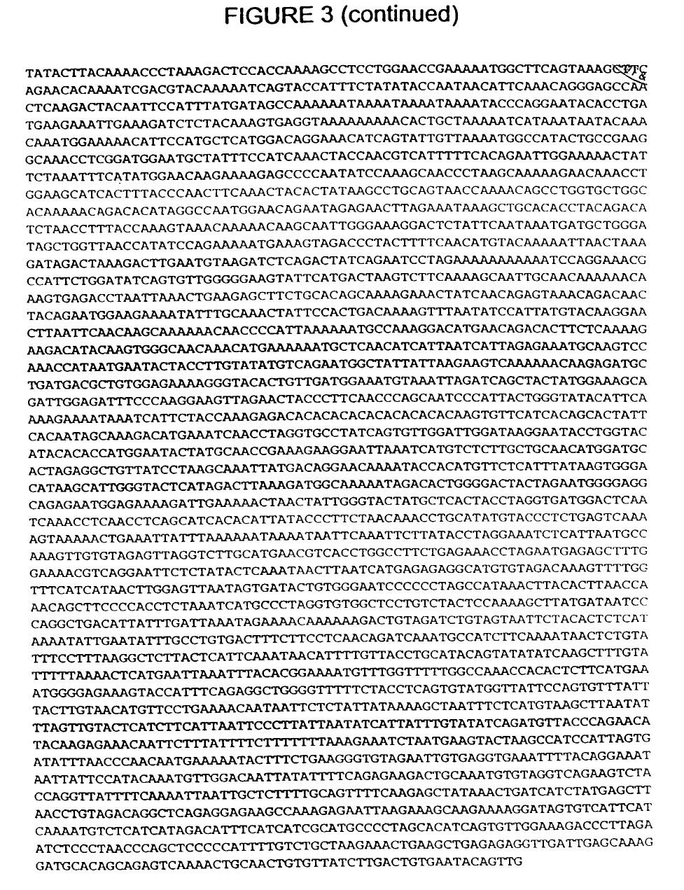

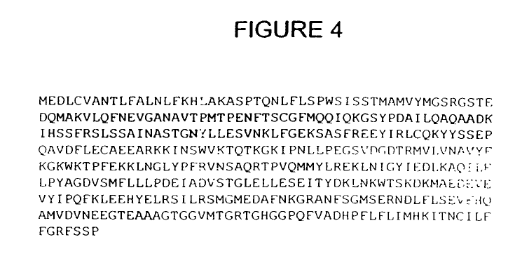

Specific reference nucleotide (SEQ ID NO:1) and amino acid (SEQ ID NO: 2) sequences for PLCG1 are shown in FIGS. 1 and 2, respectively. Specific reference nucleotide (SEQ ID NO: 3) and amino acid (SEQ ID NO: 4) sequences for PAI-2 are shown in FIGS. 3 and 4, respectively. It is understood that the invention is not limited by these exemplified reference sequences, as variants of these sequences which differ at locations other than the SNP sites identified herein can also be utilized. The skilled artisan can readily determine the SNP sites in these other reference sequences which correspond to the SNP sites identified herein by aligning the sequence of interest with the reference sequences specifically disclosed herein. Programs for performing such alignments are commercially available. For example, the ALIGN program in the GCG software package can be used, utilizing a PAM120 weight residue table, a gap length penalty of 12 and a gap penalty of 4, for example.

The polymorphic region of the present invention is associated with specific diseases or disorders and has been identified in the human PLCG1 and PAI-2 genes by analyzing the DNA of cell lines derived from an ethnically diverse population by methods described in Cargill, et al. (1999) Nature Genetics 22:231-238.

Cases which were used to identify associations between vascular disease and SNPs were comprised of 352 U.S. Caucasian subjects with premature coronary artery disease were identified in 15 participating medical centers, fulfilling the criteria of either myocardial infarction, surgical or percutaneous revascularization, or a significant coronary artery lesion diagnosed before age 45 in men or age 50 in women and having a living sibling who met the same criteria. These cases were compared with a random sample of 418 Caucasian controls drawn from the general U.S. population in Atlanta, Ga. It was determined that a subject having two copies of the variant allele of the PLCG1 gene (TT) at residue 64001 of the reference sequence GI 11345540 and two copies of the reference allele of the PAI-2 gene (TT) at residue 170871 of the reference sequence GI 6705901, or the complements thereof, in combination, is approximately 3-fold less likely to develop a vascular disease such as CAD or MI compared to a subject having any other possible combination of alleles at these residues.

The allelic variants of the present invention were identified by performing denaturing high performance liquid chromatography (DHPLC) analysis, variant detector arrays (Affymetrix™), the polymerase chain reaction (PCR), and/or single stranded conformation polymorphism (SSCP) analysis of genomic DNA from independent individuals as described in the Examples, using PCR primers complementary to intronic sequences surrounding each of the exons, 3′ UTR, and 5′ upstream regulatory element sequences of the PLCG1 and PAI-2 genes.

The presence of at least one polymorphism in the human PLCG1 gene and one polymorphism in the PAI-2 gene in the population studied were identified. Both of the variants are characterized as single nucleotide polymorphisms (SNPs). The preferred polymorphisms of the invention are listed in Table 3.

Table 3 contains a “polymorphism ID No.” in column 2, which is used herein to identify each individual variant. In Table 3, the nucleotide sequence flanking each polymorphism is provided in column 9, wherein the polymorphic residue(s), having the variant nucleotide, is indicated in lower-case letters. There are 8 nucleotides flanking the polymorphic nucleotide residue (i.e., 8 nucleotides 5′ of the polymorphism and 8 nucleotides 3′ of the polymorphism). Column 10 indicates the SEQ ID NO. that is used to identify each polymorphism. SEQ ID NOs:5 and 6 comprise sequences shown in column 9 with the variant nucleotide at the residue(s) shown by a lower-case letter.

Each polymorphism is identified based on a change in the nucleotide sequence from a consensus sequence, or the “reference sequence.” As used herein, the reference sequence of PLCG1 is the nucleotide sequence of SEQ ID NO:1 which corresponds to GI 11345540 (see FIG. 1) and the reference sequence of PAI-2 is the nucleotide sequence of SEQ ID NO:3 which corresponds to GI 6705901 (see FIG. 3).

To identify the location of each polymorphism in Table 3, a specific nucleotide residue in a reference sequence is listed for each polymorphism, where nucleotide residue number 1 is the first (i.e., 5′) nucleotide in GI 11345540 (the reference sequence for the PLCG1 gene, corresponding to SEQ ID NO:1), the first nucleotide in GI 6705901 (the reference sequence for the PAI-2 gene, corresponding to SEQ ID NO:3). Column 8 lists the reference sequence and polymorphic residue for each polymorphism.

Column 4 describes the type of variant for each SNP. Both of the SNPs of the instant invention result in a missense amino acid in the amino acid sequence of each protein. For example, as can be seen in Table 3, one polymorphism found in the population is a change from a cytidine to a thymidine in the PLCG1 gene at residue 64001 of GI 11345540 (polymorphism ID No. G329 ul) (SEQ ID NO:5), or the complement thereof, which results in a change from an isoleucine to a threonine in the amino acid sequence of PLCG1 (SEQ ID NO:2) at amino acid residue 813. The second polymorphism is a change from a thymidine to a cytidine in the PAI-2 gene at residue 170871 of GI 6705901 (polymorphism ID No. PAI-2 ul) (SEQ ID NO:6), or the complement thereof, which results in a change from an asparagine to aspartic acid in the amino acid sequence of PAI-2 (SEQ ID NO:4) at amino acid residue 120.

The nucleic acid molecules of the invention can be double- or single-stranded.

Accordingly, the invention further provides for the complementary nucleic acid strands comprising the polymorphisms listed in Table 3.

The invention further provides allele-specific oligonucleotides that hybridize to a gene comprising a single nucleotide polymorphism or to the complement of the gene. Such oligonucleotides will hybridize to one polymorphic form of the nucleic acid molecules described herein but not to the other polymorphic form(s) of the sequence. Thus such oligonucleotides can be used to determine the presence or absence of particular alleles of the polymorphic sequences described herein. These oligonucleotides can be probes or primers.

Not only does the present invention provide polymorphisms in linkage disequilibrium with the polymorphisms of Table 3, it also provides methods for revealing the existence of yet other polymorphic regions in the human PLCG1 or PAI-2 gene. For example, the polymorphism studies described herein can also be applied to populations in which other vascular diseases or disorders are prevalent.

Other aspects of the invention are described below or will be apparent to one of skill in the art in light of the present disclosure.

Definitions

For convenience, the meaning of certain terms and phrases employed in the specification, examples, and appended claims are provided below.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene, including the PLCG1 or PAI-2 genes, can differ from each other in a single nucleotide, or several nucleotides, and can include substitutions, deletions, and insertions of nucleotides. An allele of a gene can also be a form of a gene containing one or more mutations.

The term “allelic variant of a polymorphic region of a PLCG1 or a PAI-2 gene” refers to an alternative form of the PLCG1 or PAI-2 gene having one of several possible nucleotide sequences found in that region of the gene in the population.

“Biological activity” or “bioactivity” or “activity” or “biological function”, which are used interchangeably, for the purposes herein when applied to PLCG1 or PAI-2, means an effector or antigenic function that is directly or indirectly performed by a PLCG1 or PAI-2 polypeptide (whether in its native or denatured conformation), or by a fragment thereof. Biological activities include modulation of the development of atherosclerotic plaque leading to vascular disease and other biological activities, whether presently known or inherent. A PLCG1 or PAI-2 bioactivity can be modulated by directly affecting a PLCG1 or PAI-2 protein effected by, for example, changing the level of effector or substrate level. Alternatively, a PLCG1 or PAI-2 bioactivity can be modulated by modulating the level of a PLCG1 or PAI-2 protein, such as by modulating expression of a PLCG1 or PAI-2 gene. Antigenic functions include possession of an epitope or antigenic site that is capable of cross-reacting with antibodies that bind a native or denatured PLCG1 or PAI-2 polypeptide or fragment thereof.

Biologically active PLCG1 or PAI-2 polypeptides include polypeptides having both an effector and antigenic function, or only one of such functions. PLCG1 or PAI-2 polypeptides include antagonist polypeptides and native PLCG1 or PAI-2 polypeptides, provided that such antagonists include an epitope of a native PLCG1 or PAI-2 polypeptide. An effector function of PLCG1 or PAI-2 polypeptide can be the ability to bind to a ligand of a PLCG1 or PAI-2 molecule.

As used herein the term “bioactive fragment of a PLCG1 or PAI-2 protein” refers to a fragment of a full-length PLCG1 or PAI-2 protein, wherein the fragment specifically mimics or antagonizes the activity of a wild-type PLCG1 or PAI-2 protein. The bioactive fragment preferably is a fragment capable of binding to a second molecule, such as a ligand.

The term “an aberrant activity” or “abnormal activity”, as applied to an activity of a protein such as PLCG1 or PAI-2, refers to an activity which differs from the activity of the wild-type (i.e., normal or reference) protein or which differs from the activity of the protein in a healthy subject, e.g., a subject not afflicted with a disease associated with a PLCG1 or PAI-2 allelic variant. An activity of a protein can be aberrant because it is stronger than the activity of its wild-type counterpart. Alternatively, an activity of a protein can be aberrant because it is weaker or absent relative to the activity of its wild-type counterpart. An aberrant activity can also be a change in reactivity. For example an aberrant protein can interact with a different protein or ligand relative to its wild-type counterpart. A cell can also have aberrant PLCG1 or PAI-2 activity due to overexpression or underexpression of the PLCG1 or PAI-2 gene. Aberrant PLCG1 or PAI-2 activity can result from a mutation in the gene, which results, e.g., in lower or higher binding affinity of a ligand to the PLCG1 or PAI-2 protein encoded by the mutated gene. Aberrant PLCG1 or PAI-2 activity can also result from an abnormal PLCG1 or PAI-2 5′ upstream regulatory element activity.

“Cells,” “host cells” or “recombinant host cells” are terms used interchangeably herein. It is understood that such terms refer not only to the particular cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “course of clinical therapy” refers to any chosen method to treat, prevent, or ameliorate a vascular disease, e.g., CAD or MI, symptoms thereof, or related diseases or disorders. Courses of clinical therapy include, but are not limited to, lifestyle changes (e.g., changes in diet or environment), administration of medication, use of surgical devices, such as, but not limited to, stents, angioplasty devices, used in, for example, percutaneous transluminal coronary balloon angioplasty (PTCA) or laser angioplasty, defibrillators, implantation of a stent, or other surgical intervention, such as, for example, coronary bypass grafting (CABG), or any combination thereof.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “genetic profile” refers to the information obtained from identification of the specific alleles of a subject, e.g., specific alleles within a polymorphic region of a particular gene or genes or proteins encoded by such genes. For example, a PLCG1 genetic profile refers to the specific alleles of a subject within the PLCG1 gene and a PAI-2 genetic profile refers to the specific alleles of a subject within the PAI-2 gene. For example, one can determine a subject's PLCG1 and/or PAI-2 genetic profile by determining the identity of the nucleotide present at nucleotide position 11345540 of SEQ ID NO:1 and/or the nucleotide present at nucleotide position 170871 of SEQ ID NO:3. One can also determine a subjects PLCG1 and/or PAI-2 genetic profile by determining the identity of the amino acid present at amino acid residue 813 of SEQ ID NO:2 and/or amino acid 120 of SEQ ID NO:4. The genetic profile of a particular disease can be ascertained through identification of the identity of allelic variants in one or more genes which are associated with the particular disease.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID NO:N is intended to include nucleic acids having a nucleotide sequence which has a certain degree of homology with SEQ ID NO:N or with the complement thereof. Preferred homologs of nucleic acids are capable of hybridizing to the nucleic acid or complement thereof.

The term “hybridization probe” or “primer” as used herein is intended to include oligonucleotides which hybridize bind in a base-specific manner to a complementary strand of a target nucleic acid. Such probes include peptide nucleic acids, and described in Nielsen et al., (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range form about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer of the invention comprises a sequence that flanks and/or preferably overlaps, at least one polymorphic site occupied by any of the possible variant nucleotides. The nucleotide sequence of an overlapping probe or primer can correspond to the coding sequence of the allele or to the complement of the coding sequence of the allele.

The term “vascular disease or disorder” as used herein refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage), due to the development of atherosclerotic plaque and diseases and disorders resulting therefrom. Examples of vascular diseases and disorders include, without limitation, atherosclerosis, CAD, MI, ischemia, stroke, peripheral vascular diseases, venous thromboembolism and pulmonary embolism.

The term “interact” as used herein is meant to include detectable interactions between molecules, such as can be detected using, for example, a binding or hybridization assay. The term interact is also meant to include “binding” interactions between molecules. Interactions may be, for example, protein-protein, protein-nucleic acid, protein-small molecule or small molecule-nucleic acid in nature.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium” refers to a greater than random association between specific alleles at two marker loci within a particular population. In general, linkage disequilibrium decreases with an increase in physical distance. If linkage disequilibrium exists between two markers, then the genotypic information at one marker can be used to make probabilistic predictions about the genotype of the second marker.

The term “locus” refers to a specific position in a chromosome. For example, a locus of a PLCG1 or PAI-2 gene refers to the chromosomal position of the PLCG1 or PAI-2 gene.

The term “modulation” as used herein refers to both upregulation, (i.e., activation or stimulation), for example by agonizing; and downregulation (i.e. inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene that differs from the predominant form in a population. A mutated gene is capable of altering the phenotype of a subject having the mutated gene relative to a subject having the predominant form of the gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenine”, “cytidine”, “guanine”, and thymidine” and/or “A”, “C”, “G”, and “T”, respectively, are used. It is understood that if the nucleic acid is RNA, a nucleotide having a uracil base is uridine.

The term “nucleotide sequence complementary to the nucleotide sequence set forth in SEQ ID NO:N” refers to the nucleotide sequence of the complementary strand of a nucleic acid strand having SEQ ID NO:N. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO:N refers to the complementary strand of the strand having SEQ ID NO:N or to any nucleic acid having the nucleotide sequence of the complementary strand of SEQ ID NO:N. When referring to a single stranded nucleic acid having the nucleotide sequence SEQ ID NO:N, the complement of this nucleic acid is a nucleic acid having a nucleotide sequence which is complementary to that of SEQ ID NO:N. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “oligonucleotide” is intended to include and single- or double stranded DNA or RNA. Oligonucleotides can be naturally occurring or synthetic, but are typically prepared by synthetic means. Preferred oligonucleotides of the invention include segments of PLCG1 or PAI-2 gene sequence or their complements, which include and/or flank any one of the polymorphic sites shown in Table 3. The segments can be between 5 and 250 bases, and, in specific embodiments, are between 5-10, 5-20, 10-20, 10-50, 20-50 or 10-100 bases. For example, the segments can be 21 bases. The polymorphic site can occur within any position of the segment or a region next to the segment. The segments can be from any of the allelic forms of PLCG1 or PAI-2 gene sequence shown in Table 3.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wildtype form. Other allelic forms are typically designated or alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the DNA sequence to be amplified and a downstream primer that hybridizes with the 3′ end of the sequence to be amplified.

The terms “protein”, “polypeptide” and “peptide” are used interchangeably herein when referring to a gene product.

The-term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, it is possible that PLCG1 or PAI-2 genes have regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missensc” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

As used herein, the term “specifically hybridizes” or “specifically detects” refers to the ability of a nucleic acid molecule of the invention to hybridize to at least approximately 6, 8, 10, 12, 20, 30, 40, 50, 60, 70, 80, 90, 100, 110, 120, 130 or 140 consecutive nucleotides of either strand of a PLCG1 or PAI-2 gene.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been genetic-engineered into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by genetic engineering, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treatment”, or “treating” as used herein, is defined as the application or administration of a therapeutic agent to a subject, implementation of lifestyle changes (e.g., changes in diet or environment), administration of medication, use of surgical devices, such as, but not limited to, stents, defibrillators, and/or angioplasty devices, and/or surgical procedures, such as, for example, percutaneous transluminal coronary balloon angioplasty (PTCA) or laser angioplasty, implantation of a stent, or other surgical intervention or procedure, such as, for example, coronary bypass grafting (CABG), or any combination thereof, or application or administration of a therapeutic agent to an isolated tissue or cell line from a subject, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

As used herein, the term “vector” refers to a nucleic acid molecule capable of transporting or replicating another nucleic acid to which it has been linked. One type of preferred vector is an episome, i.e., a nucleic acid capable of extra-chromosomal replication.

Preferred vectors are those capable of autonomous replication and/or expression of nucleic acids to which they are linked. Vectors capable of directing the expression of genes to which they are operatively-linked are referred to herein as “expression vectors”. In general, expression vectors of utility in recombinant DNA techniques are often in the form of “plasmids” which refer generally to circular double stranded DNA circles which, in their vector form are not physically linked to the host chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Polymorphisms Used in the Methods of the Invention

The nucleic acid molecules of the present invention include specific allelic variants of the PLCG1 gene and the PAI-2 gene, which differ from the reference sequences set forth in SEQ ID NO:1 or SEQ ID NO:3, respectively, or at least a portion thereof, having a polymorphic region. The preferred nucleic acid molecules of the present invention comprise PLCG1 and PAI-2 sequences having one or more of the polymorphisms shown in Table 3 (SEQ ID NOs:5 and 6), and those in linkage disequilibrium therewith. The invention further comprises isolated nucleic acid molecules complementary to nucleic acid molecules comprising the polymorphisms of the present invention. Nucleic acid molecules of the present invention can function as probes or primers, e.g., in methods for determining the allelic identity of a PLCG1 or PAI-2 polymorphic region. The nucleic acids of the invention can also be used, singly, or, preferably, in combination, to determine whether a subject is or is not at risk of developing a disease associated with a specific allelic variant of a PLCG1 or PAI-2 polymorphic region, e.g., a vascular disease or disorder. The nucleic acids of the invention can further be used to prepare or express PLCG1 or PAI-2 polypeptides encoded by specific alleles, such as mutant alleles. Such nucleic acids can be used in gene therapy. Polypeptides encoded by specific PLCG1 or PAI-2 alleles, such as mutant PLCG1 or PAI-2 polypeptides, can also be used in therapy or for preparing reagents, e.g., antibodies, for detecting PLCG1 or PAI-2 proteins encoded by these alleles. Accordingly, such reagents can be used to detect mutant PLCG1 or PAI-2 proteins.

As described herein, allelic variants of human PLCG1 or PAI-2 genes have been identified. The invention is intended to encompass these allelic variants as well as, those in linkage disequilibrium which can be identified, e.g., according to the methods described herein. “Linkage disequilibrium” refers to an association between specific alleles at two marker loci within a particular population. In general, linkage disequilbrium decreases with an increase in physical distance. If linkage disequilbrium exists between two markers, then the genotypic information at one marker can be used to make predictions about the genotype of the second marker.

The invention also provides isolated nucleic acids comprising at least one polymorphic region of a PLCG1 or PAI-2 gene having a nucleotide sequence which differs from the reference nucleotide sequence set forth in SEQ ID NO:1 or SEQ ID NO:3, respectively. Preferred nucleic acids have a variant allele located in the coding region of the PLCG1 or PAI-2 gene. Accordingly, preferred nucleic acids of the invention comprise a thymidine at residue 64001 of GI 11345540 (as set forth in SEQ ID NO:1), or the complement thereof, or a cytidine at residue 170871 of GI 6705901 (as set forth in SEQ ID NO:3), or the complement thereof. Preferred nucleic acids used in combination in the methods of the invention to predict the risk of vascular diseases or disorders comprise thymidine at residue 64001 of GI 11345540 (as set forth in SEQ ID NO:1) and a thymidine at residue 170871 of GI 6705901 (as set forth in SEQ ID NO:3), or the complements thereof. Preferred nucleic acids can also have a polymorphic region in an upstream regulatory element, an exon, or in the 3′ UTR.

The nucleic acid molecules of the present invention can be single stranded DNA (e.g., an oligonucleotide), double stranded DNA (e.g., double stranded oligonucleotide) or RNA. Preferred nucleic acid molecules of the invention can be used as probes or primers. Primers of the invention refer to nucleic acids which hybridize to a nucleic acid sequence which is adjacent to the region of interest or which covers the region of interest and is extended. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning. A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least basepairs in length includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

A primer or probe can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a PLCG1 or PAI-2 gene, and which by hybridization or absence of hybridization to the DNA of a subject or the type of hybrid formed will be indicative of the identity of the allelic variant of the polymorphic region of the PLCG1 or PAI-2 gene.

Numerous procedures for determining the nucleotide sequence of a nucleic acid molecule, or for determining the presence of mutations in nucleic acid molecules include a nucleic acid amplification step, which can be carried out by, e.g., polymerase chain reaction (PCR). Accordingly, in one embodiment, the invention provides primers for amplifying portions of a PLCG1 or PAI-2 gene, such as portions of exons and/or portions of introns. In a preferred embodiment, the exons and/or sequences adjacent to the exons of the human PLCG1 or PAI-2 gene will be amplified to, e.g., detect which allelic variant, if any, of a polymorphic region is present in the PLCG1 or PAI-2 gene of a subject. Preferred primers comprise a nucleotide sequence complementary a specific allelic variant of a PLCG1 or PAI-2 polymorphic region and of sufficient length to selectively hybridize with a PLCG1 or PAI-2 gene. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a PLCG1 or PAI-2 gene. In an even more preferred embodiment, the primer is capable of hybridizing to a PLCG1 or PAI-2 nucleotide sequence, complements thereof, allelic variants thereof, or complements of allelic variants thereof. For example, primers comprising a nucleotide sequence of at least about 15 consecutive nucleotides, at least about 25 nucleotides or having from about 15 to about 20 nucleotides set forth in any of SEQ ID NOs:5 and SEQ ID NO:6 or complement thereof are provided by the invention. Primers having a sequence of more than about 25 nucleotides are also within the scope of the invention. Preferred primers of the invention are primers that can be used in PCR for amplifying each of the exons of a PLCG1 or PAI-2 gene.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to PLCG1 or PAI-2 nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of the nucleotide sequence shown in Table 3 (e.g., SEQ ID NO:5 and SEQ ID NO:6). A reverse primer can be a primer having a nucleotide sequence or a portion of the nucleotide sequence that is complementary to a nucleotide sequence shown in Table 3 (e.g., SEQ ID NO:5 and SEQ ID NO:6).

Yet other preferred primers of the invention are nucleic acids which are capable of selectively hybridizing to an allelic variant of a polymorphic region of a PLCG1 or PAI-2 gene. Thus, such primers can be specific for a PLCG1 or PAI-2 gene sequence, so long as they have a nucleotide sequence which is capable of hybridizing to a PLCG1 or PAI-2 gene. Preferred primers are capable of specifically hybridizing to any of the allelic variants listed in Table 3. Such primers can be used, e.g., in sequence specific oligonucleotide priming as described further herein.

Other preferred primers used in the methods of the invention are nucleic acids which are capable of hybridizing to the reference sequence of a PLCG1 or PAI-2 gene, thereby detecting the presence of the reference allele of an allelic variant or the absence of a variant allele of an allelic variant in the PLCG1 or PAI-2 genes. Such primers can be used in combination, e.g., primers specific for the variant polynucleotide of the PLGC1 gene and primers specific for the reference polynucleotide of the PAI-2 gene can be used in combination. The sequences of primers specific for the reference sequences comprising the PLCG1 gene or the PAI-2 gene will be readily apparent to one of skill in the art.

The PLCG1 or PAI-2 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a PLCG1 or PAI-2 gene which is polymorphic (e.g., SEQ ID NO:5 and SEQ ID NO:6). In an even more preferred embodiment of the invention, the probes are capable of hybridizing specifically to one allelic variant of a PLCG1 or PAI-2 gene having a nucleotide sequence which differs from the nucleotide sequence set forth in SEQ ID NO:1 or 3. Such probes can then be used to specifically detect which allelic variant of a polymorphic region of a PLCG1 or PAI-2 gene is present in a subject. The polymorphic region can be located in the 5′ upstream regulatory element, exon, or intron sequences of a PLCG1 or PAI-2 gene.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a PLCG1 or PAI-2 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. No. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., (1989) Proc. Natl. Acad. Sci. U.S.A. 86:6553-6556; Lemaitre et al., (1987) Proc. Natl. Acad. Sci. U.S.A. 84:648-652; PCT Publication No. WO 88/09810, published Dec. 15, 1988), hybridization-triggered cleavage agents. (See, e.g., Krol et al., (1988) BioTechniques 6:958-976) or intercalating agents (See, e.g., Zon, (1988) Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising a PLCG1 or PAI-2 intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., (1987) Nucl. Acids Res. 15:6131-6148), or a chimeric RNA-DNA analogue (Inoue et al., (1987) FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. ((1988) Nucl. Acids Res. 16:3209), methylphosphonate oligonucleotides can be prepared by use of controlled pore glass polymer supports (Sarin et al., (1988), Proc. Natl. Acad. Sci. U.S.A. 85:7448-7451), etc.

The invention also provides vectors and plasmids comprising the nucleic acids of the invention. For example, in one embodiment, the invention provides a vector comprising at least a portion of the PLCG1 gene or the PAI-2 gene comprising a polymorphic region. Thus, the invention provides vectors for expressing at least a portion of the newly identified allelic variants of the human PLCG1 gene or PAI-2 gene reference, as well as other allelic variants, comprising a nucleotide sequence which is different from the nucleotide sequence disclosed in GI 11345540 or GI 6705901, respectively. The allelic variants can be expressed in eukaryotic cells, e.g., cells of a subject, or in prokaryotic cells.

In one embodiment, the vector comprising at least a portion of a PLCG1 or PAI-2 allele is introduced into a host cell, such that a protein encoded by the allele is synthesized. The PLCG1 or PAI-2 protein produced can be used, e.g., for the production of antibodies, which can be used, e.g., in methods for detecting mutant forms of PLCG1 or PAI-2. Alternatively, the vector can be used for gene therapy, and be, e.g., introduced into a subject to produce PLCG1 or PAI-2 protein. Host cells comprising a vector having at least a portion of a PLCG1 or PAI-2 gene are also within the scope of the invention.

Polypeptides of the Invention

The present invention provides isolated PLCG1 or PAI-2 polypeptides, such as PLCG1 or PAI-2 polypeptides which are encoded by specific allelic variants of PLCG1 or PAI-2, including those identified herein. The amino acid sequences of the PLCG1 or PAI-2 proteins have been deduced. The PLCG1 gene encodes a 1,290 amino acid protein and is described in, for example, Stahl, M. L., et al. (1988) Nature 332: 269-272. The PAI-2 gene encodes a 450 amino acid protein and is described in, for example, Antalis, T. M., et al. (1988) Proc. Nat. Acad. Sci. 85: 985-989. The polymorphisms of the present invention are missense mutations which result in the change of an amino acid in the amino acid sequence of the PLCG1 gene and in the PAI-2 gene.

As shown in Table 3, one polymorphism found in the population screened is a change from a cytidine (C) to a thymidine (T) in the PLCG1 gene at residue 64001 of the reference sequence GI 11345540 (polymorphism ID No. G329u1), or the complement thereof, which results in a change from a isoleucine to a threonine in the amino acid sequence of PLCG1 (SEQ ID NO:2) at amino acid residue 813. A second polymorphism is a change from a thymidine (T) to a cytidine (C) in at residue 170871 of the reference sequence GI 6705901 (polymorphism ID No. PAI2 ul), or the complement thereof, which results in a change from an asparagine to an aspartic acid in the amino acid sequence of PAI-2 (SEQ ID NO:4) at amino acid residue 120.

In one embodiment, the PLCG1 or PAI-2 polypeptides are isolated from, or otherwise substantially free of other cellular proteins. The term “substantially free of other cellular proteins” (also referred to herein as “contaminating proteins”) or “substantially pure or purified preparations” are defined as encompassing preparations of PLCG1 or PAI-2 polypeptides having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred PLCG1 or PAI-2 proteins of the invention have an amino acid sequence which is at least about 60%, 70%, 80%, 85%, 90%, or 95% identical or homologous to the amino acid sequence of SEQ ID NO:2 or SEQ ID NO:4, respectively. Even more preferred PLCG1 or PAI-2 proteins comprise an amino acid sequence which is at least about 95%, 96%, 97%, 98%, or 99% homologous or identical to the amino acid sequence of SEQ ID NO:2 or SEQ ID NO:4, respectively. Such proteins can be recombinant proteins, and can be, e.g., produced in vitro from nucleic acids comprising a specific allele of a PLCG1 or PAI-2 polymorphic region. For example, recombinant polypeptides preferred by the present invention can be encoded by a nucleic acid which comprises a sequence which is at least 85% homologous and more preferably 90% homologous and most preferably 95% homologous with a nucleotide sequence set forth in SEQ ID NOs:1 or 3 and comprises an allele of a polymorphic region that differs from that set forth in SEQ ID NOs:1 or 3. Polypeptides which are encoded by a nucleic acid comprising a sequence that is at least about 98-99% homologous with the sequence of SEQ ID NOs:1 or 3 and comprises an allele of a polymorphic region that differs from that set forth in SEQ ID NOs:1 or 3 are also within the scope of the invention.

In a preferred embodiment, a PLCG1 or PAI-2 protein of the present invention is a mammalian PLCG1 or PAI-2 protein. In an even more preferred embodiment, the PLCG1 or PAI-2 protein is a human protein.

The invention also provides peptides that preferably are capable of functioning in one of either role of an agonist or antagonist of at least one biological activity of a reference (“normal”) PLCG1 or PAI-2 protein of the appended sequence listing. The term “evolutionarily related to,” with respect to amino acid sequences of PLCG1 or PAI-2 proteins, refers to both polypeptides having amino acid sequences found in human populations, and also to artificially produced mutational variants of human PLCG1 or PAI-2 polypeptides which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length of PLCG1 or PAI-2 protein are within the scope of the present invention.

Isolated PLCG1 or PAI-2 peptides or polypeptides can be obtained by screening peptides recombinantly produced from the corresponding fragment of the nucleic acid encoding such peptides. In addition, such peptides and polypeptides can be chemically synthesized using techniques known in the art such as conventional Merrifield solid phase f-Moc or t-Boc chemistry. For example, a PLCG1 or PAI-2 peptide or polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptides or polypeptides which can function as either agonists or antagonists of a wild-type (e.g., “normal”) PLCG1 or PAI-2 protein.

In general, peptides and polypeptides referred to herein as having an activity (e.g., are “bioactive”) of a PLCG1 or PAI-2 protein are defined as peptides and polypeptides which mimic or antagonize all or a portion of the biological/biochemical activities of a PLCG1 or PAI-2 protein having SEQ ID NO:2 or SEQ ID NO:4, respectively, such as the ability to bind ligands. Other biological activities of the subject PLCG1 or PAI-2 proteins are described herein or will be reasonably apparent to those skilled in the art. According to the present invention, a peptide or polypeptide has biological activity if it is a specific agonist or antagonist of a naturally-occurring form of a PLCG1 or PAI-2 protein.

Assays for determining whether a PLCG1 or PAI-2 protein or variant thereof, has one or more biological activities are well known in the art.

Other preferred proteins of the invention are those encoded by the nucleic acids set forth in the section pertaining to nucleic acids of the invention. In particular, the invention provides fusion proteins, e.g., PLCG1 or PAI-2-immunoglobulin fusion proteins. Such fusion proteins can provide, e.g., enhanced stability and solubility of PLCG1 or PAI-2 proteins and may thus be useful in therapy. Fusion proteins can also be used to produce an immunogenic fragment of a PLCG1 or PAI-2 protein. For example, the VP6 capsid protein of rotavirus can be used as an immunologic carrier protein for portions of the PLCG1 or PAI-2 polypeptide, either in the monomeric form or in the form of a viral particle. The nucleic acid sequences corresponding to the portion of a subject PLCG1 or PAI-2 protein to which antibodies are to be raised can be incorporated into a fusion gene construct which includes coding sequences for a late vaccinia virus structural protein to produce a set of recombinant viruses expressing fusion proteins comprising PLCG1 or PAI-2 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a PLCG1 or PAI-2 protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a PLCG1 or PAI-2 polypeptide is obtained directly from organo-chemical synthesis of the peptide onto an oligomeric branching lysine core (see, for example, Posnett et al. (1988) JBC 263:1719 and Nardelli et al. (1 992) J. Immunol. 148:914). Antigenic determinants of PLCG1 or PAI-2 proteins can also be expressed and presented by bacterial cells.

Fusion proteins can also facilitate the expression of proteins including the PLCG1 or PAI-2 polypeptides of the present invention. For example, PLCG1 or PAI-2 polypeptides can be generated as glutathione-S-transferase (GST-fusion) proteins. Such GST-fusion proteins can be easily purified, as for example by the use of glutathione-derivatized matrices (see, for example, Current Protocols in Molecular Biology, eds. Ausubel et al. (N.Y.: John Wiley & Sons, 1991)) and used subsequently to yield purified PLCG1 or PAI-2 polypeptides.

The present invention further pertains to methods of producing the subject PLCG1 or PAI-2 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant PLCG1 or PAI-2 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant PLCG1 or PAI-2 polypeptide is a fusion protein containing a domain which facilitates its purification, such as GST fusion protein.

Moreover, it will be generally appreciated that, under certain circumstances, it may be advantageous to provide homologs of one of the subject PLCG1 or PAI-2 polypeptides which function in a limited capacity as one of either a PLCG1 or PAI-2 agonist (mimetic) or a PLCG1 or PAI-2 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of PLCG1 or PAI-2 proteins.

Homologs of each of the subject PLCG1 or PAI-2 proteins can be generated by mutagenesis, such as by discrete point mutation(s), and/or by truncation. For instance, mutation can give rise to homologs which retain substantially the same, or merely a subset, of the biological activity of the PLCG1 or PAI-2 polypeptide from which it was derived. Alternatively, antagonistic forms of the protein can be generated which are able to inhibit the function of the naturally occurring form of the protein, such as by competitively binding to a PLCG1 or PAI-2 receptor.

The recombinant PLCG1 or PAI-2 polypeptides of the present invention also include homologs of PLCG1 or PAI-2 polypeptides which differ from the PLCG1 or PAI-2 protein having SEQ ID NO:2 or SEQ ID NO:4, respectively, such as versions of the protein which are resistant to proteolytic cleavage, as for example, due to mutations which alter ubiquitination or other enzymatic targeting associated with the protein.

PLCG1 or PAI-2 polypeptides may also be chemically modified to create PLCG1 or PAI-2 derivatives by forming covalent or aggregate conjugates with other chemical moieties, such as glycosyl groups, lipids, phosphate, acetyl groups and the like. Covalent derivatives of PLCG1 or PAI-2 proteins can be prepared by linking the chemical moieties to functional groups on amino acid side-chains of the protein or at the N-terminus or at the C-terminus of the polypeptide.

Modification of the structure of the subject PLCG1 or PAI-2 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the PLCG1 or PAI-2 polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e., isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur -containing=cysteine and methionine. (see, for example, Biochemistry, 2^{nd} ed., Ed. by L. Stryer, W H Freeman and Co.: 1981). Whether a change in the amino acid sequence of a peptide results in a functional PLCG1 or PAI-2 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Methods

The invention further provides predictive medicine methods, which are based, at least in part, on the discovery of PLCG1 or PAI-2 polymorphic regions which are associated with specific physiological states and/or diseases or disorders, e.g., vascular diseases or disorders such as CAD and MI. These methods can be used alone, or in combination with other predictive medicine methods, including the identification and analysis of known risk factors associated with vascular disease, e.g., phenotypic factors such as, for example, obesity, diabetes and family history.

For example, information obtained using the diagnostic assays described herein (singly or in combination with information of another genetic defect which contributes to the same disease, e.g., a vascular disease or disorder) is useful for diagnosing or confirming that a subject has an allele of a polymorphic region which is associated with a particular disease or disorder, e.g., a vascular disease or disorder. Moreover, the information obtained using the diagnostic assays described herein, singly or in combination with information of another genetic defect which contributes to the same disease, e.g., a vascular disease or disorder, can be used to predict whether or not a subject will benefit from further diagnostic evaluation for a vascular disease or disorder. Such further diagnostic evaluation includes, but is not limited to, cardiovascular imaging, such as angiography, cardiac ultrasound, coronary angiogram, magnetic resonance imagery, nuclear imaging, CT scan, myocardial perfusion imagery, or electrocardiogram, genetic analysis, e.g., identification of additional polymorphisms, e.g., which contribute to the same disease, familial health history analysis, lifestyle analysis, or exercise stress tests, either alone or in combination. Furthermore, the diagnostic information obtained using the diagnostic assays described herein (singly or in combination with information of another genetic defect which contributes to the same disease, e.g., a vascular disease or disorder), may be used to identify which subject will benefit from a particular clinical course of therapy useful for preventing, treating, ameliorating, or prolonging onset of the particular vascular disease or disorder in the particular subject. Clinical courses of therapy include, but are not limited to, administration of medication, non-surgical intervention, surgical procedure or intervention, and use of surgical and non-surgical devices used in the treatment of vascular disease, such as, for example, stents or defibrillators.

Alternatively, the information, singly, or in combination with information of another genetic defect which contributes to the same disease, e.g., a vascular disease or disorder, can be used prognostically for predicting whether a non-symptomatic subject is likely to develop a disease or condition which is associated with one or more specific alleles of PLCG1 or PAI-2 polymorphic regions in a subject. Based on the prognostic information, a health care provider can recommend a particular further diagnostic evaluation which will benefit the subject, or a particular clinical course of therapy, as described above.

In addition, knowledge of the identity of a particular PLCG1 or PAI-2 allele in a subject (the PLCG1 or PAI-2 genetic profile), singly, or preferably, in combination, allows customization of further diagnostic evaluation and/or a clinical course of therapy for a particular disease. For example, a subject's PLCG1 or PAI-2 genetic profile or the genetic profile of a disease or disorder associated with a specific allele of a PLCG1 or PAI-2 polymorphic region, e.g., a vascular disease or disorder, can enable a health care provider: 1) to more efficiently and cost-effectively identify means for further diagnostic evaluation, including, but not limited to, further genetic analysis, familial health history analysis, or use of vascular imaging devices; 2) to more effectively prescribe a drug that will address the molecular basis of the disease or condition; 3) to more efficiently and cost-effectively identify an appropriate clinical course of therapy, including, but not limited to, lifestyle changes, medications, surgical or non-surgical devices, surgical or non-surgical intervention, or any combination thereof; and 4) to better determine the appropriate dosage of a particular drug or duration of a particular course of clinical therapy. For example, the expression level of PLCG1 or PAI-2 proteins, alone or in conjunction with the expression level of other genes, known to contribute to the same disease, can be measured in many subjects at various stages of the disease to generate a transcriptional or expression profile of the disease. Expression patterns of individual subjects can then be compared to the expression profile of the disease to determine the appropriate drug, dose to administer to the subject, or course of clinical therapy.

The ability to target populations expected to show the highest clinical benefit, based on the PLCG1 or PAI-2 or disease genetic profile, can enable: 1) the repositioning of marketed drugs, surgical devices for use in treating, preventing, or ameliorating vascular diseases or disorders, or diagnostics, such as vascular imaging devices, with disappointing market results; 2) the rescue of drug candidates whose clinical development has been discontinued as a result of safety or efficacy limitations, which are subject subgroup-specific; 3) an accelerated and less costly development for drug candidates and more optimal drug labeling (e.g., since the use of PLCG1 or PAI-2 as a marker is useful for optimizing effective dose); and 4) an accelerated, less costly, and more effective selection of a particular course of clinical therapy suited to a particular subject.

These and other methods are described in further detail in the following sections.

A. Prognostic and Diagnostic Assays

The present methods provide means for determining if a subject is or is not at risk of developing a disease, condition or disorder that is associated a specific PLCG1 or PAI-2 allele, e.g., a vascular disease or a disease or disorder resulting therefrom.

The present invention provides methods for determining the molecular structure of a PLCG1 or PAI-2 gene, such as a human PLCG1 or PAI-2 gene, or a portion thereof. In one embodiment, determining the molecular structure of at least a portion of a PLCG1 or PAI-2 gene comprises determining the identity of an allelic variant of at least one polymorphic region of a PLCG1 or PAI-2 gene (determining the presence or absence of one or more of the allelic variants, or their complements, of SEQ ID NO:5 and/or SEQ ID NO:6). A polymorphic region of a PLCG1 or PAI-2 gene can be located in an exon, an intron, at an intron/exon border, or in the 5′ upstream regulatory element of the PLCG1 or PAI-2 gene.

The invention provides methods for determining whether a subject is or is not at risk of developing a disease or disorder associated with a specific allelic variant of a polymorphic region of a PLCG1 or PAI-2 gene. Such diseases can be associated with aberrant PLCG1 or PAI-2 activity, e.g., a vascular disease or disorder such as CAD or MI.

Analysis of one or more PLCG1 or PAI-2 polymorphic regions in a subject can be useful for predicting whether a subject is or is not likely to develop a vascular disease or disorder, e.g., atherosclerosis, CAD, MI, ischemia, stroke, peripheral vascular diseases, venous thromboembolism and pulmonary embolism.

In preferred embodiments, the methods of the invention can be characterized as comprising detecting, in a sample of cells from the subject, the presence or absence of a specific allelic variant of one or more polymorphic regions of a PLCG1 or PAI-2 gene. Preferably, the presence of the variant allele of the PLCG1 gene and/or the reference allele of the PAI-2 gene described herein are detected. The allelic differences can be: (i) a difference in the identity of at least one nucleotide or (ii) a difference in the number of nucleotides, which difference can be a single nucleotide or several nucleotides. The invention also provides methods for detecting differences in PLCG1 or PAI-2 genes such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a PLCG1 or PAI-2 gene prior to identifying the allelic variant. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a PLCG1 or PAI-2 gene and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biolechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster;. Cohen el al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

In some cases, the presence of a specific allele of a PLCG1 or PAI-2 gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, el al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a PLCG1 or PAI-2 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1 988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of PLCG1 or PAI-2 allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al (1979) Nucl. Acids Res. 6:3543). Such allele specific oligonucleotide hybridization techniques may be used for the simultaneous detection of several nucleotide changes in different polylmorphic regions of PLCG1 or PAI-2. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al (1 989) Nucleic Acids Res. 17:2437-2448) or at the extreme 3′ end of one primer where, under appropriate conditions, mismatch can prevent, or reduce polymerase extension (Prossner (1993) Tibiech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g.,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (USA.) 87:8923-8927. In this method, PCR is used to achieve the exponential amplification of target DNA, which is then detected using OLA.

Several techniques based on this OLA method have been developed and can be used to detect specific allelic variants of a polymorphic region of a PLCG1 or PAI-2 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a PLCG1 or PAI-2 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., (1 989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mu/at. 1:159-164; Ugozzoli, L. et al., (1992) GATA 9:107-112; Nyren, P. (1993) et al., Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the allelic variant of a polymorphic region located in the coding region of a PLCG1 or PAI-2 gene, yet other methods than those described above can be used. For example, identification of an allelic variant which encodes a mutated PLCG1 or PAI-2 protein can be performed by using an antibody specifically recognizing the mutant protein in, e.g., immunohistochemistry or immunoprecipitation. Antibodies to wild-type PAI-2 proteins are described in, for example, Tsuchiya, et al. (1995) Gen Diagnos Pathol 141(1):41. Antibodies to wild-type PLCG1 are described in, for example, Smith, et al. (1994) Proc. Natl. Acad. Sci. 91(14):6554. Other antibodies to wild-type PLCG1 or PAI-2 or mutated forms of PLCG1 or PAI-2 proteins can be prepared according to methods known in the art.

Alternatively, one can also measure an activity of a PLCG1 or PAI-2 protein, such as binding to a PLCG1 or PAI-2 ligand. Binding assays are known in the art and involve, e.g., obtaining cells from a subject, and performing binding experiments with a labeled ligand, to determine whether binding to the mutated form of the protein differs from binding to the wild-type of the protein.

Antibodies directed against reference or mutant PLCG1 or PAI-2 polypeptides or allelic variant thereof, which are discussed above, may also be used in disease diagnostics and prognostics. Such diagnostic methods, may be used to detect abnormalities in the level of PLCG1 or PAI-2 polypeptide expression, or abnormalities in the structure and/or tissue, cellular, or subcellular location of a PLCG1 or PAI-2 polypeptide. Structural differences may include, for example, differences in the size, electronegativity, or antigenicity of the mutant PLCG1 or PAI-2 polypeptide relative to the normal PLCG1 or PAI-2 polypeptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of PLCG1 or PAI-2 polypeptides. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine. not only the presence of the PLCG1 or PAI-2 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-PLCG1 or PAI-2 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., (1978) J. Clin. Pathol. 31:507-520; Butler, (1981) Meth. Enzymol. 73:482-523; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject is or is not at risk of developing a disease associated with a specific PLCG1 or PAI-2 allelic variant.

Sample nucleic acid to be analyzed by any of the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

B. Pharmacogenomics

Knowledge of the identity of the allele of one or more PLCG1 gene or PAI-2 gene polymorphic regions in a subject (the PLCG1 and/or PAI-2 genetic profile), alone or in conjunction with information of other genetic defects associated with the same disease (the genetic profile of the particular disease) also allows selection and customization of the therapy, e.g., a particular clinical course of therapy and/or further diagnostic evaluation for a particular disease to the subject's genetic profile. For example, subjects having a specific allele of a PLCG1 or PAI-2 gene, singly or in combination, may or may not exhibit symptoms of a particular disease or be predisposed to developing symptoms of a particular disease. Further, if those subjects are symptomatic, they may or may not respond to a certain drug, e.g., a specific therapeutic used in the treatment or prevention of a vascular disease or disorder, e.g., CAD or MI, such as beta blocker drugs, calcium channel blocker drugs, or nitrate drugs, but may respond to another. Furthermore, they may or may not respond to other treatments, including, for example, use of devices for treatment of vascular disease, or surgical and/or non-surgical courses of treatment. Moreover, if a subject does or does not exhibit symptoms of a particular disease, the subject may or may not benefit from further diagnostic evaluation, including, for example, use of vascular imaging devices. Thus, generation of a PLCG1 or PAI-2 genetic profile, (e.g., categorization of alterations in PLCG1 or PAI-2 genes which are associated with the development of a particular disease), from a population of subjects, who are symptomatic for a disease or condition that is caused by or contributed to by a defective and/or deficient PLCG1 or PAI-2 gene and/or protein (a PLCG1 or PAI-2 genetic population profile) and comparison of a subject's PLCG1 or PAI-2 profile to the population profile, permits the selection or design of drugs that are expected to be safe and efficacious for a particular subject or subject population (i.e., a group of subjects having the same genetic alteration), as well as the selection or design of a particular clinical course of therapy or further diagnostic evaluations that are expected to be safe and efficacious for a particular subject or subject population.

For example, a PLCG1 or PAI-2 population profile can be performed by determining the PLCG1 or PAI-2 profile, e.g., the identity of PLCG1 or PAI-2 alleles, in a subject population having a disease, which is associated with one or more specific alleles of PLCG1 or PAI-2 polymorphic regions. Optionally, the PLCG1 or PAI-2 population profile can further include information relating to the response of the population to a PLCG1 or PAI-2 therapeutic, using any of a variety of methods, including, monitoring: 1) the severity of symptoms associated with the PLCG1 or PAI-2 related disease; 2) PLCG1 or PAI-2 gene expression level; 3) PLCG1 or PAI-2 mRNA level; and/or 4) PLCG1 or PAI-2 protein level, and dividing or categorizing the population based on particular PLCG1 or PAI-2 alleles. The PLCG1 or PAI-2 genetic population profile can also, optionally, indicate those particular PLCG1 or PAI-2 alleles which are present in subjects that are either responsive or non-responsive to a particular therapeutic, clinical course of therapy, or diagnostic evaluation. This information or population profile, is then useful for predicting which individuals should respond to particular drugs, particular clinical courses of therapy, or diagnostic evaluations based on their individual PLCG1 or PAI-2 genetic profile.

In a preferred embodiment, the PLCG1 or PAI-2 profile is a transcriptional or expression level profile and is comprised of determining the expression level of PLCG1 or PAI-2 proteins, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a PLCG1 or PAI-2 gene. These mice can be created, e.g., by replacing their wild-type PLCG1 or PAI-2 gene with an allele of the human PLCG1 or PAI-2 gene. The response of these mice to specific PLCG1 or PAI-2 particular therapeutics, clinical courses of treatment, and/or diagnostic evaluations can then be determined.

(i) Diagnostic Evaluation

In one embodiment, the polymorphisms of the present invention are used to determine the most appropriate diagnostic evaluation and to determine whether or not a subject will benefit from further diagnostic evaluation. For example, if a subject has two copies of a thymidine allele at nucleotide position 170871 of the PAI-2 gene, or the complement thereof, and two copies of a thymidine allele at nucleotide position 11345540 of the PLCG1 gene, or the complement thereof, that subject is approximately 3-fold less likely to develop a vascular disease such as CAD or MI as compared to a subject having any other combination of alleles at those loci, and therefore would be less likely to require or benefit from further diagnostic evaluation for a vascular disease or disorder.

Thus, in one embodiment, the invention provides methods for classifying a subject who or is or is not at risk for developing, a vascular disease or disorder as a candidate for further diagnostic evaluation for a vascular disease or disorder comprising the steps of determining the PLCG1 and/or PAI-2 genetic profile of the subject, comparing the subject's PLCG1 and/or PAI-2 genetic profile to a PLCG1 genetic population profile and/or a PAI-2 genetic population profile, and classifying the subject based on the identified genetic profiles as a subject who is a candidate for further diagnostic evaluation for a vascular disease or disorder.

In one embodiment, the subject's PLCG1 and/or PAI-2 genetic profile is determined by identifying the nucleotide present at nucleotide position 11345540 of SEQ ID NO:1 and/or the nucleotide present at nucleotide position 170871 of SEQ ID NO:3. The subject's genetic profile can also be determined by identifying the amino acid present at amino acid position 813 of SEQ ID NO:2 and/or the amino acid residue present at position 120 of SEQ ID NO:4. Methods of further diagnostic evaluation include use of vascular imaging devices such as, for example, angiography, cardiac ultrasound, coronary angiogram, magnetic resonance imagery, nuclear imaging, CT scan, myocardial perfusion imagery, or electrocardiogram, or may include genetic analysis, familial health history analysis, lifestyle analysis, exercise stress tests, or any combination thereof.

In another embodiment, the invention provides methods for selecting an effective vascular imaging device as a diagnostic tool for a vascular disease or disorder comprising the steps of determining the PLCG1 and/or PAI-2 genetic profile of the subject; comparing the subject's PLCG1 and/or PAI-2 genetic profile to a PLCG1 genetic population profile and/or a PAI-2 genetic population profile; and selecting an effective vascular imaging device as a diagnostic tool for a vascular disease or disorder. In a preferred embodiment, the vascular imaging device is selected from the group consisting of angiography, cardiac ultrasound, coronary angiogram, magnetic resonance imagery, nuclear imaging, CT scan, myocardial perfusion imagery, electrocardiogram, or any combination thereof.

(ii) Clinical Course of Therapy

In another aspect, the polymorphisms of the present invention are used to determine the most appropriate clinical course of therapy for a subject who is at risk of a vascular disease or disorder, and will aid in the determination of whether the subject will benefit from such clinical course of therapy, as determined by identification of one, or preferably, both of the polymorphisms of the invention.

In one aspect, the invention relates to the SNPs identified as described herein, both singly and, preferably, in combination, as well as to the use of these SNPs, and others in these genes, particularly those nearby in linkage disequilibrium with these SNPs, both singly and, preferably, in combination, for prediction of a particular clinical course of therapy for a subject who has, or is or is not at risk for developing, a vascular disease. In one embodiment, the invention provides a method for determining whether a subject will or will not benefit from a particular course of therapy by determining the presence of one, or preferably both, of the identities of the polymorphisms of the invention. For example, the determination of the polymorphisms of the invention, singly, or in combination, will aid in the determination of whether an individual will benefit from surgical revascularization and/or will benefit by the implantation of a stent following surgical revascularization, and will aid in the determination of the likelihood of success or failure of a particular clinical course of therapy.

For example, if a subject has two copies of a thymidine allele at nucleotide position 170871 of the PAI-2 gene, or the complement thereof, and two copies of a thymidine allele at nucleotide position 11345540 of the PLCG1 gene, or the complement thereof, that subject is approximately 3-fold less likely to develop a vascular disease such as CAD or MI as compared to a subject having any other combination of alleles at those loci. Therefore, that subject would be less likely to require or benefit from any clinical course of therapy.

An appropriate clinical course of therapy for a vascular disease or disorder may include, for example, a lifestyle change, including, for example, a change in diet or environment. Other clinical courses of therapy include, but are not limited to, use of surgery or surgical devices. Surgical therapy for the treatment of vascular disorders, includes, for example, surgical revascularization, such as angioplasty, e.g., percutaneous transluminal coronary balloon angioplasty (PTCA), or laser angioplasty, or coronary bypass grafting (CABG). Surgical devices used in the treatment or prevention of vascular diseases or disorders, include, for example, devices used in angioplasty, such as balloon angioplasty or laser angioplasty, or implantation of a stent, or any combination thereof.

C. Monitoring Effects of PLCG1 or PAI-2 Therapeutics During Clinical Trials

The present invention provides a method for monitoring the effectiveness of treatment of a subject with an agent (e.g., an agonist, antagonist, peptidomimetic, protein, peptide, nucleic acid, small molecule, or other drug candidate identified, e.g., by the screening assays described herein) comprising the steps of (i) obtaining a preadministration sample from a subject prior to administration of the agent; (ii) detecting the level of expression or activity of a PLCG1 or PAI-2 protein, mRNA or gene in the preadministration sample; (iii) obtaining one or more post-administration samples from the subject; (iv) detecting the level of expression or activity of the PLCG1 or PAI-2 protein, mRNA or gene in the post-administration samples; (v) comparing the level of expression or activity of the PLCG1 or PAI-2 protein, mRNA, or gene in the preadministration sample with those of the PLCG1 or PAI-2 protein, mRNA, or gene in the post administration sample or samples; and (vi) altering the administration of the agent to the subject accordingly. For example, increased administration of the agent may be desirable to increase the expression or activity of PLCG1 or PAI-2 to higher levels than detected, i.e., to increase the effectiveness of the agent. Alternatively, decreased administration of the agent may be desirable to decrease expression or activity of PLCG1 or PAI-2 to lower levels than detected, i.e., to decrease the effectiveness of the agent.

Cells of a subject may also be obtained before and after administration of a PLCG1 or PAI-2 therapeutic to detect the level of expression of genes other than PLCG1 or PAI-2, to verify that the PLCG1 or PAI-2 therapeutic does not increase or decrease the expression of genes which could be deleterious. This can be done, e.g., by using the method of transcriptional profiling. Thus, MRNA from cells exposed in vivo to a PLCG1 or PAI-2 therapeutic and mRNA from the same type of cells that were not exposed to the PLCG1 or PAI-2 therapeutic could be reverse transcribed and hybridized to a chip containing DNA from numerous genes, to thereby compare the expression of genes in cells treated and not treated with a PLCG1 or PAI-2 therapeutic. If, for example a PLCG1 or PAI-2 therapeutic turns on the expression of a proto-oncogene in a subject, use of this particular PLCG1 or PAI-2 therapeutic may be undesirable.

D. Methods of Treatment

The present invention provides for both prophylactic and therapeutic methods of treating a subject having or likely to develop a disorder associated with specific PLCG1 or PAI-2 alleles and/or aberrant PLCG1 or PAI-2 expression or activity, e.g., vascular diseases or disorders.

i) Prophylactic Methods

In one aspect, the invention provides a method for preventing a disease or disorder associated with a specific PLCG1 or PAI-2 allele such as a vascular disease or disorder, e.g., CAD or MI, and medical conditions resulting therefrom, by administering to the subject an agent which counteracts the unfavorable biological effect of the specific PLCG1 or PAI-2 allele. Subjects at risk, or at a lesser than normal risk, for such a disease can be identified by a diagnostic or prognostic assay, e.g., as described herein. Administration of a prophylactic agent can occur prior to the manifestation of symptoms associated with specific PLCG1 or PAI-2 alleles, such that a disease or disorder is prevented or, alternatively, delayed in its progression. Depending on the identity of the PLCG1 or PAI-2 allele in a subject, a compound that counteracts the effect of this allele is administered. The compound can be a compound modulating the activity of PLCG1 or PAI-2, e.g., a PLCG1 or PAI-2 inhibitor.

The treatment can also be a specific lifestyle change, e.g., a change in diet or an environmental alteration. In particular, the treatment can be undertaken prophylactically, before any other symptoms are present. Such a prophylactic treatment could thus prevent the development of aberrant vascular activity, e.g., the production of atherosclerotic plaque leading to, e.g., CAD or MI. The prophylactic methods are similar to therapeutic methods of the present invention and are further discussed in the following subsections.

(ii) Therapeutic Methods

The invention further provides methods of treating a subject having a disease or disorder associated with a specific allelic variant of a polymorphic region of a PLCG1 or PAI-2 gene. Preferred diseases or disorders include vascular diseases and disorders, and disorders resulting therefrom (e.g., such as, for example, atherosclerosis, CAD, MI, ischemia, stroke, peripheral vascular diseases, venous thromboembolism and pulmonary embolism).

In one embodiment, the method comprises (a) determining the identity of an allelic variant of a PLCG1 gene, a PAI-2 gene, or preferably, the identities of both; and (b) administering to the subject a compound that compensates for the effect of the specific allelic variant(s). The polymorphic region can be localized at any location of the gene, e.g., in a regulatory element (e.g., in a 5′ upstream regulatory element), in an exon, (e.g., coding region of an exon), in an intron, or at an exon/intron border. Thus, depending on the site of the polymorphism in the PLCG1 or PAI-2 gene, a subject having a specific variant of the polymorphic region which is associated with a specific disease or condition, can be treated with compounds which specifically compensate for the effect of the allelic variant.

In a preferred embodiment, the identity of one or more of the following nucleotides of a PLCG1 or PAI-2 gene of a subject is determined: a thymidine in the PLCG1 gene at residue 64001 of the reference sequence GI 11345540 (polymorphism ID No. G523 ul), or the complement thereof, or a thymidine at residue 170871 of the reference sequence GI 6705901 (polymorphism ID No. PAI2 ul), or the complement thereof. In a preferred embodiment, the identities of both nucleotides is determined.

If a subject has two copies of the variant allele of the PLCG1 gene (e.g., thymidine in the PLCG1 gene at residue 64001 of the reference sequence GI 11345540), or the complement thereof, and two copies of the reference allele of the PAI-2 gene (e.g., thymidine at residue 170871 of the reference sequence GI 6705901), or the complement thereof, as set forth in Table 3, that subject is at a lesser than normal risk of developing a vascular disease such as CAD or MI.

Generally, the allelic variant can be a mutant allele, i.e., an allele which when present in one, or preferably two copies, in a subject results in a change in the phenotype of the subject. A mutation can be a substitution, deletion, and/or addition of at least one nucleotide relative to the wild-type allele (i.e., the reference sequence). Depending on where the mutation is located in the PLCG1 or PAI-2 gene, the subject can be treated to specifically compensate for the mutation. For example, if the mutation is present in the coding region of the gene and results in a more active PLCG1 or PAI-2 protein, the subject can be treated, e.g., by administration to the subject of a medication or course of clinical treatment which treat, prevents, or ameliorates a vascular disease or disorder. Normal PLCG1 or PAI-2 protein can also be used to counteract or compensate for the endogenous mutated form of the PLCG1 or PAI-2 protein. Normal PLCG1 or PAI-2 protein can be directly delivered to the subject or indirectly by gene therapy wherein some cells in the subject are transformed or transfected with an expression construct encoding wild-type PLCG1 or PAI-2 protein. Nucleic acids encoding reference human PLCG1 or PAI-2 protein are set forth in SEQ ID NOs.:1 and 3, respectively (GI Accession Nos. 11345540 and 6705901).

Yet in another embodiment, the invention provides methods for treating a subject having a mutated PLCG1 or PAI-2 gene, in which the mutation is located in a regulatory region of the gene. Such a regulatory region can be localized in the 5′ upstream regulatory element of the gene, in the 5′ or 3′ untranslated region of an exon, or in an intron. A mutation in a regulatory region can result in increased production of PLCG1 or PAI-2 protein, decreased production of PLCG1 or PAI-2 protein, or production of PLCG1 or PAI-2 having an aberrant tissue distribution. The effect of a mutation in a regulatory region upon the PLCG1 or PAI-2 protein can be determined, e.g., by measuring the PLCG1 or PAI-2 protein level or mRNA level in cells having a PLCG1 or PAI-2 gene having this mutation and which, normally (i.e., in the absence of the mutation) produce PLCG1 or PAI-2 protein. The effect of a mutation can also be determined in vitro. For example, if the mutation is in the 5′ upstream regulatory element, a reporter construct can be constructed which comprises the mutated 5′ upstream regulatory element linked to a reporter gene, the construct transfected into cells, and comparison of the level of expression of the reporter gene under the control of the mutated 5′ upstream regulatory element and under the control of a wild-type 5′ upstream regulatory element. Such experiments can also be carried out in mice transgenic for the mutated 5′ upstream regulatory element. If the mutation is located in an intron, the effect of the mutation can be determined, e.g., by producing transgenic animals in which the mutated PLCG1 or PAI-2 gene has been introduced and in which the wild-type gene may have been knocked out. Comparison of the level of expression of PLCG1 or PAI-2 in the mice transgenic for the mutant human PLCG1 or PAI-2 gene with mice transgenic for a wild-type human PLCG1 or PAI-2 gene will reveal whether the mutation results in increased, or decreased synthesis of the PLCG1 or PAI-2 protein and/or aberrant tissue distribution of PLCG1 or PAI-2 protein. Such analysis could also be performed in cultured cells, in which the human mutant PLCG1 or PAI-2 gene is introduced and, e.g., replaces the endogenous wild-type PLCG1 or PAI-2 gene in the cell. Thus, depending on the effect of the mutation in a regulatory region of a PLCG1 or PAI-2 gene, a specific treatment can be administered to a subject having such a mutation. Accordingly, if the mutation results in increased PLCG1 or PAI-2 protein levels, the subject can be treated by administration of a compound which reduces PLCG1 or PAI-2 protein production, e.g., by reducing PLCG1 or PAI-2 gene expression or a compound which inhibits or reduces the activity of PLCG1 or PAI-2.

A correlation between drug responses and specific alleles of PLCG1 or PAI-2 can be shown, for example, by clinical studies wherein the response to specific drugs of subjects having different allelic variants of a polymorphic region of a PLCG1 or PAI-2 gene is compared. Such studies can also be performed using animal models, such as mice having various alleles of human PLCG1 or PAI-2 genes and in which, e.g., the endogenous PLCG1 or PAI-2 has been inactivated such as by a knock-out mutation. Test drugs are then administered to the mice having different human PLCG1 or PAI-2 alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Other Uses For the Nucleic Acid Molecules of the Invention

The identification of different alleles of PLCG1 or PAI-2 can also be useful for identifying an individual among other individuals from the same species. For example, DNA sequences can be used as a fingerprint for detection of different individuals within the same species (Thompson, J. S. and Thompson, eds., Genetics in Medicine, W B Saunders Co., Philadelphia, Pa. (1991)). This is useful, for example, in forensic studies and paternity testing, as described below.

A. Forensics

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of polymorphic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−X)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=x⁴ +(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus: cum p(ID)=p(ID1)p(ID2)p(ID3) . . . p(1Dn).

The cumulative probability of non-identity for n loci (i.e., the probability that two random individuals will be difference at 1 or more loci) is given by-the equation: cum p(nonID)=1-cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the putative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father ) is given by the equation (see WO 95/12607): p(exc)=xy(1−xy), where x and y are the population frequencies of alleles A and B of a biallelic polymorphic site.

(At a triallelic site p(exc)=xy(1−xy)+yz(1−yz)+xz(1−xz)+3xyz(1−xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non-exc)=p(non-exc1)p(non-exc2)p(non-exc3) . . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

C. Kits As set forth herein, the invention provides methods, e.g., diagnostic and therapeutic methods, e.g., for determining the type of allelic variant of a polymorphic region present in a PLCG1 or PAI-2 gene, such as a human PLCG1 or PAI-2 gene. In preferred embodiments, the methods use probes or primers comprising nucleotide sequences which are complementary polymorphic region of a PLCG1 or PAI-2 gene (SEQ ID NOs:5 and SEQ ID NO:6). Accordingly, the invention provides kits for performing these methods.

In a preferred embodiment, the invention provides a kit for determining whether a subject is or is not at risk of developing a disease or condition associated with a specific allelic variant of a PLCG1 or PAI-2 polymorphic region. In an even more preferred embodiment, the disease or disorder is characterized by an abnormal PLCG1 or PAI-2 activity. In an even more preferred embodiment, the invention provides a kit for determining whether a subject is or is not at risk of developing a vascular disease, e.g., atherosclerosis, CAD, MI, ischemia, stroke, peripheral vascular diseases, venous thromboembolism and pulmonary embolism.

A preferred kit provides reagents for determining whether a subject is or is not likely to develop a vascular disease, e.g., CAD or MI.

Preferred kits comprise at least one probe or primer which is capable of specifically hybridizing under stringent conditions to a PLCG1 or PAI-2 reference sequence or polymorphic region and instructions for use. The kits preferably comprise at least one of the above described nucleic acids. Preferred kits for amplifying at least a portion of a PLCG1 or PAI-2 gene, comprise at least two primer pairs, at least one of which is capable of hybridizing to an allelic variant sequence of a PLCG1 gene (e.g., thymidine in the PLCG1 gene at residue 64001 of the reference sequence GI 11345540, or the complement thereof) and one of which is capable of hybridizing to a reference sequence of a PAI-2 gene (e.g., thymidine at residue 170871 of the reference sequence GI 6705901, or the complement thereof).

The kits of the invention can also comprise one or more control nucleic acids or reference nucleic acids, such as nucleic acids comprising a PLCG1 or PAI-2 intronic sequence. For example, a kit can comprise primers for amplifying a polymorphic region of a PLCG1 or PAI-2 gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a PLCG1 or PAI-2 gene of an individual who does not have a vascular disease, or a disease or disorder associated with an aberrant PLCG1 or PAI-2 activity.

Yet other kits of the invention comprise at least one reagent necessary to perform the assay. For example, the kit can comprise an enzyme. Alternatively the kit can comprise a buffer or any other necessary reagent.

D. Electronic Apparatus Readable Media and Arrays

Electronic apparatus readable media comprising polymorphisms of the present invention is also provided. As used herein, “electronic apparatus readable media” and “computer readable media,” which are used interchangeably herein, refer to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the polymorphisms of the present invention.

A variety of software programs and formats can be used to store the polymorphisms information of the present invention on the electronic apparatus readable medium. For example, the polymorphic sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the markers of the present invention.

By providing the polymorphisms of the invention in readable form, singly or in combination, one can routinely access the polymorphism information for a variety of purposes. For example, one skilled in the art can use the sequences of the polymorphisms of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a vascular disease or a pre-disposition to a vascular disease, wherein the method comprises the steps of determining the presence or absence of a polymorphism and based on the presence or absence of the polymorphism, determining whether the subject has a vascular disease or a pre-disposition to a vascular disease and/or recommending a particular clinical course of therapy or diagnostic evaluation for the vascular disease or pre-vascular disease condition.

The present invention further provides in an electronic system and/or in a network, a method for determining whether a subject has a vascular disease or a pre-disposition to vascular disease associated with a polymorphism as described herein wherein the method comprises the steps of determining the presence or absence of the polymorphism, and based on the presence or absence of the polymorphism, determining whether the subject has a vascular disease or a pre-disposition to a vascular disease, and/or recommending a particular treatment for the vascular disease or pre-vascular disease condition. The method may further comprise the step of receiving phenotypic information associated with the subject and/or acquiring from a network phenotypic information associated with the subject .

The present invention also provides in a network, a method for determining whether a subject has vascular disease or a pre-disposition to vascular disease associated with a polymorphism, said method comprising the steps of receiving information associated with the polymorphism, receiving phenotypic information associated with the subject, acquiring information from the network corresponding to the polymorphism and/or vascular disease, and based on one or more of the phenotypic information, the polymorphism, and the acquired information, determining whether the subject has a vascular disease or a pre-disposition to a vascular disease. The method may further comprise the step of recommending a particular treatment for the vascular disease or pre-vascular disease condition.

The present invention also provides a method for determining whether a subject has a vascular disease or a pre-disposition to a vascular disease, said method comprising the steps of receiving information associated with the polymorphism, receiving phenotypic information associated with the subject, acquiring information from the network corresponding to the polymorphism and/or vascular disease, and based on one or more of the phenotypic information, the polymorphism, and the acquired information, determining whether the subject has vascular disease or a pre-disposition to vascular disease. The method may further comprise the step of recommending a particular treatment for the vascular disease or pre-vascular disease condition.

E. Personalized Health Assessment

Methods and systems of assessing personal health and risk for disease, e.g., vascular disease, in a subject, using the polymorphisms and association of the instant invention are also provided. The methods provide personalized health care knowledge to individuals as well as to their health care providers, as well as to health care companies. It will be appreciated that the term “health care providers” is not limited to physicians but can be any source of health care. The methods and systems provide personalized information including a personal health assessment report that can include a personalized molecular profile, e.g., an PLCG1 and/or PAI-2 genetic profile, a health profile, or both. Overall, the methods and systems as described herein provide personalized information for individuals and patient management tools for healthcare providers and/or subjects using a variety of communications networks such as, for example, the Internet. U.S. patent application Ser. No. 60/266,082, filed Feb. 1, 2001, entitled “Methods and Systems for Personalized Health Assessment,” further describes personalized health assessment methods, systems, and apparatus, and is expressly incorporated herein by reference.

In one aspect, the invention provides an Internet-based method for assessing a subject's risk for vascular disease, e.g., CAD or MI. In one embodiment, the method comprises obtaining a biological sample from a subject, analyzing the biological sample to determine the presence or absence of a polymorphic region of PLCG1 and/or PAI-2, and providing results of the analysis to the subject via the Internet, wherein the presence of a polymorphic region of PLCG1 and/or PAI-2 indicates a decreased risk for vascular disease. In another embodiment, the method comprises analyzing data from a biological sample from a subject relating to the presence or absence of a polymorphic region of PLCG1 and/or PAI-2 and providing results of the analysis to the subject via the Internet, wherein the presence of a polymorphic region of PLCG1 and/or PAI-2 indicates a or decreased risk for vascular disease.

It will be appreciated that the phrase “wherein the presence of a polymorphic region of PLCG1 and/or PAI-2 indicates a decreased risk for vascular disease” includes a decreased or lower than normal risk of developing a vascular disease indicated the presence of two copies of a thymidine allele at nucleotide position 170871 of the PAI-2 gene together with two copies of a thymidine allele at nucleotide position 64001 of the PLCG1 gene, or the complements thereof, or the presence of a threonine at amino acid position 813 of the PLCG1 protein and the presence an asparagine at amino acid position 120 of the PAI-2 protein.

The terms “Internet” and/or “communications network” as used herein refer to any suitable communication link, which permits electronic communications. It should be understood that these terms are not limited to “the Internet” or any other particular system or type of communication link. That is, the terms “Internet” and/or “communications network” refer to any suitable communication system, including extra-computer system and intra-computer system communications. Examples of such communication systems include internal busses, local area networks, wide area networks, point-to-point shared and dedicated communications, infra-red links, microwave links, telephone links, CATV links, satellite and radio links, and fiber-optic links. The terms “Internet” and/or “communications network” can also refer to any suitable communications system for sending messages between remote locations, directly or via a third party communication provider such as AT&T. In this instance, messages can be communicated via telephone or facsimile or computer synthesized voice telephone messages with or without voice or tone recognition, or any other suitable communications technique.

In another aspect, the methods of the invention also provide methods of assessing a subject's risk for vascular disease, e.g., CAD or MI. In one embodiment, the method comprises obtaining information from the subject regarding the polymorphic region of an PLCG1 and/or PAI-2 gene, through e.g., obtaining a biological sample from the individual, analyzing the sample to obtain the subject's PLCG1 and/or PAI-2 genetic profile, representing the PLCG1 and/or PAI-2 genetic profile information as digital genetic profile data, electronically processing the PLCG1 and/or PAI-2 digital genetic profile data to generate a risk assessment report for vascular disease, and displaying the risk assessment report on an output device, where the presence of a polymorphic region of PLCG1 and/or PAI-2 indicates a decreased risk for vascular disease. In another embodiment, the method comprises analyzing a subject's PLCG1 and/or PAI-2 genetic profile, representing the PLCG1 and/or PAI-2 genetic profile information as digital genetic profile data, electronically processing the PLCG1 and/or PAI-2 digital genetic profile data to generate a risk assessment report for vascular disease, and displaying the risk assessment report on an output device, where the presence of a polymorphic region of PLCG1 and/or PAI-2 indicates a decreased risk for vascular disease, e.g., CAD or MI. Additional health information may be provided and can be utilized to generate the risk assessment report. Such information includes, but is not limited to, information regarding one or more of age, sex, ethnic origin, diet, sibling health, parental health, clinical symptoms, personal health history, blood test data, weight, and alcohol use, drug use, nicotine use, and blood pressure.

The PLCG1 and/or PAI-2 digital genetic profile data may be transmitted via a communications network, e.g., the Internet, to a medical information system for processing.

In yet another aspect the invention provides a medical information system for assessing a subject's risk for vascular disease comprising a means for obtaining information from the subject regarding the polymorphic region of an PLCG1 and/or PAI-2 gene, through e.g., obtaining a biological sample from the individual to obtain an PLCG1 and/or PAI-2 genetic profile, a means for representing the PLCG1 and/or PAI-2 genetic profile as digital molecular data, a means for electronically processing the PLCG1 and/or PAI-2 digital genetic profile to generate a risk assessment report for vascular disease, and a means for displaying the risk assessment report on an output device, where the presence of a polymorphic region of PLCG1 and/or PAI-2 indicates a decreased risk for vascular disease.

In another aspect, the invention provides a computerized method of providing medical advice to a subject comprising obtaining information from the subject regarding the polymorphic region of an PLCG1 and/or PAI-2 gene, through e.g., obtaining a biological sample from the subject, analyzing the subject's biological sample to determine the subject's PLCG1 and/or PAI-2 genetic profile, and, based on the subject's PLCG1 and/or PAI-2 genetic profile, determining the subject's risk for vascular disease. Medical advice may be then provided electronically to the subject, based on the subject's risk for vascular disease. The medical advice may comprise, for example, recommending one or more of the group consisting of: further diagnostic evaluation, use of medical or surgical devices, administration of medication, or lifestyle change. Additional health information may also be obtained from the subject and may also be used to provide the medical advice.

In another aspect, the invention includes a method for self-assessing risk for a vascular disease. The method comprises providing information from the subject regarding the polymorphic region of an PLCG1 and/or PAI-2 gene, through e.g., providing a biological sample for genetic analysis, and accessing an electronic output device displaying results of the genetic analysis, thereby self-assessing risk for a vascular disease, where the presence of a polymorphic region of PLCG1 and/or PAI-2 indicates a decreased risk for vascular disease.

In another aspect, the invention provides a method of self-assessing risk for vascular disease comprising providing information from the subject regarding the polymorphic region of an PLCG1 and/or PAI-2 gene, through e.g., providing a biological sample, accessing PLCG1 and/or PAI-2 digital genetic profile data obtained from the biological sample, the PLCG1 and/or PAI-2 digital genetic profile data being displayed via an output device, where the presence of a polymorphic region of PLCG1 and/or PAI-2 indicates a decreased risk for vascular disease.

An output device may be, for example, a CRT, printer, or website. An electronic output device may be accessed via the Internet.

The biological sample may be obtained from the individual at a laboratory company. In one embodiment, the laboratory company processes the biological sample to obtain PLCG1 and/or PAI-2 genetic profile data, represents at least some of the PLCG1 and/or PAI-2 genetic profile data as digital genetic profile data, and transmits the PLCG1 and/or PAI-2 digital genetic profile data via a communications network to a medical information system for processing. The biological sample may also be obtained from the subject at a draw station. A draw station processes the biological sample to obtain PLCG1 and/or PAI-2 genetic profile data and transfers the data to a laboratory company. The laboratory company then represents at least some of the PLCG1 and/or PAI-2 genetic profile data as digital genetic profile data, and transmits the PLCG1 and/or PAI-2 digital genetic profile data via a communications network to a medical information system for processing.

In another aspect, the invention provides a method for a health care provider to generate a personal health assessment report for an individual. The method comprises counseling the individual to provide a biological sample and authorizing a draw station to take a biological sample from the individual and transmit molecular information from the sample to a laboratory company, where the molecular information comprises the presence or absence of a polymorphic region of PLCG1 and/or PAI-2. The health care provider then requests the laboratory company to provide digital molecular data corresponding to the molecular information to a medical information system to electronically process the digital molecular data and digital health data obtained from the individual to generate a health assessment report, receives the health assessment report from the medical information system, and provides the health assessment report to the individual.

In still another aspect, the invention provides a method of assessing the health of an individual. The method comprises obtaining health information from the individual using an input device (e.g., a keyboard, touch screen, hand-held device, telephone, wireless input device, or interactive page on a website), representing at least some of the health information as digital health data, obtaining a biological sample from the individual, and processing the biological sample to obtain molecular information, where the molecular information comprises the presence or absence of a polymorphic region of PLCG1 and/or PAI-2. At least some of the molecular information and health data is then presented as digital molecular data and electronically processed to generate a health assessment report. The health assessment report is then displayed on an output device. The health assessment report can comprise a digital health profile of the individual. The molecular data can comprise protein sequence data, and the molecular profile can comprise a proteomic profile. The molecular data can also comprise information regarding one or more of the absence, presence, or level, of one or more specific proteins, polypeptides, chemicals, cells, organisms, or compounds in the individual's biological sample. The molecular data may also comprise, e.g., nucleic acid sequence data, and the molecular profile may comprise, e.g., a genetic profile.

In yet another embodiment, the method of assessing the health of an individual further comprises obtaining a second biological sample or a second health information at a time after obtaining the initial biological sample or initial health information, processing the second biological sample to obtain second molecular information, processing the second health information, representing at least some of the second molecular information as digital second molecular data and second health information as digital health information, and processing the molecular data and second molecular data and health information and second health information to generate a health assessment report. In one embodiment, the health assessment report provides information about the individual's predisposition for vascular disease, e.g., CAD or MI, and options for risk reduction.

Options for risk reduction comprise, for example, one or more of diet, exercise, one or more vitamins, one or more drugs, cessation of nicotine use, and cessation of alcohol use. wherein the health assessment report provides information about treatment options for a particular disorder. Treatment options comprise, for example, one or more of diet, one or more drugs, physical therapy, and surgery. In one embodiment, the health assessment report provides information about the efficacy of a particular treatment regimen and options for therapy adjustment.

In another embodiment, electronically processing the digital molecular data and digital health data to generate a health assessment report comprises using the digital molecular data and/or digital health data as inputs for an algorithm or a rule-based system that determines whether the individual is at risk for a specific disorder, e.g., a vascular disorder, such as CAD or MI. Electronically processing the digital molecular data and digital health data may also comprise using the digital molecular data and digital health data as inputs for an algorithm or a rule-based system based on one or more databases comprising stored digital molecular data and/or digital health data relating to one or more disorders, e.g., vascular disorders, such as CAD or MI.

In another embodiment, processing the digital molecular data and digital health data comprises using the digital molecular data and digital health data as inputs for an algorithm or a rule-based system based on one or more databases comprising: (i) stored digital molecular data and/or digital health data from a plurality of healthy individuals, and (ii) stored digital molecular data and/or digital health data from one or more pluralities of unhealthy individuals, each plurality of individuals having a specific disorder. At least one of the databases can be a public database. In one embodiment, the digital health data and digital molecular data are transmitted via, e.g., a communications network, e.g., the Internet, to a medical information system for processing.

A database of stored molecular data and health data, e.g., stored digital molecular data and/or digital health data, from a plurality of individuals, is further provided. A database of stored digital molecular data and/or digital health data from a plurality of healthy individuals, and stored digital molecular data and/or digital health data from one or more pluralities of unhealthy individuals, each plurality of individuals having a specific disorder, e.g., a vascular disorder, is also provided.

The new methods and systems of the invention provide healthcare providers with access to ever-growing relational databases that include both molecular data and health data that is linked to specific disorders, e.g., vascular disorders. In addition public medical knowledge is screened and abstracted to provide concise, accurate information that is added to the database on an ongoing basis. In addition, new relationships between particular SNPs, e.g., SNPs associated with vascular disease, or genetic mutations and specific discords are added as they are discovered.

The present invention is further illustrated by the following examples which should not be construed as limiting in any way. The contents of all cited references (including, without limitation, literature references, issued patents, published patent applications and database records including Genbank™ records) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., NY); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### Detection of Polymorphic Regions in the Human PLCG1 and PAI-2 Genes

This example describes the detection of polymorphic regions in the human PLCG1 and PAI-2 genes through use of denaturing high performance liquid chromatography (DHPLC), variant detector arrays, polymerase chain reaction (PCR), and direct sequencing.

Cell lines derived from an ethnically diverse population were obtained and used for single nucleotide polymorphism (SNP) discovery by methods described in Cargill, et al. (1999) Nature Genetics 22:231-238, incorporated herein in its entirety by reference.

Genomic sequence representing the coding and partial regulatory regions of genes were amplified by polymerase chain reaction and screened via two independent methods: denaturing high performance liquid chromatography (DHPLC) or variant detector arrays (Affymetrix™).

DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266).

Generally, the analysis was carried out as described in O'Donovan et al. ((1998) Genomics 52:44-49). PCR products having product sizes ranging from about 150-400 bp were generated. Two PCR reactions were pooled together for DHPLC analysis (4 ul of each reaction for a total of 8 ul per sample). DHPLC was performed on a DHPLC system purchased from Transgenomic, Inc. The gradient was created by mixing buffers A (0.1 M TEAA) and B (0.1 M TEAA, 25% Acetontitrile). WAVEmaker™ software was utilized to predict a melting temperature and calculate a buffer gradient for mutation analysis of a given DNA sequence. The resulting chromatograms were analyzed to identify base pair alterations or deletions based on specific chromatographic profiles.

Detection of Polymorphic Regions in the Human PLCG1 and PAI-2 genes by SSCP

Genomic DNA from the cell lines derived from an ethnically diverse population as described in Cargill, et al. (1999) Nature Genetics 22:231-238, was subjected to PCR in 25 μl reactions (1×PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the above described pairs of primers under the following cycle conditions: 94° C. for 2 min, 35×[94° C. for 40 sec, 57° C. for 30 sec, 72° C. for 1 min], 72° C. 5 min, 4° C. hold.

The amplified genomic DNA fragments were then analyzed by SSCP (Orita et al. (1989) PNAS USA 86:2766, see also Cotton (1993) Mutat Res 285:125-144; and Hayashi (1992) Genet Anal Tech Appl 9:73-79). From each 25 μl PCR reaction, 3 μl was taken and added to 7 μl of loading buffer. The mixture was heated to 94° C. for 5 min and then immediately cooled in a slurry of ice-water. 3-4 μl were then loaded on a 10% polyacrylamide gel either with 10% glycerol or without 10% glycerol, and then subjected to electrophoresis either overnight at 4 Watts at room temperature, overnight at 4 Watts at 4° C. (for amplifying a 5′ upstream regulatory element), or for 5 hours at 20 Watts at 4° C. The secondary structure of single-stranded nucleic acids varies according to sequence, thus allowing the detection of small differences in nucleic acid sequence between similar nucleic acids. At the end of the electrophoretic period, the DNA was analyzed by gently overlaying a mixture of dyes onto the gel (1× the manufacturer's recommended concentration of SYBR Green I™ and SYBR Green II™ in 0.5×TBE buffer (Molecular Probes™)) for 5 min, followed by rinsing in distilled water and detection in a Fluoroimager 575™ (Molecular Dynamics™).

Sequencing of PCR Products

To determine the sequences of the polymorphisms identified, the regions containing the polymorphisms were reamplified using flanking primers. The genomic DNA was subjected to PCR in 50 μl reactions (1× PCR Amplitaq polymerase buffer, 0.1 mM dNTPs, 0.8 μM 5′ primer, 0.8 μM 3′ primer, 0.75 units of Amplitaq polymerase, 50 ng genomic DNA) using each of the pairs of primers under the following cycle conditions: 94° C. for 2 min, 35×[94° C. for 40 sec, 57° C. for 30 sec, 72° C. for 1 min], 72° C. min, 4° C. hold. The newly amplified products were then purified using the Qiagen Qiaquick PCR purification kit according to the manufacturer's protocol, and subjected to sequencing using the aforementioned primers which were utilized for amplification.

Results

Several SNPs in each of the PLCG1 and PAI2 genes were identified. Two SNPs in the PLCG1 gene and four SNPs in the PAI2 gene were selected for further analysis. The two SNPs in the PLCG1 gene were in strong linkage disequilibrium with each other (p<0.0001). The four SNPs in the PAI-2 gene were in strong linkage disequilibrium with each other (all pairwise p values<0.0001). Table 1 lists all of the SNPs analyzed in the PLCG1 gene and PAI-2 gene. Table 2 shows the measure of linkage disequilibrium between pairs of SNPs in each gene.

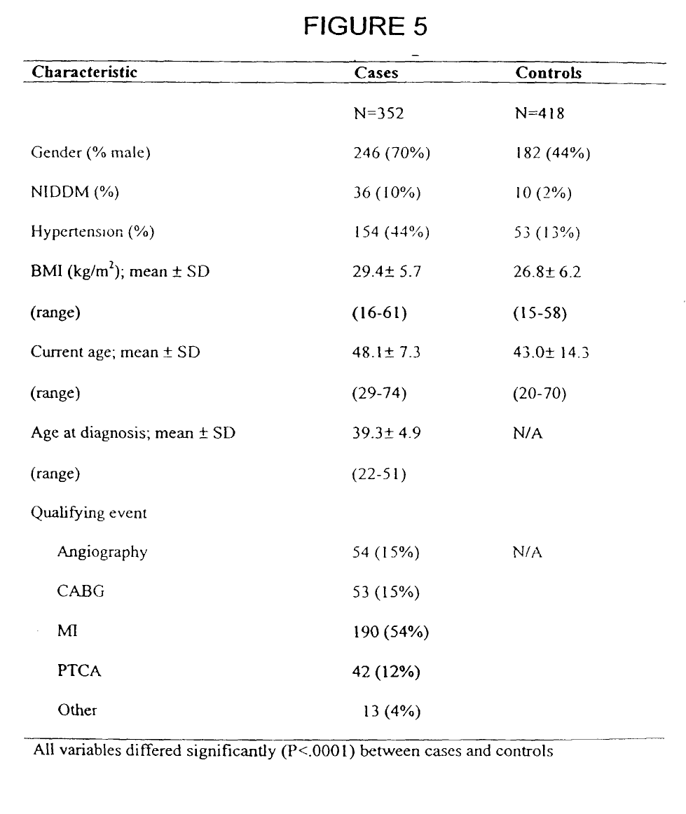

Further analysis of the PLCG1 and PAI-2 SNPs included genotyping of the SNPs in large patient populations to assess their association with CAD and MI. A total of 352 U.S. Caucasian subjects with premature coronary artery disease were identified in 15 participating medical centers, fulfilling the criteria of either myocardial infarction, surgical or percutaneous revascularization, or a significant coronary artery lesion (e.g., at least a 70% stenosis in a major epicardial artery) diagnosed before age 45 in men or age 50 in women and having a living sibling who met the same criteria. The sibling with the earliest onset in a Caucasian subset of these families was compared with a random sample of 418 Caucasian controls without known coronary disease. Controls representing a general, unselected population were identified through random-digit dialing in the Atlanta, Georgia area. Subjects ranging in age from age 20 to age 70 were invited to participate in the study. The subjects answered a health questionnaire, had anthropometric measures taken, and blood drawn for measurement of serum markers and extraction of DNA. Demographic characteristics are shown in FIG. 5.

TABLE 1

| | | | | | | |
|---|---|---|---|---|---|---|
| | | | | | GI number/ | |
| | | | Amino | Flanking | nucleotide | |
| Gene | SNP | nt. change | acid change | sequence | (nt) position | SEQ ID NO: |

| | | | | | | |
|---|---|---|---|---|---|---|
| PLCG1 | G329u1 | c/t | I/T | ACGAGCTGA | GI 11345540 | 5 |
| | | | | CCTTCAcCAA | nt 64001 | |
| | | | | GAGCGCCAT | | |
| | | | | CAT | | |
| PLCG1 | G329u3 | a/g | S/G | CAGGAGTTC | GI 11345540 | 7 |
| | | | | ATGCTCgGCT | nt 58599 | |
| | | | | TCCTCCGAG | | |
| | | | | ACC | | |
| PAI-2 | PAI2u1 | t/c | N/D | AAATAATcC | GI 6705901 | 6 |
| | | | | CCTGTGGA | nt 170871 | |
| PAI-2 | PAI2u5 | c/g | S/C | TTTTCGGCA | GI 6705901 | 8 |
| | | | | GATTTTgCTC | nt 164736 | |
| | | | | ACCCTAAAA | | |
| | | | | CTA | | |
| PAI-2 | PAI2u4 | c/g | N/K | GCATAAGAT | GI 6705901 | 9 |
| | | | | AACCAAgTG | nt 164762 | |
| | | | | CATTTTATTT | | |
| | | | | TTC | | |
| PAI-2 | PAI2d17 | c/g | non-coding | TGTTTTTTTC | GI 6705901 | 10 |
| | | | | TTCCTgTCTT | nt 176579 | |
| | | | | TGCTTCTAG | | |
| | | | | AT | | |

TABLE 2

| Gene | SNP1 | SNP2 | D' | P value |
|---|---|---|---|---|
| PLCG1 | G329u1 | G329u3 | .96 | <.0001 |
| PAI2 | PAI2u5 | PAI2u4 | 1.00 | <.0001 |
| PAI2 | PAI2u5 | PAI2u1 | .99 | <.0001 |
| PAI2 | PAI2u5 | PAI2d17 | .80 | <.0001 |
| PAI2 | PAI2u4 | PAI2u1 | .99 | <.0001 |
| PAI2 | PAI2u4 | PAI2d17 | .80 | <.0001 |
| PAI2 | PAI2u1 | PAI2d17 | .77 | <.0001 |

One SNP from each of the PLCG1 and PAI-2 genes showed strong associations with CAD and/or MI. These SNPs were a change from a cytidine (C) to a thymidine (T) in the PLCG1 gene at residue 64001 of the reference sequence GI 11345540 (polymorphism ID No. G329 ul) and a change from a thymidine (T) to a cytidine (C) in the PAI-2 gene at residue 170871 of the reference sequence GI 6705901 (polymorphism ID No. PAI2 ul) (see Table 3, below). Because other SNPs in PLCG1 and PA12 have been demonstrated to be in strong linkage disequilibrium with these specific SNPs, G329 ul and PAI2 ul, these other SNPs could be used as surrogates, e.g., markers, of G329 ul and PAI2 ul to predict risk of CAD and/or MI in a subject.

TABLE 3

| | | | | | | | Genbank | | |
|---|---|---|---|---|---|---|---|---|---|
| | | var | Type of | | | | Accession/nt | Flanking | |
| Gene | PolyID | freq | variant | Genotypes | Ref | Var | position | Sequence | SEQ ID NO. |
| 1 | 2 | 3 | 4 | 5 | 6 | 7 | 8 | 9 | 10 |

| | | | | | | | | | |
|---|---|---|---|---|---|---|---|---|---|
| PLCG1 | G329u1 | .25 | Missense | TT | C | T | GI:11345540/nt | GACCTTCA | 5 |
| | | | (I/T) | TC | | | 64001 | tCAAGAGC | |
| | | | | CC | | | | G | |
| PAI2 | PAI2u1 | .22 | Missense | CC | T | C | GI:6705901 nt | AAATAATc | 6 |
| | | | (N/D) | CT | | | 170871 | CCCTGTG | |
| | | | | TT | | | | GA | |

### EXAMPLE 2

### Statistical Analysis

All analyses were done using the SAS statistical package (Version 8.0, SAS Institute Inc., Cary, N.C.). Differences between cases and controls were assessed with a chi-square statistic for categorical covariates and the Wilcoxon statistic for continuous covariates. Association between each SNP and two outcomes, CAD and MI, was measured by comparing genotype frequencies between controls and all CAD cases and the subset of cases with MI. Significance was determined using a continuity-adjusted chi-square or Fisher's exact test for each genotype compared to the homozygotes wild-type for that locus. Odds ratios were calculated and presented with 95% confidence intervals.

Genotype groups were pooled for subsequence analysis of the top loci. Pooling allows the best model for each locus (dominant, codominant, or recessive) to be tested. Models were chosen based on significant differences between genotypes within a locus. A recessive model was chosen when the homozygous variant differed significantly from both the heterozygous and homozygous wildtype, and the latter two did not differ from each other. A codominant model was chosen when homozygous variant genotypes differed from both heterozygous and homozygous wild-type, and the latter two differed significantly from each other. A dominant model was chosen when no significant difference was observed between heterozygous and homozygous variant genotypes.

Multivariate logistic regression was used to adjust for sex, presence of hypertension, diabetes, and body mass index using the LOGISTC procedure in SAS. Height and weight, measured at the time of enrollment, were used to calculate body mass index for each subject. Presence of hypertension and non-insulin-dependent diabetes was measures by self-report (controls) and medical record confirmation (cases).

Two SNPs, one from the PLCG1 gene and one from the PAI-2 gene showed statistically significant differences from cases and controls for CAD and/or MI (defined as p<0.05). CAD and MI odds ratios for these polymorphisms are shown in Table 4, below. Individuals who are homozygous for the variant of the PLGC1 SNP G329 ul (i.e., TT), or the complement thereof, are approximately 1.5-fold less likely to develop CAD and/or MI than individuals without this genotype. Individuals who are homozygous for the reference allele of the PAI-2 SNP PAIu1 (i.e., TT), or the complement thereof, are approximately 1.5-fold less likely to develop CAD and/or MI than individuals without this genotype. Individuals who are both homozygous for the variant of the PLGC1 SNP G329ul (i.e., TT genotype) and homozygous for the reference allele of the PAI-2 SNP PAI ul (i.e., TT), or the complements thereof, are approximately 3-fold less likely to develop CAD and/or MI than individuals with any other combination (odds ratio CAD:0.39 (0.22, 0.68); odds ratio MI:0.32 (0.15, 0.70)).

TABLE 4

| | | | | | | CAD Odds Ratio | MI Odds Ratio |
|---|---|---|---|---|---|---|---|
| | | | | CAD | MI | (95% confidence | (95% confidence |
| Gene | PolyID | Geno-type | Controls | cases | cases | interval) | interval) |
| PLCG1 | G329u1 | TT | 87 | 50 | 29 | 0.66 (.44, .98)* | .72 (.44, 1.12) |
| | | TC/CC | 326 | 284 | 151 | 1.00 | 1.00 |
| PAI-2 | PAI2u1 | AA | 235 | 163 | 80 | 0.75 (.55, 1.02) | 0.62 (.42, .91)* |
| | | GA/GG | 153 | 142 | 84 | 1.00 | 1.00 |
| both | G329u1 | TT (G329u1) | 56 | 18 | 8 | 0.39 (.22, .68)* | 0.32 (.15, .69)* |
| PLCG1 | and | and | | | | | |
| and | PAIu1 | AA (PAI2u1) | | | | | |
| PAI-2 | | vs all other | 328 | 272 | 146 | 1.00 | 1.00 |

*p < .05

Possible combinations of these alleles are listed in Table 5, below. As discussed above, individuals who are homozygous for the variant of the PLGC1 SNP G329 ul (TT) and homozygous for the reference allele of the PAI-2 SNP PAI ul (TT), or the complements thereof, are approximately 3-fold less likely to develop a vascular disease or disorder compared to individuals with any other possible combination.

TABLE 5

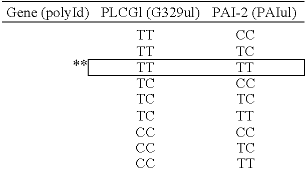

**3-fold less likely to develop CAD and/or MI compared to all other possible combinations

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for diagnosing or aiding in the diagnosis of a vascular disease or disorder in a subject comprising the steps of determining the PLCG1 and PAI-2 genetic profile of the subject, thereby diagnosing or aiding in the diagnosis of a vascular disease or disorder.

2. The method of claim 1, wherein determining the subject's PLCG1 and PAI-2 genetic profile comprises determining the identity of the nucleotide present at nucleotide position 64001 of SEQ ID NO:1 and the nucleotide present at nucleotide position 170871 of SEQ ID NO:3, or the complement thereof.

3. The method of claim 1, wherein determining the subject's PLCG1 and PAI-2 genetic profile comprises determining the identity of the amino acid present at amino acid residue 813 of SEQ ID NO:2 and the amino acid present at amino acid residue 120 of SEQ ID NO:4, or the complement thereof.

4. The method of claim 1, wherein the vascular disease is myocardial infarction.

5. The method of claim 1, wherein the vascular disease is coronary artery disease.

6. A method for predicting the likelihood that a subject will or will not develop a vascular disease or disorder comprising the steps of determining the PLCG1 and PAI-2 genetic profile of the subject, thereby predicting the likelihood that a subject will or will not develop a vascular disease or disorder.

7. The method of claim 6, wherein determining the subject's PLCG1 and PAI-2 genetic profile comprises determining the identity of the nucleotide present at nucleotide position 64001 of SEQ ID NO:1 and the nucleotide present at nucleotide position 170871 of SEQ ID NO:3, or the complement thereof.

8. The method of claim 6, wherein determining the subject's PLCG1 and PAI-2 genetic profile comprises determining the identity of the amino acid present at amino acid residue 813 of SEQ ID NO:2 and/or the amino acid present at amino acid residue 120 of SEQ ID NO:4, or the complement thereof.

9. The method of claim 6, wherein the vascular disease is myocardial infarction.

10. The method of claim 6, wherein the vascular disease is coronary artery disease.

11. A method of diagnosing or aiding in the diagnosis of a vascular disease in a subject comprising the steps of determining the nucleotide present at nucleotide position 170871 of the PAI-2 gene and determining the nucleotide present at nucleotide position 64001 of the PLCG1 gene, wherein the presence of two copies of a thymidine allele at nucleotide position 170871 of the PAI-2 gene together with two copies of a thymidine allele at nucleotide position 64001 of the PLCG1 gene, or the complements thereof, is indicative of decreased likelihood of a vascular disease in the subject as compared with a subject having any other combination of these alleles.

12. The method of claim 11, wherein determining said nucleotides comprises obtaining a nucleic acid sample from the subject.

13. The method of claim 11, wherein the PLCG1 gene has the nucleotide sequence of SEQ ID NO:1, or a portion thereof, and wherein the PAI-2 gene has the nucleotide sequence of SEQ ID NO:3, or a portion thereof.

14. The method of claim 11, wherein the vascular disease is selected from the group consisting of atherosclerosis, coronary artery disease, myocardial infarction, ischemia, stroke, peripheral vascular diseases, venous thromboembolism and pulmonary embolism.

15. The method of claim 14, wherein the vascular disease is myocardial infarction.

16. The method of claim 14, wherein the vascular disease is coronary artery disease.

17. A method for predicting the likelihood that a subject will or will not develop a vascular disease, comprising the steps of determining the nucleotide present at nucleotide position 170871 of the PAI-2 gene and determining the nucleotide present at nucleotide position 64001 of the PLCG1 gene, wherein the presence of two thymidine alleles at nucleotide position 170871 of the PAI-2 gene and the presence of two thymidine alleles at nucleotide position 64001 of the PLCG1 gene, or the complements thereof, is indicative of decreased likelihood of the subject developing a vascular disease as compared with a subject having any other combination of these alleles.

18. The method of claim 17, wherein determining said nucleotides comprises obtaining a nucleic acid sample from the subject.

19. The method of claim 17, wherein the PLCG1 gene has the nucleotide sequence of SEQ ID NO:1, or a portion thereof, and wherein the PAI-2 gene has the nucleotide sequence of SEQ ID NO:3, or a portion thereof.

20. The method of claim 17, wherein the vascular disease is selected from the group consisting of atherosclerosis, coronary artery disease, myocardial infarction, ischemia, stroke, peripheral vascular diseases, venous thromboembolism and pulmonary embolism.

21. The method of claim 20, wherein the vascular disease is myocardial infarction.

22. The method of claim 21, wherein the vascular disease is coronary artery disease.

23. A method of diagnosing or aiding in the diagnosis of a vascular disease in a subject comprising the steps of determining the amino acid present at amino acid position 120 of the PAI-2 protein and determining the amino acid present at amino acid position 813 of the PLCG1 protein, wherein presence of a threonine at amino acid position 813 of the PLCG1 protein and the presence of an asparagine at amino acid position 120 of the PAI-2 protein is indicative of decreased likelihood of a vascular disease in the subject as compared with a subject having any other combination of these amino acids.

24. The method of claim 23, wherein determining said amino acids comprises obtaining a protein sample from the subject.

25. The method of claim 23, wherein the PLCG1 protein has the amino acid sequence of SEQ ID NO:2, or a portion thereof and wherein the PAI-2 protein has the amino acid sequence of SEQ ID NO:4, or a portion thereof.

26. The method of claim 23, wherein the vascular disease is selected from the group consisting of atherosclerosis, coronary artery disease, myocardial infarction, ischemia, stroke, peripheral vascular diseases, venous thromboembolism and pulmonary embolism.

27. The method of claim 25, wherein the vascular disease is myocardial infarction.

28. The method of claim 25, wherein the vascular disease is coronary artery disease.

29. A method for predicting the likelihood that a subject will not develop a vascular disease, comprising the steps of determining the amino acid present at amino acid position 120 of the PAI-2 protein and determining the amino acid present at amino acid position 813 of the PLCG1 protein, wherein presence of a threonine at amino acid position 813 of the PLCG1 protein and the presence of an asparagine at amino acid position 120 of the PAI-2 protein is indicative of decreased likelihood of a subject developing a vascular disease as compared with a subject having any other combination of these amino acids.

30. The method of claim 29, wherein determining said amino acids comprises obtaining a protein sample from the subject.

31. The method of claim 29, wherein the PLCG1 protein has the amino acid sequence of SEQ ID NO:2, or a portion thereof and wherein the PAI-2 protein has the amino acid sequence of SEQ ID NO:4, or a portion thereof.

32. The method of claim 29, wherein the vascular disease is selected from the group consisting of atherosclerosis, coronary artery disease, myocardial infarction, ischemia, stroke, peripheral vascular diseases, venous thromboembolism and pulmonary embolism.

33. The method of claim 29, wherein the vascular disease is myocardial infarction.

34. The method of claim 32, wherein the vascular disease is coronary artery disease.

35. A computer readable medium for storing instructions for performing a computer implemented method for determining whether or not a subject has a predisposition to a vascular disease or disorder, said instructions comprising the functionality of:
obtaining information from the subject indicative of the presence or absence of the polymorphic region of a PLCG1 and/or PAI-2 gene, and
based on the presence or absence of the polymorphic region of a PLCG1 and/or PAI-2 gene, determining whether or not the subject has a predisposition to a vascular disease or disorder.

36. A computer readable medium for storing instructions for performing a computer implemented method for identifying a predisposition to a vascular disease or disorder, said instructions comprising the functionality of:
obtaining information regarding the presence or absence of the polymorphic region of a PLCG1 and/or PAI-2 gene, and
based on the presence or absence of the polymorphic region of a PLCG1 and/or PAI-2 gene, identifying a predisposition to a vascular disease or disorder.

37. An electronic system comprising a processor for determining whether or not a subject has a predisposition to a vascular disease or disorder, said processor implementing the functionality of:
obtaining information from the subject indicative of the presence or absence of the polymorphic region of a PLCG1 and/or PAI-2 gene, and
based on the presence or absence of the polymorphic region of a PLCG1 and/or PAI-2 gene, determining whether or not the subject has the predisposition to a vascular disease or disorder.

38. An electronic system comprising a processor for performing a method for identifying a predisposition to a vascular disease or disorder in a subject, said processor implementing the functionality of:
obtaining information from the subject indicative of the presence or absence of the polymorphic region of a PLCG1 and/or PAI-2 gene, and
based on the presence or absence of the polymorphic region of a PLCG1 and/or PAI-2 gene, performing a method for identifying a predisposition to a vascular disease or disorder associated with the polymorphic region.

39. The electronic system of claims 37 or 38, wherein said processor further implements the functionality of receiving phenotypic information associated with the subject.

40. The electronic system of claims 37 or 38, wherein said processor further implements the functionality of acquiring from a network phenotypic information associated with the subject.

41. A network system for identifying a predisposition to a vascular disease or disorder in response to information submitted by an individual, said system comprising means for:
receiving data from the individual regarding the presence or absence of the polymorphic region of a PLCG1 and/or PAI-2 gene, and
based on the presence or absence of the polymorphic region, determining whether or not the subject has the predisposition to the vascular disease or disorder associated with the polymorphic region.

42. A network system for identifying whether or not a subject has a predisposition to a vascular disease or disorder, said system comprising means for:
receiving information from the subject regarding the polymorphic region of a PLCG1 and/or PAI-2 gene,
receiving phenotypic information associated with the subject,
acquiring additional information from the network, and
based on one or more of the phenotypic information, the polymorphic region, and the acquired information, determining whether or not the subject has a pre-disposition to a vascular disease or disorder associated with a polymorphic region of a PLCG1 and/or PAI-2 gene.

43. The system of claims 41 and 42, wherein the network system comprises a server and a work station operatively connected to said server via the network.

44. A composition comprising an isolated nucleic acid molecule comprising an allelic variant of a polymorphic region of a PLCG1 gene, wherein the allelic variant differs from the reference sequence set forth in SEQ ID NO:1, or a portion thereof, and wherein the allelic variant is associated with aberrant PLCG1 activity, in combination with an isolated nucleic acid molecule comprising an allelic variant of a polymorphic region of a PAI-2 gene, wherein the allelic variant does not differ from the reference sequence set forth in SEQ ID NO:3, or a portion thereof, and wherein the allelic variant is associated with aberrant PAI-2 activity.

45. The composition of claim 44, wherein the polymorphic regions are located in an exon.

46. A composition comprising an isolated nucleic acid molecule comprising the nucleotide sequence of SEQ ID NO:1, or a portion thereof, further comprising the nucleotide sequence of SEQ ID NO:5, or the complement thereof, in combination with an isolated nucleic acid molecule comprising the nucleotide sequence of SEQ ID NO:3, or a portion thereof, further comprising the nucleotide sequences of SEQ ID NO:6, or the complement thereof.

47. A composition comprising an isolated nucleic acid molecule comprising the nucleotide sequence set forth in SEQ ID NO:1, or a portion thereof, wherein residue 64001 is a thymidine, or the complement thereof, in combination with an isolated nucleic acid molecule comprising the nucleotide sequence set forth in SEQ ID NO:3, or a portion thereof, wherein residue 170871 is a thymidine, or the complement thereof.

48. A kit comprising probes or primers which are capable of hybridizing to the nucleic acid molecules of any of claims 44-47.

49. The kit of claim 48, wherein the probes or primers comprise a nucleotide sequence from about 15 to about 30 nucleotides.

50. The kit of claim 48, wherein the probes or primers are labeled.

51. A method for determining the identity of one or more allelic variants of a polymorphic region of a PLCG1 gene and a PAI-2 gene in a nucleic acid obtained from a subject, comprising contacting a sample nucleic acid from the subject with a probe or primer having a sequence which is complementary to a PLCG1 gene sequence and a probe or primer which is complementary to a PAI-2 gene sequence, wherein the sample comprises a PLCG1 gene sequence and a PAI-2 gene sequence, thereby determining the identity of one or more of the allelic variants.

52. The method of claim 51, wherein the probes or primers are capable of hybridizing to an allelic variant of a polymorphic region of the PLGC1 and PAI-2 genes, and wherein the allelic variant differs from the reference sequence set forth in of SEQ ID NO:1 and does not differ from the reference sequence set forth in SEQ ID NO:3.

53. The method of claim 52, wherein determining the identity of the allelic variant comprises determining the identity of at least one nucleotide of the polymorphic region of a PLCG1 gene and at least one nucleotide of the polymorphic region of a PAI-2 gene.

54. The method of claim 53, wherein determining the identity of the allelic variant consists of determining the nucleotide content of the polymorphic region.

55. The method of claim 53, wherein determining the nucleotide content comprises sequencing the nucleotide sequence.

56. The method of claim 53, wherein determining the identity of the allelic variant comprises performing a restriction enzyme site analysis.

57. The method of claim 53, wherein determining the identity of the allelic variant is carried out by single-stranded conformation polymorphism.

58. The method of claim 53, wherein determining the identity of the allelic variant is carried out by allele specific hybridization.

59. The method of claim 53, wherein determining the identity of the allelic variant is carried out by primer specific extension.

60. The method of claim 53, wherein determining the identity of the allelic variant is carried out by an oligonucleotide ligation assay.

61. The method of claim 53, wherein the probe or primer comprises a nucleotide sequence from about 15 to about 30 nucleotides.

62. An Internet-based method for assessing a subject's risk for vascular disease, the method comprising:
a) analyzing biological information from a subject indicative of the presence or absence of a polymorphic region of PLCG1 and/or PAI-2;
b) providing results of the analysis to the subject via the Internet, wherein the presence of a polymorphic region of PLCG1 and/or PAI-2 indicates a decreased risk for vascular disease.

63. A method of assessing a subject's risk for vascular disease, the method comprising:
a) obtaining biological information from the individual;
b) analyzing the information to obtain the subject's PLCG1 and/or PAI-2 genetic profile;
c) representing the PLCG1 and/or PAI-2 genetic profile information as digital genetic profile data;
d) electronically processing the PLCG1 and/or PAI-2 digital genetic profile data to generate a risk assessment report for vascular disease, wherein the presence of a polymorphic region of PLCG1 and/or PAI-2 indicates a decreased risk for vascular disease; and
e) displaying the risk assessment report on an output device.

64. A method of assessing a subject's risk for vascular disease, the method comprising:
a) obtaining the subject's PLCG1 and/or PAI-2 genetic profile information as digital genetic profile data;
b) electronically processing the PLCG1 and/or PAI-2 digital genetic profile data to generate a risk assessment report for vascular disease, wherein the presence of a polymorphic region of PLCG1 and/or PAI-2 indicates a decreased risk for vascular disease; and
c) displaying the risk assessment report on an output device.

65. The method of claims 63 or 64, further comprising the step of using the risk assessment report to provide medical advice.

66. The method of claims 63 or 64, wherein additional health information is provided.

67. The method of claim 66, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

68. The method of claim 64, wherein the PLCG1 and/or PAI-2 digital genetic profile data are transmitted via a communications network to a medical information system for processing.

69. The method of claim 68, wherein the communications network is the Internet.

70. A medical information system for assessing a subject's risk for vascular disease comprising:
a) means for obtaining biological information from the individual to obtain a PLCG1 and/or PAI-2 genetic profile;
b) means for representing the PLCG1 and/or PAI-2 genetic profile as digital molecular data;
c) means for electronically processing the PLCG1 and/or PAI-2 digital genetic profile to generate a risk assessment report for vascular disease; and
d) means for displaying the risk assessment report on an output device, wherein the presence of a polymorphic region of PLCG1 and/or PAI-2 indicates a decreased risk for vascular disease.

71. A medical information system for assessing a subject's risk for vascular disease comprising:
a) means for representing the subject's PLCG1 and/or PAI-2 genetic profile data as digital molecular data;
b) means for electronically processing the PLCG1 and/or PAI-2 digital genetic profile to generate a risk assessment report for vascular disease; and
c) means for displaying the risk assessment report on an output device, wherein the presence of a polymorphic region of PLCG1 and/or PAI-2 indicates a decreased risk for vascular disease.

72. A computerized method of providing medical advice to a subject comprising:
a) analyzing biological information from a subject to determine the subject's PLCG1 and/or PAI-2 genetic profile;
b) based on the subject's PLCG1 and/or PAI-2 genetic profile, determining the subject's risk for vascular disease;
c) based on the subject's risk for vascular disease, electronically providing medical advice to the subject.

73. A computerized method of providing medical advice to a subject comprising:
a) based on the subject's PLCG1 and/or PAI-2 genetic profile, determining the subject's risk for vascular disease;
b) based on the subject's risk for vascular disease, electronically providing medical advice to the subject.

74. The method of any of claims 72 or 73, wherein the medical advice comprises one or more of the group consisting of further diagnostic evaluation, administration of medication, or lifestyle change.

75. The method of claims 72 or 73, wherein additional health information is obtained from the subject.

76. The method of claim 75, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

77. A method for self-assessing risk for a vascular disease comprising
a) providing biological information for genetic analysis;
b) accessing an electronic output device displaying results of the genetic analysis, thereby self-assessing risk for a vascular disease, wherein the presence of a polymorphic region of PLCG1 and/or PAI-2 indicates a decreased risk for vascular disease.

78. A method for self-assessing risk for a vascular disease comprising accessing an electronic output device displaying results of a genetic analysis of a biological sample, wherein the presence of a polymorphic region of PLCG1 and/or PAI-2 indicates a decreased risk for vascular disease, thereby self-assessing risk for a vascular disease.

79. A method of self-assessing risk for vascular disease, the method comprising
a) providing biological information;
b) accessing PLCG1 and/or PAI-2 digital genetic profile data obtained from the biological information, the PLCG1 and/or PAI-2 digital genetic profile data being displayed via an output device, wherein the presence of a polymorphic region of PLCG1 and/or PAI-2 indicates a decreased risk for vascular disease.

80. A method of self-assessing risk for vascular disease, the method comprising accessing PLCG1 and/or PAI-2 digital genetic profile data obtained from biological information, the PLCG1 and/or PAI-2 digital genetic profile data being displayed via an output device, wherein the presence of a polymorphic region of PLCG1 and/or PAI-2 indicates a decreased risk for vascular disease.

81. The method of claims 79 or 80, wherein the electronic output device is accessed via the Internet.

82. The method of claims 79 or 80, wherein additional health information is provided.

83. The method of claim 82, wherein the additional health information comprises information regarding one or more of age, sex, ethnic origin, diet, sibling health, parental health, clinical symptoms, personal health history, blood test data, weight, and alcohol use, drug use, nicotine use, and blood pressure.

84. The method of any of claims 77, 78, 79, or 80, wherein the biological information is obtained from a sample from an individual at a laboratory company.

85. The method of claim 84, wherein the laboratory company processes the biological sample to obtain PLCG1 and/or PAI-2 genetic profile data, represents at least some of the PLCG1 and/or PAI-2 genetic profile data as digital genetic profile data, and transmits the PLCG1 and/or PAI-2 digital genetic profile data via a communications network to a medical information system for processing.

86. The method of any of claims 77, 78, 79, or 80, wherein the biological information is obtained from a sample from an individual at a draw station, wherein the draw station processes the biological sample to obtain PLCG1 and/or PAI-2 genetic profile data, and transfers the data to a laboratory company.

87. The method of claim 86, wherein the laboratory company represents at least some of the PLCG1 and/or PAI-2 genetic profile data as digital genetic profile data, and transmits the PLCG1 and/or PAI-2 digital genetic profile data via a communications network to a medical information system for processing.

88. A method for a health care provider to generate a personal health assessment report for an individual, the method comprising counseling the individual to provide a biological sample; authorizing a draw station to take a biological sample from the individual and transmit molecular information from the sample to a laboratory company, wherein the molecular information comprises the presence or absence of a polymorphic region of PLCG1 and/or PAI-2; requesting the laboratory company to provide digital molecular data corresponding to the molecular information to a medical information system to electronically process the digital molecular data and digital health data obtained from the individual to generate a health assessment report; receiving the health assessment report from the medical information system; and providing the health assessment report to the individual.

89. A method for a health care provider to generate a personal health assessment report for an individual, the method comprising requesting a laboratory company to provide digital molecular data corresponding to the molecular information derived from a biological sample from the individual to a medical information system to electronically process the digital molecular data and digital health data obtained to generate a health assessment report; receiving the health assessment report from the medical information system; and providing the health assessment report to the individual.

90. A method of assessing the health of an individual, the method comprising: obtaining health information from the individual using an input device; representing at least some of the health information as digital health data; obtaining biological information from the individual, wherein the information comprises the presence or absence of a polymorphic region of PLCG1 and/or PAI-2; representing at least some of the information as digital molecular data; electronically processing the digital molecular data and digital health data to generate a health assessment report; and displaying the health assessment report on an output device.

91. The method of claim 90, wherein electronically processing the digital molecular data and digital health data to generate a health assessment report comprises using the digital molecular data and digital health data as inputs for an algorithm or a rule-based system that determines whether the individual is at risk for a specific disorder.

92. The method of claim 90, wherein the individual has or is at risk of developing vascular disease, and wherein electronically processing the digital molecular data and digital health data to generate a health assessment report comprises using the digital molecular data and digital health data as inputs for an algorithm or a rule-based system that determines the individual's prognosis.

93. The method of claim 90, wherein electronically processing the digital molecular data and digital health data comprises using the digital molecular data and digital health data as inputs for an algorithm or a rule-based system based on one or more databases comprising stored digital molecular data and/or digital health data relating to one or more disorders.

94. The method of claim 90, wherein electronically processing the digital molecular data and digital health data comprises using the digital molecular data and digital health data as inputs for an algorithm or a rule-based system based on one or more databases comprising (i) stored digital molecular data and/or digital health data from a plurality of healthy individuals, and (ii) stored digital molecular data and/or digital health data from one or more pluralities of unhealthy individuals, each plurality of individuals having a specific disorder.

95. The method of either of claims 93 or 94, wherein at least one of the databases is a public database.

96. The method of claim 90, wherein the digital health data and digital molecular data are transmitted via a communications network to a medical information system for processing.

97. The method of claim 95, wherein the communications network is the Internet.

98. The method of claim 95, wherein the input device is a keyboard, touch screen, hand-held device, telephone, wireless input device, or interactive page on a website.

99. The method of claim 90, wherein the health assessment report comprises a digital molecular profile of the individual.

100. The method of claim 90, wherein the health assessment report comprises a digital health profile of the individual.

101. The method of claim 90, wherein the molecular data comprises nucleic acid sequence data, and the molecular profile comprises a genetic profile.

102. The method of claim 90, wherein the molecular data comprises protein sequence data, and the molecular profile comprises a proteomic profile.

103. The method of claim 90, wherein the molecular data comprises information regarding one or more of the absence, presence, or level, of one or more specific proteins, polypeptides, chemicals, cells, organisms, or compounds in the individual's biological sample.

104. The method of claim 90, wherein the health information comprises information relating to one or more of age, sex, ethnic origin, diet, sibling health, parental health, clinical symptoms, personal health history, blood test data, weight, and alcohol use, drug use, nicotine use, and blood pressure.

105. The method of claim 90, wherein the health information comprises current and historical health information.

106. The method of claim 90, further comprising obtaining a second set of biological information at a time after obtaining the first set of biological information; processing the second set of biological information to obtain a second set of information; representing at least some of the second set of information as digital second molecular data; and processing the molecular data and second molecular data to generate a health assessment report.

107. The method of claim 106, further comprising obtaining second health information at a time after obtaining the health information; representing at least some of the second health information as digital second health data and processing the molecular data, health data, second molecular data, and second health data to generate a health assessment report.

108. The method of claim 95, wherein the health assessment report provides information about the individual's predisposition for vascular disease and options for risk reduction.

109. The method of claim 108, wherein the options for risk reduction comprise one or more of diet, exercise, one or more vitamins, one or more drugs, cessation of nicotine use, and cessation of alcohol use.

110. The method of claim 95, wherein the health assessment report provides information about treatment options for a particular disorder.

111. The method of claim 110, wherein the treatment options comprise one or more of diet, one or more drugs, physical therapy, and surgery.

112. The method of claim 95, wherein the health assessment report provides information about the efficacy of a particular treatment regimen and options for therapy adjustment.

113. The method of claim 95, further comprising storing the molecular data.

114. The method of claim 113, further comprising building a database of stored molecular data from a plurality of individuals.

115. The method of claim 95, further comprising storing the molecular data and health data.

116. The method of claim 115, further comprising building a database of stored molecular data and health data from a plurality of individuals.

117. The method of claim 116, further comprising building a database of stored digital molecular data and/or digital health data from a plurality of healthy individuals, and stored digital molecular data and/or digital health data from one or more pluralities of unhealthy individuals, each plurality of individuals having a specific disorder.


SEQUENCE LISTING (6 sequences)

SEQ ID NO: 1 - DNA, 146547 bp, Homo sapiens
gatcttcaaaggttggggtttgatagtgccttgaataatttttaactttatattgccagcggaagaagcattctctttttagatttaaaaaatgtagatacaaatattaggggttttatttttagtgaaacatttcaaacatacaggaatagataattatgtaatgaacactcgtatgtccaccatctggctttgtaaaatcttaaaattatgtcttatgtgctcaattgttttatttcataaaagatactgataaacatagctgaagtcacttgtataccattcaccttcttccctgtagattactatgaactcggtctttttattctcatacatattttttgtatttttgcagtatatttatgtgttcataaacaatatgtaattttacaatatgtaacacactagtaacatactaatttaaaacttgtttttagtttacaatatgttgtaaactattgtaagctaaagacatattgtacaacctattgtaaaataaaaacaggttttagttttaaattaggtatgttactgggatcattctgcaacttgtttattcctctccagctttgattttgtggttttattatcttaacctacacttttaattaatccattttatttgttacatggtattctattatatcataaaaacttatctattctgttggtttttgttgttggtcatttgagaccatgtcttcgctctgtcacccaggctggagtacagtggcgtgatcttggctcactgtgacctctgcctcccggattcaagtggttttggtgcctcagcctcctgagtagctgggattataggcgtgtgccaccatgcccagctaatttttgtatttttaatagagacgggatttcaccatgttggccaggctggtcttgaactgacctcaagtgatctgctcacctcagctgcacaaagtgctgggattacaggtgttagccaccataccctgcctctattctcttgttaagaggcatttagcatggttatacagtctcttgccctcataaacagtgctggaagaaacacatgtttcttgtgtatatgaatgaaaatttgttttatacattagatatttccaaattgttctcttaagtacttcagtttacatcattactctcctcctccctcccctcccacccccacccacaacagtattcctctttttccatatccttgctaatgtttctaaagttttgctttttacatttgggtcttagatccactagaatgtatttttgcattgggatgaagttgaaacctaatatattttccaaatgagtaaactgttgtcacagaactatttagttgtattacctcctctcttgtatatcagatatatctacatatatgtcagactgtttctgggctgtctgtcctctttaattagttcgtgtatctgtttctgcatcagtagcatactgtcttaactactgtagctttataaagtctattgagtaggacaagtttgtttcattcttcaaaattgctttggctattcttggccctctgctgtttcatattaactttcagataaacttgtcaaattctaatgaaaactgttgataaacttgttgattaacaaattctaataaaaactgttgagatttttattggaattgcaatacatttatagattaacggagaaagatattgacaatacaattgagtttccaattcacgaacatgttatacctctccattaattcatgtcttttgaatgtatccaccaatatggttttgtaattttcttcataaaggttttacatttaaaaaattcttatttttaagtgatcttatagtttttattgctaatgtgaatgagatttttttccattatgtttctgttggttattcctgaagtggtaatgcttataattttggggtgttggtcttgtatctggcagcagatacaagaggtgctcagagttgttcagggttgctgaactcttagttctaaaagtgtctgtcatttggggtttctatgtagataatttaattatctataaaaacagttcttcattttcagttcatatatttcatatttctttaagttttaatttttatttttaaacacaattatccataaaaccctaaccctttccctagtcaacagcagtcacagccaaatgttttattaattgctatactcagtgtttcttgtatctcataccttctggggtttcttgtcttgttgaaatacaccctttaatgtttctttagtgaagacccaacagtggcactcactcacctttgtttacctgaaaatttctttattttcatcttaattcatagtctgtcttttctccagtcaaggaagtgtcttatagggaagattctggtttcactatgctgtatccagggatatatatgtatttatagatagacttttaatctgaggactaatgtattttatcctacagtattaccaatcattatttcttccataacttctagaccattccttttgtacttcttttttagagtcctattagatgagtgttgacacttttcaatctagacatcttttttaaactatattttcatactctttgtctctttaggtctgattttttaagttcaggggaatatttcattttgggtgagttgtagcactcacttccaattcactaattctaattatatttaatctacaagttattccatctataatttatttcaattaccactttttgttttcaaaatttctaattttatatctgattttgtttcatttttgttttataatttcatgttctttctagattttacatctttttatgcatactaaacatactcacttgaaagtctttgtaagattgttctataaaatgttacctgaagtgaattcatgtgctaattactgttggcagtttttctgagccattttccttgtgtcttttgaaatttctgtttgtaagcccttttaactgggaggtgttttttgcttatgtactgttctcttttctttcccttcttctccttctctgttcattactttttgtcttggagggagtctcttagcccagctcagcccaagctaataaggcccctgacttcaagtccccccttcttgcatggagcaagaagtgatcctcctgtctcagcctcccaaagtgctgggtaggcgtgagccaccacatccagcctagagcacttacattgctacatatttggagcaaacataattaaagtatgacttactgtgacccagcagtcaataacagatatgtttttgtaatagaatgttgaagggtggtaactggtggttttacttcatggagggctttaggtcggctatatgggaagctgccatcatgatgcttttgttctacacatttctgctcagcctccagagatgtctcctattctaagagtcgtctttgtttactcttgtgtctatattttacttacttaattccctcacataatagtgtatcattgtatcccatctgtgctctatcaagaaatgtagcacaagcatgatgtgaactatgaattttcgacaatttaaaaaattaaaggaatcattcaggggtaaaaagcacaaagttgtagaacagctgaagtgtgttctttttatgtatcctgtctttgccccctttctggaaacttggattggcctacttgcagtcactgtcacagttacactacttctagcctcaaaggtcattgaaaggattgggatttttgagaaccacaataacacttgctctaggtactgaaaaggttgtcctgaactcattcatatctttttatgttcatcccagtttgtttttgcttttccccccaggattttgattacacagacatcttgcaagcatatttccattctttctctcccactctgtgcctggtgcccgtaggatctgaaatagcattggaatgacagctagccttctggaacctgcagtttcaaccaaagaaactctgattccagataacccaggagtttcttatcctgcataatctaggagtttacaaaaaggctgcttatgggtactcactgctaagtacaatccactgccaagtttggaagcattacctgctctcacaacccagggaaagtagcccttagtaaaattcaggttttttcaaaccagccttaaccttacaaacagaatttatcatcatgatttttctttcaaatgcagtacagcaaactttaggatcaatgttgacatcgtattgcgtttagtcctgttaaaatactctcagtgccaaatacacacttctgccttttttctctttagagtctagaattctgtggggttccaaaaatgtatgtgcttacaaccttttatatgtaaccacctagggattaaagggctaaatgtgaaggaaagtatctttacaatattcatcttgggaagctctcattttggtgagttctattagagaaaataagacacaagctcattaaaaatatatatatattctgagcctggcatcttggtataaaattcaaaacgttttaaatctatttattagtaagactaacatagacctaagtgtgaagactaaaatattaaagcttgtaggtgatatcatggttgaatatgaccttagagtagattccttaggacagaaaaacaccaacaagagaaacaattgactaaatggacttcattaaaattaaaaatttatgttcaacaaaagacactgctaaaataaatacagaatttacagactaaaagaaaatatttgcaacatgtttatctgaaagggcttatatccagactatacaaagaattcctgtagctccataataaaaaaacagacatcccatccgcataataaaaagagcaaaagacttgaacatatactatacaaaagaagatacaggaatggtcaataaataagcaaatgaaagagtgctcgacatcactaattatcagtggagtgaagatttaaccacagggagatacgaattccagttctaggtatttttacctcaagtgaaataaaaacatttatccatgaaaaaacttgtacaagaatgttcatagcagtgctctattcataagagtgcaaaactggaaacatcccagaatagataacacattatggtatagtcatagaatatactatatttatcagcagttaagaagggaactacttatgtattcaacaacatggatgaatcttaaaaacatggttaagggccaggtgcggttggtgcatgcctgtaatccaagcactctgggaggccaaggcgggtggatcacttcaggtcaggagttcgagaccagcctggccaacatggcgaaacaccgtctctacaaaaaaatacaaaaattagccgagggtggcgcacacctgtaatctcagctattcaggaggctgagacatgggaatcgcttgaacctgaggggtagaggttgcactgagccgagattgcatcactgtactccagcctggtgacagagcgagacgctgtctcagagggaaaaaaaaaagttaaaaaggcagacagaggagtctatattgtatgattctatttatataatctagactaggcaaaatttatccatgatgatagtaatcagattgaaggatcagtggttgcttggtggaaattgactgagaagggaaatagtcatgtaagaatactggcaaatgtcctcttccccattacaaacgtctgagaaagttactagaaaacagaatccaatttgccttctcacccccaactttgtaatcttgaatccagatcctgattccacccaaccactctgctgaatggatagcagctatattagagagacaatatagtgggcctactagttttaaaaagtcatccaaagaatgtcatctacaatcagttaaattagatttttacccattactgaagtgtgattgaattagggaaagaggagaggtggtcaaaggcagccttctccagtggattccttgtcttcttgagatggttttgtgagaccttgcctggaagacctaggtggatagtttattttctaaaagtagtcaagaaagcttgttcagggcattgaacactgcagggacaagcatacttcttggaggccagccatctcctgctggatctaagcggggtggcttctgcctccacctccatctcagcttatttgagagaatcctagatccttggtgcagaagcccagccagcctgtgaagaaaagttggtctggtagtatcctgtgaatgacgtgcgggttgggtttttctaaatctcgtttacctgactcctaccttacattccagtggtgtgactaaggaactgagcaaacgtaaccctaggggactttgagacaggctttggtagtccatgtgcactagagtgaggggaaagaggccactccagagttgggtactctactgttgaaggtgagccttttgtgtccaatttttccgtgcatgtgtaagaacagttgtcatggagatttactactgacttgtgcatgaggacagcttatatctcaccttttcttattcaccctgttaaatgtcaaaatgtactaagacttaggtattttttctgtgtcaccaactactaaatcacccacccatgatagcaggtagttactgaaactcttgtagccaaacaagtgcataacactggtacacatgatgtgagagtgcagatgactccttagcttaactgtgtaagcttttaatttctgagtacttttaggattgagaagtggaagacattcttgctctaggtatctcatgcttctgcacacccagaaggctggttgcgcccccaccccaccccccgcccccttttttttttttttgatagggtcatgctctgttgcccaggctgaaaggctggttatcttcaccacaggtcttatgtctcaaggaccacgggtccacgtcatagtcaggaaccaggcatgtgtgggcagaaatcactaccactttcctttgttgcgggtggggaaattaagagcagagattcttggattcttaaattccttctctatgtttatgtcatctagtctgttttatgctgttttatgtaattccctgcttctggatttcttgaatttttttccccttctcacttgtcataattggctcttgcttgtgagtctattttcattcagcttggtagcagaagtcttcttcactattgtttagattcagttttcacatggttttagtaacccttcattgggtttagaatcaacttttaacccttccaccaaaagctcttatttttttaaaggcttcccagaaacatagtttctctcaatctcatcaagaaatgcttttaaatgcagaagttagaaaatggtgaataattatggctgttcctggaagtcattgtgtccttagtaattacatgttgtagtctttggttctgcttctttgacagctccaccacactgtaatttaatggaattcacttagatttaatggaattcagtcctggtagaaaactgctattgggtcacagagttggcagcaaggctgtgaaattgatgtgggaatgggggtgattatgtgctttgtgttgacagaagttagtttttgggacatcccatgtgggtttttcccaggaacataaacattttagagaattagagctagaaactttcttctgggaaagccttagaagtcagttgtgcttggcagtgagatgggacaatgtgcattcttaacaggttaaatgtgccaaactctctcttcagtactattcttttttacgtttgcttaaggtcaagttcctgctcttttgttcaaagtcttgctttgtcacccatggtggagtgcagtggcgtgatcttggctcactgcaacctctgcctcctgcgttcaagcaattcttgtgtcttagcctccctattagctagctgggattataggcttgcgccaccttgcctggctatattcaaagtctgtctttactacactgtcccaaagtaatctacatttgggtttgggtctttacagggtgagaaggactggcagaaatacgagactgctcggcggctgaaaaaatgtgtggacaagatccggaaccagtatcgagaagactggaagtccaaagagatgaaagtccggcagagagctgtagccctgtacttcatcgacaaggtgagagcatcttcccatcggcattgtctagtgttgagcttaacaaagggagtttctgctctgccccaggccctgtgccacatactgtatatcacaactcaacatacatatatttgtatgtaaaactgaatcaaaagttgcacaaaacaatgcttagccttactgtgagcagtagattctgatctttttttaactctatttcattcctttagaaagttggttataacttataaaattgtttaaataaaccatgcaggtatcaccattatatagttttaaagaccacgctctagaggagatcaatttcttggtactcacgagaagaaaaaaaagttcatgtttttcccccagtcttgtattttaacataagttctgaattgtctcaaaattcacactagctttttgagtttgattgcccagagtagcagcactccctcatttgctgccttatttaattttttttttccttttctccatccctatttcagaggacagaatagaaagaaaaggagcatgaatatttaacatccctatagatttcatagtgagtgggggaggaagtaacactgcagaatccagagttcccaggccatgctgactgatgctgtggcttcaaacagaattgggaggcttctgttttggatgcctagaaattgcataagggtcaaaaggctgggcgcagtggctcacgcctgtaatcccagcactttgggaggccaaggcaggtggaacacgaggtcaagagaccatcctggccaatgtggtgaaaccccatctctactaaaaatacaaaaaaaaaaaaaaaaataaaaattggcgtggtggcgggcgcctgtagtcccagctacttcggaggctgaggcaggagaatggcatgaacccgggagacggagcttgcagtgagccaaaattgcgccattgcactctagcctggcgacagagcgagactgtctcaaaaaaaaaaaaaaaaaaaacacagaacgaaattgtgtaaggatcatgtctcttccattcatgctcatcttttctttctttcctgggcagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtaagttaatgtacctgtactgtctgacttgttttccattattcaacaagcatgggttgactgcttttttgtgtgctttgcactttgctgggcaccagcaaaagtgacttgagacaggcaacatggcacatgcctgtagacccagctattcagaaggctgagacaggaggatcactggagaccaaaagtttgaggctgtagtgtgctgtgatcacacctgtgaatagccactgtactccagcctgggcaaaatagtgagaccttgcctctcatattaaaataaataaaaataaatgacttgaaagaggggagaggtattatttgatatcttgtgtacctccttttagtcactgttccgccctgtgctaccttcctggtaaggagtggccttctcctatagcagtattcaatggagcatgctgtagaataggcccttagagcaaactcagcatatgatttaactatatcttagtatcccctggtcatgtggtacatttgtcacttttcatctttgctcagcctttctccaactcctgactgagtgaaaggttaatgttagtccccagatctctgagcccatcactgaagagggtactatagctctgtagtcccaacctaatcttttccatagtattgggcttgaaccagagacttacagtgtgcaggatcctaataatccagcccaccagcagtcctcttcttataaagacacctcttctcataaggacaccataatcagtaatcaggttttgctggctgtggaagtactctggacagattagataacagtatgtatcaatgtaaatttttgaagctgatactgtgtaaatgtaagggaatatctatttttagaacatacacactgaaatgtttaggaccatattataagtaagtacttataaatgtactttaggaccatattataagtaagtacttataaatgtactttaggaccatattataagtaagtgcttataaatggctcagaacaaacaattatgtatatatatctgtaaatatacacatggagagagtgagcatacatgcacactgtgcaaatgatgaatggggtaaaatgttaacagtgaatctcggtgacggatgtatgcgtgttccttgtgcctttttctttgcaagtttctttaagtttggggttatttctaaagttaggatttttttcaagagtaataattaggattcacttatatcttttaggtttttaagaacctacaactatttatggagaacaagcagcccgaggatgatctttttgatagactcaatgtgagtagatgaagcacacaatgttgaagggagtcccagccagagcctcacagtacctaaaggggagggttgctggcagatgacttgggctctccctttagcctggcctgctctgtggcatcccatacactctctcttcctccctctgagtccacggtgaatctgtagtcaagagaagacacatctgctgcagaacaactgctaaagcactcagggtggggggtagatgagccctaccttttatccttccctgctcctgcagagctttcccttcactgaatgcccagccacccctgctggagacccaaaacctgtccctgatatcttaatagggaaggaaaaatttgctttattggtttgttaagcccaccaacctgggccaggcttggtggcttacgcctgttatcccagcactttgagaggtcaagtagggcagatcccttgaggtcaggagtttgagaccagcctgggcaacatggcaaaaccattatccaggtgtggtagtccacacctgtagtcccagctgcttaggagcctcagaaggattgcatgagcccgggaggtggaggcagcagtgaaccgagattgtaccactgcactccaggctgggtgtcagagtgagaccctgtcatacacacacacaaagattgaaatacttcatagagaggcatcatgtaaccctgtattctggaattcttttcctctttccctaacttcccacttgtagggccataaacttgacaagattgtgactgcactggcataaattactcctagggctgccagaaggagcaggtagtatagctttgacctaaatctgttgctttgtctcctccagactggtattctgaataagcatcttcaggatctcatggagggcttgacagccaaggtattccgtacatacaatgcctccatcacgctacagcagcagctaaaagaactgacagcccgtaagtattgcttggccagatagggcccacacccctactaatggtatccggtgaccttgcttatctaaggcctagagtcagttctactttttttccctaccattgtggtcagacactttttccctttagacctctagtagcgagataatgctttgttgtataaacataggatcaatgctgtttcccccttcccaccccaacttcaagattaatttcatggaatgctgacagcttttcacctgctacaaaaatgcctgcatttgtgttttatacatatattacttaaaagcccacagagtgaagacagtgctgtgatgttctgttaaattggagagagtaaagttgtcatggttaccagaaaaatacaggaacaagtcctttccttgaggtttctgtcatttttttcccataagagagtagatactttctatagctgatttttagaatattatcaccaagatccacgggctttttttttcctggagcccagtcactattgaatttccactgccctctgtaaatacatcagatggccttagaatatgaaccagaaaagccagaggtaggtatgttgagggtcagtattttaaggtggtataaagaatgccacactatacaaataaaaacaatttttttggatggcatttttaatttttctttttaaataagatactaccaggccatgtgcagtggctcacacctgtaatcccaacacttgggtagccgaggcaggagaatcacttgaacccaggagttccagaccagcctgggcaatgtagtgagaccttgtctctactaaaatttctaaaaattaggcaggtgtggcggtgtgcacctgtcatcccagctacctgggaggctgagcccaggagtttagggctgtaatgagctacgaccataccaccgcactccagcttgggtgacagggtgagacgctatctctaaaataataataataaccataataatactaccaactgtggcttaatttttttttttttttttttttttttgagacggagtcttgctgtcacccaggttggagtgcagtggcgcgatctcggctcactgcaagctccgcctcctggggttcacgccattctcctgcctcagcctcccgagtagctgggactacaggtgcccgccatgtcgcccggctaattttttgtatttttagtagagacagggttcaccgtgttagccaggatggtctcgatctcctgaccttgtgatccgcccgcctcggcctcccaaagtgctgggattacaggtgtgagccaccgcgcccggccaaaatttttaaacttttaacgtcccttcaattcctcgttctgttctggctacattgggtggagaggtggggaggtaggaagtggccagtatagaacttatgaatatgggcaacaaaccatagtaatatcagcagcacctatgatgagcatatgggtggggaggaagcaaggagatgaagggtccctagccaagttccctgagtaaaaagaaaggtgtagattttattatattacctagactcagagttcctcttgtgataggaatttacccaggccaagcttacatataaacttcctactatcattttcttaagactgttgactcaaattttgaccttagtccttgggggcaatttgaattaatcatctgagtaagataagagccagcaaaatcatggggacgaggcactgggggaagacatactgtgtgttcacttttggtgtacaaactgaccctcttgctaccatgttcctttctttacagcggatgagaacatcccagcgaagatcctttcttataaccgtgccaatcgagctgttgcaattctttgtaaccatcagagggcaccaccaaaaacttttgagaagtctatgatgaacttgcaaactaaggtatcttggataaaatgaagggaactgtgtctgctgtgggcagattatctgcgaatgagaggattcagggctgagatatcagcaggccagtgctgggtctgttgtagaaggtctatgctaaagataaacaaatggaaatatgatagtagttaatctctgatcaagatactgaatatcagagtatccattattcaagaaatctttattctacgcatagaagttctatactggccactccctattttgaaaaactaactttggtgtacattctaattgctaattactgtccttattgtacatgaggaaagtggatttaaagagagtcttcataattccttgctacagcccagaaatacagccatttccaaattagattgtttcaaattgaaaagatgatgaaacaattatatatttaaagtctttcaaatcctagcatagctatttttatttttacgcattccacatgaatcatatttttatatagttcatcatcaaacatctgttgaatactggccgtgtgccagacaacttgctgccctaaagggaaagactggtgtgcaaataggtgctattccttgtccctgagcctctttcctcctcctaactccgcctggtactgagcttttaaaccataaggcacttaacatttgatgatgaacatttttgtgttagcagtgacttcataactaattcatcctaacatggtggtccacagtgagaatttgcagatatttcattatagaacagatacacagggttaaaaaggaaacaaattatattgaaatacagttatcaaaatacattttaaagtccgataatagatgtgcttccttattaaggtcttgaggtggcctgtagtaaatttcaaggtagtaattaattaatatcaatgatactttaagatttctgcagcaattcaaatatgaaatgaagatgtctaacatctattgatgataaagtcataagtgctgctgctattactatggtttgttccccatattcataactgaagaagaaatactaaatttcagctagaagctgggtgcagtggctcacacctgtaatctcagcattttgggaggttgaggtgggagaatcacttgagctgagtaattcaagaccaacctgggcaacatagtgagaccctgtctgtattaaaaatttaaaaataaatataaataagttccaaaaagaggttattgaaattatagattttttctcatccaagttcatggaattctatacttgtacaactggagtttaagaactactactgtggaatgagactgcattttaaggcccagaatattttccatttgttgagatttccttaagataagtacccatgagtaatatagctggttcaaaaggtacacacaacttataatttatgctacagatgccatctggctttcttgaagagacgtacatgtccacagtgaatgagtatacatccatagacctttgattccttggatcatatgattggcaggcaaagaactctatagacggcccatcctagcagctcacatttgctgccactctgttaaatgctggtcatctggaattgtttattctcctcatgttatcattacaagccagaaataaaagcatgaatattcaggtgctgtttacctaattcaggaaaacccctcccaggagcatgaggaggtcttttgttgctctcgctctggaaccgggtttgggcagcccaggctgcctgccagatgagccctccaaacccaggattcctctcctgtgtgcccacaaagcaaacacacgcccctgtttgccaagtgaatcaatggagtctttgatgctttgagagaatttgacatagcctgccaatttagcattcacagaaatggttaaaagatcatggtttgctacatttctatcctaaaaagaatctggctagtggtgtctgtgatgcttttgtatatagaatcagtcccctcagtttggctttggacaacatagatgttggaagtaagccattcatagtaaaacatatttagtgatcctctcacaatggtggcctctgtttccaatcttgggctgtgtgtatctaacaacaaacaattccaggttcagtgagcagcccagtgtgtgtcagacatgttatttactccgtagaggttgtcttcacctcacatagatgaccataggtatggggttctcctgccctctgagtagcgtgagctcagtgacaggggctctagctccatgttgttatccactgctgtctctaagcaggaatctgagaagaacttagattcaccaggggtgcttcaaagattctaacaagcaagaaaggctgaaactgcccttgtctcttcccttatagatatggattccatgagagttcacatggggaaaaaagctccacagctaaaagtgttttcacatccattgagtgacaactaaggtccttcccacctcagacttctctcctttttttttgagacggagtctcgctctgttgcccaggctggagtgcaggggcatgatctcggctcactgcaagctctgcctcccgggttcatgccattctactgcctcagcctcccgagtagctgggactacaggcacccaccaccacgcctggctaattttttgtatttttagtagagacggggtttcaccgtgttagccaggatggtcttgatctcctgaccttgtgatccgccctcctcagcctccccaaagtgctgggattacaggcgtgagacaccgcgcctggtgaccgctctccttttaaaaacagaatgccaaagctaagccctgccatgtcctggtttgggacagactggctgagctgtttggcttcctgggtgtcttttaattgccagtttgtagccccttgagtactctcttcagtgcatccattttccttgagaagctggcaccgccttcccagagtactggtgggatctgaggaacgagctggggttgcccagtagatgcaagtatcctgcttcagtttcctaaagaggagctcctacagtgttcttagaatgaaagctaagaactggcacaggatctatttgataaacagctgtcatttgtagccatcctcccagatgtatgacagcagagtgttctctgatggaaagatcagcacccagattcccagccccagcgggttcctttccctgagcccctaggttactgccctgtaaaacccttgtttctttccagatcactgaagaacaaatagttgaattcactagtccttgtgcatcttctccctgccttcatgaagccacccttgtttttttttatctgacaaaccactgacagagacagcctggtccagataacatcttggtttcaccttctcaggtggagccatttttcctctacagctcataaccttaccactattatttcccctagattgatgccaagaaggaacagctagcagatgcccggagagacctgaaaagtgctaaggctgatgccaaggtcatgaaggatgcaaagacgaagaagtatgtacctggtattgtgaaagttggggctggtagagaaaagtgtgcagcatctgtcagggcccctggggccctggcttttcgatggtttctgagaaatgtcttttggaaatctctatactagggcttttattgactcaaagtggcaggatgggtacagtgtgctcttgtctagagcccaggcctggttcttgaggactttgctattcttctagggtagtagagtcaaagaagaaggctgttcagagactggaggaacagttgatgaagctggaagttcaagccacagaccgagaggaaaataaacagattgccctgggaacctccaaactcaattatctggaccctaggatcacagtggcttggtaagtgttgagccctccttgagctcctgctgctagcttaagaaaggtggagggggttccgagagcactggtggccttcacatgccattcctaagctacacactttagtcctctggggaaacttctggcttcagctgtgtacaagttactctggttgctgaaccttgtctgtaaatgcattgcaacattctggttgctcctaaatggtcagtgctgttacactgccttgtagtgtatatttttaagaggcaggtgccatccctattcctaatggaactttaggacaggaatggaaactcttagcttctggaagaataggaaagagcccatccatctctacacttcaacaaaacttttcgttgaatttttttgcagttgaaggtaggaacaagagataaagatgagaatacagatctgagcaacttacacagagaggcagagggacctttaacaaaagcagataaatactaggtttctctgtgtgtcacacacagtgtcacataacatctgaaacaagtggctttgttatggaagatgtttagtttgagctgttaagttctgagcataggtggagatatcctcccctatggcacttgctagtccgggctaaagtttccatctaggtcttttgtacctctttctgctcgttttgccttgtttggtgctagagatttggttagctcttaaaaggcaatatagtatagtggttaagaacatggagaaaaactgcttgggttcaaatttcaggtcacttaccagctagctgtgtgagaaaatcagtaaattacctaacgtctctgcctcagtttcatctgtacgtttttcacttgtacatctgcagaagcacctactttagagatcactgccaagattaaatgagttgatcacataaaacctttagaacatgcttggtgcactattaatttgcatcctcactagacagatgtgaaagagaagatggaacatctgaccctgggcctcagatatgggccattgctgagtcaccctaatcccccccttatttctcctttgtttgcaggtgcaagaagtggggtgtcccaattgagaagatttacaacaaaacccagcgggagaagtttgcctgggccattgacatggctgatgaagactatgagttttagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcctgacttgagtgttcctttttaaatgtgaatttttatttctttttaattattttaaaatatttaaacctttttcttgatcttaaagatcgtgtagattggggttggggagggatgaagggcgagtgaatctaaggataatgaaataatcagtgactgaaaccattttcccatcatcctttgttctgagcattcgctgtaccctttaagatatccatctttttctttttaaccctaatctttcacttgaaagattttattgtataaaaagtttcacaggtcaataaacttagaggaaaatgagtatttggtccaaaaaaaggaaaaataatcaagattttagggcttttattttttcttttgtaattgtgtaaaaaatggaaaaaaacataaaaagcagaattttaatgtgaagacattttttgctataatcattagttttagaggcattgttagtttagtgtgtgtgcagagtccatttcccacatctttcctcaagtatcttctatttttatcatgaattcccttttaatcaactgtaggttatttaaaataaattcctacaacttaatggaaacttaagtgtctgcctctttgttacaaagggcctcaggccagagttggggctgggacttagtgtgggatgaggtctcaccactcaggtcagcaagagtagattcctcccaggagcagatgaggcagggcctggcctggaaaggagtgttgtgtgcctgtcctgctgctgtgggtgtgggttaaagaggatccaaagtccatatccttagataaaagacaggaaaggaaggaagggtgcaaaaaatccacagtaagaggtgtggtgcaaagacagcatcggaggcctagcgtaggtaaagtatattgagcaggatgttgcagctgtatttgagtcaaaagttttggagagctttccatgtgtactggccagaagcagaagggagctgctttaggttttttcacgtggctaagcttacaatcaggatggggagaattaaggttccagatcccagaagccccttcccacatgggatgcaaacttctccctgcattgactgcttaattgaaaaaggcagtgtggtgtggtctctgaccactttctggtgctccagttaccctagctggctatggtctctacaaaaattgttcctttgctcatcctaaattataggtaagtctggaattcagaatgaaagtgaggtctctgtctaggtcaaatggactgcagggagcaggaaggccagaagggtcaaaattggtaaattaatggaagggccctgggccctctagcttgttagcacgagcaggtaggtgtcttcggggaggcgaacccatagtctctgttctccctgaggctggaggcagggctggtactaggcccacaacagagaatcctggaggtccttggcagcctgtatttttattaaccagtgaaagaggcagcattcaccccagggaataagggaggttatgtagccatcactgctttggagcagagagactccagccaggtgtttttgaattctgtgttgaggtagctgggccactgaatgtagggaatacaacagagaggctcttttggtacccatttgatacactgtggctggtaggtaagtaggttggtgtaagtggacctcagatgccacagctaggggacaacctttggtccaaaactagaagtcaggtaaggaggccttgcaggcagaagaccttgctgatgaacaggaagtaatgcctacctctctggcaaggacctctcttgcaagtctgagggcagatgctgggcaacaagatgctgctcaacacaactaagaagggcatcccagacagtggcagcaggcctgggagaccaaagccagagaatgggaagctgacctggccactggggatgaaagaagaggctgaacaggtaagtgggggccagatgtaaaggaccttgaaggatgcttaacccaggagtcaagaataaacttatccacttgcctagtccagtaggttttacttccagccacgtccaagaacaggccagatgggcagggtctgagaaactccctcccatcaggcctttctatgatgcccgagttcggtgtgggcaaagggcttccacgtgctacccaagccagatatctctgtgctgacttggcttttctcctctcttacctcaccttcatctcccatcccccaaatctggccagcaagtcctgtctctccattctccctgccaaaagctgtcaaatttcagacctctgctacattaaaacatgaaaagtaaaaaaccttcacaaccctcagccctttccagccactacctttcccttttttcccaaacaagcacctgcaataagtcatcaccatccagtcattcccccacccactgcaactagcttctgtcccatgagcccacaattccttctcttcactctgaccaatgtcgccaatgactagataaccaaaccaatacatgggctctccattctgcgtcttagagtctttgtggcactggacactgttgaccactccttccatcattttgagggtacgatgatttttagcaactttgctgagtggtgcaactattaaccatgtcagttttagaacattttcatctccctgtaagatcccttatgctcatttacagttaatccacattcccactcccggcctccactaacctttctaaatttgcattctgaacatttcatgtaaatggaatcattcaataggtgaccactaccttctctctgattttcacaatgccaccctctcctggttttactcctgtccctatcaggctttttctgagtcaccttttctatcaggtcctaagtgccatgattcttacttcagcatagctgatttcatttgcacctaaaggtgatgacttctctttctctctagcctagagcccttttctaagctccagactcacattatcaactacacctctgcagggcatgtctacgggcacctcaatctcagcgtctctagaactgagctcagtacccaccaccccaagcctgttgggcctgggcttcacatctcagggaaaggcaccagatcctctgtcccccaacccagaaactagggtagccaccatccttcactcctctcgtttccacacccagtaaattactccaaaatcatctcctcaataccataaaatttattttctgaaaaaattttactccttagaaattcttcaaataatgagacactatcaacttgttttgaaccaaaaagcactttttagagaaaacacattaccctgaaatgacctcagtcaggccagttttggatgcttataagtagtcccctgacaaaaatcagtaaacaaggagacaaagaaatttaacacagacttagtaattttcacaatcccaagaattccattcagacaagcctcttagaaatcactaaatagcaaagacttttttagattttataattgtactgtgtgtgtgtgtgtgtgtgtgtgtgtgtgtgtgtgtatacacacacaattttttttttttttttttgagacggagtctagctctgtcacccaggctggagtgcagtggcgaaatcctggctcactgcaacctctgccgcctgggttcaaaccattctcctgtctcagcctcctgccaccacgcccagctaatttttgtatttttcgtagagatggggtttcaccatgttggccaggctggtctcgaactcctgacctcatgatcttcccgcctcagcctcccaaagtgctgggattacaggcgtgagccaccgtgtctggcccacacaaatgtttttaaaggagtcttttaggccaggagcagttagctcatgcctgtaatcccagcagtttgggagaccaaggcagaaagatcacttgagcctcggagcccaaggctgcagtgagctatgactgtaactgcattccagcttaggtgacagtgagattctatctatacacacacacaccacacagtcttttagaaaaattgagatagtaatggataataagagttgaggtttgggtcaaaataatatgggaggggttagtgggtagaatttggcatgaaacaaaagtggccaccaattcattattgaagctagatgatgggtatgtcggcattcactatactatacacgtagacacatttgagatttttctttttttttaattttattattattatactttaagttttagggtacatgtgcacaatgtgcaggttagttacatatgtatacatgtgccatgctggtgtgctgcacccattaactcgtcatttagcattaggtatatctcctaaagctatccctcccccctccccccaccccacaacagtccccagagtgtgatgttccccttcctgtgtccatgtgttctcattgttcaattcccacctatgagtgagaatatgcggtgtttggttttttgttcttgcgatagtttactgagaatgatgatttccagtttcatccatgtccctacaaaggacatgaactcatcattttttatggctgcatagtattccatggtgtatatgtgccacattttcttaatccagtctatcattgttggacatttggcttggttccaagtctttgctattgtgaatagtgccacaataaacaaacgtgtgcatgtgtctttatagcagcatgatttatagtcctttgggtatatacccagtaatgggatggctgggtcaaatggtatttctagctctagatccctgaggaatcaccacactgacttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttacatttctccgatggtcagtgatggtgagcattttttcatgtgttttttggctgcataaatgtcttcttttgagaagtgtctgttcatgtcctttgcccactttttgatggggttgtttgtttttttcttgtaaatttgtttaagtgctttgtagattctggatattagccctttgtcagatgagtaggttgtgaaaattgtctcccattttgtaggttgcctgttcactctgatggtagtttcttttgctgtgcagaagctctttagtttaattagatcccatttgtcagttgtggcttttgttgccattgcttttggtgttttagacatgaagtcctgcccatgcctatgtcctgaatggtaatgcctaggacctcttcaaggagaactacaaaccactgctcaatgaaataaaagaggatacaaacaaatggaagaacattccatgctcatgggtaggaagaatcaatatcatgaaaatggccatactgcccaaggtaatttatagattcaacgccatccccatcaagctaccaatgactttcttcacagaattggaaaaaactactttaaagttcatatggaaccaaaaaagagcccgcatcgccaagtcaatcctaagccaaaagaacaaagctggatgcatcacgctacctgacttcaaactatactacaaggctacagtaaccaaaacagcatggtactggtaccaaaacagagatgtagatcaatggaacagaacagagccctcagaaataacgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaattaattcaagatggattaaagacttaaacgttagacctaaaaccattctttttctttttttgagatggagtcttgctctgttgcccaggctggagtgcagtggtacaacctcggctcactgcaacctctgcctcccaggtttaaacaattctcctgccttagcctcctgagtagctgggattacaggcatgtgccacttgtaatgctaatttttttgtagttttagtagagaccgggtttcgccatgttggccaggctggtctcgaactcctgacctcaggtgatctgcccacctcagcttcccaaagtgctgggattacaggtgtgagccgctgcacccggcccagccaccgtacctggccaagatttttctttaaaaatgtcttttttatttagacacagggtctcacctctgttgcccaggctggagtacactggtgtgatcatagctcactatagccttgaactcctgggctcaaacaatcctccgacttcaacttcctgaagagctgagactacaggtgcatgctaccatgcctggctaatttttttatttttatatttttagagacaaggcctcactatgttgcccaggctggtctccaactcttggggtcaagcaatcttcctgtctcagcctccccagatactgatattataggtgtgagctaccatgcccagcagagatttttcataacatgtttaaagcaatacagtaactggttattttgaagattattagtatatatccatagaggaattgaggggggaaaagcaacatcttaatgttatactaaaaggtaattatgcttaggttacaattttcagtgataacatcacatactaattcagaaagtattccctttgcagcaacatggatgaatctaaataacattatgctgaacgcagacacaagagtacatactgtatgattccatttatgtgaaattccagaataggcaacactaatccgtagtgacagaaagtcaatcagtggttgctggatggggacagaggagactgactgcgaaggcgcaccaggaaacattttggggtggtggaaatattctctatcttgattgggtggaggttacatgagggtatacatttgtcaaagcacaaattttacacttaaaaatggatgtattttatatgtaaattattcctcaacaaaaattattttttaagtaaaaaaaaaatctaagtaaaaaaagttggccgggcacggtggctcacacctgtaatcccagcactctgggaggccaaggtgggtggatcacctgaggtcaggagttcaagaccaccctggccacggtgaaaccccatctctactaaaaatataaaaaattagctgggcgtggtggtgcgggcctgtaatcccagctactcgggaggctgaggcaggagaatcgcttcaacccgggaggcggaggctgcagtaagccgagatcacaccattgcactccagcctgggcaacaagagcgaaactccgtctcaaaaaaaaaaaaaagttgtttgctctctctctcaacttagaagggagggaaagtgatgatatgctttgagagattctattagatgactcaaaaagcgactcatggtgaaaatacagaaatccaagattcatgtgaagagttgaatatagtttactgaaaatgtgagtgtggggaaaatcaatgttcgtgaatcaatatattgttttaaatatgtgatttaaagtatgtacctgtagctaaataaactattatatgtggacatttgcatctctaaaaagtttatattttagttgcaaaaaaaaaacaacccacaaaacaacttggagtgaccctatgatgaaaatgagtgtttgccttgtatctggagtggccatctctttcaggcatatacagtctctcactttgctctatctccacagacactaacaggtttcagtaaagccaagtgtcccttctcagtgctccaatggcccccatcacactgaattgtcatggtctgttggcatttctgtacccactgctggagtgggaacttgttctgactcagctgtgctgtcttagtgctctagaggatgcatccgtgaagcgtgttgcaaggaggagagagaaaaacagaaaagtgcaggtctgccctggcctgggctttccccttctcttcctttattgccatatgttgcctcagtgatgcagtcacagtgtccctgccacctttcagaagaagatacatctgcagcccagctggagaacctcaacatctgggtcccactatgggggccaatcagcagagatacaccaaggtcaatggagacagcagtccctgggagcagccgagccaggtgtgattttgccttgagagagaacacgaggctcttgagggacagataacacaacccaggtgaggaggaaagcaggtggacatgacctaagaagtgtgaggcctgatgactaagttggagccacagatagatttgggggccaggggtcaggagcctaaacagcaggttgtgggtggagttcaaggtgcactgaggttagttcttagcaagtagtactggctgtgtgctgaggtgcaggctggctcacaaacccagaggaggggctgttcagatgtctaacacgagaaactgagtaaaaattaaagaatggtttaggccaggtgcagtggctcacgcctgtaatccctgcactttgggaggctgcagtgggtggatcccctgaggtcaggagttcgagaccaacctggccatcatgatgaaacccccgtctctactaaaaatacaaacaattagctgggcatggtggcacgcgcctgtaatcccagctactcaggaggctgaggcaggagacttgcttgaacccgggaggcagaggttgcagtgaaccgagattgcgccactgcacttcagccaggacgacaagagtaaaaaaaaaaaaaaaaaaaaatggtttaaagaaaaaggacaatgttttacacatggatcaaattagacactgcaagactataaatctgcacccttctaatgtgaaaagggcttcactgagctatacttgtatattatgccctcgatattcactcaaggccattacaaggaacaaatgaaaacggcatcagcatttgctattaaccagttgagaacattctccactatttcaactaaataagtatccaggtgagaaacttcttttataatttaaccccagaaattttttttactccaaagaaccagttaacagaatagaaagtgttattgttttggctatcaggaagctcagccatgtttaattctcaatcacatcagctacattaacccataaaccatattatcgtcctcctccttataatctcatctcctatttctatattttattctctatttttaagtactcaaaacatttttaaagggctgaaaaagaatacacttgtggtagacatagcaacgtgccacccagatccccttcaaagaaggacttgttgacccaactattgggagagctgtcagcagaaaatcttcagctgtcagcccctcgaagatcatcttggctgcagagtgccatctggcccaggctaggctcttcccaggacattacaactcctttccaggctagtgccagacaacagagatcctgggagttggctgagactgtcatcaggcctgcatctcagtttagttttccctgtgcccactcctgtttctttccagtacctcccagagtgttgatcccagggccactccccaataaacatactgcacactaacctgtcacagagtccacatccaaagaacccaacctgtgacagcaccaaatcaaaagagactcaggaggctgaggcaggactgcttgaactcgggaggcggagattgcagtgagccaagattgtgccattgcacgccagcctgggcgacgagagagaaactctgtctcaaaaaaaaaaaaaaaaaaagagagaaaacatatcaggtaggggtattttacaaaggagagaggatatacaataaagtgttcatacatacatgggaagcaaaacagcactgactctggggccagactacctgggtttgaatcctgccttttagctctgtgcctcagttttttcatctggaaaatggggataataagagcacttatagggctgtaatgaggaagaaatgagacaatgtggataaagcagttccatagtgtttactatatgtcaaccactgttctaagtggtcatttaataaacagctgtgaggccaaacccacaggtcccgaggggcagttgggctgggactctccccagactctgggcatgggcacaggtacagtgtaggccagggcatgctgaagcatgcatgttcacagaaataggcctgagcagggctagcatgggtgacgtcacaggcgtgtgcttgtgtgggtgctgggctgctcctcccagacagcgcagctgcacgtattgccattcacacccacgcacccatatatatgctgagctgggggaagcccacacagaccacaccctctgcatgccctgtttttcctgcacctcctttggtggaaggaggcacctgaggcctgggatgctggcccagaagtcccaggaggaaatgggtcctgttccctgattacaggcctggcaacgggctgaaaactcaagctcttgttggcagggatttcttgagcccctagaggtccagacccagaggcaaaagtaaaagcaaggaagccttaccctgcatgttaggaagctcccaaaaaaatggagaaacggatgtaaaaagtgcatcagagcccacaggctgccggagtcagcacagtaagctcaatgagggctgcagcacttaagagggtgcctcctgtacaaaacaggatcagacagccagagaggggccccattcagcccccgtctgccttcctggtctcatcactccctgtcccctcctcagtcaggaaactctagctttgtaaccctaaattcaacaacctcttcctggcctcagggcctttgtacatgctgttacctctacttggaatgtacttttctccaacccacctttaataattcacctcgccatcgactcacttcctttagacctctgctgaggtctgaagagatctcgacccaatgtttttccttctagccctcatcataattgtctttatgtaacagagtgtttatttaatgtctgaactcccagagagctggcgctgtgtctctcttgtgctcctcatgttgtcaatgcccaacacagagaagagcagggtccacagaacttcaagcctaagccagttcaagccacactctgtgagcactcacgttctgctggattccctgtgcccaaggccagcggtgagcatgcctcccctcacccatgctgattaaccagatagcctccatgcccactgactactgaccaccaggacttacctaagccagtggatctcctgtgaggcagaaaactcatggagtcaggccgacttgggttctagttcccacctagtcatgttatgtggctttggggcaaattgtagaacttctctgaatctctgtttcctcatcagtaaattgtggaccatcattcctgattagaagataattatgaggatcaggtgtgaccacaggtgcacaccttcagcacagggtctacgctagctccgtcccgcccacgtaaagacctgccactaggtggcagtccatctcagctaccccaccgctccttactccacagaggctggctaggacaaagggtctcaatgtcatgctgaattctgagcttcttaggggacgcacttgaggaaatcaacgttaacacacaagtttccactacagcaagacaaagtcagaagcagcagccccaggtcactatccagctgagtgggttgcctgagcacacgctcttacaaactctagtctagtcctactatctcacaattcccaggaagcctggctctttctctggcatcagctggaagctctgttgggcttctcacctctgaaatgcagtaagttgtcctgctgtgcctggcagccgacccactgcctggctcaacatgtcagacaggctccaccgcgaccgtgggtgtcctcaattttggcggtgctgacaagggtagatagattgcaggggtgagtttcaagaatggcttcagaaagacaaagagtatgaattatattccataaccagaaaaaggccttgtggctggagaatggagagtgaaggatcacttgtgagagatgaaactggtgagatgggcaggtcatatagaacctgctaggccaagcagggagtgtggattttaggtgcaatgggaattcacggagggttttagcctggatgtaggggatcatgatttaatttcattttaaagatttaaagcatgataaatccaggcaaaaccaaactgatagatccaggcaaaaccaaacaaggtgagtagtctgggtgacaggagatggggccttcagcttgtgggctgagtgacaaggtcttgatggattggaggtgaggcaaagggaggaatcaaagttagctgcaaggtttttgaccttccaacttagagaagtttagcgccgcgttctgagaaagtgaagactgtggaaggaatcgtttggggagggaaggcgagctgtctaagggggggggatgtggaacatgcagtgggatgctggggcctggaacgtaggggagatgcaggtttggagtcaccggtgcctgtgttgttgggcaaagccaggggaaaggtcaggtcgccttgggatctggcctagcactgagtgcagaggaagagaagcttctgagatcgagaggcagcggctacaggaggaggaagaaagcaaggcggggaaaaccaaagttcaggagagcatttgcaggagggcgctgcggggtagggtctgagtcggagccccgaatcggactcgagtcctgtcgcgtggggacggagcgtgcagaggcccatgcgagggacagacacaaaggccctgggagctgcaggtcagaccactggacagcgcggccgcggacctaacgcccctgtaaggggtgctcacgcttggggggaactctccgaaagagaggaccactgaaagccaccctggcagcaggggcggaacagacagacgtgggtctggccgtcgccagaactgcctgcggaccagccccgcgctgccgggggggcgggacatgtcggctgtccatcagtgcccgcgaccaatccgcaggcagccccgcccccgcccctcccgggagagggcgcgcggaggacccgccgccgccggtgtgaggcggcccgtcctggctcccttgtccgggaagcccgcccaggtaactgagtttggccgggcattcccggaggacgcgccatcccctcacgtcccgtcccggtcgctgcatgggcggtgttcgggacgcgggggctgcgtctgtgcgggaccgcggggtagcggccgcccgtgcgagtacgcctgactgacgcgccggccgccgggcctgcggcctgtgggcggggctgccgtgcgtgacagggccgctcgtggccgccgggctgtgtccggggccgcgtgagaaagctctgcgggactgagggctgggtgggtcgaccgggaacggcgcgcgctcccgccgccatcgcgcctccgtccccgcctgcggcctgtctgggggtcgcggggcgcaggcgcggcgggccgacgggcgggggtcctccccacgggtgcgcgggcgcctttgttcccggcgccgaagcggggtggggcctcagggcagccccgccccgccgcgctgaggccccgccccgtgtccgcctgcaggagccgccgccgggtcccgctcgtctgccgcctcagcctcagccccaacctcagccgccgccgttgcgcttgctcccgggcggtcctggcctgtgccgccgccgcccccagcgtcggagccatggcgggcgccgcgtccccttgcgccaacggctgcgggcccggcgcgccctcggacgccgaggtgctgcacctctgccgcagcctcgaggtgggcaccgtcatgactttgttctactccaagaagtcgcagcgacccgagcggaagaccttccaggtcaagctggagacgcgccagatcacgtggagccggggcgccgacaagatcgagggggccagtaagtgcgcccacttcctgcctgggcccgccccgcgcgggggtcgtgggagcccggcccgactgcttgcaccccggccggccgccccagcgacttgggcaaactttcgggccctcccagactccctccgggccccgcccccgcttcgtctcgggtggtcactgggggcggggggcatccgggtcctcggtcacctgacaggacaccccccctcccccagctggggggagtgttccaggcgctttgccctgaggcctaaaaatcctcgcgggctggagacctgcggtgcaggcatcgggccccccagaccctgggagtggtggcggcgtcggcgaggggagctaaggcagtggcccccaccctgcacgggaacctggggcctgaccagacggtccccgcccactctttatccagaaagagcagtctgtgaaactctccgggcccccaggctgggctcttatttgcaaaggaatctttgggttccctaagtagaacttaggcagatgttgggtagggctggtcttggagcagagctgggcctactcatctccctctgggggagagggaggaaggtggtcttgtttggtttggagaagcctgagagaagccagtggctggaatttttttttttttttttctgctacttttgtcccaaagctgggttttggaggcctatgtgggtgggcagacactcaatgccagggcagggactccccttgccctgggaagcacgttcccaggagatgggccccataaaggcctgccccagcgccttggagtcaggtattcttctcaggagtgcagctagtttgtgagtctagccctttgtcctctgtccggtggcttcagggagcgggagcggcttctcttagtttcctgccttttcgccttcccaggcctgagcttggctgcttttgcgctggtctctagtgggaggagggagttgggggattttgaaaaaccccagatctttgagagttgttccacttagcgcgtgcctgtgctgtggtcggatgggcactctgctctgccctccattcccccgcctctcagtgtgaagagatggtgtgtctgcaactctagactgggttgcttcttgcagggacccccggagactagggttagagagacccaggctcagaaccaggtctgtgtgaaggagggagatcaggaagtgacaccccaagggggggtcccacaaaccttcttgggtatggtgtgcatgtgtgtagatagttgaggagattgggaggaaggaaggtcttgtggtgaggcagccaaccatcctcagctacaaactccagatctgacacagatctaatgcagctgctcaacttagaaatggcagcccccttcctcagtgcccccactgaccctgtcagccattgggagaacattttagtgacttgagagcctgtggattccaaagtaaaggaagaatcgacactgcaaagagctgagataaccctgggaacatcactcaggcatcaggctccaggcatggtccccttccttactccctatgctgtgttgagttggtggcactggctggaagttctcagagatctcctgggtaacagactgcattttaaaccacgcatattagcaggcaataaatgtagcacagtggttgatggcaagtctggatttgaagcccatatattcattgtgtgtttcccggccagttacttaccttctctgagccttgatttcctcatgtgtaaaatgggggtaataatatatccagtatatatggttgctccaggatcaaatgagacaattcatataaaacatctagcattgtctgacacataaatgtttaataaatatgagctgttattagcaataacattaacaatagaccgtggctgcccttcaagaccctgaactgggaatggtgagggggtgaggtgtggctataggtgaggatgaaggtgcaggaagggaggaggacggtgaccaaattcacatttcacttagattgctactcagaaaaaggcaggaaacagttttgttttgccccaacccatgtgcccactcacccacccaccatgtagaacagtgtttctgtagagaaagtgtttgcagttgtgcacatttgactcctgggtgtgacctttcttgtttgtaagttggggcctgaacagaaaagaggggtgcagagaggggatgatttatcatgcctcatgtatttgggggatggaggcaggcatacagaggacctgataaccccctacccaggggtgagcaggtgggtcagccagaggtgtacaaattcagtgatctcatcagttagctgggatttaacaggcctggagagtaggcagaggatgtaggaggaccctggcttttggattttgggggctcagattgaaaagagcctggatgaaaagtctggtcgatgcttgtgtgttcagacttaactaatggaggcttccactgaagccgtgccctgcttctacccattcgtgggctctccttgttggtgggttctgatccctgcggcctctgaggaggttggactgccagccgggttttctgccacctggactcttgcccaagcacctcaggtatctggcctttcctgctgaccctaccagggtaccagcagtgcctgaagcctggcctaagagagagacactgtgctaccttcagggtgttggtagacctgagggagagacagtgtggtttaaggagcactcatactgatatgtaagaccttcttctgccctgactctgtgccctctcttgtcagcagttgtgaaatggagctgacacctcctgatttctagggtggtcatttggctcaaaagcggggtggggaagtgcttaggtgttgacaagtttggttgtcctggtggtatgacaggaaccaggttggtgtttggccttttatgttgcagatactgacataaatgaataaaatgcagttatgtagagttccagctgagtgctattgagacctttcgtcccattttacagatgagcaaatggaaatggagaatcgtttgtgcccaaggtgttgtgtcagtgtgtgccagggcctcagctggggccagctcactagtcagaggctgactagcaaccccaagcccatgttccctgtggtccacgtggctcctctgtgagacacctttgtgctcagaccatgcattacagggcaaagaccactttggcttttgtatcccaagacagcctcagcctctctaggctctggctccacactttttcttctggtgttttctgagttgagatggttcagactcgtgtttaatgaggttgtccatttgagggtctttgtgggcccagtaaacactggtgtagacactgtggaggtactctgaatactggctgagcatgtccatggggacttctgaggccccaggcttctgtctttggctgatccttcactagatacttttctagatgccaggccctctgaggtgaaggagtatgggtgcttgcttttagggaaatgaaaacaggcaatgacaattgtgagcagtacctttgatgaagagggtgagggtggcggaagaaaacagctaactgccttgatgaggcaggcgggctcagaagggaggtagtagtggaaccagctggctggggcttgacccctgtggctggggagcagaccacaccaggaggcctgctccgagggctaggcccacagcacgggagagggcaaggcccaggccggtgtcatcaagggttttctttgtactggctgttactcccagtgggagaggggcaggagcacatgtccggatctccagagctagccatccctgcctgtgtatttcctgtctgatgtagttttttccaagggaagtcagaccttttagtgtttcctcagcaccccaggcttgccccgatgtgtgggtttccaggcccccccgcccagctgggatcctctgtttcctgtgaacctaggaaaagggcctgcttcacagataactggaggagctgagcacacccagtcctgggacccacaacacctccagcttctctccagactcttgtgtgtgtgtgtacacagatgcacaggattcagttggagtttagagatcttggctaccagctttgctctgggtagcttcttggaatgaggcacatttattaatatttgcaataagtattattatacctccccatttgcccagtgctaggcaccaaggattcaaaagtttattgagacatggtttgtgccattgatgaactcttggaaaagacagatgtagaggcatatgattataaccctgtgtgttacataccataatataaaggcctctaccaagaactgtgaggagctctgccaggaaggcattaaaggagaggtgacctttgaggtaggccttgaattactgaggcacctgtctgtgccgaggctaaggaactgggaatcactctgtgcaggggtagggaacagatatggtgtggtagagaggaagctgggactggttggagacagctatagccctagtgtgccatgtccagcagattacactcagtcctggaggcgacgggagtcagcggaggctgttgtgggactgacttgcctctgcctgggagaccacagtggcagtggtttgtcaggcaggtgttggggagggaggccagtgagctcctacggtttgcacagtttgcaggagagagctgctgagggcccagacaagaccttcttgctgaggcagggtggggatgccatggtggtagatgggagaagggctgggtcttgaggggtgagacagagcttagtagttaagcatttgggccctagactctgcttgaattaaaatcctgttgtgcagcttacttgctctgtgaccttaaagcatatgactacctctctgggctgcttatctgtaaaaaggatagtattaaatggcatttattttataaggtttttgtgaggatttgatgagaacctgtataaagtgcttagcacctgtataaagtgcttagcatggagtgtggcacagtgtaagtgcttactacgtaggatccctgacaggacagagatgaggattaagttctcaggaggacctggtggggaggagttcgaatgcacctgctgtcctaaggggctggtgcctggccaccaagccctttgattcagaggcaaggaggcccgcccatcctctcttgcagctctcagcagtccctctattgtcagggctctttcccactgctggttttgcagggagacagggcagttggctggaagcagctgctttgggtggtaaccccttctgtagagtgaaggtatgatattgtcctatttctaagtgcctagcccaggacgctatctggagagatcttgtctttgaggaagtgctctaatgccacagcctcgatgtggtagggcctgcctctgctgtcactgttctgtgtcagtctccgttaactcacagcatctcttcttactctgtgccttttcctgtgggatgtggcttcaaggagaccacagatgataagttggaggaagggagtgaggcctgagattgcaaaggtgtgatattaggagactgatttttccccctttccacttgctttgttagaactagagaagtcactgcagactgtgaggagagtcctaggagagatgggtgtgggataggaggatgggaatgttctggtgccccttgtagagtgccttgttgagaggagcatgggaacagtgcgtccttggagggaatctctgagagcagcatgtttacaggtggcaagactcaaggtggagccacgggtgaggagtggcttcaggctaccattgaccagatagtcccaccttgctggtaattgagctgaagtgcctggagatttgtggctagagctggtcagatttgggttcaggtctcagctcttactgtgaccttgggcagattacttttccctgtctgaaattgatctttagctgtaaaagtaatttctcctcacagagctgtatattaataataagggagattatatataagagtaagagattttatgtgaaaactacccagcacaacatttgtcacttagtggccacttaacagtattcatttcctttactctcacctttgttaaccacagagctccaatcttttccaccactaagctcggccacaagtggctgctccaacaacctgggctaggagtctgtttcctacttgctttctgtgttccttccctgagtctgtacacagaggactggggcatcaaaaaactctggaattggaatagcatattgggagttcatggaacattttctcacctgttgtattgtttgaacttcacagcagctccatgaatccatggagggcaggagttactatccctctcttacagaggagaaaaccaagccctaagggtaaggtcacatagcaagttcatggcagatcctaggatttcagattcctgctatagtattcagtttattctcccattctggggaaaatgataataggatggaccgttctataggaacagagggttattttgttttgttttgtttttcttccctctggccacatgtgtttgaccagcctagacttccatctgtggacaagctggactcattgcctcaatttcaaaacccgtcctgcactcatccttgccacaacaccggtgtcgagatgcttggcccacccagacctttcccacactcctacatgggggtgaatgtagccgctgattgggggtgggacacagaagagcctcaccctcttgactttctggtttgtggtgacacttccgcctaagcttctacagaacatgggaacctggggctccatgccccaaatatcctgagaagggcttttccaggattctctatgacaactcaagagcatgcattcattcatgtgctctttaatttgtcatacatttgttgtgggccttgctactgtgtgttgggcacactgagctggatgtgattactccagcctctgacttgttcatggtggcagtagatctctagcgtggtttgtgttccatctcaccatacctactctccttaaaaaaaaacaaaaaactagctttcttgggcctcagtggctcattgaataagtgagagacgacttgtgcttccctggctttagactctggcatttgctaactgtgccaagcactgtggagaccagtagtggacataaggttagggtagatacagccagaaaggagataaataggcagacagataaaattcttataggatttggtgagttctatgaaggaaataaggcaagaggctgctctgtcagggtaggtgagggtgtgctgtgataacaaatgatgcccacatatcaatggctggcaacaaggaaggttaatttcccctgtttgctatgtgtgctaacctacaccaccgtccctggagagtggcctcagtggttctgggtgaggcctctagtacagaacctgaacaccctgggcaaactaggctggagagggatttggagtatggaggtttagaaggaattaatttctatgacttaggatgcatgtggtgagtgtggctttgctgttgtggtagagtagagtgctgcctgtgtgggtcaagagactgtcttggctgacatctttgaaataggtatatttgtgtggcccttgcctaattgtcatctctgccatagctgcccccgccttccccaccaacccatggccctacttgagctgcctgctgagcctactctgttgtggccacagtatcctcctgctgcccagagtttgagtttctcgctacttctgccactcatcacacttgcttcccagagctttgctgggtgtgctgaagtctccagagaaagtggggaatatgggtgcctagagacttctagggggtgttttcagcccctagagagtagagaagtgtgagaaggggttggtccccgaatattctgtagttttggggaagaagcaatggggacagtggagttggttgccttaagagaggctatggtcccaagagatgggacttggagagtctctacttgcattcctggctaagcccttaatattctcgcacccctccttgcaagactagttccttttgaggtggttcttggctagcttttgagaggctgcagtggctgtgtgcccaggcttaagtcctagctttctccccactctttacctcctaggaaccgggcccatcctgggggaggcagggaggagctcctttttatagtccacatgtaggctttgtgtataggctctgctgcttccttccggccctggcccttcctggtgttggccggtggccctggcttcagcaagactcaggatgcagagtaaacggggcagggcgttctcctacagaagactggcatccctttttactgagcccaggcctggcacagaggaggctggagttaggaagcaagaaaacgaggtgacccagtccttgatcctgagaagcttgtgatctggaccatgcagaaaaccgctaatgcccagggctggcaggagacagctgtgcctaaagtgctgtaggctgggcctgagggccaagatggaatcgcttttctccctgtagcagcccatcctaccccctggggtccctccacctgccagctggcacacaggtgttgggtaaagcatctgtacaaaggctgtgaggggcctgggcgcaaggcagcaaggcttgggtacggaggagagtgggtggtcttactgatgttctgggccctgacagtgaacagctggccaaactggtgaggagttgggccatggcgagtttggccacttgtttgtgtgtgtttagttcttcctgggcacaggtggggagttcactttggggttggcttcctgttttcttggacacctggggatatggttgagttaaaatgttctgagggaaatgattgttgagggcatgggcttgggtcatagtgtccttggcttggccttgcccatggtaggagtcgggtattgaccacactgggttctggctgtgtaggtgtggatgtgtggtgggagaattccaaccccaccttgacttcagcctaaggcccagagagtgtgggcctgagttcacagggtgactaaccctaggaaggagtgaaggagagctccagttggccataggtcccacaggtttctcagcttacttgggggcaggaggtgagctaaggaatacaactccattccctcagttgggaggttgggcagcgatgggggcaggggtgatggtccttctctgaggcactgaagacccactttatggtcaccctattctgcctgatagtccaccacctgtttggaggcagctcccctcttcctgccctagtcacttcctccaccttcaacagtaattgagtataatgtgcctgtgttagcttgtccatcctaactgggactgtggccttttcttgggcgttcttagcatatctaccaaggaggttggaaaaggtaacatatgtcactttgttaaggattttgctgagggccttttgaggccagcagtgaaacacagagcattccctccagtgagaacagaatgtctgttgccagactgatcacaagaggatggcctggttctggcctgagaactggtctacaccggaagcatgaagctccatacaatcatgtgtgctagtaaagtttgggcaagcagctgtcctaagcaggtaaaacgataggttattcaacttcaacttcagagaaagaaatccaaggtagccatctctctcagtaggggaggtgagttcaaagaatttaagctgggctgggcacggtggctcacgcctgtaatcccagcactttgggaggccaaggtagggggatcacgaggtcaggagttcaagaccagcctggctaagatggtgaaatcccatctctactaaaaatacaaaaattaaccaggcgtggtgtcaggcgcctgtaacccagctactcaggaagctgaggcaggataattgcttgaacccgggaggcagaggttgcagtgagccaagatcgcgccattgcactccagcctgggtgacagagcaagactctgtctctttaaaaaaaaaaaaaaatttaagctgggctttctgggcaggcagtgagtgaatgagatccctggcaccttggagtgagatcagagtctagatcactcactggggccctgtttcccaggaagcaaaaagggaacaggagagtggcttggcttgggctggcatggcgcaaagggagtgcagccagatgctgtggcggctgggtcaagtagctctaaaacaggagagcctagtgggttctgctgaagcaggatgtctcagcaagcaggcctctgacctgacgacttccggtcattatttagaacgtctccagccccacagatttactctgttggaggcttaaggactaaaccaacatttactgtccaagaggtggcagtaacacagaaaccaaagaggcaggaaccaaggttggggtggtgagatgtcatggggttcagggagtggcagaggggaaggcctctattctccctttcccacttgtctccctacctcagcccctgttgctctgacgcttcccccaccactatgagtcctaggcccctgttgtttccttgtaggagggagaccccagatttttatttcttctcccaacccattccatttcctctcccttgtgagagggctctgcagagacagggcagcagagagagacttggagatatgctgggctgtggtcaggagggcctcatttatctagtgagcaggaacaggttatatagcagtgatgctttcatagtatttttcttgaataaggtccccaaaaccttattaagttcctggtaggcatggagcattgaagatggcagacatggtgcagtctctcaaggagtttacgctctagtggagaaaacaatcagacagccttaaaatacatgtatggctactataataaatactgtgaagcaggagtgcaccacgaaacctggagtcctctccatcctccccttctgtctgtgctggccctgcttggccacttccaacaaggattctgaggcattacagagcacacgtctagggtacaggcagtcttggctttgaagtattgttggctttgccactcaccaactgccaccttaggtaagttaattaacctctttgtgctcaatttcctcactcgggaattggggaccctaatacttacttcatagtgttatttgcaagtataaaatataaggcagtttttatcgttgtaaggcagtcttatcaagagctttatgcaatgtctatgcaaagtaaatatgcaagaaattagtgttaggatgccagatgctgctgttgctcatcgtctcttcccacccagcagaggtagtaactacttcatcctctacttctcagcttcttatagtatttgtctgtggactctaacgtaagctatctagcataatactcatatctaatttttccttccacattcaagctccttgaggtacttgtattccaagtatctagtttaatgtctctcatgtaggtgctcagaacgtatggttaattttgaataataaagccagtgggacttctcagagggctggattgggggctggtgagagaaaaggagatgcgaaggatcattctgaagtttggagtttgggtacctgtttgaatggtgatgctcttagctgacccagggggtatggcaagaggccttggttctggtgagagagagaagacagtgtttggttttgaggagttgtctttgagacgttttgagatgaagagtggggatagcaagtaggcagctgaattgaaggatctgaagcttaagaggagaggactaggctagagacctgtttgtgaattatctgtacataggaggtaattaagggtgtgggcatggctgacactgatgaggaagaaggcagagcaccggaagagaggaccttgacctgagccttgaccaaggccattgctaaatgcaggtaggcctgtagcgcacaaagaagcatgaaaaccaggcaaggttgtgcaggagaaatcaagggcagagcttcaaggagagagggcgtgatcaaccatgttgattgctctgaggtgtcaggaaggaggaccatagataagtgatttatgggtcaggcacggtgattcatgcctgtaatcccagctctttgggaggccaagactggcgagtcatttgagcccaggagttcaagaacagcctgggcaacatggcaaaacaccatctctgcaaaaaatataatcagcctggcgttgtggtttgcctgtagtcccagctacctgaaaggccaaggtgggaggatcacctgcacctggggaggtagaggctacagtgagccctgttggtgccactgcactccagcctggacaacagagtgacttatgggccaggcatggtggctcacacctgtaatcccagcactttgggaggccgaagtgagaggatcacttgaggccaggagtttgagacaagcttgggcaacatagtgagaccccatctctataaaatataaagaaaaattttaaaagtgacttatacccattgctctggttctccaagtagcctgagtgtactatgtgggggagcattttatgagggacttttgaggtcctgggcatccctgagacatggccctggtgggtccccagtctctctcctgtagctaggagcctggcatacacaggccatctagaaagaacttaggcagcatcagtctgcaagggaggcagagtttggagcacactagactcctgacacagaggctgacccatggctgcctggagaatcttctgggcttgtgcagaaaagttggggaaagccacctttccctactgtggcagtcaaggcttggtataggccatcttccctcccttctcatccatgatactctgaatgccctgtttgtggaacttagtggactcctgcacctctgtcttggtttccatgaacccctctgcccaatgtgtgcttgtcagccctccagacctggcttttacagagcactccaagacattgctctacacacactcagctccacagacacctccctgttgttctactcgttgcatgacctcatagttatctgcttccccatctgtttcctccatcaggcaggggttttctcaaaggcaggaactcaaatgccttattgatctctgtttccctaggacccagcccagggcctgacatggagtagatagatgcccgataaatgtttgatcagatgatggaatgaatgactgcataagccgaatagatcactgctgagtgctcttccagctttaatttctctgggtgggacctcctttcccttccttcctttggattctgttcttacctctgaaataggtgagattaatttagagtcctagggaagagggctgcctgcctggatggtacaaaggctggctgtggctgctctgtgtttctctgtatacccagtgtcagggatagcagctgctgtggggacactgcagagtgtcccagttctgccatgactcaccatatgaccatgacaagaccctttggaccctctgtttcctcattgtttcaaagagatttgtctagaatctagaccaagcttgtccaactcgctgcccgcgggtctggtttgaaggcagctgctgcacagtaatgggaaggacagtaagccccaccatcgccttccctttgccatataaccctgtgacctcagaagtccctgaacccagatccttgtttgtaaaatggaaagggatagctacccacccctccactttgaccttcctttggtggcagccagatgtgctcagggaaacctcagatttgtgagtatgtttctactgagaggcaggaggaggaatattatagtctcaggaagagggaggaatgagagggagaggagaactctcaccctttccacaggctttgggcatatgcagccagcttggaaatgctgaccatcctgtgacctggctactattgcatcaggcttccttcccacctgtgtccaggtcctatctttgtctcttctttggctacctccatccttttccttcagagctgcaaaaaccttgccaaagccgtatggtcctgatgggtgcagagcccctgtgcctctggctgcctgtgaagggggatctgcagtgcagtgcctgccgtaccccacagggagttgtataggaacctggggatggggtgactgaggtagggggccagaagtggcccagatgatactcacttctgctggaacttttctttgtccatctttcagcatcctctggtggagaaggcttagctccattaatgctctcctaggtcctggctgtgtattttctttggtatgggagtggggaactgggagggactcatctgtttgccacagactacatgtggagagggtgattggtgagaggtcattttggagatctacacacctcttctgacccctgggaccactctcctgaatctcctcatggtcttggcttcgggcccagcagggcgtgggtgctcggcatcctgctgcttttactgtttttcccaaagctctgttagcttcccgtggttcccactggctataggaatctttatgtaaggacttcccagtgtttccagactgtatttgcccccaggggtcacattccccccaactatgcctgctgctgctttttctgttcccgttgttacctcactgaccactgcattttcagtatttgttgtagcccctcagcctctgtggcccactgtgccacctcttccaggaagccatccttgataccatctttctggtaccatttttgagcatctgtggcctttgctgcctgttagataattagctctggaatctaactcccatgtgtttactcctgctcttaagtactctgaacattttgaggcctccccaggccctgggtttgtgttggactggtagggaatggagagaagagtcaaatgtggtttcttccttggaggagctcataggccagtggcctaagacaccagggaagcaccagcaggtgtgacaagtggtgacacacaagagaggagggggcacctagtgtgcccctggcctgggcctggtgggggcttaagtgaggaggtaggagtggcttgtgcaaaggctgaggcaggaaggagtatggggagacatgggctggtgtctgtgttgtgttaattggacttggacccttttgaggagggctgggctttccctgccttcctccagtgccctctgcagagtggggcacacaggccttgaggggttctggggtccacaggccacaggtgggggtagtaaaaacccccagaagggaaggaacctgtccaggaaatcttcgcttacctctttcaagctctttagattttaaagagacctttcatatttactatgagttgcagtttttttaagttacaaaatttctttactccatgcatgtttaaatggatagtttttgctaccatttacatcaattagcttacacactttgggttttgtgtttttgttttgtgtttttagagaaaggatctcgctttgttggccaggctggagtacagtactgtgatcatagctcatagagcttcctactcatctgcagcctcaaacccttgggctcaagtgatctttcccaccgcctcccaagtagctgggactacaggcatgtgctaccatgtctagctaagtttttttaatttttattttttgcagaggtggggtcttcctctgttacccagatgggtcttgaactcctggcctcaagcagtcctcttgcctcagccttctaaagtgagcgtatgcactttataaagggaaaaccctctctgaagagaaatatttcagacagggcatggttgctcatgcttctaatcccagcactttgggaggccgaggcaggcgaatcactggaggtcaggagtttgagagcagctggccaacatggtgaaaccccgtctctactaaaaatacaaaaatcagctgggcatggtggtgtgcatctgtaatcccagctacttgggagactgaggtatgagaatcacttgaaccccggaggcagaggttgcagtgaaccgagattgcaccactgcactccagcctgggcaacagagcgagacacccagacccagagaaatatttcagtgaaagcatgtttcatgttcagaaatttggggtacttttggttatggttttcttggccatatcttcatcatttggtgggtttagttttcaatatgtcctcctgtccctttctgattaaatcacttctgtatctcttacctcattggcatctcactgtagtccaatgaaggaggtgccaggagtggtgattctattttgcagatgaagaaaatgaggctcagagagatgggtccaggcctcctgcccaccttgccagtgcttttgttcataatacctctctacaccctttttgcctgctgagcggttaacaggcttataaatttgggggacctctgtctgttgggctatttggaagtggcttttttgagcacaggggatactaggacacaggttccaacaagaaacttaggtctaatttagggtcttggatttgatgattgggctccctctcccccttctagggcctgccatgtgtctgccccctagggcaggattggctggctctcactggctgtgctctgaggagccctgcctttgctcttctctttctcttccctagggttcctgctgtccttgaactctgccggggcaacaggaatactgcccttatgcacatcctcgtggtggccagacagggtggttatcccactttactggacaggaagagagctagtggcaggcctgtgacttggcctgatgtctcgggggagtcggggtgggagcaagagtcaaagacaggaagcagccctaccttcagaagagccaggcacacagatgggtcctttgtggttattgtcttctccctttcccaggttccagactggctgcccgccatatagcttgtcccttcgacagcaccctctctccccactcagtggattgggtgctgtcccagggtgggggcttactctcctgcagaggcaggttctcagctggccctgagggttgtatagcttcacagctcccagagtgcttgggcatagggtggctcagatgaggaaacaggctcctcttttaaagggaaagcaactgccattactggcatgtccaaaagggggatgctgtccaggactgcatggcccagaagctccgctctgagtaactgctggtctctaatctctgtggtctcaggccaggagtctgtccctgggctgagaagggaggggagaagagggaagagatggaaaaggaacattgctcttcccagtctccctggcctcgtcatataaccttttattttacaaattcagtcttgtggcattttgctttaatgcttagagctggccccaggtgccaggcagcaggcacatttccttctgcccaaaaaacaaaacacacatgaatctgaggttgggcgtggtaagggcaggccaaggctgaccgtcctatgtggatatctgtgaggtagtgtgcctacatgtaagggtctgttgtgggtctactcccacgaaagccttcatcttacgatgaaccagagaggactgcagacttgccaaggcctagctatatgcttggagccaggatttgaaacttgatctttggggatgcacgtgcctcagactattggtgagtgtgctggtggggcactgcaggctcaggaagcagcgtgtctgcatatgcttatctgtacacaagtctgtaagagtcccccttctccccatacaatgcccccatccctgccaggcttctttttgagctggcgaaatagctgcttgtattcttctcttcatggatcatctttatttcacccaagtttgggtttgcacctagtggagccttggcaagtggccttatttgtctccactactgccagcttttgtaggctccatgtcaatggcactaggcacacaggtagacaggtgggctgtctccagacttctctattaccagcacttcccagcatggccctcacagcaaccatgtgaggtaagaggaatttccagtttcctgatggggaaactgagaccaaaagagtgagggactgccaaggcctcatagctagaatttaagagctgtgtggttctcctcactaagctgggaaggggatggtcagggaaagcttcttgaaagagcaggagaaggcagatctccatcagggcagggagcagagcaaggccccaggattctgcctggggcaccaggaagcctggcttatgagaaggttctcagtcctcagagctgacatgtgatatacagtccaggcacaggtgtgtgtggtccctcattacttcagagccccttaagaaagactgaatctatttcttcatcagagtttgtgagcgattcaatataagggactaaatccccctcactgccccaagcagcttatcttgtcactgtctaccatagccacatttggaatggtaatgcccaggccattcctgaagcccaggcagcattcactgtgggcttggggacatggtgccattcctgaagcctgggcagccatcaccatgggcctggggacgtggtgctgggcagaatgaatatgctgcagccctgatgggctgtgctggctgggctttgccttcttgggtcaaggcgagcctgggcattaggcctttgtcattatgctatacatcaggcttctgggctgctacttgtgggatcctgcccttgttctcaaagagttcctccttctcactgccccatcttcttcatagtgacggcctcttcgtacccctggcttttcttttccccagagttcccctgaatgttgtcctctcatggttctgagttggggaggagtgctaggtcatggatttttgggtataacgtgtgactttcctactcccctaagtagttctccccttctcaagctcctaagttctgggaaggtctcttttcctgccctcctgactaatggcagagaggtctgacccagactgtgccagactggatcattcgctctgtcacctgccaccagggcagaggccattcctctctggggagtctccttgtgaatttctggggaccagctctggctctctcaagaaagaggatgctgccatatttgttctgggaggcaggaagatgaagggtgaggttgggaggtagcacaggatgtggctttcaagaacctagtagagcatctcactgtgctgccagggccaggctggtgccttcttacggaagacagtacaagggatcaggtgtggccaacaggaacttgggagcattggatgcctggcgtggagctgcgctatgcttacctggttcctatctcaggcacctgttctgccttcaactgatctcaatggaaatatgtgcaggagtgcaggcctgttgactctgtgtgtgtgtgtgtgtgtgtgtgtgtgtgtgtgtgtacagtcacactcacctttgcaagaaggtgtgggagggggatgacagcagctccacaccggaagagcagactggtttctgtttcaaaaggcagagtttgtggttgttctgcagtttctgatctagccatttcccctgatggaagctcctccttctgtcattggaagccctgtgttctcggttaggtcttttatggcccagttctccctgcccctgtttctacccgagtataactgttcatttttgttccccatccctatttggttttccctccttctgcctgtgtacctgcctttttcatttcttcagtcattaagcaagccctggaggggccctgcctggccttaggtatgtgagcatgtatgtacgtgtttgtatgcacgaggcatcaagtgtattagtaccacaggaaggagcttctcagttgggcccttggtggaagagaagggagccggggaggtggacatttccagactaggaatcaagatgaaatagggatgggaagcaacagctattcttgccctaggtgtggcttagtgtggatggagcagagagggtgagagggcaaaagctggggaggtaaattcctgagccctgtcacaaagagccttcaaggccatagaaggtgtttgttgtttgttctccttagactggcagtgggcagcctcgggaggggcttgaatgaggtctaggggggtcagaataatgttttatgaagagccagaagactggggaaggatggctgatgggtgagaaccaggggtcagggaaaacagcagaaaaggcaggactctcagctggaggacaggctggaggggtttgctgaggggacaggtggttccaggtagaaccagaaatgtgtcggggagctagggttagggccagaagcaatgcccactgttccaggtgggatagatgtggagttttgtgacagttgctagagggacaggacaggaagagagcatctgggggagttgggccgttaggacattccaggcaggttggtgtgcagttcgggctggaagggctcctggggtgggggctggccaggcaaatgctggcactgcatctggctccgaagttaccaggccagtctgggtttagaggattttaagaggcttcagagcttggggagtactcaaagaaagtcatagctgttggctaagccaagcttttttcctttgacctaatctaccttaaatcggtgatttctgggctggaatgtctgccctccagccagcaggcagcccctccctgtgctggagagtgtgtcagagcctgtgcacctttgctgagggagcagaggagtccattggatgggccccctccatgtcccacatatagtgctgggtgtttctagctgttacgacttttgatcttaccaacatctttctgatgctattgtgttactcttatgttactctcttcacaggtgtagcagtgaggtcagagtggctgtcactttattgagatcacatagctaatgtgaggtagagctaggattttagttcagccagtctcacccagagctcgtgcatgttttttctaccaccctgccttctagagaaactcagtcaagccctttgctttacacatggtaaaactgagactctgagaagttaaataaattattcatcattgcctcaagttttagtggctgagggcaggtcatctgctattcaagcccgtgtacctctctgactatgccttgtgaaggccacgggagcatgcaagggccttggatagctaagataaggaaatggtcccaggcaaaatggtggtctgggtccaggatggttgctgctcttggacagccagtgccttggtcaggggatggggtggggattaggccccttgctgagacttgtgtggggatggctttagcagttaggcatttcagggatgcaccagtcctctgggccagagcctactttctcccagggatcctgaggcccatcttgacctacccagccctgcctctgggactttatgatttgatttgtgtgtaccttgtgtcaggaagtggtcctcatgaacagccttgggtccctatttcttcttctggagtctagctcttttgcaggtcaaaatgtctcagtgagggaacaaagatagggaagggacaccccacagaaccacctttcccccatatagccctctctgacatctcagcccttacttagcaaaggcctatccctagactcaacccagccctcttattaccagagtgactgagtggtcttggctctgcctctggggttcctccctgagagagagtgtaagaatgaggaaaccaggctgccctcctttcggtgttgactctgggagaccccagcttgatcttggcctctttgctaccttcctagttgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaaggtgggagttaagggggtagaggaggtagaggatagttaggggaatgcctgctggctcctgcccagtgggaggtatgtgccctcggggcagctattgataccttgctcacagccacatctgaggatgaagtgaacatgtggatcaagggcttaacttggctgatggaggatacattgcaggcacccacacccctgcagattgagaggtaagaaccactcctgaaggggttagggctgggagcattagggaccagggggacagggacagcagacctttgtgtgcccagacatctcccaggcctgactgtagatggagaagtggccctcacctcaggcttctctctccaggtggctccggaagcagttttactcagtggatcggaatcgtgaggatcggtaagtactgagctgtggctgtagcccagcagggtggggatgggcatccagaaccttagccaggcctctaagtagctgcccggagagccagaggacccaggggaccttaagtggggccaggagggtgggcagaaggttctgccacgtgtagctttctgcacaaagtcctctggtggccttggtgtgagctggtgagaggagccagccacatgctgtgtgccctgccactatgggcagagactggatgtgtagaactggctttgggtgtcctggaggtgagggtggccagtagcgtgtgaagggtgccctgaaactctggctctgagacctggtatggcagccagaggggccaaggagagaagggggagcaaagaccagatagtgtgggtgctgcaccatggtgaggtcctttgacctgaaagaaatgggaagccatggaagggttctgaggagaggagtgatgtgacctgacttgggttactcaagatcactctgcctgctgtgtggagagctcagttaggagctgtttccacactccaggtgaaaaatgagagggcttggatcatgatggtaatggtaggaagtggctggattttggccatgttgtattttgaagatagcgccagaaagaatgtccttaacagattgattttggggtgtgagaggcattgaggataatagtatgctgtttggcttgagcaactggaaaacagagtcaccattgactgagatgagaagatcatgggactagcccatttggagggaagcttgggttcagttcgaggcatgttaagttttctgatgcctattagctgtccagggagagatgttgagtaggcaggtggttagagtgagtttaggtacatacacagtaggggctacagattttaacttgggagtatgtagataacactgaaagccacagggctgggtgagatcacctagggggcaagcagaccaagaaaagggcagggtccagtggctgatcccagaccacccccatattagagattggaggggtcagggacaactagtgcggtaagaggagaacccccagagtgttgagttctggcagcccaaggaagaagacagtggtgaaatctgaccgttgacccttggatttaacatggtgagaagagcggtgtcaccctagcggtgaggggctcaagggaatgagagggagcaagatgactcttgcaaggagttttgctatggaggggagcaaagaagagggcagtagctggagagctaagcaggggcaagacaggtgtttttggtggaagaagtaacaagctgttcgtatgctgatgggaaagagaccacagagggcaagatcggtgatgcaggcagttgcaaccagaaggcctctgtttcctcactggaaaacaaggaccctgatagcacctacctcctgggtgactgaggatcagaggagggggcgcctatatagagccagccctccctccgcagtgcttgatccactcagtgtggcgctcagccccctcggggaattttggtgcctcctcccaggctcttggggcccagaggaggtggtcccaccctcccctcccctccttgacaccaccttccaggagagaaagccccccccttcccccttcttgtccatcctgttgccaggtccagcagtgtccccagcctcacatcctgggcaggacacccctggagagcaggggcgcttgttctctcagtttctttcctggagccaggccctgtctagccccaggaggtgcccagttgccagtcattgtatttggtggagcatgcacctcctccttcctccctctcatgggttagtgggttctgaggaccaaactcccagcatcccagtgggcagcaggtagggacctgctgccatgctgcccccaacagggaagttgtgagtgagccctgccttctgtcttggataggctgactgccaccttggcacaggcctcagcagtgccggtaggaggggaggcctggagtgtggtttttcttcctcccatgcctgacccactttactgaatctgttcacatcttccttgccagaattacaggttttttttgtttgttttgtttttgttttttttttttttttttttgctcagatgaggaactgaggtccaaagaaaggaagggactaacctcaccccatgggttctgatccagtcttgccctcaggcctccaggttcccagaatagtgttgtttacctgtccagccccaggtgggctcgacccacaggtcagaggtcatgagaagctggatgagaccactggggatgtccctgttttctcagtatatcagccaaggacctgaagaacatgctgtcccaggtcaactaccgggtccccaacatgcgcttcctccgagagcggctgacggtaagtgccacccagggctgtctgtagatgggggcaggggaagccaagagcccttcagctgggggcctgactgcctgactggcactcctgctctataccatgcaggacctggagcagcgcagcggggacatcacctacgggcagtttgctcagctgtaccgcagcctcatgtacagcgcccagaagacggtgcatgagccacctgccctccctcaacccctgcccctgctcttccaccccacaccccagctctgcctgcctcagccctgctgccctgcttaggggcttttgggtgccatttctccagttcttctcctcttgaggcctgcccccatctgctgctctagcctgccttcttactagcccatttcccacatggcctcccaggggtcttgccctgaccaggttctgtttcctgcagatggacctccccttcttggaagccagtactctgaggtttggtttggagtggggaggtggggttttccctgggcccccttcatctctccactgggcgattcttgatccaggtgggaggcagtgggagtagggaaccatgctggaccattctgggaagctgtgctggctgggagttgggttctgccttccgtggggcaccttgttgtctgttgaccatactagctagcttaccttctctccctgcagggctggggagcggccggagctttgccgagtgtcccttcctgagttccagcagttccttcttgactaccagggggtatggctgggctgacattggcccaggctggtaggttgtggggggctgggctcatccctgactggaggcttctctcatcccctgccctccctaccccatcaggagctgtgggctgttgatcgcctccaggtgcaggagttcatgctcagcttcctccgagaccccttacgagagatcgaggagccatacttcttcctggatgaggtgagcccgatgtttcacccattttttgtcaagagaatgagtaggggtgaccaggaccccacccgggctccaggagctagacgctccttagggatgccaccttgtttctacctactgtgcaccttgcccacccccagttgggacagagcactctctctcctacccccaacctaccatcttgggttggacagggcagggactcactgtctcttcccttccacatgtttctggacagtttgtcaccttcctgttctccaaagagaacagtgtgtggaactcgcagctggatgcagtatgcccggacaccatgaacaaccctctttcccactactggatctcctcctcgcacaacacgtgagtgtggctccttcaggcccacccagcttcttccccaggagggcccatctgaccatacctacctgcctctccttgcctatccaggtacctgaccggggaccagttctccagtgagtcctccttggaagcctatgctcgctgcctgcggatgggctgtcgctgcattgagtgtgcgtggggtccagggctgggggagggaagatgggaggcctgcccgcttgaccatggtgatgttgctccccagtggactgctgggacggcccggatgggatgccagttatttaccatgggcacacccttaccaccaagatcaagttctcagatgtcctgcacaccatcaaggagcatgcctttgtggcctcagagtgagtcggaggctggatgacccaggggttaacttggctccaggtctctcgttctagagggacagagggcagaaagactcctcaaatgccctgtcccctctccctcagcctttcatctttgtccttcctcttggcctctcctcgtcacctgctccctgcttgagctgttgcttcccaagttacactttctgtttcctacgtgttgggcccactctcttcttcatgggtcctttagactgtagaacacatgctcttctcatcttcagaaaacatgccctggtttctttaggccagtgtcctgccaactcctttgccctcacagcccagaagattgtctctgttccctgtgtcccattccttcaattctgtttactgcttaaactgtggtcactggtctcccttagctcaccagggctctaacagctaactttggaggcttctcctctctcctggcctctttggtgtgaaacatttttcttgcactgctgagcagcctccataattgggccagctcgggactgcatcagttgccaccctttggtttccactgctgccacagctgtagaacccctctctgccccaccagtggctatggcctgcctcttttctgggatagtttttacagacaagaagcccccaggcccttggcttccaacagctcactgtgaggggctacttagacccagagaattgcagaatctgtttcactgtgcttgtcccccatcccgcaggtacccagtcatcctgtccattgaggaccactgcagcattgcccagcagagaaacatggcccaatacttcaagaaggtgctgggggacacactcctcaccaagcccgtggagatctctgccgacgggctcccctcacccaaccagcttaagaggaagatcctcatcaaggtggggtggcgggcttattgcggaagccccacacttctcagtgccttgcccaggccatggcttcagctgttgggcctaaacctgggtgaggaggtggggtgaggactggggtctgcattgccctgttctggttgcccctacagcacaagaagctggctgagggcagtgcctacgaggaggtgcctacatccatgatgtactctgagaacgacatcagcaactctatcaagaatggcatcctctacctggaggaccctgtgaaccacgtgaggactgggccaggctgggggtggtaggccagtgggtgtgaggaccctggctcacaagtccctctttggtctgttccaggaatggtatccccactactttgttctgaccagcagcaagatctactactctgaggagaccagcagtgaccagggcaacgaggatgaggaggagcccaaggaggtgaggaaccagctcaggtctgggggctgggccaggtcaggcctgggccagggtcacagtatctttgctgttgccttcccctgacaggtcagcagcagcacagagctgcactccaatgagaagtggttccatgggaagctaggggcagggcgtgacgggcgtcacatcgctgagcgcctgcttactgagtactgcatcgagaccggagcccctgacggctccttcctcgtgcgagagagtgagaccttcgtgggcgactacacgctctctttctggtaacacttcccatgcagatgcgtatgttcagtcagcgtgtgtacacagacatcacatcacccagagataatcagttaacatttgagcctttgatccaggacaataattaggctttacatggaacataatttcacctacatacacacacacactctctgtctcaccccccccccatacccctcccttttcggttcatttgaagcccacacctttggttcatgtgactgcccacacctgagctcctcaggagattggcctccctccttgaggctccctccttgagttccaccctcatttggggtggaacttggtctttggggccctggcctgttttccccagcctccctcactctgtgtcttccacaggcggaacgggaaagtccagcactgccgtatccactcccggcaagatgctgggacccccaagttcttcttgacagacaacctcgtctttgactccctctatgacctcatcacgcactaccagcaggtgcccctgcgctgtaatgagtttgagatgcgactttcagagcctgtcccacagaccaacgcccacgagagcaaagagtgagggaagggcctgggggcggacaaggcagggcagggccatgggtggtgctggccgggcctgactctgcctgttctcaggtggtaccacgcgagcctgaccagagcacaggctgagcacatgctaatgcgcgtccctcgtgatggggccttcctggtgcggaagcggaatgaacccaactcatatgccatctctttccggtgaggggtgtggcactgggttgtggggccttgcttgggtctgagctgccctgaccctgtgtgactgttttgtccttgtgaagggctgagggcaagatcaagcattgccgtgtccagcaagagggccagacagtgatgctagggaactcggagttcgacagccttgttgacctcatcagctactatgagaaacacccgctataccgcaagatgaagctgcgctatcccatcaacgaggaggcactggagaagattggcacagctgtgagggggctgtggtagacggggcatggcaggggaggcaggagagacccagaatcttaccagtctctggatgtgtgtaacagcaagacctggtgtgttgtagaagttcgtgggagggcccctgactccagctgggagccacagtgtgggtaccaggagggtgtctgcaggaggggacatctgagcagcatctttaaggatggggacaggcacataagcagagggttcctcatgagtcaagatgtggagtgaggaggttctggggctcacactgggagaggtgcacacagtggaaactcatccacctgggcttggcctggactctgtcctagggcagatgagatgaggctacccaagagtggttgtggagcctccgcctggtggatggtatggagggcagagccacaggaggtgagaccagtgagggaaaaagtcaggacccatggcagcacagctggtgataagggccctggacagaaggaaggggtctaggaccagaagatcagttgagagactgctgtcgtcagtagacagggctggctggggagagagaggcctgttgtccactggcacctgggactcaagtgatggagaggagcggcaggaggaatgtggggagtgggaaggcttttcctgactttgggctcagcagtgaaccaacaagccactgaactaaagcactgaatatgggtagtctgtgaagggcccacctgcacatagctcagaaatacttgggagtttgggcatttaggacctgagagattttagaatcctgggctgggccttacatgtaagaatgtgaagaaaggaaagggaacagtgaggccgggcctgaggcctctgaagccgtctctactgaggtcgaaggatccctgtggatcaggtgcaagtttgctgcactgggggaaagggaagctgctccagaaaccagtagctgctttctacctctgggctctggggcattaacatatcccattgtgtcctgtttccaggagcctgactacggggccctgtatgagggacgcaaccctggcttctatgtagaggcaaaccctatgccaactttcaaggtacagctcaggcctctgggcataggaagctggggagggtccccagctgcttggggcttcatttctgtgttctgggccatctgtggtctttgtggagaagtggtggttgtggttttccgaggcccaagaggttgtgagagatgtgtggttggtgagcgaccgcaggtctcccagggccgacccactccctagcccctaccccttaagtcagcagtgaccctttaaactgctcctgtgggtgacccctgtttccttcctgggcagcagcggggtaggagctgggaggcacactggccactcattccccggcctcatggagccctgttacacaggttcttttttgttttgcctttttctctaaaaaggcctgggccctatttctcaccccctcccctccacagccaggggacctggaaggaagtttttctttccacttcacagatgagtggtagggtcccatccaggccccatatgcttccaggtcccggggaggcactcagggccactgcagaccctgccttcttggcctgccagccctgactcctgggagcggattcaaggttctccctgtggcccagggtggggcttaaggttccacctgggtcccgaggtatttcactggcagaggccctgcctctctgatcatatctgtcctggagcttccctgcagggcagtaacaagaactataactagggcctgttgatggcagtgtcatctcccactggcctgaccccaggcagggaagcccaagcacatgtgtgtgtgcacatgaagccccaggttggcagtggcagggagagcctttctgctctgactggtgcttctcacctctgcagtgtgcagtcaaagccctctttgactacaaggcccagagggaggacgagctgaccttcatcaagagcgccatcatccagaatgtggagaagcaagagggaggctggtaagccagtggtgtggtaggcccagagtccaagggccccagtggagctggggccccaaagacatgcatttgtgatgtgcttgtctcctgtgtgcaccagcagtgcctgcctcaccctggcttaggcatgggaacccctaccaaaggataccctcctcatgggagttcggggtggttgctggaggtcagcaccctgtggctcccacaggtggcgaggggactacggagggaagaagcagctgtggttcccatcaaactacgtggaagagatggtcaaccccgtggccctggagccggagagggaggtaagaccagaaccacctgagtaacagcattccctattcccagattctccttggcatgcccccatcacacagtcccaagcacgcacgtgcatgcacagacacctgtcacatgggctggtcgcacagcccatgtggccatgcacgtagtctcccatatacctgtgctacatttggcagtcacaggtacacccacaagatgtatttgtcccatgcacacggatatcccctcacacacatatgcagtagccatgctgaccattggtgggctttgcttcccacagcacttggacgagaacagccccctaggggacttgctgcggggggtcttggatgtgccggcttgtcagattggtgagctcccatctgtttctcttgcccactgttccctagggtgagattcttctttgtatctctttcctgctcctagggaggaagctgatggctgaaccccaaagtctgccctcactccaagctctccccatgctctggacatccccttgacaccctgggctccctttgtcactgtcagctttgacccctgcatgtttacctatgccaggcactggggaacccgtcagaatcagcagaacaaagttgatgctgctggttggctgacatccccaggcctaatgggttgtaccacagggatgtgacagaggcttggagatggggtggagagggagacacggcctgaggaagaagcatgagtcagagctgagttggaacactggctccaccacaaccagcagtgacttgtttgagttgtagcgtcgtcatctgtcaaatgggccagcagagtagtccttcccctcatggggtgtgggaaggaggaaatgggatgagatagaactcatttgagccagtgcctgcggtgtggagtggggtggagggggtgagatgtctattcccagctgttatctgctctcgccctcccagccatccgtcctgagggcaagaacaaccggctcttcgtcttctccatcagcatggcgtcggtggcccactggtccctggatgttgctgccgactcacaggaggagctgcaggactgggtgaaaaagatccgtgaagtggcccagacagcagacgccagggtgagattctgctggaaccttctggggggcagtgtgtgggcccgtcaggcagctgaggcctccagctcccagcacaggccccctcagagcagtggggcagccgatggcctatgctagataggccacagcccctgccttagctagggattgagaagaaacagacacataagatgcaatgtggtgtgtttaggttgtgttgagtttgagatactgatgggtcgttgaagtgggtctgcttagtgggcaattggatacacaagaccagaggtaagaggagaagtcctgggctggaagcaggagtggagtgggggctgaggcatagaagggatgaggtggtgtcgcataggaagagaacttgggatggattgagaagctccatatgagaaggcatgggcgaggaaaggagcccaggaaaggcaggtcagacagactgcaggccactggtgagggagagggtctgctgtgttgagagctacagaggtaaagacaggaacgataagaacttgcaagtgccctttggattcagcagcagctaatagcccttgtgagacttttgagctgcatcagcagcatggctggcattagatggcagtgggcagcagggagtctggaatggcaaaagcagagcctttgtagggcctgtccttgagggagagaacaggtgactggggtttgagcaggctctcatgcagatggctgaaaggagcgagcagcgtgtgggaggagatggagcccaagcttagctggtaggaccagccttctggagaaggtggcctcttcccctgagcctgggggaacagagaagtattctcaagggttagggaattcctacttggtggccctttcaccaagtgcatttggtagatgggtctgtagctgctttggagagggcacccgcaagccaaatagagaagggatagggtgcttgccaggctgtcccctagaaggaaaaggtgctggcataccaggtggttgaaggactggatctgggggtctaggctgaaaagggaagagtctgaatgtaagagccactgaggccgggcgtggtggctcacgcctgtaatcccagcactttgggaggccgagatgggcagatcacttgaggtcaggacttcgagaccagcctggccaatatggtgaaaccctgtctctactgaaaatacaaaaattagccgggcatgttggccggcacctgtaatcccagctactcgggaggctgaggcaggagaatcgcttgaacccgtgaggtggagattgcagtgagccgagatcacgccactgaactccagcctgggcgacagagcaagactctgtctcaaaaaaaaaaaaaaaaaaaaaaagccactgaaagaaagtcctccaggagaggatgggttggggtgcttgtaagtgggcttctgtagagatttggcatggagatttgccatgaagagtgggtggttatatttgagttgggggcgctgaggcagtgaggatcatccttaggacacagatggaaaccaagccaggggcctgtatccccagtgcagtcacaggtaacacagaagaatcatttttaccccagcatgaggctgggtaataaatggtcagaaagtgtcatttgggagtgtggccaattccaggatccctggaaggtcactgggaacatgggtggtgatgaggagtctgtatcccccaccccccctaagagggttggagggcaaatgtgtcctctgggacagaaaagaggaaagatgatttcaggatgaggctgaggacagagctccctcatttactgttgggtgtggtgtctggaaggtacaggggaaggtgggagaggggcccagagcacctgcagtgtgggcatgcccaaggttggcaggggcttccttctcctgggcagggctgtagcctggggctacagggccttgtgtgtgtcaccagctcactgaagggaagataatggaacggaggaagaagattgccctggagctctctgaacttgtcgtctactgccggcctgttccctttgatgaagagagtaagggccagggcccaggcggggtgtgcatgtgcctggagggcctggtgggtgcaaaaagagtatttaggtatccccccaacacttcctgggtgggcgggctctgtaagtgttttccctgtttggcccagagattggcacagaacgtgcttgctaccgggacatgtcatccttcccggaaaccaaggctgagaaatacgtgaacaaggccaaaggcaagaagttccttcagtacaatcgactgcagctctcccgcatctaccccaagggccagcgactggattcctccaactacgatcctttgcccatgtggatctgtggcagtcagcttgtggccctcaacttccagacccctggtgaggaagtcccctgtgaggagggtgaggaggggcactgtggggcagctggactggaatacaccataatctgcctcttccagacaagcctatgcagatgaaccaggccctcttcatgacgggcaggcactgtggctacgtgctgcagccaagcaccatgcgggatgaggccttcgacccctttgacaagagcagcctccgcgggctggagccatgtgccatctctattgaggtgggtgctgctcatctgggcttcagggtaggaaaggggctgcttgccgttggagtctgtttatgttgagttctccaaaagtaggtattttcaggcatcaaggtggtcagggtgggtggggcctgagctgagtctttgaaggggtgaagatagcatgcagtcactgtatgcatctaggacgtgcagagccatggtgtgacttgttctgatgacttgttttgttctgatgagatcttttttttccctaaggggaggtcatttaaaagatttctgtgttaaaatagtaatggatttaacatgaagtagattgcaaaactcacatggaaattttggctaaagctcagacttcagataaactacaggcagcagctgctctggacagcttagggtccctgatgtcgtgagggactccatgggcagtgtcccgggggcccagcagagggcgcgctgcctccactccacagatgctgactgagcctccgcagtggggaattggagggagcaggaaggacaatcccaggcccttctttgtctgcctacaggtgctgggggcccgacatctgccaaagaatggccgaggcattgtgtgtccttttgtggagattgaggtggctggagctgagtatgacagcaccaagcagaagacagagtttgtgggtcagtctgtcttcccagtcatcctcctcatcctgctggggcactgcaagcctctccccaccagtcatcccatcctctcccacggtgacctgaagccttttgtcgttgccttcacagtggacaatggactcaaccctgtatggccagccaagcccttccacttccagatcagtaaccctgaatttgcctttctgcgcttcgtggtgtatgaggaagacatgtttagtgaccagaatttcctggctcaggctactttcccagtaaaaggcctgaagacaggtgaggaccattcctggaggcagtgcccctgcaatcttgctggcagggtggggctgggccccttgctctgtccctcgtgggctgagggccaggcttttcctcctcctaggatacagagcagtgcctttgaagaacaactacagtgaggacctggagttggcctccctgctgatcaagattgacattttccctgccaaggtatctgcagcaggggtgggctggcctggggtaggtgggaggagagccaggcagcagcctctagaagtgcagaggagtcattgaccctcttgtgcacctggcttcgttgaagcaggagaatggtgacctcagtcccttcagtggtacgtccctgcgggagcggggctcagatgcctcaggccagctgtttcatggccgagcccgggaaggctcctttgaatcccgctaccagcagccgtttgaggacttccgcatctcccaggagcatctcgcagaccattttgacagtcgagaacgaaggtgaggaagatggaggggtgctagagccaggaaggcagtggctaggtcctccttcttcagtgtttctttctcctgggtagaaaagttgtaatattgtctggcattgggctgcaaggccctgcctgccagtaaggacactcttcccttctgtccagggccccaagaaggactcgggtcaatggagacaaccgcctctagttgtaccccagcctcgttggagagcagcaggtgctgtgcgccttgtagaatgccgcgaactgggttctttggaagcagccccctgtggcggccttccgggtctcgcagcctgaagcctggattccagcagtgaatgctagacagaaaccaagccattaatgagatgttattactgttttgggcctccatgccccagctctggatgaaggcaaaaactgtactgtgtttcgcattaagcacacacatctggccctgacttctggagatggatccttccatcttgtggggccaggaccatggccgaagccccttggagagagaggctgcctcagccagtggcacaggagactccaaggagctactgacattcctaagagtggaggaggaggaggagccttgctgggccagggaaacaaagtttacattgtcctgtagctttaaaaccacagctgggcagggtgagaagctagatgcccctgcagtttggccctggagccagggcagaggaatgtagggcctgcatggagaagggttctgccctgcctgaggaggaggacacagcacaagggcacattgcccatggctgggaacatgacccagcctgaaagatacaggggatcatgttaaaaatagcagtattatttttcgtctcaatggtattgtaactaagttatttactcctcctgctcctcacccctgtagggaaaccttggagaggagagtggcaggtgggctgcctgctgtgttaagaggacttagtttgtgatgtaaggcactgtcaggaatggggggcgggccagggtgggaagagaagaaatagcagagcctattttggtgaggttttttgtttttaagtcaaagaagactcagtatgctttccctgaggaatgaaaaagggattgaggagttgcctgactcctgggtgggtggggtacaggcagttaggtgctgaatgaagctgccatccttgctgcagcttctaactggtaaaaagatccagggatggagatgggaaggttagaaaggcagccctcacctctgaggacagaggccggggtccaggcccgtgggcgcaaaggtgcctcatagcatagccagcattcagcacacacaaacctactgcccacatttgggctcagggttggccatttgctagttctgctgccctcttaagatctgactgccaaataaatcatcctcatgtcctttttcctttgacttgtatgctctttcgggggctcaggaaagcctgttgcatgggacatgctcactagaaacagtcgccagatgattattctgcagtagaagcaggtaggaaatttctggaaatttctcaggttaagcagcaagagctgtaacccctcctctgggctaacaggagttgtgggtccactctctcctgcccaccctctgagggtgtgtctgagcagagtacatcctgcctgggttctttgtggccagccagttttggtggtgagctggaaacaaccagagcccctttccagttccacagaaacctctcctttcaaaaatgttgcattcagttcgttaacactgccaggtgccattctgattgcctctaggacttggcagctgaaatctctgggccctttcaacacagttgaaaggcccttctcttcctgaagctctgtttccatagttggctgtgctgggatggaacaaaaatgacgccacacaaaaaatttaagatagatccggttccgtggatgacatgaactgatgatagccagtatcaaacagcgatagtgctcaggttcttgtgtgactttcttttacagcttcacagtcccagttcatagagaggagggtggctttttcccatacacaagaaggtggtgggtgggaattcacctgggccctcatgatccatgtttcctctctaggtttttatggcctggagagaaaggtttcctatcagagaaggaagaggactgtgtaggcccttctgttagggcccatccacgttggttaggacgtccttggcgtgtttgttagtgttgacccttttagttttcatcaatacgtatctctatttgctgaacaagtgtctttctggaacacaacctggggaacagacagctctgcctttcttaaggcagctttgtagacctgaggctcacctctcttgggctctgtaagacattgcctttgcctcactgcaaatgttttgattgttcttcctagtggaaaacgtgccaactctcaagcaaatttaaagatcattttcacttcaagattcctccagacctgacaaatgttgattgacccagaaggcagagtgtttgttggtggggaagacccttacttggggccgaatgctttggcatcaggagttgtttgccccatcccatgcatgcaggccgtgtccctacagtgcgacagctcagggttatgacctgtggagtctcaaccctaacagcacattagcaccacttggggagcttctgaaaaatactggtacccggggccttagcataggtattgttcaaaacctctcaaggtgatttaaatgagtaacaagggttgaaaaaccctgattttggcagcacaaaccaaaagagcaggcagggccgggagagggaagtcagtggtaccagaaggtagatgggctcccttgcaggctccttgttctcctgccatcaccagtagaaccttctggctgacagaccagggacaagtagactgggttcaaagtgacagacctttcactttcaacagctttggctcagcagacatgtacacatacaaagtagagcctacaaggtcaggggcattctgcccccgccacaggactaaagactgccctgcgggaagatggcaggagcagtttctgacctcagttgagtatctgtggccatgagcagaaaaggcaggggtctgcctcctgaccaagcacatcttgaacatcacctgagagcttgaacatcactaaggacctagacactcacttgtctttcaacttgagcccatcactcaccatgtgagttctgttgagggtgggtagaaagcaaataggttcaggttatcccaccagactaactcggtgaatgaaaggatcatgccttcttcacattttaattaaatggatcaagcacagtctcagactgcaactcctgtcttctctgggtactttagtaattcccaggggacttgtgcttaggggttaatctaggcattctgttccctgcctggaggaaccccacagcaacccccctggctggccgctcctgcgaattccacccatagtatcccaagtgagtcactgcctgagtctacccactacagcattcagctgattggctttgccagttgcatgccagtttggtctagtttccaaggacctggtttttgggattcaacacacacagctgcttccccatgacataagctggactcctattctggtgtccagaatcactgcctggctccactgtaggtgatttcatttcaaaggaccctttttccttctgtcccaggccttcagggccaccccaacaaaattcaaatgctttgggtcttagaagctggggctttagtctttttcctccagaaagcccctgggtccttatgcatattccctaacccccgacaccgccccagcaaatgacaggctatgcttttcagcactctcacaggaaacagaaaagaacttgccagtgtcctctcaccagatgataccaggtttgactgtttgcctttgtctatttagcataatttctaaaatatcatagttttctcatgtagaagccagtgtggtgatctcagcaatgcagaagacagtgagttggaacttctctggtgagctgagggaaactgccccactcatccactggagtttaagatccacagaagtgggcaagaacaagtgaaatgtttttggcttttttcctgactcccccacaaaataagtcagcagatgtgaaatatatttatgtttttatttaggaataatcaaaaggtttgaagtaccattgaggcccatttcaaattgtacaagcaatttgtccgtgttcccctcccttatccaaatccacagatacaaaatctaggaaatgtgcaatttgcatcttagaaatagcagcatccccacaggggacctcgtgaggcctggagattcagccccaagagggcaaccccactcctgcctcagcacgaccacagtccaaaccgaagttaagttccatttttttgttaaaacgtttacccagggtaaagaattcccattctacccaaggcttaagtgatagagtatgctgttttctcggtttggtctctcacacctggccatggcaggagatgggaccctcttgcagagtttggctaaagtttcattgtgcctcaaaacaaacaaaaacagggcagttgcctcttacttgtttagcaccaagatgatcaccttttcagccatctctcctgacagcagactgctcaacagtcactccccagtcttattgcccagaagtccggccacagggaacaaggctttgtgctgaagaccaaaatgcaaatgggtgctagaaaaccaggaatcaagaccttgtcaggaggcatctacaggcctttggcaacctccctgacttccacaaaggctgccctcagtaaaactcaatgaacaatggccagcaggggagaaaggaaggtgggcgtgttttctctttaataacaattatggcacaatctgaccctcctaacacttctgaacaaagcaactgctcagaagcacagcgggaaccctctacacaggggtgactgcttctcccctggccccgacccagtctaaccgagaacaaccggattgctgtgctgggtccctgctgaggagtagggactggcgctcctcactcctgctgacaatctcatgtggggctctggccctgttccaagcaaactgcaagaacgagaccaaacagtatgggcaggaaccagggatgagtgacaagatctccctaaacaaattacaggattcataggtaccatgtagatttttcttttaaatgtatttctactcaggtttgattcttgggaaagaccaagcacttgtagctctctattcagtgactaaacaacctgacatttcacttttaactccagtcacattaagttttttttttttttttgagacggagtttcgctctgtcgcccaggctggagtgcagtggcacgatctcagctcactgcaacctccacctcccgggttcaagtgattctccttcctcagcctcctgagtagctgggattacaggcgcgcaccaccacgcccggctaatttttgtatttttagtagaaacggggtttcaccatgccgatcaggctggtctcgaactcctgacctcaggtgatccgcctcccttggcctcccaaagtgctgggattacaggtgtgagccactgcgcccagccaagtcacgttaattttgaatcatccacttacggaaaggaacagaagttaaaaagcattaaccaagaaaaactattcattaacacatacaggtgctcatgtgtgtgtgtgcacgcgcacatgaacacacgtttcaagccagtctcctgaaggaagaggcactaatggcaggtaaatgctcaggctcctctcagtaccctggggctcaccgcctatccaagtggacagcagggaaggtgcctagagtccccaccagagcggctgccttcctcccttcgctcagctgccagagcagccacgaaagccaaattctcagggctgtggaagctttggaccagcaacaacaagctagtacctatatgttctttatcatcctcaaagtcctgcgaacccaaagatagaactttcagaaattgaggaagggggtgctttcccctgtccttgtccatatggtaaccagggtctgcctagtctcatccccagcctctgggttccctcccctgctctagacttcagggcagttagaatggatgactctgctggatctagagctaaatagaaacttctttgccatagaaacttctctgttgaaggactgcgtccccattcaaagaggtcagatttgtgggcaggcaagagctcaggaatgaagaaccaaaaaggaggctctcaacccctctccaaggccagacgctctctgtactcccattaacttcacaaccaagtgcagacgctcggtgagttcagagaccacttggttagccctgccccacccctcctggtgtgacctggcactgggaacacacagattgcagcagaaacctgttggttccatttgcactcagagcaaaccaagcagcaccttgattgccacttttcctccagccactctgtcaaaatcaaaactggttgcctcaccagccctggtgtggcatggcccaaccatgtccctgccgccccacttaaatggccagagaaaactacatggtgggtagatgaggttgaagccttgcttggggaagggacttgagcacaggagaaaggcttgagctccacaactgcccatttgctcagacaatggagcttttctgagatcaagtgctccccaaactacatgggagtagccccccgtggtctgggcaggctggctgagagaggtctgtgctctcaggtaaaaggagagaaggaagctgggtcaagagaccctgaagaggagggtccgtgtctggagtgaggggaactgagccctgctggctacctgacctgggtgggctgctggctttgccctggaagacttgactatgtgggccttcatgtgtctagaggatttttcttctccagcatataactaccttgagaatggatgtgatatgtgaaaatttaaatttaatacaaaagcacatctgcaaatggtgagtcaaactggtgaaatctcccaggtaaactctgagcaagaaaaaagagaagatgactttaggaaaatcctaaacgaggtttggttttaaaattttcttcttggctggctctcctctcctgcaagttgcctttatccacttggggtgtgatatctgaactgaaaaagctgcaccaatctgaggatattggtccaaaaagagacaagagaccacgaggctcaagcagagagcatccacacagcatccaaccagaaaggccagtgtccttcctccatgaaaggcagccagggacagatgtcccagagccaggagagagagggagagaacacaggacctgcccctaggccactgactgagcaggataaaacccacaagggcacatctcaggctacggaatagagagatttcctggtgggacggcctcccaaggctagctggagatgagaccactagtacatgtgtgagcccgcaaggcactacccaatgttctgtccaacttcctccttgtgctactagagagcacagtgtggagtccttgtgtcaaggtgctttggtccacctgaacagccacctgtcactcagccaagagaaaggagctttaagttgtacaatcactaccaaaccccaaggccctggcaagccatccaactagggcgttctttttcaggaacccaagattctcaaaaatgacactcctcccctcccaaaataaaaaagctcctttatagtcaaaaacgaaaacaagtaatcaaaatatacaaagcatatgtatcaaatttaaatttcctttatcagaatgatcatctggtttcacctttgagaactcgatctacaaccccatgtaaaaaacaaatgggtacaagacatcagcaacaaaaaaacactaaaagatgattttactatctctgacgagtccaactgcatacagagaaaagggataagattagccactttttataggctttttcttcaggagagactaacctcacccaactctcaactctgcagcccacaaatgccttctactgaaggcaacaacacaatttaacccttaggcagtgtgcaaaaccacagtgagctgtcctgtgacagggctacatacctggctgctgccatcccacatcccccactgccactgggctagtgtgggtgtgtctctgtgcacgactggctggattcagggttttgcccccaaacccttgagtacctcctacaatgtccctttaagcacctgcgtgatgactgcctccctcatcatcgcttttgatcatctttcaaggataaggtccagagcatactcactcactctcatccaacagcagaggaagaaggatctgtccagactgctgggctccttacatgggcattcttgccttttgtagtgtagccctggctgttgggcagggctaacatctataagaaaaacataccagggcaggcagcactgtacttttccacctgaaagatgagctagtgcccatggctgggtcactgtaggtcctgcatgaacttgtccaccaactgtgttttgctgtttgggttgtaaattaaccgtgaatacaagtaaccaagaaaatcctctccacagaccaactcttcagccagtgcatgcagggctctggaggtgatgccagcaggatctggaggtgaggcactgggtggtttctcagacacccggagatggcttcagcctgggccgccccagctgcctagtgcactggtgtccctccaacacaggctactcccccaacacccatgtgccaccaagtttctcaactgcacacgccagatgcgcgttttacgagaatcaaatgagtatttatactgtatatgtgtgtgtgtatatatatatatataaattaatctgcacgtatataaacactaaaaggagaaattccaagtgtctgttctctgaaggaaagcaatcactcgccattcttggcaattcctcaaaggtttttgagaccaaacatctttactccccaaattaagaatacaaaaataggctgcaaaacttgccaagtactcacaagtccctgtttgagacaaataagatgtggtccctattgggacagggagggcagaagatgacactttagaagaacatgattttggtttcaaaagcaagacctctcctggaaggcagaaggggccaccgccatgcttgtccttggagccacatgagcacggtgccctgccaggtccagttccctgggtaggggagaaggacggtccctaaaacaccaccagtgcttcttgcccaaagcccttgccctctctctggggtcttctagcactgtcactctgcccagcccaggaggtgtctgtgcccacgaagcctggtacttgtggcttggcactctgggaagcaggtggtttctaagaaatgagcggagctacaggtggacaggtctttagttcgcccctctttaccagctactctcagagcaaccggtcttggggaaattgaggatttaccatttagaacagctctgtaagcaagttttctaattttcaaaggcaccatttcctgtaattttctcaattcaaaaaaggacatgagggttaggctaaagtagagttcttgtttccaagtttggggacataaatataaaactttaaaagcatcctcacaaggaccagtgatgcccagcacccttgggcatgcatccagcacaccggccagaccctggggtgggagccacgggcactctgggtgttctgccagggatctctgcatgcaagtctttgtgctaaagaccaccctacctctgataattccaagaagtcaacaaatccctccagccttcacccttactaatgggccagtgtgttattccttcccaaaccataggaggctctgaaatgagtgtggagaaagggctctgaaaaccgcagtggtcaaagcttcaggctgtggctatagatttccacttacgtccctaccccatgtcttttttccatctttctctcactccaaacatgtctagattaaaaaaaaaatgcaggcacataggtcagcacattaggcttaacaacaaatgtttctttagttgatctgataactgaaaagttatggtccatgattcaaactcccatgaaaaagcagaattacggtaacaactaaagagaatggtaaagcatccaacgctaatcacattgccagtccattttttgaactgagggttacagcaggttctgcactgagtagcccacaaggcaaagcaagcctgacatgaaacgtagccggtcatcacttaatgcagcagagatctcttcaaaggtactgcttccttgaggaacacaaagggggcacaaaggggttccagacgtagctttgtgcctataacttccctgcctgactgtggaaggtgaggggcacctgtgacccagcaatccccagagaacagacacaggtcatttttagagatgacgtaactgacaatgcactgcttggctaaccaaagtttctacgtaatgacagtgttgataatctgatgttttatttaacatatagaggtggatgtatatgttaaaagcttgattctgtgtctatgaatatgactatgaactctgaactattttatccattggaaggtaaaggaaaggtcctacattgtgggaggaagaactgatgagaaccccatcttgcttgctgcttgcttggtggtgggcaggccgagggtagcggcaggctctgggctgtctgcgaggattctggaagctctcccaggtgcccagcagccgggcatgggaacggcatgtggcagcagagagtcgggtttggctcttccacgtggcaggcggtttcccagactggccagtccttcacattaatcagatcaaattcagtctgtttctgagaagaaaacacatgcctgtcactctacggcagctgccaccacctgccccccaggcagcctggtccttgctataccagggtggcatcactggctttgaggacctcttaattccatgagcaatgaatggccttctgccacccactcccccaccctccagcctgagggaatgtgggttaaggacaggatgctggctcaaccttgtaaggccatcagactctctgagagaccctgctaaaccctaggcctagcagcaggagcagtggtttggcctctccaggcccactaaatccccctagctccccaggcttccgccaccaccgtctctggagtcctgtcctaaaatgcttcatcctgccctctccttccaggctctccagaacatactgttccaatataatagcagctgggtctagttctgatatgtattctactgatgtcttcacttcatttgtaacttgcaaccctaacatcaaagtcaaagtaaggctgatatcagagaggttcatcatctgtgattcataaagaaatggtccttaattgcagctggctgagaaagccatgagaaaccaggtacagataaacagattggcccttcctgtacccacagttacctgtctcagacagaagagaacaatgccaatggcaatggccaccccctgccattcctgctctgtcccacaaaggacagcaacatgtcactttgtgttggatttagtagctgaggggagccaatgcttgtcattcaaatccactcatcacatttccatgttgactttaagaaaaaaaccatggtaaaatataaataacataaaattcaccattacgacaattttaagtgtacaattcagtagcattaagtacatttacaatgttataaaaccatcactctatttccagcaatttttcatcatcctaaacagaaattatatatccattaaacaacaactccccatgtcctccaccccttaggccctggtaacctctattttaccttctatcgctatgcctttgcctattttagatacctcatgtaagttgaatggtacaatatttgtctttttgtgtcttgacatctcacttagcaaaaatgttttcaaggtccatccatgttgtaggatatatcagaattttattccttttttggctgaataatatcttactgtatgtacacacacattttctttatttatctggtggtaagtacttgtttccgccttttggctactgtgaataatgccactaagaacactgatgtacatgcatatgtgagtccctgttttcagtccttggggcgtatacctaggagtgaaattgctggatcacatggtaatattctatattcaccttcttgaagaaccacaaagtcgtttcccaaggagctgtaccattttatattcccaccagcaatgcacaagggatccagtttctccacatcctcccaacacgttgttttctctgggtgtgtgtttgtaacagccatcttaagtgtgaagtggtatcacgctgtggttttgatttgcatttccctaatgccaaaggttcgtcttttcatgtacttattggccatttgtatattttcctatgagaaatgtctaaatcatttgcccgtttttgaattggttttgttgttcagttttaggagttctatacatattctgaatataaatctcttatcagatgtatgattttcagctcttttctcccattctgtgggttctttcattcttttaacagtgttctttgaaacacaaaagttttagattttgacgtaatccaaattattattttcttttgttgcctatgctttgagtgtcatacataagaaatcattgcctggccagatggggtggcttgcacctgtaatctcagcactttgggaggttggggaggaatgcttgaagtcaggagttcaagaccagcctgggcaacatagcaagatcctgtctacacacacacccacacacaaaatagtaataataaaattagccaggtgtgcacacttgtaatcctagctactcaggaggctaaggtgggatgactgcttgagcccaggagtttgaaactgcagtaggtttgattgtaccactgcactccagcaacagagcaagaacctgtctcaaaaaaaaaaaaaaaaaaaaaaaagtaataattgcctaatcaaaaatctggaaggattctattttcttctaagtattttatagctttagctttttttagttctttgatctattttaagtaaatttctgtatatagtacaaggtaagagtataatttcatacttttgcatgtatatatccagtttcccagcaccttttggtgaaaagactgtcttttcccattgaatggtctttgcacccctgtcaaaaatcaattgaccattacgtggggatttctgggctttttattttattccattggttttgtatgtctctccttatgccagtaccacactgttttgattactgtagctttgtagtacaatttgaaatcaagtgagtgtgactccttcaactttgttttttctcagtttgtctatttggggtcccctgagattctatataaactttaggatggattattcttttataattgatagtttacatatttatggggtacatgtgaatattttttacatgcacagaatgtgtaatgatcaagtcagcatatttagggtatccatcaccttagtatttattatttctgtgtgttggtaatatttcaagtcctctattctagctactttgaaatatacaatatgttgttgctaaccatagtcacactagtctgctatttaacattagactttgtttcttctatctactataagtctatacccatttaccaacctcttttcatttcccccttctactctcacacccttcccaacctgtgctatctatcattctattctctatctccatgaaatcaagatttttagctgccacatatgagtaagaacgtgtatttgtctttctgtgcctgggatattccaatgaacataatgacctccagttctattattttttgtctttttaataatagccattctaactgggataaaatgacatctcattgtggttttgatttgtatttcccttttgatttgtgatgttgagtgttctttaatatacctattggccatgtgtatgtcttcttttgtgaattgtctattcatgtcttttgtccccccacccacccccaccttttttttttttttttggacacaggatctcgctctgtaacccaggctctggaatgcagtggcacaaccacggctcactgcagtctcaacctcctgggctcaactgatccccccacttcagcctcctgagtagctgggactacaggtatgtgccactacacccgactaatttttgtagagatggggtttcaccatgttgcccaggctggtctcaaactcctgggctcaagtgatccacctacctcggcctcccaaagtgttgggattataggtgttagcctctgtgccccaccttttgcccactttttaatatgattttttgttgaattccttatatattctagatattagtcctttgttggatgaatagttttgcaaatattttctcccactcaacatattgtctctgtgctgattatttcctttgctatgcagaagatttttaatatagttccctttgtctatttttgttttagttgtgtttttgaggtcttagccataaaatcgttgctcagaccaatgtcctgaagtgttctgcactgttttcttctagtagtttcataattcaaggtcttatatttaaatctttaatctgtcttaacttttgtatatggtgagagatacaggtccagttttattcttttgcttatggatatctaattttcccagcatcatttattgaagagggtgtcctttcctcattgcatgttcttggcacctttgttgaaaatcagctggctgtaaatatgtggctttatttctgggttctattttcttttcctttggtctgtgggtctatttttataccaataccatgttgttttggttactatagccttgtgatatattttgaagtcaggtaatgtgatgcctacagctttgttctttttgcttaggattgctttgactattttagctctttcgtggttctgtacaaattttagggtttttttctactcctgtgaaaaaatcacattggtattttgatagcaattgcattgaatctgtagattgctttggacagtgtggccattttaatattaatttttccaatccataagcttgggatgtctttccatttgtgtcctcttcagtttctttcatcagtgttttacagttttccttgcagagatctttcacttccttgattaaatttattcgtaagtatctttttttatttttggtagctattgtaaatcagattaccttcttgatttctttttcagctattttattattggtgtatagaaactctgatttttgtacgttaactttatatcctgccactgtgctgaatttgcttaccagctttaagagttttggtggagcatttttggtttttctaagtatcagatcatgtcatctgcaaaaagggacaatttttctcttttccaatttggatgccttttctttctcttgcctgatttctctggctagaacttccagtactatgctgaataggagcggtgaaagtgggcatccttgtcatgttccagttttcagaagaaggattttcagcttttccccattcagtatgatgctagctgtgggtttatttatggcctttactatcttctttctatgcagttttctgagtgttttttttttatcatgaagcaatgttgaattttatcaaatgctttttctgtttattgagatgaccacacgatttttgctcttcattctgttgatgtgatgtattgcattaactgaatggtgtatgttaaaccatccttgcattataattttttgagacagagtttcgctcttgtagcccaggcggaagcacaggggtgcaatcttgtagcccaggtggaagcacaggggtgcaatctcggctcactgcaacctctgcttcctgggttcaagcaattctcctgcctcagcctcctgagtagctaggattacaggcatgtgccaccatgcctggctaattttgtatttttagagatagggtttcaccatgttggccaggctggtctcaagctcctgacctcaggtgatctgcccaccttgacctcccaaagtgctggaattacaggcatgagccaccatgcccagccaaggattattgatatgtgaggttttgttattgttatactgttgttttctagttgttttatatatttttttcctttttcttttgtttggcattgtactctggtagttttctgtagtggtagcatttgagtcttttcctcatctgtgtgtttgctttgccagtgagttttatacttttgtgtgttttacatagtgttaaatgtcatgttttcacttccatgtttaggactcttgtgagcatttcttgtagggccagtctagtggtgatcaattctctcagcttttgcctgtcttggaaatactttatttctccttcatttatgaaggataattttgctagacatagtatccttggcttactttttttccccttcagccctgtcaacatattatctcattttctcctgatctgtaaggtttctgctgaaatccactgttagtctgatggggcttcctttatagttgcttacacacatttctcttgctgtccttagaattatctgactttagagagtctgactataatgtgccatagagaatacctttttgcactgtatccatttagaggctatcagggcctcctatatctagatgtctaaatctcttgtgagatttgggaagttttcacctattaatattatttcattaaataggttttctcactctttcattttctcttaaccctctgggatcccaataatttagatatgtagttgctttattgtgtcccatatgtcacaaaggctttgctcattttttattcttttttatttctgagttatgtcaaaagacctgtgttctagctctaagacttttttttctgcttgatctaatatattgttgaagctttcaattgtattctgtactttgtttcatgaagtcttctattccagcatttgcttttttatgacatatatctctttgataaaattatcattcatttcccaaattgtttttctttttcacattctcttacatctcattgagcttccttacactcaataatttgaattctttttctgggattttgtgagtttattttttattggaatctattgcaggaaatttattgtgttccttctgaggtgtcatatttccttgctttttcatgtttgtgtccttatgttgatatctgcacatctggtgtaacagttgtttcttctaattttttgcatttgcatttgtaagggaggactttttcctagacgtatctatggtgttgtttgggtaggacactttggctttgattcttggtatgtgcagtagtgtagtctctgtatgatttctttggctataaatagcatcagtggtatctgtgatttcctcagtggctgagtagttactagtggaagctatggtaaaattttgctaaggactggaatgccaggtgggccattctccaggcaccagtggtagcagcagtggacagagcatgcctgtctttggacctcagaggagcatacaatggcaccagtgttagtgggttcaagcaggctgattcttgggctttcgggtggcttactcagatgtcagtagtggcagcagttcccaggcatgtgatcaggttctcaggcccctgggcagctggcgtggcatgggagatggcagtgacagtgttgaaatgaccctctgggtcccaagtgatatgtgcttgtgttggcagtggctgcaacaggctgggcggcccattctgcaggcccacaagtggtacatacaggtagtagtggccaagtgggtaagccccaacctcaggtacccaagaggagtattcaagtgccaatggtggtggactaggctggacaatcccccagcccctagactgtgtgctctggccacggaggtggtggtgtaaagccaggctgggcagtctcatcttcaggctccctatgatgtgtgcaggtgccaaccaaggtgggcaggtgcagggcaatccctaggccaccttcagaatgcttggatagggagtggcagtgctgtactactaccttccattggagagcacagggttgctggccagtggggaatgcatgtgccactcacacctcactcagctccagtggcacttgtgcctcagcccctgctatggtagctggcaccatagttgtacctcagccacaacaacaggagcacatgccttgtagtgcttcagccccagctgcagtaacccagtcacttatgcctcagctctggggaaaacagtctgcagttctcttacacctcagccctggcactcagggactccaggacagtgtacagtcttttggggggtgggctttaaatggcatcttgctgtagctgttttgttctcaggaagtgtgggggacccagtgcgagctccgttctggagcagttccattgaacaatctcctggcacctcctatgttaagtttcagggactgcaagggctgaggagctctcccgtggctagaactgcagaatcctttcatggtggaaatgtggaccactgagggtctctcacttaccttttccccatgctggggggtctctctcagttcccagccaatcccagctgaacaggctgccttgcttctttttccttccttactttgtttcctgtctcttttccgtgaattccagtgttctgtcttggataatctattcaaagtgtgattatctactctctatttttgttcttaatggaagaggcgagtacaaaatgcctctagtcagccattttggttgggtcttcctggattttcttatttctgtaaaaatgcgactatgcttttgatagattttacattgactctgtagtttgctttgggtcacgtctacatcttaatatcaatccatgaacatgggatatctttctatttatttaggttatctttaatgtttttcagcaacattttgttgttttcaagatacacttctttggcctccttggttaaatgtattcctaagcactttttctttttgatgcccttgtaaatgataccatttttaaaattttcttttcagattgttcattgcaagtgtataaaaatacgaccaatttttttagacagatagggtctcactatgttgcccaggctggcctccttgaattcctgcctcagcctcccaagtagtgggactacagttatgtcactgtgcccagctaaatacaaatgattttttttgtttttatatcctgcagctttgccaagttcatttattagctcttaactatttttgtgtgtgtgtggattcaagttttttttttgttgttgttgttgtttttgttttttttttttgagaaggagtctcgctctgtcaccaggctggagtgcagtggcatgatctcggctcactgcaagctctgcctgccaggctcaagtgattctcctgcctcagccctaaccctgagactaaaggcacatgccaccatgcccagctaattttgtatttttagtagagacggggtttcatcatgttggccagggtggtctcgatctcttgaccttgtgatccacctgccttggcctctcaaagtgctgggattacaggcgtgagctaccacaactggcctcaagtttctacatatatgatcatgtcatctgtgaacaaagataattttacttcttcctttcgaacttggatccttttatttattttttttgcctaactgctctggctagaacttccaatgctgttttgaatagatgtggcaaaactgggcatctttattttattcctgatctcaggggaaaagttttcggtctttccccattgttgagtatgatgttaattgtgggcttttcatatatggtcttcatcatattgtcatattcctagttactgagtgtttattgtcatgaaagcaagttgacttgttagatgctctttctgcttgaattaggatgatgtgttttattctctttattcttgtaatgtggtgaattacattgatattcatgtgccaggcaatccttgcattgcagggatagatccctcttgctcatgaggtataatccttttagtatgctgctgaatttggcttgctattttgttggggatttttgtatctatattcataagagatattagtctgtagatttcttttcttgtagtgcccttgtcttgctctggtgtcagggtaatggtgacctcatgagttaagaagtgtttactcctcttcaatttttttggaggagtttgaggagaactggtgtgttaattcttctttcaatatttggaagaattcaccaggggtcctagacttgtcactgttggaatgtttctgataacaatcttcttaccagttattgatatattttggttttttatttatttgtcagttgattttgggagagtgcgggtttctaggaatttcatctaggttacccaatttattggcacataactgttaatagtagtctcttataatatcatattcatgtgaaaatatcttatattcatgtgaaaatgtaacatcccctctttcctttctgatgttagtaatttaagtcttctctttcattcttatttaaaggtttgtcaattttgttttttcaagaaccaacttttggtttcattgattctctatatctgctctaacctttatttccttccttctggtaactttgggtttcatttgctcttttctagttctttaaagtgtacatttagtttactgatttgagatcttaaaaaaagtttttttaagacatggggtcttacgctgtcacccaggctggaatgcagtggtgcaagtatagctcactataacctcaaactcctggtaaacaatcctcccaccccagacacctgagtagctggcactacaggaacatgccaccacacctggcttattttttacttttttgtagagacaggatcccactttgttacccaggcaggtctcaaactcctggcctcaagtgatcctcctgccttggcctcccaaagcactgggattatagatgcaagccaccatgctcgactgagattttttattttttgagaaggagtttcgctcttgttgcccaggctggagtgcaatggtgcggtctcagttcactgcaacctctgcctcctgggttcaaattattctcctgcctcagcctcccaagtagctgggattacaggtgcctgccaccatgtccacctaatttttgtatttttagtagagacagggtttcactatgttggccaggctggtctcgaactcctgacctcaggtgatccacccaccttagcctcccaaagtgtggggattacaggcataagccactgcgcccagcgtaagttctcttttaatgtatgcatttacagctacagatttacctcttaggattgctttcactatgtgtcataagttctggtatgctgtgttttcatttgtctcaaggtattttttactttcccttatgatttctttgacccattagtttttaaatagtgtgttaatttccacctatttgtgtatttccacttttttattttcaatgtctagtttcattccactgtaactggaatgtactctgtataatctcagtttttaaaaatgtattaacacttgtttcatggcttaacatacggtttatcctggagagtattccatgtgaatttgagaagactgtgtattctgctgttgttgggtggtattctgcttatgtctatttggtctaatttgcttagagtgttgttgaaatcctctatttcctgattgatcttctgtctggtttctatatccattatcggacatggaatattaaagtctccaactatttttgtctttctccctttaattctgtcatttctgtttcatatgttttggagctctgttaggggtatatatatatataatatatataaataaatatataaaaataaatatatatttataataaatatatttttatttcatatataaatatatatcttataaatatatatatttatataacataaatatatatttatatatattatatattatatataatatatatttatataaatatatatattatatataatatatttatatataatatatatttatatattataaatatatattatatattataaatatatattatatattatatattatataaatattatatataatatatattatatataatatatataaaaatatatatatttatatatataaatattttatcaatataatatccttgtctcttgtaacctttcttgatttaacatttattttgtgtgcctttttcaaaagaggacactggaggtgaagaaggaagctcttgactctcctaaagctgatcccaaagtgaaggctttgaaggccaagaaggcagtgctgaaaggcatccacacagccaccaagaaaaaaaaaaaaaagattcacacatcacccaccttcctgtggcccaagacattatgactctgaagacagcctcaatatcctcagaagagcactaccaggagaaataagcttgaccactatgccatcatcaagttcccaataaccagtgaccactgagtctgatgtgaattagatagaagacaacaatacacctgtgttcactgtggatgttaaaagccaaaaagtaccaggtcaggctgtgaagcagctctatgacactgatgtggtcaaagtcaacacattgatcagggctgatggagagaagaaggcgtatgttcaactggctcctgattacaatgcttctgaggttgccaacaaaactgggatcatcttggctgagtcttgctggctaattctaaattatatatatataacatgttatacatatattttatatataattgtatatatttaaaatacatatatattttatatataattgtatatatttaaaatacatatatattttatatataattgtatatatttaaaatacatatatattttatatataattgtatatatttaaaatatatattttatatataattgtatatattttaaatatattttatatataattgtatatatttaaaatatatatattttttttctccagaaaaaacagattattttgtctggtgataatatagccacctcagctgtcatatggttactattttctaggtgtatctttttctatccttttactttcaacctccttgtgtccttatatctaacatgagtctcttaagacagcatataaacactatttgtttaactccaatctgccaatctttgtttttaatagaagagcttaatccctttacatttaatgtgattacagatcaaggatttacttctgccatttggctatttgtttttaatatgtcttatatgtttttgattcctcaattccattactacctccttttgtatttatttgattgttcacaatgtactgttttgattctgtttttacaatgcactaatttgttagttttactatttagacagattacatcttacattctatgcccattaatatagatttataattttttaatgaatttgtcttttaaattataaagcacaaagagaaaaagttataaaccaaaagtacaatagtactggctcttatatttacctctaaagttatcttaactagtgctctttttttttttttttttttttgtgatggagtcttgctctgccccacccaggctggagtacagtggtgcgatctcggctcactgcaagctctgcctcccaggttcaagcgattctcctgcctcagcctcccaagtagctgggattagcaccaccaggcccagctaagttttgtattttttgtagagatgggttttcaccatgttggccaggctggtctcaaactcctgacctcgtgatccacccgcctcggcctcccaaagtgctgggattacaggtgtgagccacggcacctggcctattttttcttatggcttcaagtttctgttcagtatcctttcatttcagcatgaaggactccctttagcatttctggtagggcatgtctactaataatgatctccttcaattttatttggaaatgtctggatttctctttcatttttgaagaatagttttgccatatacagaaatcttggtttagaggttttttttttttctttttttcacgactttagatatgtcatctcactgcctttgggcttccaagatttttgatgaggaatcggctgttaatcttgtcaaaaattctgtgtgacgagttgcttctctcttgctgctttcaaaatcctgtcttcaggttttgacaatcctaatataacatgtcttggtgtggatctgagttcatcctgcttggattttattgagctttttggatgtgtagattcatgtctttcatcagatttgggaaattttctatcattatttctttaaatattctttctgtcccttttctttttctgcgattcctatatgagtatatgagtatgaatatactaatgaatataataactatatgaatatatgagtacattagtatgcttgatgatgtctcacagctctctcaggttttttttcctaaatattttttttctttcttctcctttggataatttcaattattttctcttcaaattcactgttctcttcttttgcatgcccaaatctttgttgaatccctctagtgggttttccatttcagggattatactttttagctctagaatttgtttggttcctttttataatttctatctctttattgatattctcattttgttcatgcatggttttcctgatttcctttagttcttcgtccatgttttcctttagctcgctgaacatacttaagatagttgatttaatgtctttgggtaataattccaatgtctggacttcctcaaggatggtgcctgtcaattttttttcctgtgaatggaccatggttttctatttctttttatgttttgtaattttttgttgagaactggacattatgagtattacattgtggtaactgagaatctaattctgtcatgcctcagagattgctgatttttgcttattgagggctggagccaactatttgtgacttttccaactatttttgtgaagtgtgtattctttgcagtatggggtcagtgaagtttctgttgtgatatctctgtggttaaccagtgacctaagatttccttaaatatctggcttgccaaaaggaggaataaagaaaaaaaaaaataggtactgtctctttaaatccccttgtggctagaaagctccctctgcccaagggcattgagacaatgatcagcctctgcactggccccctcagtgattaaatgcagtaatcagaaatcaaaaaactcttttttttttttttttgcaatggagcctcactctgttgcccaggctggaacgcagtggtgtcatcttggctcactgcaacttctgcctcccaggttcaagtgattctcctgcctcagcctcccaaatagctgggattacaggcactcaccaacatgcctggctaatttttgtatttttaatagagacagggttttaccatgttggccaggctggcctcaaactcctgacctcaagtgatccacacctcagcctcccaaagtgctgggattacaggtgtgagccaccacacctggccacaaaaacttgacttttggaggacaaagttgttattgcctaccctggcaccaacagtctgcaccaggaacattttccacagttgcctgctgcagagcttgcggatgctgaaacaaaaaaaaaccatcaaaatttgtaagcctctccatcaagctcttccctggatgttgcaagtgttcaactagacttctgagtcccaaaacagttgcttgagacaattactgccagctcaactgctttggtggaggaaaagattcctggtgctgctactgccatcttccataacatcattctccatgtcagctttcgtaatcaacttgttcaaacatcctcatctctcttgagcatgacaccctggcctactggtatattacaagaagcacagagctgggtataatataaggaccaacttacaaacttggacaaagacttgtaggagagactacctgctctgcattataaaaaagttaaactagccataaaaacctgttgttctgactccagggtgtgccatcagctactttataaagcaggctgtgagctgtgggccccaaaggaggggagtgttttgttgttgccgtattgaaaacatggctggagttggaggaggctgtaagggagccagtggcccttgtactcctaccctaagtgctgaccagggctttgggctccaccaggtgtttgctctcactgtgggctttgtggaactggattttgccttagtatgttaatatgagaagaacaggctttctgcaaatgctaaacagatggatgacatggtactccccgcccccaacccaccaaattatgggccaaaaaggaagtctctttttccttaattcagtaaagaaacaatagcataactatgtttcaaactgttgtgttcaaatgaatttaaaatattcagagaaagaaaaagaattttgtcctttttaagacatcctaagtagaaagcaactggggtggaggagctgggatttgccaggccaaaataaacaaacaacaatggttggctattgtatggaaacaatgcaacaccatccaggaaattctgtgggatttctgagggataggccactatggttggccctggggatgaggtaccaaacacaaggccccacactgaggcagctcatgccaaagtcaccacggaaccatcacattgcccaacaccacaaatagtgtctcctgtaccccctcccacattgccaactcagctagcaatgctgccattttggaacccccaatcccttcagcttctacaatactccgccctcctggctctcacctgagcttctggctgccctctgatggggtctctaatgagaacacaaatgtcaaagggtaaggagggaagggagaggctgggaactcagtgaccaggaacctagaaaatcaacacgtaataacatgacttgtctgtggcttcaaaactatgtcttaaataaaaataatcttctatgatacattttgctttcgagtacaatccagagaaactgaggaacaaaattcaagcttttgattccatgtatgtcaaagaaggaaggtgattttccattatactaaacatttcttctacatcaggcataaccagagaaacacaagactttcatcatcagaaaaaagcacattatgtatctaaggacaattaaaggctctctttccttttcatcaatttgagctgaatttcaactcatgtcctgttgctgtctcctacccagtggctgtggatctgcccgactcagaggatgttttgacctgttctcctattgctaagaaatggctagaaaagcttggacccctttagggtcaccatgttccttcccctgcgcaggtttttaaaaacacatgaaacaaaaaacagctctcaaccgtttatcatattatattcctatggcttctgctgctagttgtcctctgaagcagccaggcggttaatgctgctgccaggcagccttagagacagcagatgcccctgtgcagtaaattcagtgcccaaccataagtgcctcctccccttacccacacaggatagccatggcccctgtggactccttaccgagctgacgtccagcctggggactcgatgtgtcaaagggctctgagctggcctcagggacacggggctcccacgactcactgttctcagacacctctgaataggtgtcacccatccctttgtgaactgctgtgagctcaccagtaccagggccctggtcctcactgcctgtgtctgccacggctctggtctcctcccccattttctcagcaaaccactgcttgacctgctgggagctcatctgggtcttgtcacagaggttctgcaggtcctcttcatacagcatcttgtgtgtcatgtaatagtcctgcaggagctcccggttgccaggggcaatgaccagtagccctggtgggaagttgcctcgcttatagtcttcgtaccatttgagttggccgttcttcagtgcgtacctgctatctccaaaccagcgcaccacctctggccgtggcagacccgtctgggccatgatggagtcatagtcctggttgcttggccactgtgtctggacaaagagctgccgcagcaagtgccgctgctgggcagtctttttgcagctcactttgcctgggagctgctctgccagtttgtttgactcatcatcctcagggtcacaggcacccagccctggaatctcgttcctgccattggcttcagtgaccctcaggttcttcaggttgattttaatggggctgactttgcgctctgccaagatatggctgctgggcatttccagagagccattttcaccagagacccttagctcactggccaaatcctcttctccaccctcatcctcagcagcctcctcttcctcctgagaggcattctcctcagccttcttggtctcctcagcattcacttttttccgtctctctgaaaaccagctatcaatttctcgtcgggtcattttggtttcacttctcaggcggtccagttcctcatcaagaggaagagggttttgtgcaaaactgctctccagggctctgagctgctcaggggctctctccttgtatttggttggtgtgaagtcaggagtctggtgccaagattgtcgtttggcagaagggtgggttgctagtgtggctgttgtcggaatgcaggttacctcagggaccttggacgatggggagaaggacacctctggcacagagtcaatgatgatggaactgtgatctccaggtatcatcgctctggagcccttcaagttccggcagtggtatctacgatcactgaaccatttccgcacctctctggtactgaggcccgtcacttttgtgagatgttcaacttcgctctgccctgggaactggttccgacagaagctccctttcagagctgacagctgttcatgagatttcttatttttgtagatgctagcatcaaggaaggcttgggaggttatgctggggcaggccgtgaggagcgactgggccgcattgaccaccttcacagctgacgttgtatttgaacacacagtgttaatgggtgccacacctggctgcttggggacggatgtcaccgtgagggggagaggggaacttgttgcttgcaacccattggccatcaatggctgagtgaccagaagtccccctcctgtaccctctggctgccccacaacgtgacctggaagagcggcctggatgagatgctggacattgccagcactggcgacgagtggggtatttagaaccgtaattgtgggctgaggcacagactggatgactgtattgaacatctttttccgggcatcctcaatctcctcaggggaccagctgatcccctgcttcagcctttgggctgtgaaccagatcttgagctgttcttctggatacttggtcaccacagtcaaatagcagagctcggctttggtggggtaggggaacttgtggaaggagttcttcaggaagctgttagagtccatggctgcattgtacgttggaatgctgctcagggggatcatcactttgggaagggccttggccgtgggcagtggctggtggacatggtgttgggcatgcactgggggctgctgctggagggagaggaactgtgctatgccagctggcaaaactggcactgttcctatcaggggcccgttggcggcatgggggttttttgcagagctggcagatgcctggctgactggaactgccccattgatgaaggaatggtccccctctctcacctccatttctccagtcgacagctttggtaaggcctcacccacaggctggctagggacattctccttgagtgtatgaatttttttggcttcagctttgcctttcattatcttcatgattggagttttggtaatgatgatttctgcctgtccatcagccccttcagcactgggctcacccgctaggtcaggagtgctggtgctctcagggatgctctgctccacaaccacatgattgtctggcttggccacgttccacacaaagctggcttccccggagtgacatgtggcattgtgcaaggaaagcccctcaggggtttttgccagaaaactgcacccactgcatacaaaggttgggtctttattaaagtctgtgtgctctgagttcatatgtcccacaaattgggtcatgtcatgggatctgaaatcgcagtatttacaggaatataaatagccatctaaagtgctccgatgcccattggccagtgtagagccatcagtactgctggggttctgtgctgcctcactgctggcagcagatgcttctgggggcagatcctgctggggtccttcaggcaaggtctcagcgggctgggcctccatgctggcatcttgcaacaccacagtcttcactgggatcatgcatggtgtggtggatttcctcttgctggccatggtgacaatcactgggtgatcagcagttgagagcttgtcccataaggggcctaacaatacaagttccagcttctcgatgagggccaaagaaacagtttctaaatgcagctttttcagttgtttgcagaaagcaggttttccctattcaatctaaggaaagggagaaaaaaatgattaagttctcttaagtgctttctgtatatttctcggatacccagacacattccactgcttgccttctaccatctaggtcttttcacagtaagtttaatcagcaacttggatatgaagtctaagttaggaaccccctaatgacaaaaactggaccctgaagaatgacctggggatggtccaataccaactgatagacttaagcaacgtatttttgtttcttccccaccaccccctacatatccctgcattaagtaattcagattaaaaaacaaaacgccctctttttggacctccaaattaattctgaccatgtcagagtgagaacaaaagatcctagtgtaattcttgtttacattttggaccaacacgcggttttcttttcatagccttcccgcatatgagaacaagatgacgcctctgtctgtcccaagccaggcccagggaccaagttagctgcagtgtgtgaacggaaggggtggggtgggcgggggtggctaggaagaccggctgctgtaagagccaggcctctgtagcctttccttgcctgccgcacttattccaggccacagaaaagcgatcaccttacagttactggctgttatgtatcccggaccactgttactgagatgaaaccaaaggtcagagggtcagggaatccacagaaccagtttgtggcccagcaagctatacattttctgtgtacgaccactcaaagggtggtctcaccttgagatctgagaacagttggactgcctccccaagtgggtccctgacccctgagtagcctaactgggaggcacctcccagtaggggccgactgacacctcatacagctgggtgcccctctgagatgaagcttccagagggaggatcagacagcaacatctgccgttctgcagtatttgctgttctgcagcctccattggtgatacccaggcaaacagggtctggagtggacctccagaaaactccaacagacctgcatctgagggtcctgactgttagaaggaaaactaacaaacagaaaggacatccacaccaaaaccccatctggacgccaccatcatcaaagaccaaaggtagataaaaccacaaagatggggagaaaccagagcagaaaagctgaaaattctaaaaatcagagtgcctcttctcctccaaaggaacgcagctcctcacctgcaatggaataaagctggatggagaatggctttgatgagttgagagaagagggcttcagacgatcagtaataacaaacttctctgagctaaaggaggatgctcgaagccatcacaaagaagctaaaaaccttgaaaaaagattagatgaatagctaactagaatgaacagtgtagagaagaccttaaatgacctgatagagctgaaaaccatggcatgagaactacatgacgcatgcacaagcttcagtagccgatttgatcaagtggaagaaagagtatcagtgattgaagatcaaattaatgaaatgaagcgagaagtttagagagaaaagagaaaaaagaaacgaacaaagcctccaagaaatatgggactatgtgaaaagaacaaatctacgtctgattggtgtacctgaaaatgacagggagaatggaaccaagttggaaaacactcttcaggatattatccaggagaacttccccaacctagcaaggcaggccaacattaaaattcagaaatacagagaacaacataaagatactcctcgagaagagcaactccaaaacacattaattgtcagattcaccaaagttgaaatgaaggaaaaaatgttaagggcagccagagagaaaggtcgggttacccacaaagggaagcccatcagaataacagcggatctctcggcagaaactctacaagccagaagagaagtgggggccgatattcaacattcttaaagaaaagaactttcaatccagaatttcatatccagccaaactaagcttcataagtgaaggagaaataaaatcctttacagacaaacaaatgctgagagattttgtcaccaccaggcctgccttacaagagctcctgaaggaagcactaaacatggaaaggaacaactggtaccagccactgcaaaaaacatgccaaattgtaaagaccatcaatgctagaaagaaactgcatcaactaacgagcaaaataaccagctaacatcataatgacaggatcaaattcacacataacaatattaaccttaaatgtaaatgggctaaatgcaccaagcagacctaactgacatctacagaactctccaccccaaatgaacagaatatacatttgtctcagcaccacagcacacttactccaaaattgaccacatagttggaagtaaagcactcctcagcaaatgtaaaagaacagaaattataataaactgtctctcagaccacagtgcaatcaaactagaactcaggattaagaaactcactcaaaaccgctcaactacatggaaactgaacatcctgctcctgaatgactactgggtacataacgaaatgaaggcacaaagaaagatgttctttgaaaccaatgagaacaaagacacaacataccagaatctctgggacacatttaaagcagtgtgtagagggaaatttatagcactaaatgcccacaagagaaagcaggaaatatctaaaattgacaccctaacatcacaattaaaagaactagagaagcaagaacaaacacattcaaaagctagcagaaggcaagaaataactaagatcagagcagaactgaaggagatagagacacaaaaaacccttcaaaaaatcaatgaatccaggagctggttttttgaaaacatcaaaattgatagacagctagcaagactaataaagaagaaaagagagaagaatcaaatagacacaataacaaacaataaaggggatatcaccaccgatcccatagaaatacaaactaccattagagaatactataaatacctctacgcaaataaactagaaaatctagaagaaatggataaattcctggacacatacaccctcccaagactaaaccaggaagaagttgaatccctgaatacaccaataacaggttctgaaattaaggcaataatcaatagcctaccaaccaaaagaagtccaggaccagacagatccacagctgaatctaccacaggtacaaagaggagctggtactattccttctgaaactattccaatccacagaaaaagagggaatcctccctaattcattttatgaggccaacatcgtcctgataccaaagcctggcagagacacaacaaaaaaagagaattttagaccaatatccctgatgaacatcgatgcaaaaatcctcaataaaatactggcaaatcgaatccagcagcacatcaaaaagcttatctaccacgaccaagttggcttcatacctgggatgcaaggctggttcaacatatgcaaatcaataaatgtaatccatcatataaacagaaccaaagacaaaaaccacatgattatctcaatagatgcagaaaaggcctttgacaaaattcaacagcccttcatgctaaaaactctcaataaattaggtattgatgggacgtatctcaaaataataagagctatttatgacaaacccacagccaatatcatactgaatgggcaaaaactggaagcattccctttgaaaactggcacaagacagggatgccctctctcaccactcctattaacatagtgttggaagttctggccagggcaatcaggcaggagaaagaaataaaggttattcgattaggaaaagagcaagtcaaattgtccctgtttgcagatgacatgattgtatatttagaaaaccccatcgtctcagcctaaaatctccttaagctgataagcaacttcagcaaagtctcaggatacaaaatcaatgtgcaaaaatcacaagcattccaatacaccaataacagacaaacagagagccaaatcatgagtgaactcccattcacgattgcttcaaagagaataaaatacctaggaatccaacttacaagggatgtgaaggacctcttcaaggagaactacaaaccagtgctcaacgaaataagaggacacaaacaaatggaagaacattccatgctcatggataggaagaatcaatattgtgaaaatggccatactgcccaaggtaatttatagattcaatgccatccccatcaagctaccaatgactttcttcacagaattggaaaaaactactttaaaggtcatatggaacaaaaaaagagcccacattgccaagactatcctaagtcaaaagaacaaacttggaggcatcatgctacttgacttcaaactatactatactacaaggctacagtaaccaaaacagcatggtactggtaccaaaacagagatgtagaccaatggaacagaacagagccctcggaaataataccacacatctacaaccatgtgatctttgacaaacttgacaaaaacaagaaatggggaaaggattccctatttaataaatggtgctgggaaaactggctagccatatgcagaaagctgaaactggatcccttccttacaccttatacaaaaattaattcgagatggattaaagacttaaatgttagacctaaaaccataaaaaccctaggagaaaacctaggcaataccattcaggacataggcatgggcaaggacttcatgtctaaaacaccaaaagcaatggcaacaaaagacaaaattgacaaatgggatctaattaaactaaagagcttctgcaaagcgaaagaaaccaccatcagagtgaacaggcaacctacagaatgggagaaaatttttgcaatctacccatctgacaaagggctaatatccagaatctacaaagcacttaaacaaatttacaagaaaaacatcaaagaaccccatcaacaagtgcacaaaggatatgaacagacacttctcaaaagaagacatttatgcagccaaaagacacatgaaaaaatgctcatcatcactggccatcagagaaatgcaaatcaaaatcacaatgagataccatctcacaccagttagaatggcgaccattaaaatgtcaggaaacaacaggtgctggagaggatgtggagaaataggaacacttttacactgttggtgggactgtaaactagttcaaccattgtggaagacagtgtggccattcgtcaaggatctagaactagaaataccatttgacccagccatcccattactgggcatatacccaaaggattataaatcatgctgctataaagacacatgcacacgtatgtttattgcggcactattcacaatagcaaagacttggaaccaacccaaatgtccatcaatgatagactggattaagaaaatgtggcacatatacaccatggaatactatgcagccataaaaaatgatgagttcatgtcctttgtagggacatggatgaagctagaaaccatcattctgagcaaactatcacaaggacagaaaaccaaacaccacatgttctcactcataggtgggaactgaacaatgagaatacttggacacagggtggggaacatcacacaccacggcctgtcgtggggtggggagagggacagcattaggagatatacctaatgtaaatgacaagttaacgagtgcagcacaccaacatggcacatgtatacctatgtaacaaacctgcatgttgtgcacatgtaccctaaaacttaaagtataatttaaaaaagaattataagtaatcttcacatatgcttttaagtatttttttaaaagaaaggaaagaagaaagaagaggaaaccagaaacaaataatcattacactatccagacattacataacatcccttacctgtgaatttagatgtattgaacttttaataaaatagattgacttcacacttttaaaatgctaagaaagaaaacagctaaggcagaaaactaacctgaaaacaaaaccaatggttttatatataaaagaccgaaattaaacagttgataactcaaaaagaaataataataataattgtttcacaggatgccatgagaaaagggattctgtgagccctattactaatatagaatgataatcatcattattacttttcgttttagcaaatcataatgctctaatgactaaaacaatagtaattctaatactaaaatcaagttatcagtatttggatttgatacaaagatgtaaagaaaattaaaatatctctattcaaattctcagtaaataaacacggctattttatgatgaaaataatttttaaaactttgaaggtgagggtaaacataaacccactctaacaacttctcaccccttcccccaccaatttgaactggcatgcacacttttttacttaatttttaattataaagataaaatgtaatttaaataaatttaatgtctataatttggtataccttctcatttagtccagatattttattcatatttttatcacaatgagcacacatttgtgcttttttcaccaaacattacgttataaacctttttgttacacaagcttcataagtttaacatttaatagctacctaataatgtaccacaatgaatttgacttatccacccctgctgttgaaaagcatgccaatctcatgggtgacagtgaaacaaacatctctgggcatacaggatgtttttcttctatcaaattatttcctgaggttaagtgcacaggaatgggcttaccatctcaaagagcatcaacatttctaagctcctttgtatatatcattggcaaacattttaaatcaagtgctttatgatcccaaatcagggaaactaaaaataatacagttatggacccagtgagcaaatcagattttcagacaagttatggaagacatcaagtagaatatctgaaagcacaaacgacaaaagaaaaaacagataaattggatgtcatcaaaattaaaaacttgcattcttcaaaggtcaccatcaagaaagtgaaaagatagacaatccacagaaagaaaaggtttgcaaatcatctatctggtaaagtatatccagattatataaataacttacaactcaaccatagaaagataatttttaaacgggtaaaggatttgaatagacacttctccaaagaagatatacaaatggccagtaaagcacataaaaaactgctcaacatcattagttattaggaaaacacaaatcaaaaccacaatgagataccacttcacacccaccaagatggctgtaattacagcgacagataacaagtatagatgaggatgcagagaaactggagccctcacgtattgctgatgaaaatgtaaaatggtatagccactttggaaaactgtttggcaaacaaaatgttaaatatagagttgccataagatccagcaatttggccgggcgcagtggctcacacctgtaatcctagcactttgggaggccaaggcgggcggatcaccagaggtcaggagttcaagatcagcctgaccaacatggagaaaccccatctctactaaaaatgctaaattagccaggcatgttggtgcatgactgtaatcccagctactcaggaggctgaggcaggagaattgcttgaacctgggaggcggaggttgcagtgagccaagatcatgccattgtactccagcctgggcaacaagagctaaactgtgtctcaaaaaaaaaaaaccaaaaacaaaacaaaacaaaaaaaacaaaacaccagcaatttaattcctaagtatatacacagtataaataaaaacatgtccaccccaaaatatgtacacaaatgttcacagcagcattattcaaataatggccaaaaggtggaaacaacccaaacatctatcagctggtgaatgggtaagtaaaatatgatatatccatacgatgaattcttcagccatgaaaaggaatgaagttctgatgcatgctgcaacatggatgatcctgatgctaagtgaaagaagtcagacacaaatgtcacatactgtatgattcccatttatatgaaatatccagaaaaggcaaatccatagaggcagatagtagattagtggttgccagggactgagagtaggggagaagggggagtggctgctaatgagtcaccaggcaatgaaaattgttctgaaattagatagtggggatggctgcacaactctgaatatgataaaaacctcaggaaggacatcgaaaccaagaaaggtagaggacagtaaattatagcactcagttctatagaagagggtcagcatttacatttctaaggtttcaatataggtgacacagttctgtgctacttcctgactcaccttggtaatatttactgtggccctagagaagctgaatggcagtgcctgtgaacatcaggtttgccagaggagagggaagagagatgcagacctactacctggaagtagcaacaacacaaaggaaaaagagcatgagcacaccagaaggccaggcagcagaactcaggagaagctaactgcagagccctagcaacacaacacaagcactccaggcacagggccagaactgctatttgtaatggggactgggattcactgaggaaaaggtatatagtgttcctggtattttacttactaaggctgagaagtggacaacatgaagatggatttgatttttggcaatagtcccaatgtcattcaaaattaagtcttggccaggcacagcagctcacgtctgtaatcccagcactttgggaagccaaggtaggaagatcatttgaggccaggagttcgggattagcctgggcaacatagaaattgtaaaaaattagtcaggaacaatggtgtgtgcctgtagtctcagctactcaggaggctgaggtgggaggatcacctgagcccaagagttcagagttacagtaagcaaggatcatgccactgcactccagccaggatgacagagcatgaccttgtctcttaaaaaaagaaaaattaagtctcaaattagccctaaattatcattcgaataaatgactaagaagggtaccactgctaaccatgttctgggtagatggaccattgactataagcaattacaaaaaagcagtagtggcagcagtggcagccacagccagtgtttactgagtgtttcatattgttggaagtgcttttatacacatcccctcatttcactcataataaacccaagaaaaggcactataattattctcattttacagacaagaaggacaaggctcagaaaagtgcagtgactgcctggagcgcacacaactgggagacgacagaagctgaataagaacccaagcagtctcgctccagagcctgcaacttcaaccactccacacagtgagctctctgagaaacctccagattgttgcaataagggctacagacctcttgaagtaatattttcctccagcatttattaagaaaaagttcaaacaaatggcaaagttgaaagagattgacagtgaacacccatatccccaccacctggatgcttactaaggtggctgtttttagcaacattacccatttaggtaagcttgggtttatttgttatgtatttttaaaggggttaaaaatggtgcttaaacagcaaagttctaattatctagaaaatcattcatccaaaagacactcatggagcatgtctgataaatgaggcactaccatgtggaaacatgctattgtctaagtcaagccctagtgaagtgagctgatagaaatgtaaggtgcggccaaaagagattcagaaggaaaggccaggtgatgtttgcattttgtcatttttgtacaatcctttacatgccccatcttgaaaccttctgccttgtaaatggatctttaaatggggccagagcagtggctcacacctataatcccagcacttggaaggccgaaacgagtggatcacttgaggtcaggagtttgagaccagcctggccaacaacatggtgaaaccccgtctctactaaaaacacaataaattagccaggtgtggtgatgtgtggctataatcccagctatcgggaagctgaggcaggagaatcacttgaacccaggaggcagagattgcagtgagccaagatcgcaccactgcactccagcctgggacacagagtaaggctccgtctcaaaaaataaataaatacaaataaaaataaaaataaataaatggaaaaataaaaacaaaagtgcataactttttttaaaaatctgtctttgggaattggtaattgctcaattcactaaattagcaaagctatagttaatagccactatatttcagcactgcagtatacatatgtgagttttttagataaaaatcttaaagtgtttatctttaaattatagactgaattaggaactttaaaaaattctgaggaatgaattggaagaacagtaagaattcaatgaaggaattcaaatacttagaaagtaactcttgacagactcttccaagcgttatgatagtcagcctgcagccctcttgaacatatcacccagtatttatcattaaaaacggaaataggccaggcgcggtggctcacgcctgtaatcccagcactttgggaggccaaggagggtggatcacaaggtcaggagatcgagaccatcctggctaacacggtgaaaccacgtctctactaaaaaaaaaaaaaaaaaaaaaatacaaaaaaattagctgggcattatggcgggcgcctgtagtcccagccactcaggaggctgaggcaggagaatggagtgaacccaggaggcggagcttgcagtgagcagagatggcgccactgcactccagcctgggcgacagagcgagactgtccaaaaaaaaaaaaaaaggaaataatggtagaagttgacagaattacagaaacataactatagtgggagattttaagataatattctaaagatcaagtggacaaagaaaaaagatgtaaagaatgtgactactatgaggttgatctgacagacatgaatatagtcttgagtattctatagtaattgacttcacattctacaccatgaaacacatttaaaagttactcgtgatgtataacagaccataaataaacctcaacccattccaaaataaaaattcataggtagcattccactatttagtgctagcaacaaagacttactttgaaatttatcctttttactttgtttcaaattatgctctaagtggaaatttctctctcggaaagaaagaaaagtgaaaatccttcctattcacccccagagataaaccattgttaacagtgcagtatatatgatttcaggcatgtttcctaggcatagcttactataattaattcttttaaaaaaccaaaaatggaattgtatttttacatcctattttgaccctgagttttcgtctcacaattatttaatttcaatatctttccatgtcaccatctctagtttacctcagtcttttaaacaacttcagcattctaggttatggatgcatcatagtttatttaaccattctcctgataagagacatttgcatttctccaggtttttaactattacaatgactcggtgaccatccctgcacatatttttatacatttgagaatttctgaaatactgaatttcttacaagtaaaattgcagtagcatttaaaatattgttagcaaaatacaaaattgtctttcaagtaaactgtactgattttcgctgttaccaccaaagaactgaggatgcactcactatcactgcatattcttgctattttcagtctttgttaatttgatatataaaacactaattactagtatttggaatctacatttctttatgagcaaggctgaatatcttttcacatgtcattgacgattttgtataaaattatcttttgtccattttcctatgggatgtcaagtgttctccatatattaagaaaatgggctattctttacctgtcatagatgaaactggggtttcatctccattttttggagcccatctaccacaggaggcagtaaatgacattaaagaattactgttaatgttgctatgtatgataatggtatttcagttatgttacaaacaaaagtcttaatcagttggtgatgcacactcgaatatttatgggtaaaataatttgttatataggatttactgggaggaaaaacccaaccccctagaaaaaaatgagggatgggtagaaatacatgaaataaatttgaccatgaacttataattgttcaagttgggttgggatatatatggctcatatatatgctcttatgtacatttgacattttcataattaaaaaaaaattaaaagctcgagacatattttaaaaagaaatcttgtatcataaatgaagcttaaatttatatttttaaaaaacattctgttttaaatgtttctctcttcccttcctttggaaaatgctgaccttggaaacaaaaatcagatgcagccacccaccctactgctgcccaagccaatcctaagtaaatcagttggcttgctgggaagtttccatgaccgaaaagaaaacagaattcacttcaatttcatcatcaaagtccacgaaagcttgcaaagcacttccatatccttctcaatccatcctcatagtctcctgaattggttattattggccttccctcattgtagtattgaggagctatcgagcaagaaaggggaggtgatatgaccagtcacacacgacatatttttggcagagccagatattctgattctaaattcagtgttctcttttagactatattcattctgaataccaactaaacaatacatccactacctgtaactattttgacatttttttcttacagagggttcgaagattttacaacctgaactattctagtgttttgttttgttctttaaaaaggcttgacaatgtagtcctagctacttgggaggctgagatgggaggatctcatgagcccaggagtttgaggctgaagtgagagctatggttccgtcattgcattccagtaatgtgggcaacagagcaagaccctgtctcctaaaaatgaaaaaagaggctggaccagtggctcatacctgcaatcccagcactttgggaggccaaggagacaggattgcctgagcccaggagtttgagaacagcctgggcaacataaggagactccgtctctacaaaaaatttaaaaaatagctggcatggtgacacgcatgcttgtagtcacagctaattgggaagctgaggcggatgactgcttaagcctgggaggtcgtagctgcagtgagttgggattatgctactgcactttagcctgggcaacagagcaagaccctgtctctaaataaataaataaataaataaataaatggtaaaaaggcttgacagttattatgatccttagtatttataacacttttataccgtcagaagagggtctgtattgcttactcttatgtattaatagagaactcttttaacatgaggcttggtgattagaccacggtaagtcagtaggactctataacaaaaagaacatgtggaaaactgtccttcctcaggaatgtgtgtgggcaggaccttggcagtccagtaagtggatgtgctggttgcaattgtcagatttctgctgaaaacaaaaaagttacaaagaactcaagtctaaatcctcaaatgtcaaagcaatgtcaagcccagagctggcctcaatttgccctctggttacttaagggtcacaataatttctgcatttagtaatcaccatcagccaagggctcaaggctagagctctctacttgtttttccccctcaaacagtgctgttttttttttttttttttttttttgagacggagtctcgctctgtcgcctaggctggagtgcagtggcgcgatctcggctcactgcaagctccacctcccgggttcacgccactctcctgcctcagcctcccaagtagctgggactacatgtgtctgccaccacgcccagctaattttttgtatttttattagagacgggttttcaccgtgttagcgagcatggtctcgatctcctgacctcgtgatccacacgcctccgcctcccaaagtgctgggattacaagcgtcagccaccgcacctggccttccccctcaactcttatttttgtctaatcaccaacagaaatagcccactctagtcagatattatcacctctaaagtaaacattaatttgttctttctcaagctcagaaggtgattgtgatataaatacaaaggcctcagaattaaagagactttagagatcatatattctaaccactcacctttgcatgaattttaccttaagtaccactaccaggtggttaggatggcctatacctgaagatctccaaagatagggagcttggcacttattgacactagaaagatgtttgaaatggttccttgtagtaagcaccaaaaacaacaaaaccttcctataagttttatttgctaaatttgttttttctctcaaatagagttgaggacttactatgtgctaggcactgggtatacaaggatttttgaaaagatgctagctagccctataactcaagaagctaacagtctgttgtggttattaagctatccaccagggctctgggccttgccctccactggatgcattgaatttccctgcctacctgaagttaggtgaaaccaatgaaacgtgaacaaatgtgtcacctccaggggaaaagttttatgagaaagtataggctttgcctcactatttattctctggcctgcaactggttatgttccagatggcggctactctatcagcctgagtcctaaaagaagaacaatagctgacctgccatgtacatgcagtatgaacaagaaataacctttgtcactttaagctactgaggtttttggagtagcttgttactgcagcccatcctcactgatacagagtctgaagacagacaaacaaaatcatcagcaaaatgatcaacaaagtcatcaacaaaatgatcaacaccaatcagacaataagctgagtgttgtagtggaagcatgcacaaagttctgtggagcataggaaaagactttctacccacctctgtcttccggggaacttggggtaaggcctcatagaggaggttgcttttggattagagctcgaaggatgaataaatactgccaagtagataagttgagaaaagagtgttctagacaaagggaacactttatacaaaaaccgtagagggaggagaaaacatggcatgattggagagctagaaaggattcagttttgctattgtacaaatctgaatgtggtagatttcctatgaagctactacagccgaagctttggggaccctcacttcacttgcacaggcaccttccaaagcctctatctaactttatatctttataattttattcttagatttttttcttaggagggtcccctttactaatgttcaagttccacaaaagctggatctgcttgtgatggatcaggttgcaggaataggaggtgattctaggtatacaaacaagatatttgactttctatatgatacagttttttttggttttttttgttttgttttgtttttgttttgttttgttttgaggcagagtcttactctatcacccaggctggagtgcagtggtacaatcacggctctctgcagcctcgacctcccagactcaagtgatcctcccgcttcagcctcccaagtagccagtattacagatgcgtgccaccatgcccagctattttttttttgtagagatgaggtttcgccatgttgcccaggctgatcttgaactcctggactcaagtgacccacccgccttagcctcccaaagtgctgggattacaggcatagccactgtgcccagccgacacatttttcaattggaaaaacgtgattttttggaaaaaaagaatcatgttccctaaaagtcttcttttcccctaaaccaataatcctcagttcctgtactttgtgtgactgggacagagactccacaccactctccactgttcttctctgaatgtgctcttatttgtctatagccttctcaacagtggcatctaaatctaaatctaattctccagatgtagtctgagcgtatttatcaccttccttaaacttactcatgtcatatttaatctttcacaaaagtttttacccaaacgatgccacattatttataatgtgagattcccctgaaaaatatggatcaatgctcttggatttaatccacagcaccaggacttgtaccaaaatacaatacctgcttgtttcaatttaggtttggcagaatttgaggaacaagtttgcagagaaaagatagtcaattcatttaacaaatgtttactaaatgccttttctatgccagacatgccagaatatggggaagacaaaaaaaaatagatagaaatctctgccttcctggaatttacattctagtttgggggagagagataataaataaaataagaaaatgaaattatgtgtcagagcaaaaagtggaagaaaagcatagcagtgaaaggctgaagaaatgtggagcatggcaggggtgagactgcaatttagatatacagtggtcagaagagaactcactgagaatgtgacactggagccaatactcagaggtgtggaagtgagccatctgggtaagtgaggaagcacactccaggcacaggcatcagctggtgcaaagaccttgaggtgagaatgtgcctgacatgttcaaagaacagcaaggaggccagcagagctgggcggcggtagtagagagacagtagtatgagacaaggtcaaagaggctgtggtagggggcagggaggggatccatgcaggtccactgccatgatctgagtctctctgaaatgggcagtgccaagagagggctttgagcagaaggttggcgtcatccgacacgtgtccttcagcatggctctgctggctatgttgagaacagactgtaggaaaacacagtggagaagtagggaaacttgtcagacatcaatctaggcaggagatgacagcagcatggaccaggatggtagagatggaggtaagaagaagtggctggattctggatatgttttgaaagtaaagccgaaaagacttcctaaaggaatggataggaggatgtgaacattgtagccacgggaatcaaggatgactccaagatttttgatctaagcaactagaaggatagtgttgccatcaaccaagataggtttaaacctgggaagctttgtgaaaaaagcaaagcaaatctgtgctgctaaaactagaaggtccttttccaggggaaaaactggattgaactggtttggtcacaagtgttgtaggaaattaatgggtcagactgctaatgagcaaagaggctgtgtagtgccaatgttcccaatgaaaagactctggtctttgccaaaagctgctcaaatctacagagaaactacagaaatgggggttggggggaggctgggaccaaatctagggtattataagagccaagtagttagaaattgcactctctgaggtgtgagacagcgcctttcaaagttaaacatgttactttaaatgtacttgcatcctaaatgttgttccaagtactaagtaaattactttattaaatttaaaactaaacagtaaaaaagaaattgttctggatgtggttttacttctgagattacatttgttttgttttaattgaaattttccagtgtgggccagatagtttacaggctgaatagcttgcaggctggtgagctcacttgaatttctcaagaacatttgttaaaaaaaaaaaaaaaagaaagaaagaaagaaaaattctctctggctgaccactgtggataagatgttccttttacctcttccaggcttagtctgtattgatgattttcttcaatggcctcactacaaagtccagcaagatcttacacacaagaaaaaatgctcaatttcactcacagtaagagaaatacaaattaaaattactctgattatttttctttcacttactttggaaaggcttgaaacattggataataatatcctgagttggcaagggtttgcagaaataggcactctcatacactgcttgctggagtgaaaattggtaagacttctattaagaagtatttaacaagaacaaaattataaaagctcattccctgactcaccagattctgcctctaggaaggcagcctgctgatatactttgcacaatggtaagtcatacatgtataaggtaatctactatcagcatggtaatggcaaaggactggaaataacctaatcactcatcaacaggagataatcaaatcaattatggcacacttatacaatggagtaacacacttgtgaaaaagaacaagggaggtgctttttatgctgctatggattaaccttcaagctatcttgatatatgttaagttttttaaaaagcaaagcacagaataaaaattatacgctagaaatacctcgagaaaaatatgtgagaaactaataatagtgtttgcccctggcgaggggaagagggagatgtgtttgagggaagcttttccctgtagccccttttaaattctgaacaatgttaatgtgctatgtattcaaaacaaaaagttaaaaattgaataaatacataagtggaaaaaatgttagcatatttcccaaaatacatgggttgttacaaataacaagtgagaaagtgagaagctaatggcgtcagagtagaggtccttacagagtatgtaactgggtcaaataccaagtagtgctcctttacatttcaaacttctaatagctacagatgagcttttggtgtaacttctgtaggggtgcatgtgagcacttcacttaaaactatatattatatattgcatttgcctctggccataaagccatagttcaaaaataacttagagatatccaatatacaaaacaaaaaataaataaataaaagaacaagcaaaacaaaaaaaatacccaaaatggaggagtgggacaatatatgcagtccaacttctttcctaattaaaaaatcctgcagcaatctgttatccagtctccaactgagtatctcccaaatgtgggagctaattatcatttatgacaatttattacaaagcccctttcttagttggacctggttcccagcttttagatttgtcttttggacataaatcaactctttttactacatgacgcatcaactctcttttctatatatcagcttttcacacagttgaagatcatttcctcttttagtatttacttctgcagctaagtgtcctgaattcattgcacattctctctatacattctaatttgtctgcatttctcacaaaatgtggcaattagaactgaacaaagcattcaagatgtagcccaaccagaagagaattaaatggaagctgtgttttcatcatcctattaccatgactaatacaaccaaaaactgagctagatacaagttctattctccccctcacagaggaggactttgaggtgaagtgacccagtgggacaaggaaacactgctagaattaaggaggattaggattctttcatcacaaggccatttggatacacgttttcagacactcatttataattccaacagaggacttctgagctatcttcccctttgcctctttgaaatctaccttcttaagaaaggaatgttagactttctctgaacatcctatctaaatgtctttgggggctcccccaaatccaagctgaattattctctatcacagcatcctaattgtttctttcataacatttatcaaaatttgtctggatcttcatctgattcttttcctgcttattgtctttcactttggctaggctgtaagctctgtaagtaaggtcagggcctatatccactgtttccgtagcatatagcacaatgtctggcacataataatcgttttcagtgagtgaatgggtgaataaacggtagctgataatggagttaagactctaacatcagacagagccctaacactgtggactgtgtattcctgggcaaatgacttaagtcctcacaatccatttcctcattggtaaaatgaggagctattaatacctacctcatagacctgacatgacattcggtgaattgataggaagtacacaaaatgatttagcatgctgtctgatatacaggaaacactcaataaatcatagttactgcagctactgctactgttctaattaccaccacatcccactcagaggctggccactgaaggatgacaattaactgtaacagctagaggtacatcagctgttttacccagcctacctttctttctttttaaaaaattgtgataaaaaacacataacataaaattcattgtcttaaccatttgtttttttcttttgagatggagtctcactcttgtcgctcaggttggagtacagtggcgtgatcttgactcactgcaatctccgcctcccaggttcaagcgattctcctacctcagcctcctgagtagctgggattataggcacctaccaccacgcagggctaatttttgtacttttagtagagacagggtttcgccatgttggccaggctggtctcgaactcctgacctcaggtgatcagcctgccttggcctcccaaagtgctgggattacaggcgtgaaccaccgcacccagactgtcttaaccatttttaagtgtacagtaaggtagtgttaagcacattcacattgttgaccaacagatgcccagaactttttaatcttgcaaaagatgcaaatactatacccatcaaataactattccccttactctcttcccccagcccctggcaactaccatactactttctaagagtttgactactttagttacctcatataaatgaagttatatttcaagatgtagcccaactagaagagaatgaaaagcaagctgttttcatcatcctactaccactactaatactaccagaaacctagctagctaaaaaaagtacttatcttgggctgggcgcggtggctcacacctgtaatcccagcactttgggaggccgaggcgggtggatcatgaggtcaggagatcgaaaccattttggctaacatggtgaaaccccatctctactaaaaatacaaaaaattagccgggcgtgctggcgaatggcatgaacgcgggaggtggaggttgcagtaaactgagatcgcgccactgaactccagcctgggtgacagggcaatactctatttcaaaaacaaaagaatttcctttcattttaaggctaagtaatattccatgatatgtatatatcatattttatttatccattcatccactgatggacatttaggttgcttccccctcttggttattgtgaataatgctgcagtgaatatgggtgtgcaaatatctctttgggatcttattttttattcttttgggtatacactttttcttgtttaagctctccacaatcctatgatgctggcagagcagcagaaatcacaggagtcatcaattcgtctttaccttttttgatcctgtcatcaaattatcttcctctcaagctcactccctagtcctttctgataacctcctggttcctctattttcaagtgaattcaacaggtcaaatttaaaatttattttacttgtccaaaagggaaggaagctattatacctttcctatccctttctgttggaagaactctaggttcctcatccagagaagggtactgccccaggtctgagcagaatgaagctgcaggtactcagggtctaagccccctggaacaatagagggctatggctctaccttagtcggcccaggacagtttctaaaatgtttcctttatgagtactgttttgaatgctgttaatatttcatcaggaagtatcagtttcccttgacaggtaagattaagtcctattcactgggttgatcagccttatcattacagccaggatatttctctgcaaatccccttccaatcttatgctgcccaactaatgggaccaactggatttgctcaataaagttctgtattttcccagagctcttcattttcacattattgtcctggggtggggggcagcagaacagagatgttgacagccaaatggaagatgcattgggaggcttctgaagatacttgttctgtctttgagaaaccttagagcatcaaggtgaagcaaaaggaaaggagaggaatagtgctcagtacctgagccttctctaatctaaaacgcaataaagtcaatcataaatcctgaaaatgagagtggctgtgatggtgcaaaatataaaactccaacaactatttatcaggaatgaagtacatatatctccctattcgcatctcagccttcctcctgcaatatgtgagtgagtgtgcatgaacccacactcaccagtgatacactcaggagagtactggggaagagttaacttccgctgcaagatagttagcatatttatttcctttccatgagacttcaggagcctaaatcagaagtttctaagagcttctcttgatgtcatatattctgagaatggatacatctaagaactgatgtaatgtgtctgccattatgaatttggtaaaagttctatttatctgtttttaaacaaaaggactgctttaatgttacatattggtttgttattacatattggctggttatttgttattggttgtttctatgacacacttggacaagaaaatttctagtccaagtatatcatagtggtattacttgagacaccacttaaatctcataaaaatagaaaagatcccctaactggcctctcccatcttccccctcccccatgccagactgcctctgtcccacttattcaacttccttgctcatcaatctttggtgcattgcaggccccattccttcctctccttctgcccaaaccacttgcaccaaggtcatcagtcaagtggctctttcctactccttcaagtctcaaaccactcaggacccaaaggaagttaagacccacacctctataacctctccaaagggagcaaactcatgcccaggactttgcaatctcctctacccaagagatttccaaatcacccttgccagccctgacctatctaccaagttccaaatccattacttccaactgccattgatccctgcaggttttaaaaaactacatgcataaatttctggctccgttgaccctggcactcacccttccaaaaaacgaaacaagcaaataaaaaatatagcctaggtctacttcctaaatcttcatttcacctctcaccatttgtcttagtccgttcggggtgctataacaaaaacatcatagactggatggcttataaacaacagaaatgtatttctcacagttcttgaggccaggaagtccaagatgaaggtgctggcatattcagtgtctggtgagggcctacttcctcatagacagacaacttcccactgtcctcacatggcagaagggaccagggagtgtctggggtctcttttataaggccactagttccattcatgagagctctatcccagtgacctaattaccctccaaatgcctcacctcctaagacaatcacattagggattaaaatttcaacacgtgaattttaggagatacattcagtccactgcaccatacatccagatttccaagcatacttatttttttcctagtttcctttattcacatctctcttttctcttttcccacatgaaatgttaatggtctcggaagcacttaacatcttttcttggtagagggacttttagaaattgtggtaaaatacacataacataaaatttaccatcttaatcatttttaggtggagagttcagtagcattaagtattaacacagtcacattgttgtacaaccatcatcaccatctgcctccagaactctttgccttataaaactgaaaccttacacccattaaacaataactcccatttcctgcccacccctgactcctgacaactaccattctgctttctgtctctgtagatttggctactctaggtattttacataagtggactcatatagtatttgcctttttataactgatcaatttaacttagcataatatcctcaagtttcatccatattatatgtcaggatttacttccttttaaaggctgaataatatccccctgtatgtatatacaacattttatccatctatcaatgaacacttgggttgcttctaccttttgcctattatgaataatgttgctatgaacatggacatacaatctctttgagtccctactatcaactcttctgggtaactacccagaagtggaattgcttaatcacatggtgattctatttttaatttcttgaggaattgccatactgttttccataacagctgcaccattttacattcccaccaacagtgcactgagttcccaagagttctccacattcatggcaatatctgttattttctttttttttcttcagagttgccatcctaatgggtgtgaggctgtatctcactgtggttttgatttgtattgcctaattatattaacgttgagtatttttaatgtgcttattcatttgtgtatcttctttgaataaatgtctattcaagtcctttgaccattttaatcaggttgtctggggttttttgctgtttttgttgggttgtagaaattctctatatgttctagatattacccctttattagatatatggtttgcaagtgtcatgaagcttttctcctctgtcttcttctaagcattttatagttttagatcttttgtttaggtcttcgattcattttgagttaacttttatatatatggtgtaaggtaaggatccaactctattattttccattagatagccagttttctcagcaccatttgttgaaaagactgtcctattcccattgaaaggtcttggcatccttgtaaaaaatcatttgaccttatgtgtgaggatttgtttctggtctctccattctactccattggtctatatgccttcattaacaatttttaatattaaatagatgttgaaataatattttgcatatattgggttaaatatattcttagattttttttttttactgtttcaatgtggcccataggacacttatcacgtattggctctcattgtatttctgttggacagcactgcctagatgcttcagactggagccaccgggcagtgtggagagcaacagctaacgtcatgggattagatgatcatctgaaagctcctctcctcttgtgtttatcattatagtacttagggagcaggatatcactgtggctgagaattgaggcttctgagtcaggcaagtccaggtttcaagacctgccctgtccaacacagcagccactagccataggtactcatgtatgtactgagtatctgaaatgtggctaatgtgaattgagatgtactgtaaatgtaaaatacagaccagatttcaaagactcagtaccaaaaaaagtaaaatatctagttaataacttttatatcaattacatgttaaaatattagtatttctgataaattgggttaaataaaatgtattattaaaattaatttgacttgtgtttcttagtaggctattttttagatcaagttcacagcaaaactgaccagaaggtatagatatttcccatatacccactgccccacacatgcatagcctactccattatcaacattccccaccagaatggtacatttgttacaactgatcaatctacactgacacatcattatcacccaaagtccacagtttacattagagttcactcttggtgtacattctatgggtttggacaaatttataatgatatgtatctacaatattataatatcatacaaaatagttctactgctctaaaaatcttctgtgctctgcctgttcatccctccctcctccctaactcctggcaaccactgatcatttactgtctctatagttttgcctttttcagaatgtcatatagttggaatcatacaggtatgtagcctttttggattggcttctttcacttagtaatatgcatttaatctccccccattgctttttattttttgacttgttaattttgattttttaaaaatgtggctcctagaacattttaaatgacatatgcagctcaccttatgtttctgctgaacatttctcagcttggcatgggcagtttttctcagcatggcatcttcctgtctttccagtcactcaagtccaagcctggttgtacttggtttgctgcaacctaaacacaccagcctccttgccttcacacatactagagttccttcattagagttttctttaaccccctccctcacctgaagactaattcatgctcagacccaattcaaatacgacatctttagtgaagcctttctaaacttccactgtacctaaatcattttttatagcacattctaccttataatttttttgtatgtggctgactttctctccagattgtgaaatctttgaattaggccccatgttttgttcatctttgggtcctcaatccttagtacagagcctgacacatggtaggcaatcagagatgttaagttaataaatgaataaaatgcaggaaaattaacatatcctctaataagagataacatattcacttagaatcaaatagcttagaaaagaacctcaaatgccataaacaagggtgtaaaattctatcagccaacagctaaaattattcacaagctaaaatttgtattcagtcaaagcaacacattaatcaataatatttataaagtatatcacagtgaaaagatggtaaagcacctcctaatggttaatgatagtgattggcctggagatgtggaaactagctatggaaactagatggttaaaacaaccacattccgtaccattagacgctgacagcccagaccccaccaaaatagaggcagtgaaacagcctttgaagttgttttcaaaggaagccaccaagatgattaaagggttggaaaatgagatttatgggtaatggccacagaaacgaagacaattttgcctaggcaagagaaaaggccgaagggtgacttaataaccatcttcaatcattgttattacacagagaatgatgaccagatggtctccattttcaccagagaaagaatattgaaaatggacttacgctgcagcataaaaaaaaaaaaaaggtctagttttaaacaaagaactcaatggttaagaactagtatacagcaaagcagacctggggagtgtgtggtgtagccatgtctgaactcatctgttatgctgggaaagcagcaagtgggtagggtgtgttcaggtcttcaacagtcctttgttgagggcaaattctcaactggctagtcctcaaatgaatcacgtagcatacaaccacaggagtggaaaagaaagagatatgaaaattgaaacacagcaccctctggtgtacatagctgtcttctaatctaggaaaattcttcttttttttttgagacggagtctcgctgtcgcccaggctggagtgcagtggcgcaatctcggctcattgcaagctccgcctcccgggttcacgccattctcctgcctcagcctcccgcgggggactacaggcgcccaccacctcgcccggctaattttttgtatttttagtagagacggggtttcaccgtgttagccaggatagtctcgatctcctgacctcgtgatccccccgcctcggcctcccaaagtgctgggattacaggcatgagccaccacgcccggctggaaaattcttgatatgtatcagtatttttcaaaatgtaagctccaaaccatgacagagtcaggagaaacttacacaaggctggtcttagggttcgtttaatcgctacatccatctttatcaagcacctgcgatgcatttctccctgttaccaggaggtaccaggaagcctgttacacaagcaacctcaacttctgcctatagcaaacttggattcttcccaatcaccttttgaatgctgaattcagactctgatacaactagctggcacatgaccatgcaatgctgagacatgataacatctgccagataatgtgaattttgccctgtgttcaggagtacattacatataaaagttaagcaacacctaaatgggggtgttcccttagacattctccctgctatgttgtggtgtatacacccttctttactgatctctgtctgccaaatcccagccggccactttttacatcctcctaggctttatgctgcaaaaccttcctaatatccttttaagtacaaaagacattcaatgagctcaaaaagtttgagacccaatcaagaatcttttgcagccctctcagtatctttagccatcttgttgccagatgggaacttcattcacatgacacacatacttccttcatgtgactcaccattgctaagcaactggtatgtcctaggtgaataaaacatatgccctgtgtctgagattcacagtgcagtaggggaacatggatagatacgggtgtaaatagatcagctgaagcatgagttgaagctcaaggctctacatgccaagaacagaggaaaaaggcaatggagagaccggacaagaactatcaggacatcagaacagaacttacatcctgtgagagcaggacttatgctaaaaacaagtcttaaaggatgaaaagaagttgtccaggcagacaagacaggaatatgtatcatcctaggcataaagtcccaagacatgtcaccttttgaagaatggagccatgtggcacactgtcctccagtcccaaaggtctccttgaagagacatgaaaatcccacctgaagaactcacccttgcagattgtacatagcacagacctaaatggcaacaaacaagcaaacaaaaaacgcagtacagactaggcactgatctgatggaagcgagacaggctggagtagcaaagggtctgactaagaagcttaggaggaattcctctgatacccttcttggtgccccctccccagcaacatctaacagcctcactcgggcccaacttccctgttgcactggtggcactgcattgcaattgtgggtacaagattcctcccccacaaaactgtgggctgaggcaagagttctacctttttattactacgcctctgacactcagcacatatgaagcacaagcaaaatttgatgactaagctaagtaattagttcatatgtgtgattagggtttgaaagggcagaaccttggacctatgatcccaaatacctttgtctatctagttcacctggttttgttcctcctcagaagccatctatcctccttaccttggttcatgttttcctatctctccattttatacacataaaccaactcctaccctcaaatcagggaaaagggacagatattcctgtcctcaagttaagagtgacctatgtcaaagtgaaaaacaactaagccaaaaaggtaacagggaactggcttcttgagcactcagaagaatttgagattactgcctcgcgagggaaaaagggaaacccaacccgattacagtgtaaaaataggtcactgcttgaatacagccctttggttactggctgcaagcccctttttgcttttgactccagtgtcacaagaaagtggcagcatagtcttagaataagggctccacagccagctggctgctcggccaggccttcctgctggcattctagaagcagcatgcagggccaggcacctactggggagctcagtaagcctttgtcactgctgacatgaaatacaagagttggtaagtttctttggttttgttttgaacaaactggcagatagttcaaggttgtccttgtaagtctctctcacttgcattcagctatacattgtttagtaggaaatgagtatagttcagctagttcacttatccccatgccaaaccagcaggcagcctcttttccatgagagacagttataattttgaattattgtaatgctgtacttgcagcaaactcggtgtgtgtgtgtgtatgtgcacacatggctgggagtggggggtagtgccaaaatgttggttctatggcaaacaacaacaacaaaacaaacaaacaaaaaaaccggctgtgtatagctaaaaaacatggccaagaaaaaggtagaatagaaaacatgttttattatcctgatactaaagcctggcagagacacaataaaaaaagagaattttagaccaatatccctgatgaacatcgatgcaaaaatcctcaataaaatactggcaaaccgaatccagcagcacatcaaaaagcttatctaccatgaccaagttggcttcatacctaggatgcaaggctggttcaacatatgcaaatcaataaatgtaatccatcatataaacagaaccaaagacaaaaaccacatgattatctcaatagatgcagaaaaggcctttgacaaaattcaacagcccttaatgctaaaaactctcaataaattaggtattgatgggacgtatctcaaaatagtaagagctatctatgacaaacccacagccaatatcatactgaatggcaaaaactggaagcattccctttgaaaactggcacaagacaggaatgccctctctcaccactcctattcaacatagtgttggaagttctggccagggcaatcaggcaggagaaggaaataaagggtattcaattaggaaaagaggaagtcaaattgtccctgtttgcagatgacatgattgtatatctagaaaaccccatcatctcagcccaaaatctccttaagctgataagcaacttcagcaaagtctcagcatacaaaatcaatgtgcaaaaatcacaagcattccgatacaccaataacagacaaacagagagccaaatcatgagtgaactcccattcacaattgcttcaaagagaataaaatacctaggaatccaactaacaagggatgtgaaggacctcttcaaggagaactacagaccactgctcaatgaaataaaagaggatacaaacaaatggaagaacattccatgctcatgggtaggaagaatcaatatcgtgataatggccatactgtccaaggtaatttacagattcaatgccaaccccatcaagctaccaatgactttcttcacagaattggaaaaaactactttaaaggtcatatggaaccaaaaaagagcccgcattgccaagtcaatctaagccaaaagaacaaagctggaggcatcatgctacctgacttcaaactatactacaaggctacagtaaccaaaacagcatggtactggtaccaaaacagagatgtagaccaatggaacagaacagagccctcagaaataatgccacatatctacaaccatctgatctttgacaaacctgacaaaaacaagaaatgggggaaggattccctatttaataaacggtgctgggaaaacgggctagccatatgtagaaagctgaaactggatcccttccttacaccttatacaaaaattaattcaagatggattaaagacttaaatgttagacgtaaaaccataaaaaccctagaagaaaacctaggcaataccattcaggacataggcatgggcaaggacttcatgtctaaaacaccaaaagcaatggcaacaaaagccaaaattgacaaatgggatctaattgaagagcttctacaaagcaaaagaaactaccatcagagtgaacaggcaacctacagaatgggagaaaatttttgcaatctacccatctgacaaagggctaatatccagaatccacaaagaactcaaacaaatttacaagaaaaaaacaaccccatcaacaagtgggcaaaggatatgaacagacacttctcaaaagaagacatttatgcagccaaaagacacatgaaaaaatgctcatcatcactggctatcagagcaatgcaaatcaaaaccacaatgagataccatctcacaccagttagaatggcaatcattaaaaagtcaggaaacaacaggtgctagagaggatctggaaaaataggaacacttttacactgttggggggactgtaaactagttcaaccattgtggaagtcagtgtggcgattcctcagggatctagaactagaaatgccatttgacccagccatcccattactgggtatatacccaaaggattataaaacatgctgctataaagacacatgcacatgtatgtttattgcggcactattcacaataacaaagacttggaaccaacccaaatgtccaacaaagatagactggattaagaaaatgtggcacatatacaccatggaatactatgcagccataaaaaatgatgagttcatgtcctttgtaaggacatagatgaagctggaaaccatcattctcagcaaactatcgcaaggacaaaaaaccaaacaccgcatgttctcactcataggtgggaattgaacaatgagaacacacagacacaggaaggggaacatcactcaccagggcctgttgtggggtaggaggagtggggagggatagcattaggagatatacttaatgttaaatgaagagctaatgggtgcagcacaccaacatggcacatgtatatgtaacaaacctgcacgttgtgcacatgtaccctaaaacttaaagtataattaaaaaaataaaataaaataaagaaaatatgttttattaacaaacagcatattttataatggcggtgatcaatatttattaaacacaaggttctagagggctcttctggtaggataattctaagaaagaggttccagatgtcaacttacatattcccttataaaacacagccctggcttacacagtatctggtgtacatgtctcttctctccaatgaactataagatctttgaggtaagggttcagtgctgcaccactcagctacactaatggcaatgtgcacacagtgagcactcattaaatacatcagttacagttcacttgcagttgcttgcttactaccaaaaaccggggtcaatcattataagcattcccatccaacacccaatttgttgcatggggaatgaacttcagtttgggtcccttgctattaaagcactcaatacccattatttgtttgcatttatgtcgaactcatagatgcaggatctgaaaagttaaatgaagcttaacataggcatccaaataaaattaattagaatttatatctcaactatttctaacagagtttgagtaacaagactcaacagaaggaaaacagggtaataacagagactaagagggaaaaaaaggaaggcagcagggagtgaaggctggagaagagaagaaggaaggaaaagaaggagggacagagggtggaaacatctatctaagcaagagcttcttcctcaagtcaaaaatttctgcaaaaaacaacatctttttggacaaatttacatgatgcttctaacatctgctcattgtgaaaaatgcaggataaaaacctacaaattcagtatttttagtatttttatctaaatttatatttcatcaaaacaagataaatttgaaaaatattaatacatatataggcaagacatttccttactttgcggaaatatgctcacataaataatccattctttgccaaaatagagcccttccaacacctgttccccaatctccaaggcagggtcttggtccatgggttagaatcactaaaacacacaaaatacaaggaaatgtatctattaagcaccatttggattctaagatgcaagtaagaaacctaatttgaaagggcttacataataaaggtggtttgctggcacatgcaaatggaaagttcagcggcttcaggttgagtttgagtcagttgctctatgatgaaaacaagggcctattttctctctcttttttttttttttttttttttttttgggagacggagtcttgctctgtcgcccaggctggagtgcagtggcatgatctcggctcactgcaagctccgcctcctgggttcacgccattctcctgcctcagcctcccgagtagctggggctataggcgcccaccaccatgccccgctaattttttttttttgtattttttagtagaaacggggtttcaccatgttagccaggatgctctcggtctcctgacctcgtgatccgcccgcctcggcctcccaaagtgctgggattacaggcgtgagccaccgtgcctggccaataagggcctattttctttctttctacttcgtcaaccacatcctaggggtagcttccctcgtgacagcaaggtaactccagcagccccaagcttcatgtcccaaacatccagaaggacacagagagagggccgtcttggggaggtctatcagaagactgaagagccttcttcccaaagtcctcagcaagcatgctggtggcctgcacaggtcacatgcccagccatgaacaaacctcttggcatgactgccactgggttaatcactgtgcaacaggtgtggggtttacctgactgggcttaaacaatcaagatccatccctggagctgggggtgggaccaatccttctcctaccagaagacagaaacaaacattccctgtttcctatggaagggactggggagggagggagaagagacattgtgagaaggggaacaattagaatgccctccacccttcacctggcactgaatttgaccctgtgattcccagtatcaagggaaaatatggaaatgtattttaagtgacactatttttatcgtttaacaaagacaaatgtttattcttatacagagataaattcttttgaagggaaacccaaggagaagaatgtcttggaactgtgtttaagagaaaataggtaaagcaggaggcttagtttggaaagatgcagtcacactcaataggccattccttttttttttttgagacagtctcttgctctgttgcccaggctggagggcagtggcgccatcttggctcactgcaacctctgcctcccaggtttaagcgattctcctgcctcagccttccaagtagctgggactgcatgtgcatgccaccatgccaggctaatttttgtatttttttagtagagatggggggttttgccatgttggccaggctggtcaacaggccattcctacatggatcctccagaaggcagagcatgggacagtaaatcatatcataatccagtgaagttactttggcagagggctgagatgatatagctcagatgctttcccttcaaatcatcgaacttactgcaaagagtagtaactcacatatcttagaaccccttttttaatgtttatttttgtaggtacatggtaggtatatatatttatgggttacatgagatattttggtacaggcatgtgacgcataataatcacatcagggtaaatgaggaattcatcgcctcaagtatttatcctttgaattatactgttatttgtaaatgtacgattattttctacaatagtcaccctgttgtgctggcaaatactaggtcttattcaatttttttgtacccacgaaccatccctatttcccccccgtcactcaccctactacccttcccagcctctggtaaccatccttctactctgtatctcgataagttcaattcttttaatttttagctccaacaaataactgagaacatgcaaagtttgtctttctgctcctggcttatttcacttaacataatgacctccagttctatccatgttgttgcaaatgacaagatctcattcttttgtatcgttgaatagtacttcattgtgtatatgtactacagttttctttatccattcatctgttgatgggcacataggttgccttccaaatcttggctattgtgaatagtgctacaataaacataagtatgcagatatctcttcgatatactaatttcctttttgggggtatatacccagcagtgggatggctggattgtatgatagctctatttttagtttttagaggaacctccaaactgttctccacagtgggaaattacattcccagcaacagtgtacaaggtgtcccttttctccatatcctcaccagcatttgttacttattgcctgccttttggataaaggtcattttaactggggtgaggtgatatgtccttgtagttttgatttgcatttctctgataaatgatgctgagcactttttcctatgcccgtttgccatttgtatgtcttctgagaaatgtctattcaaatcccttgcccatttttaatgggattattagatttttttcctatcgagttgtttgagctctttatatattctgattattaatcctttgtcagaaaggtagtttgcaaatattttctcccatattgtgggttgtcccttcactttgttaattgttccccttgctgtgcagaagctttttaacttgatgtgattccatttgttcatttttgctttggttgcctgtgcttgtccaggtattactcaagaaatctttgcccacttctgtttcatgggtctatgtgtctgtttttatgccaatatcatgctgttttggttactataactctgtgtataatttgaagttaggtaatgtgattcttccagttttgttctttttgctctggatagctttggttattctgggttgtttgtggttccaataaaaatttttgaattgttttctctatttctgtgaagaatgtcattcatattttgatagagattgcattgactctgtagattgctttgggtagtatgaacattttaacaatatttattcttccaatccatgaattagaccccttatgtatcatatttataaaagtaagctcaagcactttaaaatcatgacctctagagtgttttaaattactgtcattatcagattatcctctgagtgtgcatgtgtgtgtgtgtacatgtgtgcacctgtcattatttcaaattttaaaatatttgagtgctactagatgctcagctctaagagacagggtacaaggaggaagagttcaaaactatagttgtctctgccctcaagaagtgtaacaactactactaccatcaactggataaaattataggatgtcaggcactgtgctttacatttattttctcatttaatttgatcctcacaataaccctatgagctaaatatcaacttgattttccaaattgggaaaccaaggcttgaatggctaagtcatttgactcagtcacacaagcaacaggcaacagcaaggattcaaagccaggtactcccaattccaaagcctagattcttaaccacagcacatctggctgacaagaaaacacaaacatccattaagggtgaggagtagaaagagtatatttacagtgaagagctaagtcctgagatatggagtaaagtggcttaggagaaatcaaagaatgcaaatagggtgaggaggagccacatacacagctgttagctttgtgaaccctgtgaacactgatgcctaactgatcaaacacaatctgtctgctggcctctgacccatgatctactagtgtactatgtctttagccactgacagaatggaaaaccaatgacgttttgacaggccttaagacctttcaaagttatctccaaggatggaatcagctctttccctgatgtggacagacttctattctgttctagacataaggccatatatggaggaagtttgaaatcctctggccaaatactagatagtacttccattgtttaataattaagtgatttttataaccctatccctaagagcatatggcaattgtctttgcccacaggatcaactgtcactaaccttttagcttggtggaaaaagctaaatgtcttataatgtctaaaaaaatgtaataccacttcctctgtgcttgtctccatgtaccaggaagtcgtgggtctaagtggatgtggtgtccctctcatgcctcagcagtccctttacaaagcagcagacttctgggtacccaggtcggggcagacctcccaaccttcagggtatgcttctaggttcccagtgctccaagagagggagaaatacttcctctataaaataatggggagtcagaggcatatgggatttccctttgcttccatgaaattaggagaaagcaccaacactttctagtaattccacagtgaataggatgtgctaccagcccagcatcagtagggcttctcttcattctcaagaagtgtaattacctgacgtggctgcctggaattaagattgccgctatcaaggtaataagaaacagttccattcttccccatgagccaattattatcagggtcacatctggaaccttggcttcattttcccgattcaatccatatttgaatgaccactatggaccaggttctatgctaacatctcactgactcaaacagtcttaggaaatggatattatttctgtcttacctatgtggaagctaaataaagctcaggaaagttatatcttttgtcacagagctggacaattatagttggataccagactgaatctcctgacactatgttctgggctgactccactatgtgccactgactcccgtagtgctgaggtttggggccttataatattattattataataacaatacatgggatttcaatggtcatcctcataatcaagctcttactctactctcaaaggagaaagaacatatcatgttgaagggggctattagaaggcaaatgtggtaagtgctgcaatgaatggacacaagcagtttagaaaccctatgacgaggatcaactctgactggagtgaggatgaaggaaaacttaagagatgtgaagccatccactggggaggagatgcttttgaggttaacagaagctcttaaacacagagttggaagcaggcaaacactaagggattcgtggaccacctcttacatcagggtttctcaatctttttattgacatttagggctagataatcctatgtgctggggggctgtcctgtaaattgcaggatatttagtaacatccctgactagatgcccactagatcccagtagagcacactgtccccaggtcatgacaaccaaaaagtctccagacactgccaaatatcccctaggggacacagttatccctagttgagaaccagtgcctttggtgaagatgtgccagaaactggggattggctaaatgcggaggctggggaggtgcagagcaagaacaggaagccatgaaccagataggaaagagggacagatgtgtgactcaagtcctctcagcaactccctcaggtatggactctgactaccttaaagcaaacggagggtcaggcacagtagctcacgcctgtaatcctaccactttgggaggctgagccagcaggatcacttgagcccaggagttcaagtctagcctaggcaatagagtaaggccctgtatctgttttttaaaaaacacatatttaaaaaaagacaaatggaggcaaataagatcccatgatgagggccctttgatgaatgtccagtaggaccctgattgggagtaggacatttataagtggagaggatggattctagacccacgcagcttggaacagaaaaacccttactctctttctcctttcagtaaatcaaagaaagagaagacaaccagggggaatgctaatagaatagtcataatgaacctggaattcccacgcccatttgggaggattggagaggaactggataaggagaaatactgatgtgtgttgtgtctgtgactgcagtgccatccttaccttctcggccatctcctattccctcacctctcacccttggaagggttttggcccaggaaaagcaaaaggagttgggatacttacttcagggagggaggaaggaaagaaaagaagggagtagagatgagagaattctgaggttcaaaagatatctattgacagtgttcacaaaaaggaccatgtttcccagtaaaaagaaaacaaggtaaagctgaggctggggatgggggttgaggaaagggaaggggcctacaagtaaatgtttacaaaatgcccactgagtgtcaattctgggcaaggcacattgcatgagtcaccaccttcaattttcacagcatcatgtaatacctatatatgaagtggttataacaagctcctttctatagagaaggaaagtgaggttcagaggcatcttcacagactgaggggactcccagcgtggccaggggcttgtttggcagactcctccatgctgtgcctcctcacacttcagcacccacagcctcttgcctcatgcaagctgtagctgcctgcttgtctgtttgctctgcaacattagagcagaggggctctaatctgtgtcaggtgctgtgctttgagttctgtgactctagtatttccttatgtgccaggcacaggccccagtgatacaaagaagggagcaagagttaacgctgaggtgaacaaaggacccatggaagcccagctgtgtgaatttacagcacaactaatgagcacacccagttacataactgcctctcctgctctgctcctatgctcagcagccactagaggaaagcagggctgtctttcttttcctgtgctatatataattccaactgtttgggcagggaaatgtgctacagctccacagctcagttacattagatcttaattttttcttaaaaacctagcaatttctaacagtgatgccacaaagataataatacagtaatcttaagattgagatactttacccacatttcctgcacttttctaccttgaaaacactcattatttctggttccactttgagaagcagcaagcaaggaaagggtgaaaatgtgagatgctgaagagacagcttgcaacattccataattacagccttcaatgctggaacctcaagtctccaatatcaaatggaacagtacttccccttggtatctaactaggagctgatccattaaccaggaatatatttttgatatagagtaggccagttttgtttctggttcttttgcatctcctttcctaggccctaaacacagactccatgacctcatgacagtagaatgaatgctgcataaagagaatcattcattcgtcctgggacttcactgaagatgatttttgcttagtaagcctcttcttcagatataatactaaaaggtatgttttctgagacctattaaagcacacatttactaaaatttacattaagttcatgtcagtcctaaaaggttctctaatttctgaaagccatagtctgttattctaatgtgaattgttacacacacatacagacacaccttgaacattttaaaattacatttatttaagcatactcactcactcatactttttgccaatttggttgggctttcccctaaaccaattcttctaaaatggtgagactttaacaaataaaatgatatagctgatattttcatagggtaataggttaagaagaaaaaaatccaggcttttgaaatactaattgtattagcattaatactaatacctaggccaggcgcagtggctcacacctgtaatcccagcactttgggaggctgaggcaggtggaacacttgaggtcaggagtttaagaccagcctggccaacatgaggaaaccccatctctactaaaaatacaaaaattagccaggcgtcgtggcacatgcctgtaatcccagctactcggaaggccaaagcaggagaatcactcgaacccaggaggtggaggttgcagtgagccgagactgtgccactgcactacagcctgagggacagagcaagactccatctgaaaaaaaaaaaccaaacaaacaaacaaacaaacaaaaaaaactaatatctaaatattagcagtactatgatttatagacatgaactcttataaagagtatgtatatggctgggcatggtggctcatgcctgtaatcctggcactatgggaggctaaggctggcagatcgcttgagcctaggagtttgagaccagcctggacaacatggcaaaaccttgtctctacaaaaatacaaaaattagatgggcattgtggcgtgcgcctcatgcctgcagttccagctacttggcggggctgagacaggaggattgcctgaacccaggaggtcgaggctgcagtgagccaagatcatgccaccgcactccagcctgggtgacagagggagaacctgtttcaaaaaagaaagacttgcttaatactgggcagctcagttatagcaagtactagtcaagggtggcatgtcactgtgttcagagttggatgaacctgggttctaatctggactcagctacttcctagtgtaggtcatttgggtcttgggcaaagtattgaacctcccaaacctcagttctcttttgcacaatggaaatcatctgaatatagacaagatagggcccactataaaaattaaacaagatagtctgtgtggaagtatctttaacaggcattaagtgcctgttaagtgtctttaacaggagaattaagtagcatgttcaggctttttgcctccttcctcttcccctccactccccagtgaccaacttgcaccctgagtaaggttttgcagcaccatccttctcctgtggcttgtaaacctctgcagtaacttcagcaatctacactgcctgcaccctgccctccctgtatgctcccctgaatcacaaccatatttgcctttctagagcacatatcattgtcttcagtgattccctgcaggacaaagtctgcccacttcagcctggtatcatgatcctgtcttagtctggtccaacccaccctttctgaacagttccttacaaacccacatcttcctacctcaactctgcctaggccattcccctgcttcaagctcttacagagcctcccatggcctacagtataaattcctaactgtttcaggtggcatttgagaccgatggtatcaggtccctgcgcctctccagtttcactgtctgctactcgtttccctccttatactttacacccaagcgtaaatctacttatagtacctgaaaccccatgctgggtcatcactcttcagctttacatatactcctctctttgacgagaatactcttctcatccatttggcaaattcttcaacagacttcaaacctctctcaaatgcctctacttctgtggtacagtctgtccagaactgtgtcttgtacatttttctactactttgcacctgtgcctgtcatacaacgatttctccagtttctccggttttttttttgtaattcactgaccattattaatgtcttctctgctaaaagtataacaacactcagctttgcacagagcagttacttgggaaatgtttgcattgctgaacacaggcagcttccaatctcatgggaacatcaaatgaaatgcagaggatacatgctatacaacacagtcaagaggactgaaagcaggcagcaggagaattccacaatattcctgtttaaaatgttaaaatgttggtggctgccagagctcaggaaaagaaaatttttttatgtggttacagctcactctaacgagtcaattacattgacctaaagcccatggtgctcaaaactgtatttctctctccaaaacctcattcttttataaagctccctcctctgtggaatgaatgtgggcaatcgcttttgcagtgtttgacgatgccagtttatggctatgtttttctgttcactgaactggctcatttcttcaggagaacaaactgttctccactgaagcaggaattggctgggttctaacaaaaagatttttcccccctgacagtgtttttgcaaaagacccaagtcaatttatagccaagtaacaaaaggtcagaaaggttagtcaagcaatgggcgtaattcctgtgtgtcacagccaataaaggaaatgggtggggataaatagtatgcaattttaacctgaaattatttgtgcaaaactattcttgtaaactatacaaaattacccatgacttccactatataggatttctcactctgatttttttaaaggtacatttactttggaagaactgagatgacctcaaattcctcattcacaaatggaaaatgttaaaaactttcttcacactaatataagaaaaacttggtcattgttttgcagccctgaaacagaattgttcacataaaaaaaaaaatctcatagttaagtcacaattaaatacaatttgcatgactgaatttaaaataaattgcaaaattcccttgccaaaagggaaacaacattgaccaaaaggataggtcaagaaaagaccctacaaaagtgacagttcctctgttttaatcatgctaaaagtataacaaactactgcattattcagtgagcataagaacaagaaaagcctgcctttaaagcagacacgaagaactgacatcaccagccaagcaagcggaaggcagatgagtaatagcctgcactgggcggccaaccaggcccaggcccatcagtctgtggagtaagaaagcctttcagatatcaaggtcgttgggaggcctccttcccccagttctccaatacaaagtctaatctactcaccatttaaaacaatcttagcatccaaaagatgaatggttaaatagaaccctgtcaccgttcagacaagatggtctaaacccatttctctccccacctccttgtaagcacaactgtaaatcctgaaaataacacaaaaggcaaccaaaggagaagtctgaaagaaaaaggaaggtgaattacttagggacccaggattggaggaacaacatagcagcacatcttatgaccacacccccattcaatagaagaaggtggccgagggtgtttcccaacccccaacctagcaacagagggcagcagaggattgaatgggagttctgccgacaataccaagccagctggaagcaccaacaagggggatagctcaggaccccactaacaataaagcagtctggggaaacacttttccccccaactacagggcccccaggaagcaagctgcatcccagcaggatggagactccctcctctacctagatatcaggcagcatggcctggggaaactccttacccccatcagacagcaccaacagggactagtgggagccgtggcagcaccagataaaccaagtggaccaaaagagtactgcaaaggctctgaaaagtaaatcatcgttggaaccacaagagtaaatcccagctcacatgctgcatctaaacactgtgaccgcctgctaaaatggaagatttaaataggacccagactctcttaatagacaaaatgtccgaagtagattttaaaaaatcacctgttggccaggcgtggtagctcatgtctgtaatcccagtactttgggaggctgagatggtttaatcatttgaggccaggagttcgagaccaacctgggcaacatggcaaaaccatgaagatggactgaaaacagacagaacctccaacacttatgaaacaatagcaaaagatccaatattcatattatcagagtcacaggagagaataaaaagaagggctgaaagagtattcaaaaagataatgactgaaaattccccaaatttgctgaaggacataaacctagattcagaaggtgaacatatcccaaataggataaactcaaagaaatccacactaagacacatcttaattatatttctgtaaattaaagacaaagaataactcttgaaagcagagaaaaacaacatactacctataagggaatatcaagcgacaacaggtttcttatctaaaaccatggaggcctggccgggcacactggctcacgcctgtaatcctatgactttgggaggccaaggcaggcagatcatttgaggttaggagtttgagaccagcttggccaacatggtgaaaccccgtctctactaaaaataaaaaataataacaaataaaattaaaaaataaaaccatggaggccagaaggaagtggcacaacatttttatggttaaaggaagtttttcaaacagaaaggaaatgattaaaagaattctgaagcatcaaaagggaacagaaaacaacagaaaaggcagaaatatggggagatatatatatatatagattatcctcctgtagtcccagctatttgggaggctaaagcaggaatattgcttgagtctagaagttcacttctaccctgggcaacatagcaagactctgtgtcttaaaaaaattaaaaattaaaagggggaaggtaaagggacctgaatggaaatcaggtttccacatttcactcaaagtggtaaaatactgatc

SEQ ID NO: 2 - protein, 1290 aa, Homo sapiens
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKENGDLSPFSGTSLRERGSDASGQLFHGRAREGSFESRYQQPFEDFRISQEHLADHFDSRERRAPRRTRVNGDNRL

SEQ ID NO: 3 - DNA, 159095 bp, Homo sapiens
gaattctaaaaacagcaatagaaaagtgtctagtcacttataagagaactctcatcagactaagagtggatttctcagcagaaaccttacaggccagagagaatgggataatacatttaaagtcctgaaagaaaaaacctgctagccaaggatattataccaacaacgttattcttcatacatgaaggagaactgacgtctttcccaaacaaacaagctgagggaattcatcgccactaaattggcactacagaaactctttagtcctatgcctggaagcgaaagaataatatctaccatcatttaaaatatatatatatatataaaccactggtcaagcaaacacacaagagataaaacacaaatattaccaagacaaaaaaaccaccaaacaacaatgacaaacaatgagagaaagaaacaaaagataagtatagaaccagaaatcaattaataaaatgagaggaatatgccctcacttatcaataataaccttgaatgtaaataaattaaacctgccacttaaaagatacagtctggctgaatgaattttaaaaatgacccaactatatgctacctacaagaatctcatctcacctgtaaagacacatatagacagaaagtaagggaatgtaaaataatattccatgcaaacagaaaccaaaagtgagcaggagtagctatacctatatcagataaaacagacttagaccaaatggacctaacagacatttaaagaacatttcatacaaccactacagaatacacattcttctcatcagcacacagaacattgtccagaagagatcatatgttaggacacaaaacaagtctcaacaaatatttaaaattataaatcatatcaattatcttttcagaccacaatcaataaaactggaagtcactaacaaaagaaactttggaaagtgtacaaatacatggaaattaaataacatgtcctgggagaaattcaggatgaaatttaaaaattaaataacctgctcaaggaagaaattaagaaggaaatcaaaaaatttattgaaacaaatgaaaataaaaacacaacataccaaaacctatgcgatacagcaaaaaacagtgctaagaggaaggttaaagcaataaacacctacatccaaaaagtagaaatatttcttaaaaaatctaaccatgcacctaaagaaactagaaaagcaagaacaaaccaaaaccaaaattagtagacggaataaaataaagatcagagcagaattaaacaaaatagagacaaataaaataatacgaagggtgaacaaaaaagtttgtgttttgaaaagataacatcaataagccacttgctggagtaatcaagaactcttacacactattagtgaaaatgtaaattagtatagccactatggaaaacaatgtggaatttctcaaaaatctaaaaataaaactaccatacactccagccatttcactgctgggtatttatcctaaagaaaagaaatcagtataacaaagagatacaagcactccaatgtttactgcagcactattcacaataacatatatatgaaaccaaactaaatgttcagcaatggatggatgaagaaaatgtgtataatgtatatatatatatatacacaatatgtgtgtgtatatatatatgtatatatgtatatgtgtgtgtgtatatatatacacacacacacaatggaatactattcagacataagaagaataaaatcatgtcatctgcagcaacatggatggaactggaagtcattatgttaagtattaagtttaaaaagccaggcatagaaagacaaacactcatgttctcactcacatgtgggagctaaaaaagttgatttcacggaggtagagagtagaatgatagataccagaggccgggaagagtaggtgagtgggaagggggatgaagagaggttggttaatgggtgcaaaacatacagttagatagaagatataacttctaatgtttgatagtgaactagaatgactagaaatgacaatgacatatggcatgtaacaaagtagctacaagagaggacttgaaatgttcccaagtgatagaaatgataaatattcaagatgatggatatcccaaataccctgacttgatcattacacattttatgcatgaaacaaaatatcacattactccataaatatgtaaaatattatggatcaataaaaaataatgtatttgaaaaactaacttctttaaaaaataaaactgaacccctacattacacataaaaaattgatgaaaactaaatgtgtaagaggaaaaaagctttatattaaagttaatagaaatacaggatttatatgcatatatatatttataaaccatgacttggtaaaatttcaaaagcaaaaataataaggcatatgcatgataaatttggttgtatcaaaattaaagatttctattcaaagacatatactatggatggttctcatagatgaaaaaaatgagagaagattcttatatgtcttggcaaaaaagtgaataatattgagaaaatacaatgtctaaatgtctagacatcgttaattgataggaaaatgacagtaactgcaatagaacaatggtcaaaaggtctgaataagaaactgcaaacttaggaagtatggtcagctctctgtacctatgggttctacatcagtgattcaaccaactgaggatggaaaatattcacatataaaaatgaatggttgtgtctgtactaaacatgcacagactttttttcttgtcattattccctaaacaatacagtataatcaatatttacatagagtttacattgtattaggtattatatgtaatctagggataatttaaatcacatgggagcatatgcatatgttatatgcaaatactacaccattttataacagggacttgagcatctgcatattttggtatttgcggaaggttctggatccaatctcccatggacaccaaaagactaactgtatatgaatacataaagagacactcaaacttactagaaatcagagacatgcaaatcaaagcaaagcaacatcacttgacacctattggattaacaaaaattggaaagatggataaccgatggggtgtggagacataggggccctcaagcgctgctagtgggattgtaaactgcagcagaccctgtggagagcatctggtacatttgactaaacttggtataatcccacctgcagtgtgattcagcaattccactactagttagatatgccaataaatttctcatgcagacttctaagacgacatgtgtaatatttattacagcattatttggggaagtctggtgttggaggtgagatttggcaagttcattgcaaggagaatggatagataaaggttgatggatgcccttcatagggtactacagagtaaaaggaagcaatggactagatgtacatgtagcaacatggatgaatcttataaacagaatggatgaatcttataaaataatgccaagtaacaatatttttaaaaacataatatcttatatgtaaactaatagcatttacataagttgaaaatgtacagacataaaaaataatatgcattttccaaaagcacttataaacaaaaagacatgagaatagatgcttatgttgagaaggaagaatacaaaatgggtactggtgattaaatggaatgaattaagagtattgattgaatgaatgaagagcggggattttactggatgaatgatgattcaggtatcattactgtaccttctgcatttgagcatagaaaataggaacagaaaatttgaatggaaagcaaggaattatatgagctcttcctccaaaatttacatcatataagttgaaaactgggtgaagtataagcagggagcatttttgcaatgtttaaagtcctaattcataattttgaattgtgtttttcttaaatggctccttaaatataagactatattttagtgaagccaataatgttttcatattcctattgatgtgatctacaaaggcaaaaaacaattttaaagactaatttctaatcttactttaattcctaaactagattttcaatggatactgtctttccatgacattaattgctacaaaagaatacagaaatttccccacaaataaataaatcaaaaattatttttgactttagctatttttacagacttttgtccagtttgttgtattttatataaattcatttcttacacaaatttgtacataaaagaaaaaataagaaattctccatatttgacactgatcccttctggttgattttttaatttcttgatgcaactagcaatggtgcagtgcaaccatagctcactgcagcctcaaattcccgggctcaagccatcctcctacttcagcatcttaaatatctgggactacaggtgtgtaccaccatacctgatgtatatatatgtatacatatatatatataaaatatgtatagtatatatattatatatattgcattatatatatagcattatatatatatattatatatagcattatatataattctatattatatatagcattatatataatgctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattgtatatagcattatatataatgtattctatattatatatatagcattatatataatgtattctatattatatatagcattatatataatgtattctgtattatatatagcattatatataatgtattctgtattatatatataggattatatataatgtattatatattatatataacattatataaaatgtattatatattttatatatatagcattatatataatgtattacatattatatatagcattatataaaatgtattacatattatatatatatagcattatatataatgtattacatattatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattacatattatatatatagcattatatataatgtattatatattatatatatagcattatatataatgtattatattatatatagtattatataatgtattatatattatatatagtattatatacaatatattatatatagtattatatataatgtattatatatagtattatatataatgtattttatatataatattatatgtatttgtgtatatatatatttgtacagacaagatttggctatgttttctgggctcatctcaaattcctggcatcaagaaaaccccccacctcaatttcccaaattgctgagattagaggtaccagccacaatacttggctttgagcacatttctttatttgaggtaaatctttcccttgaaaatatttgatttccaacaagtcaatttcctgatgatttttttccaggaatttatttttcactgtttcattcacatttaatgctgtttttaaaaattctagttattttcagccattcttatgaaagatgaattgccacaggggttcaccaaaaagaaatggaatgacatttatcaaagataatcacagtcattaaaattggagatactgattttacagaggatgatgactttagtccttaaacatgactatttttaatactgacatataggcattagactataggaaaaaaacatttgtcaaagagttttgaaactgcaataattctgctttcttcaatgaatatcttggcagaatcctctgggaacaaaatgtctaagagtacagattctatgacaaggacaaattctcacacatactctgattattctaacaaaataatcagagagagaagaaaaagaaacaaatgtctatttcttccaagcttcagttcagatctatattccatgattctgactaccattgcggggtaggggccagagtgagggtgtttaatcaagtttgataatgaagcagttgtcactacataaagtgtaaaataatatataaaatattagaattcagattctaaaggtaaatcaaatatgcattgcctgattatataatgagaccttttccctccctctttctctccctacctgccccttttcccgtcttccttttcttcataacacttgcagaatgatatcgtaggaaaagaccaaaatattatccttagatgaacctgaacatattgtgtgactgtgaggaagtcaccaaccttcatggatttaaataacacagctttggtatatagtgacatgacaaaattagactaaaatttcttctaaataggcaatgggtacttttgtcaccctttggaatctgaaatacaaaataaaaagcaggaagacttagccagagcagtcaggcaagagaaagaaataaaaggcattcaaataagaaaaaaaaaaaaggaagtcaaattatctctattcaaggatgatattattctatccccagaaaaccccaaagacactgccaaaagtctcctgtaactaaactttagtaaagtttcaggatacaaaatcaatgaacaaaaatcagtagtatttctatacaccaataacattcaagctgagaataaaatcaagaatgcaataccattaaaagagctgcaaaaaaaaaaaaaaaagaaatacctagaaatacatctaaccaaggaggtgaaacatctctacaggaaccacaaaacactgctgaaagaaatcacagatgatacaaataaaaaaaaatttcatgctcatggtttagaagaatcaatattgtaaaaatggccaatactgccgaaagcaatctgaagattcaatgttattccagtcacactacccatgtcacttttcacagaactggaagaagctattgtaaaattcatatgaaaccaaaaaagaaccccaaaatgcaaagcaatgctaaacaaaaagaacaagctgaaggcatcattttacccatcttcaaactgtactataaggctacagtaaccaaaacagcatggtactggtacgaaaaacaggcacataggacaatggaacagaacagagaacccagaaataaaggtgcacacctacagccatctgatctttgacaaagtcaacaacaacaagcaatggagaaaggactccctaatcaataaatggtgctgggacagctagctggcctatgcagaagaatgaaatgactcctaccttacaccatacacaaaaattaactcaagatggattaaatatttaaacatgaaatctcaaactatcgcaccactgcactccagactgggcgacagagcgatactccgtctcaaaaaaaaaaaaaaaaaaaaaaaattcccagaagaaaacgtagtaaacacccttctggacgttggctttgggaattaatttatgactaagtcctcaaaagcaactgaaacaaaaacaaatattaacaagtgaaacctaattaactaaagagcttcttcaccacaaaagaaactatcaacagagaaaagagacaacgtacagagtggcagaaaatattcacaaagtattcatccagcaaaggcctaatatccagaatttatcaggaacttaaacaattcaacaaggaaaaccaatgccattaaaaagtcagcaaaaaacctgaacagatgcttctcaacagaagacacacaaggggccaaaaaactatgaaaaaatgttcaacatcactagtcatcagagaaaagcaaatcaaaactataatgagattacatctcacagcagtcagaatggctattactaaaaagtcaaaaaacaatagatgctggtgagactgcagagaaaaggggaaaaggaaggcttatacaccgttgaagagaatgtaaattagtctagccattgtggaaagcagtttagagatttctcacaggagttagaactattattcaaaccagaaatcccattactgggtatatatttaaaagaaaataaattttctaccataaagacaaacatgcacttgtatgttcattgcagcactattcacaatagcaaagacatggaatcaacctaggtgcccgtcagtggtggattggataaagaaaatgttgtgtgtatatatcatggaatactatgcacccataaaaaagaatgagatcatatcctttgcggtaacatggatgcagctggaggccattatcctaagcaaattaacagaggaacaggaaactaaataccacatgttcttacttataagtgggagctaaacattgggtactcatagacataaagatggcaacaatagacactggaactactagatggggaagagagggctgaaaaacaatctattgagtactatgctcagtacctgggtgataaaatcaattgtactccaagtctcagcatcacacaatatactcatgtaatgaccctgcacatgtacacctaaaatctaaaataaaagttgaaattattttttaaaattaaaattaaaataataaaataaaattagaataattattttcacttctagttcctccattaactttttgtatttaagaactggttgtttatggttgggcatggtggctcacagctgtgatcccagcactttgggaggccaaggtgggtggatcacctaaggtcaagaattcaagaccagcctggccaacatggtgaaacgctgtctctactaaaaatacaaaaattacccaggcatagtggcacatgcctgtgattccagctactcaggaggctgaggcagaagaatcacttgaacctgggaggcggagtttgcagtgagctgagattgcatcaccgtactctatcctgggcaagagagcgagactccatctcaaacaaacaaacaaacaaacaaaaaagaactggttgtttcttatctttagtgtgatcggaaagttaagagggaaattctggatcaccattcccgaaacaaaagaataactctagaaataaagttctagtcattcggctgatgtcagttggtgaaattgtacagcaagaagattttactggcattctttaacactgattgcatactctatactctaagaaattaaaactagggaacctgaggtcatttatctggacaaagggaaaaaaaaagtccaagttattcgacttagaacaggatgaatcctgacattaaaaacttcattaacaactcacactatcacacgcttttgtgtactgagacatgtaacactaaggcaaatcaattggaacggggatatgtaaacaggggtatggccacttgggtctctgaggacaaaacagcatcatgttataagagtgctcatggctcacgcctgtagtcccagcacttggagaggccgaggcgggcggatcacgaggtcaggagatcgagaccatcctggctagcatggggaaaccccgtctctattaaaaatacaaaaaaaaaaaaaatagccgggtatggtagcaggggcctgtagtcccagctactcaggaggctgaggcaggagaatggcgtgaacctgggaggcagagcttgcagtgagccgagatcgcgccactgcactccagcctggggcgacagagcgagactccgtctcaaaaaaaaaaaaaaaaaaaaaaaaaaagagtgctcttgttggaaaagagctgtttgacctgtgtggggactgaatacttacagtgtatgagggttcacagccctctccctatagccacctatgattgaacaagcaatggttggtgttaggcatcttccagacatttatattgacttccctttacaaaaaatatacgaaaggtacatgcatagatttttaaaggaaaagcaatcaaagagattgtgtgaaggggaaatgggctttgttgatatgtgggatgtacaagagggcatggagggagaggtgctcaaggaaccaaggtggtgaaggctgtagatgagagacattgtgtataaggcaggagaataaaaggcccctggaggtttgaaggtaccagagaataagaaggatccagcctggttagtacaattggctgtgagctaagcaattgaagtctcttacatgttgtgagtaaaagtcttaattttggtttcaaccatgcctttcccaatattatagctccaaaactagaccatatacattccagtttgtaggtattacacatggcattaattatttgaaaagggataatgtatttaaaatggtgacatcaaagcaaaggactttcagagagtggctttaagtagcaccttctctcccaacaaccccatatacttgctcatctggtcccaccctactccaacaattaaatgccaaacaaaggtatatgatcccaaagtgtcttccaaactccccaactcagtactgtgacacttcaatcctttatatacatggtggggaaattttaaagctttagaactcccattgcagagaggagaggaaagacaccttactctgcatttggttaaaggtagctcttttataacattgagcttcaacatttgatcatggccttacgtattagtccattctcacgctgctataaagaactgcctgagactgtgtaatttataaaggaaagaggtttaattgactcacagtttcacagggatggagaggcctcaggaaacttacaatcatggtggaaggggaagaaaacacgtcctatttcacatgacggcgggaaggagaagtgccgagtaaagcggagaaagccccttataaaaccatcagatctcatgagaactcactattatgagaacagcatgagggtaaccgcccccatgattcaaccacctcccaccaggtctctctcacaacatgtggggattatcaattcaagatgagatttgggtggggacacagtcaaaccatatcggcctatattattttgtttcctgtgttttctcttcacccttttctcacttgtgtgaatttttggttcttccccaccaggactaaaactgtgctctataaactgtaaatactcagccttataaacattaatgatgacacagaaaagagaggtgtcgagtgaataattggatgctgaaggttaaccatttagtgagttgaaaacatcgacaattggccactccataagaaagtatttccttaaagcttattttattagcgagggagactatatgcattatgttattttcttttctcaaggtctgtatttatcccaaaacattcagtgttaaaactatatcaatgctagagttcaaatctcagctctacaagatgttgactttggggaaatcacttaattggctcctcagctgtcaaaggagataaggacagtcacctccttgacgtattattgcaagaattttgtgagatgatatttctctagcagtgttcttaactaggtcaaacttgaaatttgaataaatctggcatgtacagttaagtatttattatgggcaaggatttactccagagagagaagagctgaagaataccatgggaaataaatttgaattgtttcatgatggtagtgcccagatttcaccataatgtatgagaacagacagaaacataggacaaatttgaagtttttctgcaggacaacagggaaggaagtttggcaccagaaatctgggggaataggtgctaaaaatgtgtaaactagaagagttgtctcagaacttttgggatccatcagttctgctgcaatgacactttcaataaatgaaacactgtgcaacagaggggtggattctttccttaccagtgcaaccaaagtgggcagctatgagaacgggatcatgagagcctgggaaacaacccctaagaattctcatagataatagaatagaagaaaccttgaaaatgagcctgtgttcccataggcacatgagatagaaaaggcatatatagtaaggtaacctcatttgtatgttcacagtcagtgaaggactggatgaatacaggtctcaagtgaaatacactaagcacaaagcctggtgtatagtaggtcctcaataataaatgtttgttctctttcccttgaaagacagacctcttaattaagacatcctcgaaagagatttctcaactttttcattagcatcctctggataccttcactctcattaccttcagcactatgattagcgtcaaaaagaattcttcatgtaaagagtctgtaattaggataccagggagagctcagcatctgaaaaggactgcagcacctgcctattgtctcagttgctttttatctctctaggcctgcaatatcaaaagcacccagtacaaattaaagatgcaaaaacctggctggtctcaaagttggttcatttcccgttagaaatactggctgtgcatttttcttgaaattagaacttcagaacagcacatccagtaattagtgcatgattttcttttcacttgaatgttttctgcttcttttttactattgcagcaattgggtatttgagagagaaaaataatgtctcaacgcatgcttcatttcccaaagcagggggcagagtccagattcctgatgaaagcttcaaccttttaattcagatgaagtgaagctcaatttattttgttggaaagtttgaactggtctgtgaggcagttcaacagttcaggcttactgtaattaactgtcaaataagagtccgggactaagcagagaaacgcaagtgggtcagcatcagaagggtttaaattctgatgaaattttacaagaagaatggcctttcgtggaaacattcatcccagtcaatagttcactgtataagcagcagcaagtgtaggtggttatcaaaaacacagcttgaactcccattaggcttccagtgccctccctttctggttatcactgatgctcctttactagaaacattaattcagggtttctggtaaccttgcctctgataaactcttctactggagcttgtccgcataaggagagcgagcctttgaggctgcggaagtttgaggggaatacctagaaaactcctattgctattatgctaacccacagactgcattgaatggcaaaatgcgtaagaaaaatgtccttggccgaatgcagtggctgacacctgtaatcccagcactttgggaggcctagatagacagatcacttgaggacaggagttcgagaccagcctggccaacatggtgaaacccatctctactaaaaatacaaaaaattagccaggcatggtggcacatgcctgcaatgctagctactcgggaggctgaggcacaagaattgcttgaaccagcgggggtggaggttgcattgagccaagattgcaccactgcactccagcctggatgaaggagtgagactctacctcaaaaaaaaaaaaaaaaaagaaaagaaaaagaaaaatgtcctatattgttacctatgtggttaccttccagtgtcttcatgtaagtccagattccatttagtattgctttcctcctgcttggaggccttcctttcacatttcttgtactgtaggtcagctggtgataaatattttcaatttttgtatgtctgaaaaacttcaaacagaatatggaaatgacagaccaccaacactgagcagaaaatattagaatttaatgtaggcaaatttaatctgcaacgctttccaagaccaaagcctttcatttagaaccttcatattaaaggaaagccattttgggagaaagtaaatggcatgacctttgtgacaattttcataaaaatcaatttttaaggtctccattgagctaacataaaacaaatctgcttttgacccatatactgcattaataacatccaaaaaaggctctatatctgaagtagttttctggggtaaaagaatgtataaacaaaaagagacaacctttcaaagaagaactctagatatgaaacaggaaaaaactgatgtgtttatctgaagcaggcttgtaatactatggccccaagtccacagaaatcaactcggcctcatgtcaccttcccagtgaaccttcaggactgagctttcagttttcttaaacaaactcacatgcacatgtacatatgtgtgcacacatgctcacacacacaccccacataggcagaataatgttaatagagctttgcttacagatccccagaagagaaaaatcaatttctatcttatttcttacttttaaaaagctgacctacatggctttaacattcccttcagcttggctaaactttagacaggtttgttcctaactataggtccctgacctcttttttttttcatagaacatttgctttagaaaacatgcattgtaagtcctttctctgcccgtttgagaaataaatctgatgtgggagccatccctttgaaatgtgatcataaagatagtacccctatctcccagtctctgtggaaagttcagaacctaacttcaatcagtgacaatcagcaaacacagatggcctaatcacactgaccagcctctgccttactatcctgcagtgctttctcactagctcacctcagggattacaagctctcctgccttttgtttcagtgcagttgagttccatctctctccttcattgcaatagtcctgaataagtcgaataagtcttccttgccatttttcacaagtatccagtgcaatttctctttggcaaagcccatacatgtattttccagtaaaaagataatgtgaacccaagtacctgacagtttctataatttccatcctatttcttagaaatcaccgaattaaatgaaaaaaatgtggaaacaattaaagttctttgcaaatctatcaggtactttatctagattctgcttgtcttgcaaactctgactacttctgtaagtacataagaaagacttaaatcacacactagttccctagagtgatgggtataattttatggagtatatagaagctagaagggacctagatgttcatttaatccagaattcaagagccaaagtcatctcagagatgttttgtttgctcattacaatgttagaagatgaaaagaagaggattgaaatgcttctggtgggacacgccgtccggtttatcccagaaaatgccctattttcttatacttggctgctccccagaccatatttaccgtctttccttatccatgaaaacctttggggttgttatgcttaaaccctcattttactttttaaatgaagttatatgctcacggaagtaaaataattttgctcccagtcttgataagtagaaactgaattcagtattccagaagtgccagtgcaatacttctcaaccttgattggatatcagaatcacctgaagagccttttaaaaatatctacacctgagacttacccacagcattctaagtacaccggtcttggatggggccttcgaattggccatttccggtgtgcagctaagacgaaaacccattgacacaatatattaccagactgtcttggagattctgtttcagaatgtagactgggagaaagttgctctcaataaataaatttttcattattttgataaatttttcattactataggtactaaagagattaaaacattcaatgattttattaagaattctatgccaataagttcagttagataaaattgacaaattcctccaaaaaacaatatcaaaacatcacacaaaattgtgaatatgaataatcctctcattaataaagaaatgaaatttttcattaaagcctttatgtgtgtgtgcacagaccataaacaccctacatgctataaaactatacaatcttggaaaaataataaaatccaatattattgatggttttaaaaaatgatctccactaacaatagaagggaagtacctaaacgtgataaaaagtatccttaaaaaatattgaaagctttttctctgttactgggtgcaaaaaaaccaaaatgtttacctacatgcatcactgaggttcaataagcactggagattctattcggttggtacaaaactaattgcagtttttgcctttttattttatggcaaaaatcacaattacttttgcatcaacctagtaagtaacccagtaaggcaaaataaaactgcatcaatagctatatataaaacaaaagttttaaagactttttttttttttttgagacagagtctcgctctgtcgcccaggctggagtgcagtggcacaatctccgctcactgcaagctccgtctcccaggttcacgccattctcctgcctcagcctcctgagcagctgggactacaggcgcccaccaccacgcctggctaattttttgtatttttagtagagacggggtttcaccgtgttagccaggatggtccggatctcctgacctcgtgatccgcccacctcggcctcccaaagggctgggattacagacgtgagccatcgcgcccagcccaaaagctataaagattttcaaagaaaatattgaaatgaattgtagttgtatataccagcaagaaacaaaatgaaattttagaaccactttcaaaagtattaaaaatactaaatgtaaaggaataaacctaaaaaaaatgcaaggcctttatgtaggaacctatgaaatgttgctaagataaatgaaagaatatttgaatagagatatttatcaagcctatgcattaaaaactcaatatgtaatatgtcaattgtaatacgtcagttctctacaaactgagctatagggtcaaggaattctaaccacaatcccaattagagagtgaatttgacaagtggattatacaatttatatgaatattcaaagggccaagaatattcatgacacctttgaaaaggacaagatgtcaagattttatctaggttatgacatcttataataaaactacagtaatgaagatattgcaatattagcataaggatagagaatatgcccatggaggaagataagagtgtgaaaatatatgatgcatatataaacatttaattttttaataggtggcattgcacacgcatgggataatagtggtctttattaatagttctgggacatttttggtatgcatcaggaaaaaataatgaagctaaactcctatatcacactattcataaaaactccaattagattatggaaatatatgtgaaaagcaaatcaaaatgttttcttagaaaataatgccagagaatattctcatgaatttaggataagggcagtttatttataattgaaatgcacagagcaatcatcatgagcacattaaaattaagaaatcatgcttatcaaataaatagcatcaagaacgtgaaaaggcaagccatacaaagataaaagatgtttgctaaatatatgtccagcaaaagattagtgggtgccagaggtcagaaaaccttttctgtaaaaggccagatagtaaataattcagactttgcaagccatacagcttctgtctcaactactaaactctgcccttgtagcatggaagtaagtgtggctgagttttaataacactgcattcaccaaaatatggcaaatcagatttggcccatagtccagagtttacccattattggtgtatagaatatataaaaggttgcttcaaaacaattaaaaaacaattaaaagactggcaacccaaaataaagaagaaagacaggaaggaaggctcttaaacagtgttaatcaccagataaatgttaactaaaatcataggaaatattattttataccctccccataactaaaattagaatgaaaataccaagtgttgccaaggatttagagtaagcaagcagaactgaggagtaacgtactgatgttgagagtgtaaattagtaccactactttagaaagttcattggcattattgactaagatacataaacctcacaacccttcaactccacaccaagatataccacaaagaaatgaatgcatgcatgttcccaggacatgggttcacagcatccttgttcataggagcccaaagcaaaagcagcccaaatgtccatcagtcgtgtagataaacaaacactggcatatcatttaatggaatgcttcatcgtaattaacacattacagctattttgcaacaatgtacactgatctcacaaacatgatattggataaaagaagccagtcaaaaataatatttgctttatgtttccatttaaatacatttcaaaactaaatcaaatttcaatcgtgtttgggaaggcacacttagtaaaggtaggaagaaaacgtatgaagacaagcaaggaaatgattagcatggaagtcaagataatggttaactttgaagggaagggaggaatttggattggaaagggtgcacgggggttctggatactgtcaactctatacttcctcatctgcatgatacatacatgagtgtcagctttataagtcatttatctgtgaccttacattttacacagtttcctgtatacactaaaaagacagtttagaataaagttcatgtagtataatttctaatcgtgtaaatatataaagatagaatggtatttatggagcattaaaatgtctattaaaaagaagtgccaaaatatattaacagggagattattcatcaataataggatttgatgtaattaaaaataacttatatttgcttatgtttttatgttttctactctgatcttggattgcttagattatataaaacaataataatgagaaaaatatttccccaagtgaaatgcacatatctaccttctgcttgaataaaagctaggtggtattgtaattaacagtatttaaatataatttcacccaaaggagtcttaagttctgatcttgtttgcttagaaatcaaaatgtgaggaaatgtgggtaaccgtacttatccttcctctaccttctctaggtctttcttttctctacaacaagcataaacatgcatatgtgggttgaacaaaataaactgggttttcccagagtgtggcccactatatgtgaatattttaagtcctgtgcatatcacacacacccacgcgtgcgtgcacgcacgcacacgcacacacagagtatggtgcttcaaagagttaatgcacacatatttcgtgtagaatttgcagacgtgtgatctgaagtgcaccctcatgaatccaatgccctcctgaggaataatttgaacagaggaagagttcctctatacagtaaggtacttggacttcctatgaattcaaatacactgaggtcgaagtgactttgtaaaaagtatctgaggcagctcttagtgcctctgggaaaactcagtctaataatacgtgttgaatatttttacatctttatacagtttataatatctttgttttccccagcctttattattacttagtatacttctctattcaatacttaccaaaccctgaggctacccaataatagtgtaatgttcttcacttcagtcaatgaactttaacagaaaccaatgcattttaactgaatttttgtttatctcaaggagcacattttaatgaaatcaatacattcttgctgaaagccacatgaatccattggaaagaatacattgttaccgaaaccaatatacacttttataatgtctgtatgttttagcgtaagcatagccatacattttagctgtaaccaatatatttctgcaggactgataccgagaaagccttatttcatagtgaagtcaattcagtcttacagaaactaatactgtattccatattcctgagccagcagtgtcaggctaaatgaagactatgatttatattgctgaaaaatgtaaatatttccagaaatgactttaaaataaaagtggttttgaaagtatataaagtattttaatatcttgattagagttacaaaagcagatggagaaaagtttttgaatggaaaagttaagagccttgtgtacaatgtgtgtttcagttaaatatgcataaatatatacactaaaatatatttattattatatttaggtgttagttgctgaattttcaaaaactcctgtttatttcattgaatgcccagaaaaaggatatccttcaagaaaaatcagatgttaaaatatttagagatttcctgtcctagaaataccacgtcttcatccaacctaaaaatgacacaaactcttaatagtcttcctctaattcccctcatccttcaaaaatgtgaataataatagaattattactagtcaaaaactaatagaggaacaccgaaggaaaaacattcaagcgatgactcaacaataaaatggtcaaagaaaaatgtttaaaacaagtcatacagatttgtgtatatgtgtgcatgtacgtatgcatgtttggtgaatttgtacttaccactgcaaaagtctcttggcctgaattatgtaataccattttctacactgagattggcctgctatactttgtacttagcccagacagaaattctaaaagcattacctgttagaaaactaacattttaaaattcaaatgtgcttgtatttagaagtgactggttatatcccaagacattatctaattgggagagtaaccttgttgtataatttatccagagttggaaaagcgagggcagtctgcttccctagacatctttatttgcctttttagtttgctctgctaatgacatttccagaggatgaagaaaatgaagaaaaaagtcatacaagagaaagtataataaacccaaaaatatttttaaaagcatctctcaggaaagcatgaatcctagttaaaattaaaacatttctaatatatttacttgacattcatataagcaatataaaaacacacaagccatctgcttgacgtttttgattaggaaaagtagataaaacaccaaaatttaaaagtaacaacattattcttgctaaataaataaattagtaaaggaaaggaaaaaatattacacagagcagatactaaagcaaaaaaaataggacaggaaacttttttctcttacaaaagcaattatttaaatcactcaaagttccttggcttcaaggcttctgtgtaaacttgcatcctctagctgtgtctctgtcactgcaaagatcccaactcctaagtagctcttacttttccccggggtttatttttcttaaaggggtacaattcaaatgcagcattctgctttaaagatcagccttgacaataaatacttataactttctaaattgggtgtgtggactccagaaatgaaggcaggcagtgagtctcaaaagcacttctaatacaagaaacatcacaaaaataagtgctatattctcttacttcccttttacagatccctagtttttgttcaactgaacaatattctggctgggcatgatggctcacgcctgtaatcctagcactttgggaggctaaggcgagtggatcacctgaggtcaggagttcgagaccagcctgaccaacatggtgaagccctgtctatactaaatacaaaaaattagcttggcatggtggcacatgattgtaatcccaactacttgggaggctgaggcaggagaattgcttgaacccagaaggtggaggttgcagtgagctgagactgcaccattgcactccagcctgggcaacaagagtgaaactctatgtcaaaaaaaaattaatattctgaagtttaatcattttagcctttgattcatgcactttgctgtcaagcttgtctgctacttggatgctggcctcactgaagccatttctagcatcatttcatcgggcctcgtaactgttaaactgggcaactctgccctctccatgtgccccgagctattccactcaagcttggtctcttaccttctagctttcaacttctgcatctgctgtttattcacccacattgaagaaactcccatcattcttaatcattgagcaccacaacattccaatcatcaaagaatggcttgaaatagaacctccttcacaaacccttcccaaggcatgttacctttttggggggaataagctgtaggcacttattatatcctgtcttgcactcacacattgctcttcacatccatcaaagtctcctttcttttcatttcacacccactctgtaactggcctcctctgtcctcacttcactgtgtcaagcagtcaagcagtccctaagcagtagcaacaaactgctatagctggtaggttggtgcccacactttcataagttcaagcagaaaggatgtaatgattccaaatgaatttcaacagtttactgctcacagcacagaaggcctgggtttcttgttcacatgagttcccctaactagctttgcaagtaccacaggggtgaggtcagtgctgcacacacatggatctgtgtcacagctgaggactccaaaataaggagacctggatcttatctatgggtcactggccaacctgctagtcctcccctccagggagaaacaattacctttattatctagaatgctctagtgatgggaaggagacattattacgctggaatgattccaggaagaaaaagatctgaatctctctggacggaaacattgtctacaacttcccaaggttgtccaatatgcaaataaccttgagcaaacatcctttgctcagaagacacgtgcaaatgccagaccatggcgaataatctccctgcatactcccatacccagcccagatctcacggcttatcatgtcaacacttttcttgccaatagctgcagctccctctacaccctgccctgccacacacaccttgccaacactgattatctagcagttttcctagtcactttttcatggatttcctgaggcaaggttaggagaaaaaccttcccatagcaaagattgatggcatctcaatttagttatctccaatttcaggtagaccctcaaaacttctccacaagccttttctggtctttgatcaggttgttttcccctccccacattggctatttcaaaccctcatatctcctacaggccttcacaactccctctaaatcgatgtgcctccaaccaaacaaaattatctacaccatagacccaacctaaatgctcaatccctatgttatcaggcagacttgcttcttgttttgccctaggtttatgctttaatctcctacgtcctattgtgtccggaattggtgggttcttggtctcactgacttcaagaattaagctgtggaccctcacggtgagtgttacagttcttaaaggcggcgtgtcctgagtttgttccttctcatgtttggatgtgttccgagcttcttccttctggtgggtgcatggtctcactggcctcaggagtgaagctgcagacctttgaggtgagtgttacagttcataaaggcagcgtggacccaaagagtgagcagcagcaagatttattgcaaacagtgaaagaacaaaccttccacagtgtgaaatgtgacccggttgccactgctgccttgggcagcctgcttttacttccttatctggccccacccacatcctgctgattggcccattttacagagagctgattggtctgttttgcagagagctgattggtctgttttacagagagctgattggtccgttttgacagggtgctgactggtgcatttacaatccctgagctagacaccaaagttctctaagtccccactagattagctagacacagagcagattggtgcatttacaaaccttgagctagacacagggtgctgattggtgtgtttacaatcctttagctagacataaaggttgtccaagtccccaccagattagccagatacagagtgctgattggtgtgtttacaaaccttgagctagacacagagtgctgattggtgtatttacaatcccttagctataggtaaaggttctccaagtccccaccctattagccagatacagagtgctgattggtgcattcacaaacattaagctagacacagagggctgattggtgcatttacaaaccttgaggtagacacagagtgctgattggtgtatatacaatcccttagctagacataaagtttctccaagtccccactagactcaggatcccagctggcttcacctagtggatcctgcaccgggctgcaggtgcaggcagagctgcctgccagtcccgtgccgtgcgccctcactcctcagcccttgggcagtctatgggacaaggcgccgccgagtgtgctccacggcactcgtcggggaggcttgggccgtgcggtagcccacggggaggggagggggtgggggaggcttgggcatggcgagctgcaggtcctgagccctgccccacggggaggcagctgaggcctggtgagaatttgagcacagtgccagcactgctgggggacccagcgcaccctccacagctgctggcccaggtgctaagcccctcactgccccaggctggcagctccggctggcagctccaagtgcagggcctgctgagcccatgcccacagtgcccacaagcctgtgcagccccagttcccacccgcacccctccctccacacctccccacaagcagaggaagccagctctggcctcggccagcccagagaggggctcccacagtgcagcggtgggctgaagggctcttcaagcgcggccagagtggtcgccgaggctgaggaggcaccgagagtgagcgagggctgccagcacgccgtcacctctcactatggctgtgtctccacccaaatctcatcttgaagtatagctcccatcatccccacatgtggtgggagggacccaatgggaggtaattgaatcatgggggcaggtttttcttatgctgttctcatgatactaaataagtcccataagatctggtggttttatgaagggcagctcccctgcacgtgctgttttgcctgccgtcatgtaagacgtggctttgcttctcctttgccttccaccatgactgtgaggtctccccagccatgtggaattatgaattcactaaaactcttttctttataaattacccagtctcaggtatgtctttattagcagcatgagaacagactaatacaatgtcattcctttccaagacttgctccatcaaattttcctctccatcctgcctctccatcttctttatttcatattttccctgaaattcaagctggctaaatttctttttatcttaaaaataatcagtcttaattatgtctcctgtgctctttattgatcatcccaccatctatctttctttcatagctaaattccttggcaaaaataatataaactaacacttacctctctgcccgctgctttcacaccataccaacctgctttcctacagaaaaatggccatccacttagtggctgcaatggacaaaatgtttttgtccccccaccaatttcatatgttaaaaccttaatcctaagtgatgattttaggacatggggcatttgggaggtaattcgtttatgaaaactgaaccctcatgaatgggattagtgcacttataagaggcctggccagatccagtagctcacgcctgtagtcccagcactttgggaggctgaggcaggtggatcacttgaggtcaggagatcgaggccaacctggccaacatggggaaatcccgtctctactaaaaatacaaaaattagttgggcgtagtggtgagctcctgtaatcctagctactcaggaggctgaggaagaagaatgacttgaacccaggaggcagagcttgcagtgagctaagattgcaccactgcacttcaacctgggcaacagagtgagattccatctcaaaaagaagaggccagaggactagctagtgctctttttactgtgtgaggatacagggaggagccagcattctgcaaactggaagaaagctcccaccagaacccaactatactggcatcctaatctcaggcttccagcttcaagaaccatgagaaataaatgtctgtttctaagcaacccagtccgtagtaatttgttatagaattctgagctaagacaatggccaaatcaaacatacttttcagtccatttcctacatttcccttctgtggtatgacatgctgttgatctccacgtctttgaaatcacttctccaccactacactataagctccttaagagagaaaccatgtcttcttcatctttatgtactcagtcccccacagagtcaacgcacataaaaatggctaaataaatgattttttttgcaccgattatctcccttcgtattggcgctttctcttttactccattagtttaccatcctttaccttccctttaaaagctggagttcacccatgacctctcctcttccttgctctgaagccggaagactgactccctaactaaattgcttattatgccacaaaactttttctcttttgcaggcatccatggatttctgatcattactggacttaccaatctatatgtaacataacccctgacattcaacatgtttaaaattcaactcattattcctacccaccttaaaacctcctcaattcttagtgttcccaaacgtagtagtaccaccctatgctcagtcattcgagccagaattatgagacacatgcgccatatatgtactttcagggggtctcttttgatttaatctagccagcaaccctccagccagtcccatttctactgcatgtgactgtcttagttgaatcccttgtaatttctcacctaaaatattgcaactgctcttcctgttcttacctgttctcccctgttctccacacggctactaaagtgattatttggcaagtcaaacctaactgggtcaacactcagctttaacttctccccattgcttagagaataaagtgtaagctccttagcaaggcatacccagtcctattcatggcctcagtctgcctattcttctagcattgttccctgttcattctacaaagtcgttctgtgcaaaggctcattaaagcatgtgcttgttctctcatgcaaggaacttttgctctcctattcgcttcatttgcaagtctccctacccttgactgtacagtgaactcctactcatacgccacctcaaaatgagcatcatcctctcctctgtgaatggaccacaaggcatttagaagctcccttcaccctgatggatattatggtgtagatatccctacatcacagcaatcattacattgttttatagctattaatatttcttcacatccttcatgatctatgatctttgaaggaaagtgtatttgtcttttatatctttgtatgagaagtagtaccatacagtgtaacatcttttgagccaaacacatcagacattggctgaatgttctgcaaatatatctgaacgtttgtattagagatttaatttctctagtcctttgttttttaacttaaaatggaggtaaaacaaaatggagaatgcttgatacagtaccaggcacataaaggctctcaataaatgatagatgctgtcattagtggcatcaccattaccattattatcattattattattactaatgatttccactgttcttctcaaagcctggtactcctcaggtaatcattaagtgttttctgaataaataatttataattattaaatatatatatatgattaccttaaatggttatcttgttgacattgactcagttatttgctattgttcctccaataacttctaaagtgatcttttcaagtagatatttattaaatatttattgactgaatttctcagtgatcttttgcttctgtggacaaagaaagaataattatttcatttcttttttttgggtatatatatatatatatatatatgctttatgtactaccgtcatttacaattttatagaaaataatgaatgtatagagtttatatttggattcaactaaaatttgcttgctatttaaaagactgtcaatagaaattaaagttagggcttttgaaaataagcctgatttaatatacttaagcctcatgaccagtttttaaaactcttgagaaacaacttggaaatatcatatattatgcacagcctaagattaaagcacccatgtttctctctcgaactcttctggaatgtaaagcattcaagatttattgataaaaaagtaagtggggccaggtgtggtggctcacatctgtaatcccagtactttgggagactgaggaggctggatcacttgaggtcaggtgttcgagaccaactggccaacatggtgaaaccccatctccactaaaaatacaaaaacttagctgggtgtggtggtgcacaactgtaatcccagttacttgggagattgaggcaggaaaatcacttaatcccgggaggcagaggttgcactgagctgagattgcatcactgcactccaacctaggtaacacagtgagacaccatctcaaaaaaaaaaaaaaaaaaaaaaaaaaaaaaaggaagtggcatgtttgtagatttagttaattaatgttaattaagcaaaagattgtaagaaaccctagtgtggtaacatagctttgctttttgtagatatcctgatgtgactataatgctgtctaggacgctggagaaacatcctccaagtcaacatcctcctgtggtcctgttggcaccctccagtgatgctgggtagccagtgttgtgttggcacttggtatggctgctctgggtaatgatgatgatgacttcatatatgagtgtcaattgcctcatactccatattgtcagacattttgcaaaatgttctttgttaaacacattacaaatgcctattttgaccacagagtttctgagtaaagccaatcaaaaaactaccaaaagactgctaacttcaacaaaactattacaaacttcaatggctggaaagacattgagaccaacagtttcaatcccaccctttccaagggaaacttttaaagccttcatatccttctaactcacgagacatcatcccagtaatggacaagagttagaaacacaaacttgactgactgcctttcaccagaaagaatattcaaaaattagaggaatatgactatcaaaaacatttacaaagtatatagctattacttgaaagctatatctatatattttttttccttattttactatacataagaaaaagaccctttatctctgtggctactttctgacatatttctcaccgaactataacctaaaagactcctgaaaaggagatttttgcatgtggcagtaacaccagtcctgtgacagggaaagagcaacccaggcttctattcccttgtcagtcatcagaccactgtccatttccataccacatggcccatactctcatcttttcaccaaaatgagcagcattcataatttctaaatttcaaagatgtattttgtgaacttttactggagttgagtgctttagaaataaggcaaaagagtgctatcgtcagggaattcatctcaccctattgataaagctcagatcccacctgtagtgggggttcttgagtttaaaccatggtgaaaatgtgtacactgctgacctctggtggagatttttattttactcttttttggttaacttgtctttgtgaaacaagggaattttacaccaattaccttcataatagcaaacacaaaaaatatttttgaagtcaatttaaatactgtgtgggacctgtcagatgcacagatgaaattgggacagataacctatcaatttgctcgttgcttacctgctagtcactcattaacttaaaagtacttttagtgtctcttaactctgctacaaaaaagattggaagattggatattgatgtatgtccagtgtggcccatttaaaacatctgccataaccatttaaaagctgttaattcatgtccatttacctatccacacgtcaattacagctagtgaaatgcatttgtcagtttttttcccccttcccttcatctctgaaacattctaacacaaaactgtgcttgctggaaaaatctggtgattactaaattaatgttttatttttgtcttagcatgtcttttggaaaaaacacagtcctgattgtcatattcttcttatgcttaagtgcaacaattggaactcaagaatagccccatgctttgcaggatatgaaaaatagtttgaagtccaaagttccaccctccatatctaatgatgatcagtgactccgttacagaaagttttccctcagtaaacccggaggaagtgggtacttcttatattacaaagatacaatggcaatctgtctgcatgtgatttttttaaaaaacatgggacctctggattctaatttctaaagtttgagtaactttggacaagtcactatcagtcactaaatgtctgcaaatcttagcataagagacacaaagttcttggaagtaaagagaggtattgtgtagacagggactatctctctgatatatggccagagacaaaaaatgatgaaaagcccagccctgggaaggtctgagggaaaaaccaagcagaaagaacaatacagaacaagctcatttcttggaagatcagcaggttagtgggccccagaatagagggatggaaatgatcagatgggacactgaagaagtaagcagctcagaccacacagttcttatagaccatacaaagaattggaatttgattctaagtttaaaatgggagccattggaaggttggaagcaggagaatcatgtgatttgatttcagcttttaaattttcactctggaggcagggaagctggtcaggagactattgcagtaggacaatattgcagaacatgaaagtggcttggtcttagctgccgtggcataatgaataaccaagtatgaggtgcacagttaaaaacaaatttgaaatgtgttttgaaatgaaatgtgttttgaattaagttgcatgctatgacaaatattatcatttaattctcattttttagattacagaagcttagcatttttttttttttttagacggagtctcgctctgtcacccaagctggagtgcagtggtgtgatctcgggtcactgcaacctctgtcttccaggttcaagtgattctcctgcctcagcctctcaagtagctgagactacaggtgcgagcccccacacgcggctaatttttgtatttttattagagacggggtttcatcatgttggccaggctggtcttgaactcctgacctcaggtgatctgcttgcctcagtctcccaaagtgttgggattacaggcgtgagccactgcacccagcctgaagcttagatattaagtgatttgtccaatgtcacacaaccatttggttgcagatttagaatacaaacctagttgccgtggcttcatatctgttatacctgttactctagaataacacttaatagcaccaacttaacattattatgagaccatgggccacttttaagagttacagaatttttgagttggtaggaacttcaaagattatctccttccacactctcgctttacagataaggaaaccaagggaagttttgtatgtttgttttttgttttgtttttgttttttccaagttaactgactttctaaagcttgctcaacaagtaacggacagggatggcaacaagttcaagtatcttgattcttagagggatgttatttccacaacagcatagcaagtccattatttggtgctttctatgacaggaaataaagacatattttgaaagtatggagagaattttgcctacttcacagttatatccaaatttatcttgttcttcaaaataatggcatttgcggcaacctgaatggaattggagactatcattctaagtgaagtaactcagtaatggaaatccaaacatatgttctcactcataagcagatgctaatccataaagacgcaaagacataaaaatgatacaatgggctttggggactcgggggaaagggtgagatgggggtaagggataaaagcctacaaactgggttacagtgtatcctgctcgggcgatgactgcaccaaaatctcacaaattaccactgaagaacttactcatgtaaccaaacaccacctgttccccaaaaacctatggaaataaataataaaataaaataaaataaaataaaatagagaagttgatttggctcacagaaaaaaatggatattttacatggtttctatgtatataattagacatttgttccttaaaaaataaagatatttaagacactaaaacaaataaataaatttatctggttctttattaaatatctattgaacatctattttcgggcactataatagattataaagatttcaaaattaatgttatagccctctaggagattgtaatcaagtgggaaagacaaaaataatgcaaaaatatatttcaatataacatggtaaaagccaaaacacagaaataaagatagtcatgaaaacacagagaatggagcgataactaatcgtatctagaagagtgaggaaagatccaaaagtgaaattttttccaaaccttggccaataagctaggtgatgattggctttgattttaccccacgagtaatggatgctaggcagacaagaatgagattttccctcttcttgatattttcaagatataaaaaagggagaaagtatttcccaaaaattaaggaagtagaagaactgagcttgtccgtgaaggctctcctctctgatcatattggaagaaggtcataacaacattctcaggttatgtgatggtagctcattctaaatctttatcaaaaaatgcattacatttgtggttcgcatctccaaagctgtgtcatttcacagccgctacatgctttcatgctatgcatggaaaagaaactcaagggtatccatggagcagtgctactattactgtttggtttcttttctccatctacctcctctcccaggataagctcaccctcctctcattactccaaccactatctacatggctaaatctcaaattcatgtcttcaggctagacctaccagagcaagaaggacaatttccatgtggatggtgttgagggatgtctctttttcttgattgcaatttacaaagcagatatccagagctggttcagaagaaaaacctagcggctgagtcagaatgttcaaaattaagagattagatcaaaataacaaaaaaaaagtactaaatgtaaaataaagagcactgatgacctctgctttgtggatcccattataccacatacagtcttctaatataacaccagccatattttactgggttttctatttcacatatgtctctttcttgttagaaaattagctcattaagaggaaggaatatctcttgctcattttacatttctggtgcttagaccagggcctggtacagaagcactcaacaacaacaaaaaaactacatgaatgagtaaataaattaaaaaccatcctgaggtatttggggccttcatggtatctattacatcttagcagaataatagaatctctgaagcatagcaagagagggttgttaaagccctccaggtgtaaccccactcatcaaacaaccctcttcttatctgtgtcatctgaagacttagggtgatagaagactcactgtcttctaaggccatttactaatctttgaaaggcttaaatgagaaagttgtttgttttaacaagctgacaactctggcttcctcccatacattctagctgcatccttagggctttcagtgagtaaatacaatccctcagaagatgctttgctatcgatttgagagccttgtctaggccatatttgatttcagactgaacttgttacaagctttaagtcttttcattcacaaatcctaataaaagattattaaaaaaccaattgaaattgctcagcacagcaatatcgaagtgatgggttctttcttgaggtcttaccttcttttaatcaagaagaaagaaaagaactatagactgttgacagtaaccatgtgttatctactttccgcacacttcttttcttcatctaatttaattcttctaatcaaatcatggtattatcattttccctttacagggaagctaataggcattgtttaaaagccaactgttcctggttcaaagatataatgacctacatttgatgttatgttgactcaactgaaggactgtcataaattaatgtatgaattttaaaactaaatacattctttaaattgcagagaaattacctccctctaggtaggtaccatagattttggaattaaaggaatcatggctttatttcctggctttacttcttcatagctaggtaacctaaagcaggcctcgtaacttctctgggattcagtgttctaatttgtgaaattatagtagtaacatagactatgtaaggctaaagtgaggataaaatggaatcacatatgtcaaggtggcagcactatgcccagcacatgacagtggtgatgatgatgaagagaacattgagattcaattaaattgaaaatattgctgctgggaggacctttaacagttccctaatcaaactcaaactgacatcagatccagagggagcaagggttttattcaagatcatagagcaagttagcttcctgccaccattcctaggtctactctcctctctgcagtccagacaaagccctccaggtctagcatttcctcgacaggataggacagaaatgagcagcccaggcatccttggtgttcagctttcctctagcccgtgtgaggggtgcaggagagagagagagcacagtctagttctctgcacaggctctttctctggggtttcttcccttcacatttaattgctaaggacagtcacataaaaaataaataaatcttcatttctgtggggtccatctgctcccaccactcacctgcacatgaaagatgcctgctcgtgaatgcatctcaatttcaaatgaacagtcaaaaaaaatgctcccgtcaaggctgcctcacagtgggtacaaacctctgcaacagcgctcctcctgaggcagcaagttcatcctgtccattagtgaggaggataagaagggcctgtcagggctgcctccccaggacacagggccctcgggcagacagatgcgctgagtgtgaaaagaaattttcaaaagccttcctcgctcccagcctggggcagagcaaacagggctgggaccatcaaagccagctgcttgcctacaggctgacaccccagctactggggtttaatttcacattcttactctctcccgctccagttacttttggtgattatttttatcgtggtatatttgtaaaattatttttcttggatcttccctctaacacccatgtgctgtggtgatacaggatcaaaaagataggttcctatctaatcaggaaagagtaaggaattattaaaattgtcaggggagggctagggacactgaattctatttcttagatgtttccacttttcatgatccctctgtccaatttttccaatctaaagagcagagtgatgggtgcaaatcatttctctatcctcccctctttcttccctcagaagtggaggacaataataagaggtggagagggtgatgtaattttagattcaaaagagggtggtttttaagtcttcatagccagtactacacagattgctgccatataatggaattcttccaagtattttgggaggaatcagccttctgttcacacgtcaacacatatttgatattatcaaattattttataactttcaatgtgcttccatgtttatctcattttaaatcttacaatgacagcatcagatgggtgtaattattcgttacttgttaacaacagataagccagagtattcagggaaaaggaaggagaacagtttgcatcgctccattaagccttgggacattgtcagaataattgtcaaataattcttttcaactagattctctgtcaaactgcctgatacctgagcccaaggatgggagtgagggaggggagtgcagagaagggaggaggaacaagaacctgcttgtggcccccagctcacaaaaatgaactcaactccagaggaagctaaatgactaaatgtgatcctccttagagcaattatcactcagaccaaggagagaccagaaatggggaaccggggcttccagagagttgatgtccccaagatcatggcataaacacttatgtcgccatcaagatccgatctcatcagcaaccctagcaggcctccctgtgagccaaatctcctcagcctttctaggaatcagttttctcatctataatatgagatgcttagacctcactgtctttcaaatcctttttgacaccaatgttaagtggttctaggacaaatcacagcatctactcagaatgtattattttagagatttaaatggacacataagccgtaaagggagttcttcatgttgcttttcactagtcacatttttgactagcactggggttaatatctcagttttaaccttttttgcactccaagcagaaaatgccttagattggaggaatgggtattatgagggggactgtcactttccaccatcagcagcagatctggggaaggagtcacgcattatttgcagaataatggacctttctgattcacacaccctctagtcagctggtctgctcaccagggaaagcagatcaatgcagatttgggaaatcaaggaaaaacaagatggtgacactggaaagaaaatgaataaatgacaagtaaaaagagaaaagaaaggatattccaatattggcaccatgcttctccgcatatgtaatgccttcagtgcaccctccaagaaaggtatggatatctctcctttacacataaaagaactaaagttcagagagtttatgtcacctgcctaaggccattcagtgagcaaatgggtgaagtagaacttaaaaccaatctgtctgactaaatccaaaacatcacactgtgttcctgtcagtcatagtcagctttgtaaatttgtgttgtgttggataaaatatatgttaatggttatgctttgttttacactgtaattcattaaaatactttgcaattgaaataagctaaagaccttgaagtttcaaagtctactaagtaacatcattaacatggaggtaggtgataaaaatctttcccaagttccattataaaatagacttccctcctaatcacaggtagcaaaaatgatcttaaatcatgttatactttatatttgggagagttttattattttgtattgaatatttatggggttttttaaattttcacttggggaggtgggcagttcccagcatccatttctctttatttttcttattacacctatgccatttccaatctttagtgttacggtggacatgtgacccatggcccacctatcaggacagaggattcacccatctatatagataggtttagggattggtgtttgattcagtcacgaacaattggctgcatacggctttttctgagactattggaaaagaagcatgttctcaaatctgtagactttaacctgtaaatacatgagcccagaacggttaggggccaccagataatatctgagaataaagctactcaatgtaaggcagagcgaaaaaatggagaagcacccaactctgaaaaaatcaatagcaacgactgaaactatcaattttcctttttgccagtgtctgttttctgttattagtgagtaaaagagaactcactggtccaatttcattgtatagtccagattaataaagatttttagcaaagcagatttagtgtgaagataaatgaatgttttagcacaaaacatcacaattaaaacctcatggattttacttcatcacataatttcgtctctttggtaagcatcacacaagaaagaaaatgtcaggaacaacattattttcattattttgccactgataagtcttaagtaataccaaggtaggagactggcaggacttgttttctggtcacaaccctgctgaccaaaacaggatctggtccacccaggatgatgtgaagaaactggcaggaaccagcagatcgtgacaaaagccatccctagctgccctcacagctcattagtgtaagatactctcaccagcaccataagagtttacaaatgccatgacaatgccccgaaagttaccacccctttccatggcaatggcccagaagttactgactgctttccctgaaagttctacataacccactcctcaatttgcattaacccactccttaatttgcatgtaattataagagggtataaatgagtataaacacagttgccaagagcccatacattgccaactctgggtgcactgactgtgagttagccctgccctgcaagaagcagtaccattccaaagaagattaggcttgcccttgaattctttcctgggtgaaaataagaacccttccagggtaatccccaattttggggctcacctgtcttacaccagtaccacaaagtgccaaccttttttaccaatcagattaaatttttcctcaattccctctttgtgagaatgcctttcttcactaggaaatggggaccttctctacatttcttcaaatccaaagcatcagtttggctggtatccgtccaagaagggaaaatgaaaagcgtgttcagtgcctgaggatcctgtgctcattgaagcacatgctgctagcagttttttcaggtgttagagtgactgaagacctcactataatttctggactgaaaatggtttctctagatgctagagagaaagagtggcctttcttcctcagtatgatgctcaaatattgtcactccatgcccagataggcagagcttcaaatgccaaagcattctggaagctcagtgtccttcacttaggcacacacagccattccttgaggcatgcccaagctgccagggtgactgggctagggggaatctgaccatgagccacagattgtgtccagagctaagccttccctatgaccatggaaagccagagagtgcaagagagggggagttattaaggtaaagaggtcagttccagttaccatcattgatcaaagacacagctgggattttccagcgacctgaggctgtgagtgtgtgggtgggggaaggctcagaggtgctgaacaatcaaaagaccctccttcccagtagtccctgatagactaccacacattctcgtactagtctaacatatatatttttctaaagtaataggttttaggactaattcaagtttttcttataacatccagagcaggatctattttctgtgtagagtttgctttctatttcattctactttggttctccccagcattaacctctgcatcttgtactactttgttcttgtcaccaatcacacttccctgttttagaacagcattgacattttgtgtgtgcacggtgcataaaagagtacctaacccactttctgccctgtggtatctccagaaataatacatctgccaaccactctaagatattaatagcactcaaaaatgtttccctgtatgcaactccagccttgctatttattgtcagataggtattcaaaatttcacaggccacacagtctttcctcactgtcagggaaggactggcccgccatgaattgctccgattctgaatgagaagcagtgagacccaaagcagcaagccccaggaacaggtgaccttgatcagaataatagaaaatccacatgctatagaatagggcagggaaagcaactcctccacaccaaggttctattgggccccgagacaaatgcctctgctgtgacataaacagttgtaaaatatcatgcagaacttctagttccacattttgggagcacactgagaaagaaggacatgacagccgggtagtacccaccagtcggaggcaatcagacaccccggagcgtcaggaggaactgtagggcactgaaaacgggaagaaaatgaaaacttgggagggctgggcacacacacgagaaagcgaggaaggaaaatgggtttcacaaggaaacctaggaggagaactggctatcaccctggaccccatttctccagctgctgcacggaagtgacagaatggcaagagtgacctgggtgacatgcagggatgggtgcaaaagccaccctcggttcagatgaatggatctcgggtcccacatgctgtcaggactctgtgactcagtgacgagtgccctcaggatgatgcctccccaagcagctggcactgaggggtgactgtggtgacatgaaaagggggagggaggggggcatgtttaagtcacatcaaaagttgagcctggggctgggcgcgtggctcacgcctgtaatcccagaatttcgggaggcggaggtgggcggatcacttgaggtcaggagttcaaaaccagtctggccaacatggtgagacccccgcccctccatctccactaaaaaaaaaaaaaaaatagctgtgtgtgtgtgtgtggtggtgcaggcctgtaatcttagctactcgggaggctaagtggggcaggagactcgcttgaacccaggaggcggaggttgcagtgagccgagatcgtgccattgcactccagcctgggtgactctgtcaaaaaaaaaaaaaaaaaaaaaaaaaagttgggcctggaagagctgttgagtgaatgtgttgggagaatgagaaggaaatgaggtgtggcaggcctggctgacgccaggtggtgggacacgccagtgcctcccgtgctgcctgagggagaggctctggcccctgggagaagcgctaggcccggctgggatgtctggggctgtccctgaaaggagatagtaactgtttggtttgtttgtttgttttgtttttagtgtggggcgttttttgtttgtttttagtgaaactgtcaattctgagagaagccgtgagggaagcggaatctgctcctccagatggaagcggagccggagggaccgcgcggtgggaagccagggtgggcaccgcgggcagaggcgggggtgagctgaaccaccgcaaaagcgggaggagacccgggcacctcgtctgccggccggtgcaccgggggtctgcctttgacttgaagcctaaaagggggtgaatttgggactccaagagcggagatattttgtccagtcatgcagggcggtcagcttaggcttgaaattggaagactggggcaggagggagagtggccccaaactccttactcataagggatgagagttaaaaatggacacggcgtcaggatccccgtggcttaatgctcatatcctagtgagccgtctgtaatgacatttttgacgtggattatggcagtgttacaaccctgctcagaggccgcttactgagtacacctgggcttaacgggctcttccggagacattaattcagacgtgagaattagactcacgtgttcatcgcttccccgcttttctgctaacatcaaatgcagactcatgtattcagaccctgccttctacagcactcagacttagaaagtaaacgcatacttctgcagatatataacaatttaaaatatcttattttacattttttatattaaaattcatttggatgcaaacattaaaattaacaaatagagacagaagtgacatggaacagcttaaaatacaaagatataaacacttttcatcaagataaacattgccttagtgaaactggcccgattgtccgtcagaactaatgtttaccgggtttttttttgaataaacataaaaattaaacgtcccattcttaaaacttgagaaagctgtatctccatctgtcttacctaagtttctttctcgggaaaacaaacatcaggcctcccagatagtatcaaggagctgaaactcaccagatctctgaacaatgagaggccagacccttcacccaccgtgactctgaaacagaccacctgtttcctgttgagcagcccctcttccttatcccttcccaatttcttttcctacacataggtacatttcttccctactatataaaaccctgattttcgttggtcaggtggatggatttaagactgatcttccatcctctaggtcgcagcacccaaagcctttttccctaacaatactcattgtctcagtgattggcttttggtgcagaaagcaatgagacctctaccgacgggatctgtactgagcccctagcgtctgggtaatatataataagtaacttccaaggaattaaaatgtcattgcttagtacctgattttggagggttgggggtcagtggcaagggagatttctagtgaggataccagagtaaaccatgttggtttgattggcagtaacatacgtttaaaatgagagctgagtgagctacaagcttcagcagcatgtttaacgcattctttatttacatgttgtaaatgaaaagttactgcataacactttttaaaaagaaaaaaaaaacttttattcttgaaatgcccaattttagttatggatcatactaagtacaagcaaaggctattttagctgttcttaaaaccaaatgcaaaatatactggaataatttaaatcctgttataaacaaaagagataagatataatgacaactctaaaatagcaggaaaaattaactcattttaaaattagttgttagcaggtataatgaaagaagataaatttaaacaattaggaactgatgaagactgtaaaaattgctatttagaaagaataagtgagaaaatgcttgggaaacatggatatatatcaagcaatagatctgaatgacaaataccctaggctattaaggaaattagtcaagcaacttaattaacttacaatgattttttcaaagctcatggtagtttggatactggcaacatggtacccaccttcagtaaaaggttaggagaaattaactcaaatattaccagtggtaattttcccactaatcatagtcatcattagtaaaattataaggttagaaactcagcacaagataaaaagattatggcaccagagccgtaataaccctgagcatgttaaaatgaaataaagcactcagtgtggtgattcctcagggatctagaactagaaataccatttgacccagccgtcccattactgggtatatacccaaaggactataaatcatgctgctataaagacacatgcacacgtatgtttactgcagcactattcacaatagcaaagacttggaaccaacccaaatgtccaacaatgatagactggattaagaaaatgtgacacatatacaccatggaatactatacagccataaaaaatgatgagttcatgtcctttgtagggacgtggatgaaattggaaatcatcattctcagtaaactatcgcaaggacaaaaaaccaaacaccgcatattctcactcataggtgggaattgaacaatgagaacacatggacacaggaaggggaacatcacactctggggactgttgtggggtggggggaggggggagggatagcattaggttatatacctaatgctaaatgacgagttaatgggtgcagcacaccagcatgacacatgtatacatatgtaactaacctgcacattgtgcacatgtaccctaaaacttaaagtataataataataaaatttaaaaaaaagcacacataacaaaacaatattcaatattccctcaacatcattaagatagagggaaggaaaataaaatttacactggctagtgaatatgtattttttataattttagaatgtaagcactcaaacatttacttaccaaatcactcttaatttgtttagaagttcagctatgtaaattgaaatgtgtccttggcaagggaagactaatcattaccttcatgtaaaagaagtctctagtgaagggtcacaatagttgaccagagagctaatttccattagtgtataccattcactaatgatatcaagactgaacattgtgacatttggttatacctcaatagtgagagcaattttaggatttaaagtttcattgcactgggaatttgtggttaacattggtatgagtacaaaaagggtgtatctaaaacctcacagccagggaaaaacagctttaaataggaaaattggaaacaagtatttgaaaggaaataaaatctttaatgaagataaatgggtagataaaagcaagtaatcagtggaaggatattcctgagcaatgctaatgaattattcactagaatggagcccacgggctgctgggagctgtcctctccccactccacggttcccggcaaaggcaatgactataccagaactgtcttcatgcacactgggccaggccttgaggggactctgactgtggcggatttgggataactaccttaatattgttgaaaacgacagcagctagtattaacattcttgggcagaatataaaatagacacagacgccatggaggaataaagacagagccaccttgtataatagtaaatcacattcttgtgaggtttttgtcagattcaagactaagagtagctttcttctcacttccaaacaattaaaggaaaaagaccatttctctatcaaaaaaaaaaaatctaaatgtatcgatacctctaatatcagacaaacattttagtttcattgagtttctgttttgtttttcagttgtaaaatacatatggtttcctaaaagatctacttatcaagatgagtgtaaaggttaaattacctgagctatcccatggtaaagttgttcacagactttgaagttatatgcaccagttatcatccccatcattctcattagatcatgtttgtacaactctcccccaaagtattaacatatacatggaccatttaagaaacatttacctcccaatagtgtactactgtagtgagtcaggaatacggtgataaataaaacagttctctgccctgagaatttatactcttgggtagaaggtgatattttagtgcaatgtgacaatgattctgatttttttaaaatatggttgtacatgggaaatacacttattccattcctgggtcaaagaagtcttcctggaggaggtgatatctaggacaagaactaaaggatgtgtaaaacttaggaaagtaagagaaggttttgatgggcgaggacaactaggcagaataaatgacatgttcaaacacctggaagagacatttataccagatttttggaactcagcacagttcagtttaggtaaaattttaaaatgtcatcctgtgtttacagagagatgaaactggagggcttgatagaaaccacgttttcaatgactttatacaacctgcaacagaatttggacttttgctggtggtcaacagcagctgttaaggattttaagcagaccatactatgatcatattagcattattaaggcttgtattgaggatttagaataagattattgattactatagtaatcaatattgaccactgcagcaatccagatgaaatatactagtaaaatgatctaagggagaaggagtctgcaaactttcactgcccatgaagttttactataacacagacacatccatttctgtattatctgtggctgttttagtgagacaatgacagagccaaggagtcacagcagagactggcctgcaaagccttaaacatttactatatggcactgaccagaaacattgccaccccttgatctaaaaaaaaaaattatagagaaggagaaagtatttcagaaatatcaagcagttaaaatgaatcatttgtggctggttaaatgtggagtaggaagaaagataagtctaagatgtctactatggattgaatgaacaggtgattaataagctagatctgatatgccttctttaaaaatgctcaactaatatgaaataatcatgctacttgtgatcgcagcttagtggaatacaagtaacaacatactgcaatgtattaaacctacaaaattgtcagtctatatggttcaacacgtagatgaaataggccacattttatctggaatcaaaattgaaatgaagattatcttccaagtaaagttaaacttgccccttcaaatctgatttttttttcttgaatgacttccatacttgtttctctgtgccattaactatgcatgttggacaaactcaagtctcacttttctcatttgcaaaataactgcttttattatatctaatatctaaaagttcaaacattaataaatccagatagtatgattttcaattaatttacaacaaagtaaaaatatagttgaggtggctgtaaatttgagtaataaaacgtcaagagacagaaccctcttgttagggaattattactgggcttaaaaggaaggttccagactagatgcttgggaggcatgtatccagtcaagtccaaaactggactagatacacactatgtttagttagttataacttttaaaagtcaacaaggtaaaaatctccttaatggccaactttctgtaatttctaatacttctgatgccgtgatgattcacctctattgagaaactcagtgcaattactgatttgtttgtctttctttaatatatagaaccaatcagtctggttggcctgtcagttgactaatgagtatattggaaagattgagatgggtcaactgaaatatatgcctaagtcagctgctttgatttttggtgtctagagatacagctttctgaacgttccttttgtcctgcctttttcttctcatatcctggtataatgagggggaaagagaggatcttttgtggtatcacacatttggttgattataagaacactatacgtcatataaaagaagaaatcgggagaacgaaagttgtatatatagtatgtagtatcagtgttgcattgtctattacttcagtatttaggcatttgtatttaagatcgaaagtataaatattgtgagtcacgatcaattttgctttaaatcatttgggcaggtccagggcccaggcacattggcagctaaggcacctgttaagggagatgataaattgagtccttaagtccttggcagaggagctgacttagagataagagataagaaaattagtttgcaatagaatctctaatgattagttgggatggaaatcagaaggtcttctcacagtctgattacattgttttttattatcttggcaacttatttggaacagcagcatattttgcaactttctcccctcagtcccttcagtaacatattttggttgagtgaacagtaaataacaagaagaactagggacaattaactattattgtagccctatgactaatgaaaatccgtacattgcactttggggaagaatgtacttattaacatgcctgtggggggtaggttaatgttgctattttaactttgcttgaaagaaactagagtaacttacccaaggttatccagctgtcagaattacaaaagcagagttcttcccagcaaaggccccaagccaccttgtgaagcaagtcaggaatacctgcagcaccctctggccatgccccagattctaaacacagaatccatctctgcacaatgaagatccttagttagctaactgttgtacatagttactaggaaagctgaatactcccaaagcttttcacattttctttctttctttctttctttctttctttccttttctttcttgctttcttttttccttccttccttccttcctttctttctttctttatttatttatttcttctttctttctttctttctttctttctttctttctttctttctttcttacttttttaggaattttgaacccaataaaaccttcaggactggaaggagatagttttaatggtgaggtgacaattgatgaggttgatacgggagttcatgggccatcaaatcacactgtcacttatctttattaattcaactaaaatgtactgaccacttactgggtagatcagtgttgctgtggcataaatcttaacagcgtagtgtagtattaaacatggggtagatactgaaggcttttcttatataacgtgaaagagagtggtttatcctatagctactggttagctactggaaagttgtgaacagatgtattgttcaattagatttctgtgattggttctggcaataatctggaggatggagtcgaggagaagtgggagcaagaatgaagaagtaacagcctttttccaggagagaaatgatgaggacttgaattagagcactggccctaggtacagagagatgaagacagattatgagatgtatcagtacacacagtacaagtaggcaaggaagacagaagtgactggatgcaggaggcaagagtaaggagaagtctaagatgactgctgggcttctggcttggagaactgactggatgatggtgctgttcagacagactggaatcacaggaggtaaatcagatgaaggctgtatggggtgcagcagatgcaggaatggaaaatgtctttattttgaaagaaagttaagtgtgaaatgcctgcagacatctgaaggacaatttccagtaggcagttttctatacagctccagacctgaagatgaaatctgtaccagcacaaagattgtgacagtcatgatgtagttggcactcatgcatctgggccgcccttgatcttcacccagctggtgtttgatcataaatggaggttagaatccttagaactaaaaaaaaatgagagacattcattttctctttgactttgtttgtttttaatatttttcttaaagccctgaagtattctgatgatcttagcaagaaccaggaaaatatgcacttcaatatcaaggtgtgtctaataaacgttggaacgctttcataaaaccagaaaaataggaagtgtaattcatagatgttttccctgtattccctttgtttccaatggtttctctgagtatttttaatatttgccctctgcctcataagagtcgttaaaatttatctgcaaaacagtaaacaaaagtaaagaattaaaatggtcataatgatgatgagactttatatgtgtaaatcactaaacacaatgtctttagctcataattattcaataaatattttttctacccactttaatagatctacatgtttacaaaatcttctgcagaataaaactagatgagtaatattaatcaaattactagttgtaaaaaatcaattctaagcattccaattcactttgcattgatttgctcttgaaaacacttttaagggtttccttctggttcttttgtttccattcaaagcaccaagagacttgcatgcaataactgaggctttttatttttaattacagaagatttgtttgttcaggaatgtgtcttctgaaataaacctaaagttgatcattgttacattgtgaaaaaataaaattaaaaaccaagaatgttagctgaattctgatagatccattccaagttaacctgaaagtacttctttaaaataaaaaagttgtcatttgtctagaatatatgagaaggaaagctcactcattcaattcatacaggctaatggcgacaccaagtggtatcatattggaaatgtttttagtctgtttttgaacttctgaaagaaattctaacctgggacaattgtgattgtgaccagcatggtttgtaatgtagctctaaatagggtggagttttaaaattattattataactagtatttctctaataaaatttttgtctccagccaaaataagagtcaaaaaaattccatatgcacagtcatgattagtagataaatgagcaaaagtcattttacttatcctttttggaaagaaggtaggaggctgatggtgtgcaatgtgattcagcactctgcagaggctgctgctatctctaatgatcagggtttccatagggaagttcaatccagtgagttcaaatctttctttgctccttattaggtgtttgatttggggtccattatctcaaatctctcagtaataagtttttttccatatataaaatggagggtgatattaacacattggggctttgtgaagattcaactagttcatgtacaggaagtattaatacagtgcctagcattgtagaacatactgaaaaattgtgcttctttttgttaacttcatcatcatcatcaagtcaatcagattgacctatggatctctatacacaaagtttgtctctcttccttgatggaatacaataaaatttatacctgattcctaccctcataggaaacgcttgttttagaatcagtatttttgtacctgagtatttttaaagcagttttaaaattacactagaattcatatagaaagaaatcatccttggcctaatcctgactctattcctggaggtctatttgtaaccaagtcaagagaagagcaggggaacaggaagcttctgctcaaatgtcctcacgtcaccgaaaaatggagtgtgatataactgtatttactttgtaaaatgatgagcctaatatcaatttaaagaataggttcaggaagggacagtctagtcttcagactaaagcagtagagctagttaggccttgggaagagggaaaatgtgtgtggagctgtgaacagacaggttttttattggctatgggaacaccagagaggtggaaatcaaggatgatgctgctatttctcttttcataccttggcagaagaagatctacaaatctagaaggaagtgcaaaaacaattctctgttactacttctaataatgcaaatcttagggcttagagcaaggtttagaacatatcatggaaaatgagggatttgaaacttcagcagctcttcaacagtggtctcacagttcccatttgtcttccatacccataattcaggacttccaccaacaagatgtatgctcctggatgaaatctgaaaatattaactgctgtcatgtttaaggacctgtgcttagtgttgagcaagccatataaactctctgagtttcgattccataaaataagggaggcatttagcatattcctaaactgtctccctattcaaatattgaatctttgtgactttcccataaaccgctttttaatcactgacttcccttagaataaaaaagtttatctcttcctcccatgttgatttttctctctacctaaacaaaaacaaaaataaacaaataaaaaacaaacaaaaaacaaaaatgaaaaaccttactgcaatctaccctgcattgaagaattatatcataggtaggtcctgggttttatggaataaacataattttaaacctgttgtctcccaaaacacacttgtgttgtgccctaatttgggatttgggaaaaacggtctcagtagagatagctagaaagctttctttctgcacctgctgaaaaccatgtgaaccattggataagcaaagcctttcccagatgaaggacttgactgagcacttcagtcaccagtgtgcttctcagaaagggatactgggtcagctctcccctcagccctcaggcccagactcccagccctctttctgggaaggaattgtcaggtcatggacactttccaggggggaaaagtgtctagggtctgtctagtataaataagctttctaacatacagtagcagcaaagcaacttgagccactgagccctgtggcaggcgtttcctccaagccttggttctctaattgaatgtcactctttttattaggaggagaaggagattgcctagagacattgcttaacacttaagtaggctcgcctaaacccctttctctgacacaagcagcagccctggagaactgttagcaaagggcttcattctaatgagagaaacttcaaaggccccaaatgaggaaagaagtgtcgctggaaaatgttgaaatgacaaatagcggtatttccactgacaaaagatgaagatgaaacaaatgttagaacagaaaacatgattttattttgccatgctctcactggaaactatcatcaaagcccttaactgaaatgaaaaaggtatcgacataacaagcacaccagagtgaagttagatttctaacaacgaaagtcatctttgaaataagaaaagcttacaaagtaatgaaaaatctcaatgcagaaaggaaagcatatggcccaaaatagtgttgtgctgcggatttgttaggaacgtcaagctggaagccacttagggatcatctaggcaaacccttttatgtttcaaaggagaatttgagcccaaaggattttaagtaacttgctcaaggtcatataactagttagtgacttcggaattgttagcaactgccttccctacctccgtggagccagttcctgaagtctatctacaaatccacccttgcccggttactgagacagccctggtcagggctgaagttgaagttcaccaaatgctggaccctggtatttgtccacccccacaactgggcaggccgatgctctctgaagttaggtctgtctgacacatgacaacagccacagggacaaaaactttttgcccattatgcttctagaatatggtatgttctggaagaataatgggcaaaaagaatggagggctggcgaaggcatggtgcatcctaaaacctggagcctacagaattaaacattatagataggagaaaagataaaaaggcccaaaaggtaaaatttattattttaaaaaaatgtgcttaataggagagggcctgaggctgactcaagaggcgagatattcagattgctagcctttcagaaactggctaaaggatttgaatctgggtagctcaatttgccttccagatatacaggtctcacattttttcaagaataagtaggaatttttaaaaacggcatggagcctgaagagatgaacaatcttcgatcaaaggaaaagaagcagcatgcaaaatactcagaaaaaagataatctgaaaatgatgtcccgtagaatccaaagataatgtgtggaaactgtttggaatatgcaacaaaaacaagatatattatttatgaaacaggatgatcacagtgaaaggagtctggcatgaaaagactgagagagttggcatgaggaaggattgctgataggatgctcagcccacactctctcttctgaggtcttcagcgttcccagccattcccagggccctttcatccatctagaattctctcccaagcttcatacccagctatgaggctacctttgtgaaatttaaatgtatacattgcaggaacctcaattgtaacaggttcaaaatggaacccttggtccccatctcacccaaagccttcagcaagaaaggcacctcttttcagtcctttgcttatcccaaatccttgtataatttatctgccctcactattcccatacctatcctggtacattctgtccaacatgagaacctatgattcctccttgaaatatgtctctaacaatttccttttctgcatacactactccactactttcaattaatatccctcatctcttgccaagaactattaaaatgatattctcactgggcttcttgtttctatttttgcctccttttattttattctccatatagtagtccaaattacctttagacactgcaaatttaagtgtttctctcctgaactcaaaactctttaaaaggtgccaatagcatgaattatgaatataagtctttttcctggcctaacttaacaactttgatgttccaaatctaccggcctcattagtttcttttgctaccattccccttctgtttcataactctgcttccacatagacactgtttctgttctccttcaataagcatcatctttcaccatcaggattttgcacatgctcttccttctttccggaacacctttccccattaacatagccttacccaaaatttagatcttaaatggtactttctcaaagagggtttccctcactgccaatctaaatatgtctcccaggttaccttttcatggcattctttgcttcctttcttaacacttagttaggacagttaataattatattgttttgcatctcttttgcttattattataacctacactaccacaataggaaatggcacaaggagacaatccataaatagcaatctgttggcatgtgaaattagataaagaacataagcttgcctcctttacctctaaatatcctattgtaatgactgtaagaatggaaaaggaaacataccaaagaaaattggagagataggagggtattccatggtagctggaaggtttggagataggagggtattccatggtagctggaaggttttgggttgatttccgaaagtaataatgtatatggctattgatggataaatcagggtggaggaacttgctcagtacacataaagaagaatgccaggcactggggggaatcagaattctcagagaggctcatgagccagtggacactgactggagcggggggaagaaagagacagaaccacaatggatccatggctcccatctttctgttggcagagtaatcggcaacccagaattactccctaaagaaaaaaaaaattgtgaacttctttggaaagtaattcttaaaagctgccctgagagaaaaggagatttgaatgtggctgatagagcccaatagtagaactcttctaattctgaaattcaagggagtgaaggcagaggaaggggagtggaagtagctagaaggggccacccctccgggcttctttgcctgcgcaacatgcaggtcgacatatttgtccataaaaagcaggctgcccaattagtcacactttctgagacacgtgtcccatgtattcagcagtagagattacgtaaaccagctacctacctcaataatcaaaattatcattactagatatgaatggacaatcaaaatttacaaggagctgtggagaacatcattagtttgcaatgcaagaaccaagattgacactcagaagagctgactccgaaagaaacatttcagacaggaatattaaaaattttgataggatttttagctaactcaagaagtcattacacacacacgcacacacacacacacacacatacatacacagatattaaaaaggaataaccaaaaggcaagatagtgttcgtgaagtttagaacattgttgaaaaaaataaaccagaagagctacgagaaaaagttcagaaaatatctcaggatttaagaatggaaaacagaaaagttgagatgtaaaagatcaattcagagggcccagcatttgatgtgtatactttctagacagtaagatgggaaggaaataaataattaaataaattgtaccatatttcataaatgctaaaatgacctcatctctaaaatgcattattatttcacatattactaaggaaataaaagctctggcaattgaattatgatgccacattttcatccacttgaattgttattttaaaatatgtaaagagttcttttagatatgttcagacatatattttattatatattagtcttgatcatatataaaattaaatatttgtaaaataatttcaaaacatattcagtctgactcttttaaatagttacgggtcttaaggtcatatatgctcatttttcaccccaatattgcttattgtaccatcaggaactttaggacatgggcattatgaaacacttctaactgtcaccaaacataatactattaactcagattttcttccaagcttctgaaaccatttctgcaagttttgatgttggcattatttcatatggtatgtatagttaatacaatagcccagcttccacttgtaaggagggtggctgcctttcttagtccctataaagcatgggcattgctttgcaagaaaacagtaaatttcactcattcatccaaatataagtatttgcttccctaatatcaactattgcatcaccattccatttcagtaattttcagcattcacaataaattttcgacttcaaataacaatacattgtttcaaaattaatggcagctctactcaacaaatacagcaccaacatgaacttgcggtgacaacaatataggcaccaatgatgaaatacacacttgtatggacactgataattacatcaagatcttaatttcagaagcatgaagatatacaaaaatagatcttaatatgggccttaagcacagtggaatattcagctttttattaaatttctattttttatgttcatctgtccaacattgaaagaggcatattaaagtttgaattttatcttcactatgaatcatacccttttctataccaaatgacagcatttctgcttttttatttatttcattgaagtctacttttttatattaatagcttgacacctgatgtctttttgtttaaatttacttgatacatctttgcctatcatttcacatttatatttcttgtcactttatttgagataagctaactgaaaaccacatttggcaaaacactgctgttcatttccaatctaaaagatttttgtcttttagtagggaaattacacttagtttgattataaaatttacataatttagattcatctcccttagctgattttagttcttgtcacatcattttataccatgatttcttcattcttttgttaaatttctttttattgtgaaatatatcatgctcataaaacaatgtgtgtgtgtatatatacatatatatatatatatgttaatagtaagttgaataaacatctaatacccactacccagcttaagaaacagaagatcataaatgactttgaagttcttattgatctaatggatcacattcattccttcccccagatttaatactatcttgtattttgtgttaataattattttttatgcctttgccatatatatgcaccttaaaaaaacataatgatcagtttacctattcctggattttatacaaagggaattgacacctgctatttttgctctgcatgatgttttgtgttggtagctgttattaattaattttccataatgtgtggtattccatccaaagctgtacaacctcccaacagtattagtaacattgtgttctatatgaatagtgctgaccatgggaacagcgcaccagaactgagcaagatgatgactgtgattctactgtatgagtgcaatatcatttgttcctttgaataattcattatttattatttctatttatttttcttgctatgagtatgattttgcatgtcttctgaacatatatgcaagagcttctttagggtttctaactaggagtagaatatcttagctttactaagtaatgcgagatcattttcctaagtggttttaccaatgcctgttcccaccagccatgtgcaaggactgttattgatgtttgccaagatttggtatcaacagacttttaaaatttagagtccgttctctaatttctattttagtttgttttctcttattaatgagattgaacatcttttcatatgtttattggccttttattattttatcttttgtgaagtagctgttcagatctttgtctatttttcaatgtggttgtttgcctttttctcacagattctttcatggaaatacttcatatttaagataattatctttttatggttaaatgtgctaaactatcatctcttagtttctgtttgtatttttataatttttatgatatatttttaaagtaatttaaatacatgtattttaatgttgattttttcaattgtttcctcttattgttttgtcacaaatatatgacaaaaatccttcacttcactaaggttataaattattttgccatactttctggtagaactttaattttttcaacagttaacttattagcccatttatacctagtgtcccattatcggaatgctaagcttgtgggcattattgatatccaactgctcaaggtcatcatcaaggtctgattcttcacaaaaaaacttttgcagcttccggcataaatgggttgtcagcaattctttggttttcatcttttacataagaaaaatcagttttcccagcacactacactgggcgatcatttccctgcggatctctaatgccagttctttatcatctcacatttccacatatgaagagaactatgcttctgagctcagctctctattctgtgccattgatctgtttgtctatccctgtgtacacaccatgctacatcactagagttttataataagttttaatgtctgctaaagcaaatcttctcaacttgatctttttctttagaagtgtcttggttattttggccctttgcttatttatgtaaatttttaaatacatttttcaagttccacagaacacattgctaggatttggactagaattacattgcatcaataagacaagtgagggagaactgacaacttttcaactcaaggtctttctatgaatgacatgaaatagctccatttattttggtcatctttaatgacattctatagcatttgataattttcaacataacaatcttgcttattttttgttaattatcattcctaagaatcttaaggtttttgagctagtgcaaatttaatgacattctctaactattgctatttagatgcataaaaatgcaaattgctcttgtataataatgatagttactaactgtatgctgcgttctactgtaccaggtcctgagctaacctctttatatactgattaaatcctctcgacaactctatgagctaggtcctgttattataaccacgtctcagattaggagactgaagcactgagagggttcgttaattcacccttctacgagctctagcaagtgcaagactggagtttgaaccccagaatctgaccagtgtttaggctctagtgtatggcctatcgtgatttgcatctgctatgctcaaattgtttttgaattatatttgaataacttaaattttaaataattatatttgaataatagagaatttaataattctctattatttgtgatgcagtcctataatcatataatctaggaatacggttttgttttgttattcaaattattatgcttttgtatattgttctggccaaaacctagggttcactactgaataagagtaatgggccaggcatggtggctcacatctgcaatcctagcactttgggaggccaaggcagacagatcacctgagctcaggagttcgagaccagcctggccaacatagtaaaaccctgtctctagtaaaaattaaaaaaaaaaaaattagcccgcgtggtggcaggggcctgtaatcccagctactcaagaggttgaggcaggagaatcacttgaacctgggaggtggaagttgcagtgagccgagatcatgccattgcactccagcctgagtgacagagcgagactctgtgtcaaaaaaataaaaaggtactggaaatcccctctctcctttgtttattaagaatttttctcctgaatacgtgttaaagcttaatgaatgttttttccacacctcttgaagtaattgagcaattctttcattttgtttaacctgtcaacttggtaaagcataaaaattcatttttaaatgttgcaccaaccttacattttatgatacaaaatacttttcatataatagtcttataataactttgggtttgatttgtcataattaattaattaattaaaaattaagtaacaaaatattaagaacatacctattatccaacagtttagactggcttttgatgttctgttcttgtacagttcatatctgattttggtattgatatgggttgggtctgtgttcccgcccaaatctcatgtcaacttgtaatccccagtgttggaggtggggcctggtgggaggtgattggatcacaaggacagatttccttcttggtgctgttctcatgagagtgagtgacttaccatgagatctgattgtataaaagggcatagcacctctccctttctctcttcctcctgctccaaccatgtaagacatgcctgcttcctcttcacctttcgccatgattttaagtttctggaagcctccccagaagcagaatcctgtacaacctgtagaaccatgagccaattaagcctcttttctttataaattgccagtctcaggtatttataataccagtgagagaacggactaatacaggtatcaaagttatattagctttataaaattagttgaagagtacgcttttatttttaaaataatggcaagatattctttttgtaaatgtagatcatctctttcttttcgtttgctgaaatttacttgtaaaatcatctgaaactggtgttttctctatgggagaacgttaaatttttgatttaattttttaaatgtttataggctcattcaggttgtttttttccttacaaatttggttttgtaaattatacatcttcttgaagacttttctgtttctcttaggttttcaaatatattgacataaagttgtttatggtattattttattatattgtaatctctgctgcatctataattatgtctcctttttcatttttactcttttttatctgtgccatcttgtatttttttcattgcagttaatctttctggaggttattttactttttcaaagaatcaagttttcattttttagatcctctctattatattgtttgtctgctgtttcataagtatcttcttttatttttagtattttctttcctctgagtcatttgggtttattctacttacacatttctaattaagttggatgcataggtttttttcagccttgttttttctaatacagacatcaaaatctacaaattcccatgaagttccattttagctgtaccccaaaagtttttattttactttattttgtcattgaattagaacttatacaaagtactctagtcataagtatatacctagtgattttcaaaataatagctaataattttaatccatgtaactatcaccagatcaagacatagagtatttccagactcctagtcaattaccccacgctacttccatcgtctctgactgccataatacaataccacagactgggtggcttaaataagagaaatttggtttgcattcttgaggctggcagtttgcaattagggtgccagcatgtctgggttctagagaggaccctcttcctgggttgtggacagctgccatctctgtgtcctcatgtggcatttcctcggtgtgttagtctctcttcttttaagggcattaatcccacatgagggttcccattctcaagacctcatataactcaaattacctcccaaaggccccatctctgaaaaacatcaaattgggggttagagcttcaacatatgaatttgaaacaaaaacattcaatccataacacttggccacagaatatacctatgtactaaggcagccttaactttccctcagcttgactatctttaaataggtttcttcttgcctctaggtgcctgatctccctctcatcccagcactcacggaatccagatgacataaccacatggccattttatcagagcactgactttagaaaacttgtcattgtcgatgatttctctgttccttggacatgtaaatctttttaaaagcctcttgacaatttttcaatgcaggactctttcctaaggacctgggagctgtttcaaaaatcatcaaggaagatagcatcttatttccctgtttctgtgggagggtgggagaataatatcagcgggcaccttgcttgaagttgtaaaattacctcctgccatgaagatatgagaaaatttatttttcctttaggtaaggccaagtagaaaacccacatgtcctaatacctctccccaccccagttctcaaaaactctccagccctttgttttagtgaagttgagcacagactgagatctgatctctctcccctatagtaatagcattgaataaacacttccttacaggctatcttagtctgttcaggctgctaaacaaaagaccataaatttggtggcttataaacaacataaatttatttcgtacaattttagaggctgggaagtccaaaatcaaagtaccattatatttgatgtctggtaagggcttattctctgctttatagatggcaccttctcactgtgtactcacatggcagaggggcaaggtagttctctggggccccttttatgaggacatcaatcccatttatgagggctttttcctcaggacctaatcacctcccaaaggtcccacctcttaatactgatacattggatgttgggtttcaacatataaattttggtggaagacggcattcaggttataggacatgcttaactttgcttagtgcaacgtttgcttcaacactattaggcttttgtagatatggccaattttttgagtgtctttactaatttattcccaagaaaaatgtaggagaattccagttgttctacatcgttgcccactcttgatgttgtcagtggttttaattttacctactttggtagagttgtggtggtatttcactgtgcatttcatatgaatgtcttgataactaccataaccaaatccttttatatttccttcttggacatttgaatgtcttcttttgtatttattcaggtgttttgtgggacccaaagaagctcgcaggttgcaattagttatcacacatcctcctgtggcacttccacagtgcttgtcttttatgacattgcctcttttagagagtttcagccaattatttggtagaatatccctcaattcaatgccgtctgatgtttctgtacttacactcaggtgctgataatgttggcaagaatgtcacagaagaagcttgtgcttctcagtgtatcctgttaggaggcacaagatgagagtttgttacattcttggagataatgacactaaacaacagaagcattcctttaaagtttctgtagagaaaaatgttcagtgtggcaccattgctagataaaatgaatgtaaataaataagtgaagtaaaaataaataaaaatcagcaagctttcaaatttccaaatattctgaaatattaaaatatactttttatttaaaataatatacatagctcttaaaagtgatcttatcataatcttttaaatgttaaaatatcacagtttataaataggtatatgccaaaccatgaataaccacagattagtaagtgaattttagaagtagccaatttggaaataattcaaaatatagttattgtaattatttaattgccataaatttctcaagttctctagggcttttttcctcttctctataccgcttttattttatatcaattcaacttactgagaggaattttcaaggctttttttcttttcagcaagcctgttagccatgctaagcaaatactgagtgccaggttttttaacttaatgacaagtggctggaaactctggtgaatgtcttcctctatatttaagaaaatttatctgttgagaaaaacactttaaaattaccactattgtcttccggtctccaaaaaagatataggcaaaacagttttctcaaatactccataaaatttttcctttaaaaagagattattttattttaaacaagaaatcaatacccactaggaaagcaataccatgtcagagtgagaaagagagtgtgtatatcctccagaggccaaatagaatgtaattttaaaatgtgcaatggttaattctgtttatcaatttttctgggccacaggatgcccagacatttggaacatcattatgctaggtatgtctgtgagggtgtttctgggtgagactgacattttgcaatggtacaccaagtaaagctgactgccctccttaatgtgggtggcccccctccagtcaaccaaaggcctgcatacaacaaaatgtctgagtaagaggaaatttcacctgcctgacactggaattggagcatcagtctcctcctgccctctgactggaacttacaccaccagctctccaggttctcaggccttcacattcaggctggaactactccaccagctttcttgaatctccagcttgcttactgcagatcttagcacttctcagtttccgtaattgcatgagccaatttctcataacaaatacacacacacacacacacacacacacacacacacacacatttgtccctcaatatccaatggagtttggttctaggacctcccacagatactaataccccccggtgctcaagtctctgatataaaatgccatggaatttgcacataatctatgcctttacttcaacatcactgcctcagcggcttccccagtccctaccctggagatggtccccacttcactctttcccacctgcacttcctcagactaacccatcaatcactgcattgtcattactcctgtgattggcagctcctctaccaggcaaactctctaaaatcagtcccggtggccccaactcagcatcaggaacagtgctgggtaaaagggggctcgacaacgtggaactgaataaaagaattaggagggacaagaagtgcttgcacttagaagtggatttggagcgctagttttagcaggacctttcctgagaagactgaggccaggtgtcagtaggcaggtgcctgagaagagaatgcgaggagaggagatggaaatgcggtcagacggccaagaacgcaggactcccgggcactggtggctgagcgtgccgggggcttccattcgggcctgcagacacccacagaccttattatcccggccccgccggcttgggggccttctgccttccttctcaaagggcacgaccgcaggcggcggcgaccacagacggcggcgactgcaggcggcggtggggcacgagtcggcaccgagggcggacccgcggtcggggtgagtccctacctcctgggagaggctggggccgcgtgggggacccggggcggggtcggccgacgccccttccccagcacccgccctccccggcttctccaaggcctcaccccgccgcgggaagagcaggtgaccgcacgcgagggccccgcgggggacccagggctggcttgagcgcactggcctcctgctcttgcaaggaggactccaaaacgattctgaaagggaaagtgtcttgaaattaactggaggccgaaacccaggggaagctggtggtgcaaaggactcatccacgctttgggctgttacatagagaggattttggaggaggggcaagagaagttgggaccctgggcttttctgtttcaagcaagcaaagggagggtggcggactcctatttcagagtagggaggagccttgggtctttatttcttacatgtatcagggactcacctgaaaagaaaatgatgtgtgccacaagtaattagtagcattcctgaccccatatacagagccaggcgtcctaactcctgacacctaagctcttttccacccctggcgctggagccacggtgctaagtgtgagtggttctgtccctgggctgcacatgacagaaacacgtgcagactgcgtggctcagacacagagtggctggggacactgctacgtcctctatcggggggttcagaggcagggcgggcttcaggctgactcagcagcccgtgatgtcatcagagactcacattctccctcactcactgccccaacttcatcctcaggttggtcacaaggggcctccgcaactggaagcatttcacgtccaggacaagatgtccagaggaagagaacagaacaaatgacttactgaagtagaaagcgtttcccagacctgtcccccagtagatgcccttcaaagtagccagaatccgtgcattctaacttgagcctggattagggtggggcaagcaggtgtctagggtacaaaagttgagaaagcacgggtcgggcgcggtggctcacgcctgtaaccccagcactttcggaggctgaggcaggcggatcacctgaggtcaggggttcgagaccagcctggacaacgtgattaaaccctgtctctactaaaaatacaaaaattagcgactgtggtgggcgcctataatcgcagctacttgggaggctgaggcatgagaattgcttgaacccaggaggcggaggttgcagtgagcagagattgctccattgcactccagcctgggcaacaagagcagaactctgtctctaagtaaataaataaataaataaataaataaacaaacaaacaaacagaaaagaaaagaaagcagaatccttaaatcgagtgcctccttacagtagtgccttaggtccccactctgattttttaaatgatggtttcattgagatataattcacatactaaaaagcacccttttaatacgtgcaatgcagtgatttttagtatattcacagactcgtgacactatcaccactaattctaggacattataaaaaaattcacttccttagtactcattcttgattccgttaaaaaacagagattgtcagactggttaaaagtaagacccaaacatatactgtctacaagaaattcaccttgcatttacacaaataggttaaaagtaaatgaaggtaaaatgtgcaacatactattctaatcaaaagaaagctggggtgactataataacatctaacaaagtatatctcagagtggagaatattactggagatgaagaaggttatttcatcatgataaaggaggtcaaatcctacatagcaatcctacatttttattcctctgctagtaagagagattcgaaatacatgaagtaaaaccaaatgtaaccatgaagataaaaagtccacaattgttagagagagctgccaggtgttgaggtggtccattaagacactgagccaaatgggagagtgacagtcaggcttttattcatttgtgggggcaggggagagagggagattggagctggagtctggaggaaggggaagagctagtaatggaaaagcaccgagtattgagtcagagtggagaaaagtatctccaggccccctaataaggaggtcttggcagtgtcccaacagagcccctgccccataaggaggcctctgaatggaggggccagagctagaaatgcaggtaggcacagggcatggctggcccagggggtcctaagcctttgatggtgcctccctcccgagatcacaaagcactagctatgaagtctggtgggagaagggcagatttcacccaagagtagaatatctgtaattgcatatggttgggcctgaaagattccatttaggattttgtcctgaagttgtactcttcaacttcacctcttaatttttcaggatccttgaaagaagcaaagactaccctatcatggtcctaaataaatggaatttgcgggattatgacaataacccggtaattagtctgagccagcaatggaccaagttcatcattgaaatgtttccaaataggatgataaggacttcttggaagattaaacatagccatgagcccccaacaatgccaatccaggataagatgtttcaaaggtcagcagagtctgctttagagagccggactcttccactttgtcagattcattgatccaggtgcaccacgaggtgttgacagtggcacagactctccttggctggccagctggaagtccagggctatgcagctgctcaaatactcaaaccaaagagctgaggctgatctgttgtgtctccaaaactaaggtagtgttgttaatgacctcagtgatggtaagagacaggtgcacaaccttttctcactgagggacctgagcagtgggaaaaaccacccagagcatacacatgagcaaagagggtcagcagtgcttctcacccggtaagccctccaagtaaccagggtaggcttggttctggagctcatgccttactcagcctggtgcaacatgctccttgggagaggttgcttggaatatttgaaaaactcttattaatgccccattcgtgcagattacaaggctatcaaaaaggaaggtaaaggcctggtgtttagaaaagaaagaatactctaaaggtatacatcgggttgggagtgtaagagtcattgactaccataaggttcaaccccctagaagtccttcttggtaggcagtaggcagagtccctgagtggaactgaagagtctgtacaaggaggggacaagtggaggagaggagaaggtcaccaccatcaaggccaaaatagcccagtaacagacccaggctcctcttcccagttgctcccatgtggaacagcattgagttgacaactaggaagtcatttcgttcctacagtaccagggtccacatatcaggtaacacattgggtccgggtgtgttagggcacctgagatgccagaaagctccatagttgaagaaatgtccactcatgctattcagtggcatggcagccaaagatttacatatggggtcaaggagtgagtggcatatccaactactagtcaagttcagcatgatggcagctgcctgagtcaggcctacgaatgaattttgtaaggccagagtcacacagactacccatgagagagaggagagaggttagatcccacttcttgccccgtatctgagttcacaggaccaaaggttctcatgtcttattagttcaggagactcctcattgactgtgacatgggcagtctgtcccgtgccatgagcaagaatagtgctcttaattcattcttagaatggtgaacctaaatattctggcttggcttctcctggggttccatcaaagtttctatttgatatttgatatgtatcccatggggctacacggagtaaagcagcagggccaccttagggaaaatgttgagacattgaattgatggttattatctttgaagtccttgtaggcaaaattcaggcactgcaaccagggagtaaatggagaagcaagttcatgcctcccaatctgtgctgtttagtagccttcctgatctcccactgattttggtcttcaggaagggctcaaaaaggatggagggaacttgcatgatccaccatggaaactgatttgccaataattttaaaagaggggtgtgcaactgagggaaagagacagatagcccaatcaatcacacagcacaaatgagaagtgcacttagagaattgccgcagtgagaccccaaattgcacacctgagacatgtagccaggctgggattatattgtccatttccatcagccacttcaacctgttcttttcctgttaaaatatgcgatcattgctgtggcttgtagatgatacagagcatggagaatccatcatgcctcataatttgacccatgtatagtttgggctgcaaatgtatcgtttgggccattattagatacaatgtattcaggaagccagagaaataaacaaatatggttttggagggactgaagagtgttgcctgagtcagggaaacacatggggatgactagtccaaatacagagaactcatcaactacatttaggatccagcagaatgctctggatgggatgaggggactagtgtgatcaacctgcaaagaacatcctggcctttttgaccagaaggatctgtccctgagcagaatgtttagccttgctcagggtctggcgggtggattatctttttcatgctgtgttggcatcttttgaagataatagttcatgcatacagatccaggctaggatggcatcttgattcccagggcccttcctctcatggatcagaggcaatgagttgaatatctgttatctctggggcagatgacaaatcattctccatgtttagctgacatgcacggtctgctcattgccttgattggctcattgcctttgtcagcatagggagctctctggtgagcacccacaaggataacaaagagcttattaggagtagaattgaattttatccgaatgtctcatcccaagagagggatgtgttttacatgtcagcacaattttgccagtagctcaatcaaatgttcagaccattaactacagcccatgagtcagtgaaaatatagcagggctcctttgttgagggagcctgggtagccaaccatactttataaagctttgcacatttatgctgaacttgccaagggatagatctatctcaggttgtatgacagctgcacctcattccagcatagtaaatgttaatcaagagacaccgtcagtgaaccaggcttatgtgtggcccagaaggtccttgtatctgagttcccattggccaagtgaaggcagtggctcattggacgacatgcctgggagggggacaacactcctgagtccttgccattgagttgaaggccccagctgggctgggttttacctgcttctgtgtagaacattttcattcactagagaatactcttaagccacacccacctggatatttgaggcctctgctcactcataacatgattggaatttgggatcataaaactatgtcacagccgtgagtcacgtactcatgcatatctacctaggcccaataacaggacaataattgtttctcaaagtgtatgtgccttttggctgactccctttcagctgactccaggatcttataagttgaaaaacccaacaagtgtctgatgttatttttctactatgagcgatagtaaaggaactgaaatgttgttgagacctgtaagtcgaatggcaaatgtggatccatggggtccaggggaagggagggtacttggatggctgtggtagcctgttgaatgggccccattgaaacatagcagccttttctagtaacctcttagttgggtcccatcaaaatccccacaagggaaatgtgacagtaacagtagatgaataagcccacaaatcactgggcatcctttttatttgctggggcagagacagccaaaagcttggcagtcatctcctctaaacgagatgttgtgccacttgcccagattacaaccaggaaattcacttggggtctctgtctgttaatggtgtagttgggttgctgcgtgaaagtcatcagtcctcaagcacctagtccttatttggcaatgtctttgtttgggttcacaaggaggatgtcatcaatagagtgcaaggctagtgcttcctccaggcaagcaactctccccagagcataacctattcattgatggcagctatgtggggaatttaggtcactttgtgaaaggacattaaaggtggattgcaacccattccacataaaggcaagttgatcctggacttctccgtgtgggaggatgctgaaaaaggcattgatgatatcaactgtcccataccaagttccctccagtagcctctgtaagaatcaaaatgtcagatactgccaatgcaaagaatggcaccacaggcttgagccacctgtaatagacagaggatcaccaggtacctgaggccatgtgcgcaggccagacagagctactgtatggaggaacagtttctcaggaactttgcctcaagcttagcaattgaaaccagcccaattgtcccataaaactaatatttatggtttattttgaaaaagcatataaattaaccctcatagtcttaaaactcaagaaaattacagttatctgagttcctttctcaggaagccaaccatcagtgcttgcagatattatcaagcagctaaaacccaccaggtctacacatctggacaatgagaaacccgacccctcacccatcatgatggcctacgcaaccacctgcttcctgttgaccaattcctcttccttacttctccctaattcctgtttccccacacatggttacatttcttccctgctatataaattcctaattttagctggtcagggagatggatttgagacttatctcccatctcctcagctgtagcacccaattaaagttttcattcgtctttgcaatactcattgtctcactgattggctttctgtgcagtgaacagaaggacctagatggaattcctgatgttttagtgacataatgacagcagttatttttttcctctctaaagcaggtggggatacaatggcctaggcgacagtaattgggtacactcacagcctttcccatgtcccactaagatgaccctaagtgcagcaatttccatcagggattgagggtgaaggccagtaggggcacatatagacaatacatctataccaagaacacatgcagtcatgggagccataacgtaaaggcctctaggggcccaaaggtcccttcccagagacagagcaggtggtggtggtctcagaccctaaaccagagaacattatctgtttccccctcatcctggtgccagggattgtggggatcagaggtagcagtgcacagatgttcagcatggccaagaaggacatttcctcccacctcctcactgaactctgacattggctagggcttctagtcatccctggggacagagggacctcagcctaacccctagtcttgttaatttgtaaaagtctagctgtctggggagaggatcgtgaggctcttcaagtcatctccagagtcactgggagggatgacaacaagcataatgcttgaatcagtcataggggctatcaccatgcaaaccctcgtgagctgcctgctgggccccccatggtctggtatcttggtgaggaactcttcagtctcctcctttgagagcccctttattgaatcatcaggctcagagagcaatttgggaatgtcattgcctagatcgtccagtagagggcgccggatagtcatctttgcctccctgcttctgcaatctgttttcttggaagcagccactctttccatgccttaggcatttgttaaaatagctggattgtgtctgggacggactgagctggccttatgaggtttacctgggcttgataaccagcagtagtggcataaaaattcttgaaatctgagtctggtgaacagtcttatcctctgcttcccccagcccctcattgtagacccagaggacccccagggtgacttgcggggctactatagcctctgcgaagggcatccagaagtgtttttcaatggcatgggtcttgttacagtgtaccaaacctgccagatctgtcggagcccaagtagtacccagctgaggaccaatgatccccctcggaaccgaaaagcttctgtaaacaacacattagcacactctaagggtgccctgcggggtccactggcacctcccagacaccccgtggcatggctggcaagtgttctgcggtaccctcagactcttgcagatgaaccaccagcacctctactgtgccctgaaggacttttaggctttttgtttttccaaaaaggcattgtgcttaattgaccactcagctcctttgtcaaattgagagagagagagagagagaaagagagagggggcatgtatgtgagtactttcaggtgttaaggtgttgaattaggacaactagccccactgaaagtaacagtcaagcttttatttgcttactgtgataagaagaggctacccaggacatggtgccattattttccccatggaacagcaccaggaagggtcagatgacaagcagcagaggtgggagaattgtctcattgcctagaagtcccaaacgcaaggttcctgccattttgtggacccaggagccaaggggagggacaggagggatgggatgggggaaggatggactagaagtggaaaagtactgagtgctgagcttgagtgcaagagagtgtcttcaaggctcctgataagaaagtctcttcagaggctcctgaacagtgtctcagcgaaggcctctgctcccctcataaggagggctctgagtgaagctgtctgagctcggaatgtagagatgcataaaggcatggcctggctcaagggcctgagtccttgactgcagctcccactggacacttcaatgccctggctgtgcaccaaatctgtgagcagggcagcttccccccacctcccccatgcagcctgccttgtagttgcctatggtcaggcctatgggatcacatatgggattttgacctagagctggacctgtcaacaattatagttggacatgtcaacactcatctctcaacaattgctagaacaggcagacacaacattggtaattatatagaagacctgaacaacactatcaccaacttcatggaattcacatttacagagcactacaccccaaaacagcagaatatacattcttttcaagtgatcatggagcatttcctgagatagaccatatttaggggcataaaataaatctcaatatcttaaaagaatacaagtcattaaaaacatgttatctgactatggtgagattcaattagaagtcaataacataaaatctttggaaatcatcaaaactggaaactaaaaattacactttcaaataattcatgagtcaaaaaagaaatcaaaaggaaaatcagaatgtattttgaactgaataaaaatgcaaaggatatcaaaatttattgatgccactaaatcaatacttagggaaattttttatggtggtaaacacctatattaaaaaccaagaaaagtttcaagtcaattacctgtttcaccttaaaaaaactaaaaaaatacccaaaatgtagagagccagcaaagggatcatgaccaactcagcattccactggaggctatatgatcaaacagcaaactgtttatcatgaatgcaggatgtgggtaaactcacactgcacctgccgccaagaggtttgctgagggtcatcactccctggcaccaggctccttgaagttatctactggaaaatctagcgcctattgttcaaaggatgcagtgtcacaagcctgctgtgaaccaaacggccgactgacaattacccgacaatcacccccactttttttgctatctcttttacctaataaatacggaggactgaaaaagtttagggcccttgcctactagaggcaaggtgcccccgaccccttcttccaaatatactcttttgtctttgtcttttattcccgcgttcatcctactttgttcagtgcatcagggatcgtggtcggttacatcaaaaagtgaaaaaattaaatgtgaagtaatcagaaaaaggaaataataaagatcagagcagaaatcaatgttgtggacagaaaaacaatagtgaaaatcaatgaaaccaaaagctggttctgagaaacagcaataaaatttatcaacacctagtcatactgatcagaaaagagaaaaaggcacaagctactaatctcagaaatgagagcgatggtattactacagattttacagctattaaaagaaaaataagagactattataaacaactttacaacagtaaatttgatgactagaaaatgaacaaattccttgaaagacacaagctaccaaagctaactcaagaagaaatatgtaatttgaatagttcactatctattaatgaaaatagagttgcaatataaagccactccagtctcggatggtttcaccaaacattaaaggaagaaataataccaattatacaacatctgttccagaaaattgcaaaggtaggtatgtttctcaactcattctttgagaccagaattacctaaaaccaaagtgagacaaagatattacaaaataggaatagtacagactaatgtctctcatgaatggagatgagcaattctaaaaatattttaacaaattgcatccaatactagacaaaataggaaatacatcatgaccaagtggagtttctcccagtaacgcaagattgtttcaacattcaaaaattaatcccaatattaacaaaaccatatgatcatctaaaagaaacaaaaaagcatttgacagaatccaacatctattcctgatataaacacaacaaactacgaataaaagagaccttcttcacctgttaaagggcacctatgaaaagcctacagctaatatcacgtttaatgatgggagccttcgtgctttcccctaagatcaggaaccagacaaggatgtctgttttcactacttctaatcaagtcagtgcaataaggcaaagaaaaaaaaaaagctatctaaatgtgaaagaaagaggaaattttgtctttattagcagacatggaactagaaacacagctactagaactaagtgattttagcaagtttgcaagatataaggctgagatacaaaaaaaaacctgtattatatttccatatactagcaacaattaatcagaaattgaaattaaaaagcaacaccacttataatagcacaaaatagaatatacttaaggataaatctgacaaaatttgtgccagacttgtatactgaaaactacaaaactatgctgagattaatcaataaaaacctaaaagaatgagaaacatgccttgttcacgggttggaagactcaatattgctagtatgtctattctctccaaattgatatttagaattcacacagtcctaatcaaaattgcagcaggctttgtacaagtcaacaagcaaattataaaattcacagggaaagatagaggactgaaagcagataaaacaactttgaaaaataaagttggaaaacttatgtcacttaagtgttttcaaaacttattattaagctacagtaattcagacaatgtggaattgacatcaagatagacaagcagatcaatggaatccaatagaatccagaaatagaaccacatatccatgatcaactgatttttgacaaagatgcaaaggcaattcagtggaaaatggatagcattttcatcatatgcaaaacatgaactcaaaattgatcataggccaaaaaggaaaattaaatccggtaacacttctggaagaaacataggagatccttgtgactttggattaggcaaagcattccttcaatatgacaacaaaagcattaagcataaacaaagaaacaaaatgataaaatggacttcatcaaaattaagaactgctttttaaaggcactgttatagaaatggtgggaattgaacaatgagaacacatggacacaggaaggggaacatcacacccggggcctgttgtggggtgggggggagggatgagggatagcattaggagatacacctaaagttaaatgacgagttaatgggtgcagcacaccaacatggcacatgtatacatatgtaacaaacctgcacattgtgcacatgtaccctaaaacttaaagtatagtaaaaaaataaagtgtgcaaaataaagattgaaaaaaaaaagaaatgaaaattgaagccatgagctgggagaaaaatgttttcagatcacatatatgataaaagatttggtagccatatcacgagatttctcaaaatacaataagaaaacaaaaggccaggtgcggtggctcatacctgtaatcccagaattttgggaggccaaggagggcagatgacctgaggtcaggagttcaaaaccagcctggccaacatggggaaaccctgtatctactaaaaatacaaaaattagccggctgtggtggcacacgcctgtagtcccagctactcaggggtctgaggcacgagaattgcttgaacccaggaggcggaggttgcagtgagccgagatcgtgcccctgccctgcagcctggcaacagagaaatactctgtctcaaaaagaaaaaaaaaaaaaaaaacacgaaaacaaaataaagcaagaagcaataaaatatcgatgagacatttaaaaagacatttcatcaaagaacatatatggatagcaaataagcacattttggtttttttttttttttttttttttttttgagacagagtctcgctctgtcgcccaggctggagtgcagtggcgggatctcggctcactgcaagctccgcctcccgggttcacgccattctcctgcctcagcctcccgagtagctgggactacaggcgcccaccactacgcccagctaattttttgtatttttagtagagacggggtttcaccgttttagccgggatggtctcgatctccttacctcgtgatccgcccgcctcggcctcccaaagtgctgggattacaggcgtgagccaccgcgcccggcctaagcacatttttttaaatgctgaacataattaggcattagggaaatgcaaccaaaagccggtacacgctgaaagtaacaacactggtagtaccaagtattggtgagaagagagaaaaaccagaactctcctaggctttactgggaatgttaaatgatacaaccactttcaaaggcaatttggcagatccttaaaaagttaaacttacaccttccatatgacccagccattctacttctatgtatctatatacaaaagaaatgaagacatatgtccatacaaagatgtatatatataaatattcataaaaactttattttcaatagcaaaaatcttggtaacaacgtaaatgttcatcaacaggtaaatttgttcatcaacaaatcatggtatacctatacaaaaaaatgttacttaccaataaaagggatgaactattgatacatacaataacataaatgaatctcaaaataattgtactgagtgacaggagtcatactacaaagcatagatgatgtatggttccatgtatgtaaatttctttaaaaatgcaaattaatctatagtgacagaaagaagattggtagttgcctgagcagatcagtggttgggatgggacggggagagggagaaggaagagtgagaaattacaaagatgcaagaaggaatttgggagtatgatagataggttcattatcttgaatatagcgatggtttgtttcatggaaaatgtgtcagaacttattgtagaggttaaatatgtgaaatgtattctgtgtcaattaaagtgttcttcaaattagaggtggaagtgagaacaaaactcacagatgtatacctttcagtcctgtggattaattttgcccaaacctgctttggaaaacatgatccctaaatgaatgtggcctaacagatatatcctggcttggtcaaatttaagtaagagctgtaatatgaatggttcatgtaactgttaactgtcagtcaaaaacagagtcaggattaaccactgttcagtccaactcattaaagaaataataaagcaggcagcagtcagtggtggtataaacagtgacataagtgagagtacaaaccagtgtaactttctagggaccaatttggaagaagtctcaaaatgtgctcaacttttatctcaacagtactattgttaggaattttaaaaaatatattaaggatgtgagcaaatatttagtatatccaaaacagcataatatatattattagcaatctattatttgttaatagaaataattttaattactcaaactgtctaaaattagattcaattatagaaatccagccacacagtggaatactatgcacatcaaattgatgacctggaaagatcttaagggaggtttttcataagggaagaaataagctactgaaggatatgattaacttgtagtaaaatgcttataaatttctatttgcatacacataatagggaaggatgtatatcactattaacagcagtgattgctgtgtgttggaattgtgggtgacttcaatttttttggagactttgctgtagtttccaaattgctgccacaacgcaattttatttttaggcagaaaaaaattaatgtatatggaggttctttcttaactaaggggtgtgcttgagctgtgtctgagcacatatgattagttagaggactcagtggtgtgttgcgtaagttcctttggtattttgtgtgtttcagagggaaaaggaatggaaagaacagcatagcaagcttcagatatttgcctcctagctcagtcctaaagtgcattgctgagtccctgaacttgtatctcaggcttatggaaagatgagaaggagctgtcttcattacaaactgtgtgagacagggggcatgcagggctgtctatgtgacagtgacagaagcagatcactatttcagctgcacatgagacacagggtggctgcagctttactctatagggtttgcccttggctttgtgtgcagtgtcacctgggcactagtcccctgttcagcctggcagtggtggtgggcagagccgggcggatggaagagggctggctcctgaagccctgagatgctccataagatcccctgaaacaatgggcacagacttaggtgaaggcagaggcaatggcaggcaccccaaacactccaaaaccagcatagccttcccaggcccaaatttcatgcatctgcggcctcagttattctcaatggctctggaactttcagcaaagactttcacacactatccagtgcacacgctttattttggcattcaagccactgcaccaactaggtcacagggaatgttaattaaggcaagataggtagaaggaaaggagggtatgtacagcaattgctcctctttacggagaagagaacctgccacagaacaagatgcagttggttttgtggtgcctgatgaagaaaagaaaagggtggtctgcacagaatcttggctccattctgctgcaccgggaattcctgaccacagcagtggctgcggcagcctctgtgccttcctcattgacctccacgaagcacttgtgggcaaccttggacagaggcacattcttctcagttgacattccagaaaagtctgccttggcttcgtcaaaagcatcgatcattcctaatcttcgaaggaaaggctccaagtcataactctcctccagctttaatctgggaaggaaaacttgaaccttactttttgtcaacttttctgaatttgtccaggctttgaatttctcatatgtaagtgctttttccacctaggataagaaagactcaaaccttaaaatttagatgacaatagcctgatacccacacacccatttattacccctcccccaatgacatgcaattagatggtgagttctggtccaagaaaatctacagatattttcttttataaacacaccaagattttctcccaaattttcatcttttcacagttttatggagacaagtgtaaaagagctagtaaacacctgagacaggcagaaatttttaataaaaacaatttgtacctaactcctttcaaatgttaaaaacattttttaacagtttagaaaattctaaccaattcattacattaatcttcatgcaatacatactactgtgagtgggagagattgtttgacaaaagacaaagagcatggctcagaactctgcttctgagtctaatgaggggactattcagggttaaatcagagggcaactcagagtaaaagagagggaattgtgtttcatctgaagatgaagggaggcatgagaagggagcatgagacgagaaatggggaatgcaagaagacttcatcagcatccttgtttgtcctctgcctgtcctggagttagcttcaaattcttcaagatccatcactctttctatttatcaataaggtagaaagaaaaaagggagaaaggccttccactagcatctccccttccctttaacaactggagcccaggtgggtactcctctccaacgagcattgttttgattttgactctatttctagactctcctaattttctcataagtggccacaacagatgtcatccctgacttagggcagtagctttctgattccaaagggaagccaggaaaatcaagcagcccaccctcaacatcctgtgcattggtgtgccagagctgacccctatcaattgttaaaccctcagtagactaaagtcggccatggtgggagtatttaccatgtcatgagaatcagcaaactttataaatgaaggcttctcccacctcccagagctggtttacaagtgcactattagtcttgcacctcctaccttgcaagtttaccacttcccttcctggactctgttccttttctgtcacagtaagacttttctctcctcttgtataaggtaaatttcccacctgtactttagacttttgctattcatagtgtggttccaagcatctgtatcacttggaatcttgttacaaatgcaacatctcaggccacactccattgaatcaaaatctgcactttaacaagatcactagaatcatatatgtgcattagtgtttgagaatcactgccaagattgatccctacccttcttccaggacttttgtgcaataatcattgggctttcaaatagaccttccatgcagaataaagaataatattccaaataataatatatttagaactctctcttcttagcttcatcaaaacttgtccactcatatatgtatacactcaacagaaatgcatgaacatgttcatcaaaagacacacaatgatgttcacccagcactgcctgtaataaccccaactggaaactcctcaaatgccaattggtagatgaacataaagaaatgagctcagctggactccacagatactcaggctcattgcctggagcttaccacggcgaggtccgtgttgtcatcgggaagcagaatgaccatgctcagctcctcttccacatagggcagctccaggacctgggtgtgtacctcatccgcataccccattttaaacttagcttccttaaacatcatctgcactgtctttttttcctaacggaaaaattaaagttcgattactgaaaagtgggtataatttactaaaaataggatatatctaactcacagaaactctaggtgtgtataattactgtttctgcacatttatgctgatagatataaaatcttcattagtacatcttagtattgaatagaacaagctattgttccagcagatcaaatattaccaggtgagaaaaaaaaaacagtttggtaaatggagaaggacacaaaaattgacattatccttggaagaagctgtgtttgcttttttgagaacattcacagagaagctttctcatggaagcttccaactaaaaattctttatcgaagttaagataaatattcaggcccttgggctgaaggatctggttagaaaagatgtacggtccaaactctggaaagttgccttgttgtagaattggttttatggccttggctttcttaggaaggggaccttgaagagatgtgtaaggtctgctgtttttaaagcctgcatcaaatatatttttaactttcatatctttgtgcaatatgagagtatcatatttgatggatgtcaaatcccaagctaacaactattgataccagccattttaatattcaaaagggaaggtaaagttaggctttacatctgctaacttttttttcttgtgctcaggattgtataactttgacttttaaggcaataaataggcatagttgccactgatattgcaaaaaggtatctaactaaatacagagagtaatagcatcatgcaaacataattccaaggcttagaagaaattctcctccaatccaaatcatatgaccagagatggaaggtagatttgatcaaagatggaaaaacaaaactatcaataccaaaattatttttgtttctctcttccttccgccattaaagatacacaggaggcattaaattcattcagaaaagaagccaggcacagtggctcctgcctgtaattccagaactttgggaggataaggcaggtgtatcacttgaggccaggagtttgagaccaacctgggcaatatgacaaaaccttatctctacaaaaaatacaaaaattagccaggcctggtggtgtgcacctgtagtcacagctacttgggaagctaaggttggggaatcatttgagcctgggaggtcgaggctgcagtgagctgagattacaccactgcactccagcctgggtgacaaaaagagaccctgtctcaaagaaaataaaataatataatataattcagaaatagattaaaatcaagatcatgtcaaataaattattcatttttccaagagaatatttcctgtaatattactttaagaaagtagcagtatatctgaaaaatctttccctacctcgttggttttaaagagcattccccttgtgtactttctgtcaaattgctcattccactttcccttgaaataaatggcattcacaaggaccagctttgtcaggggatcgactgtcccagcatccagtacctctgaaatcttacctataaagaatatgagagtttattgcaatttgtggggaaataagcattcttttacaaaaatccctttgacacataaggcagaggcttggttgagagacaggctggagttctgtctttctgggcccctgatgaattaggcgatggtgtttagcagtgtggcatacacagtgtaacatttgtgcttaagaaaaaaatcttgaagtaggaagggcaactatcattttgccctcaatttatggatgtagaaattcaggaccagagcatttgccagacttttgctgagtgcacacaggtgacaagtggctgagttgagaacatcaccccagagtcttgattctgactatggggcagtcaaccttggcactatcatatccaagcctcaccgctattgtcattttaagccagataattctttgttatgggggctgtcttgtgcattgtaggatgtttagccagcagcatttcttcccattttgacaaccaagattgtcttcagccattatccaatggcacctggagggcaaagctacccctggttgaggatcattgctctatgcatatattcaaacctcatcagacaaggaaagctttccaaaggatgagagaacacagatctgtcaatgggagaaggcagcctctctgaagggggtgaatggggcaaaggaattaggggggtagaatagaggggcccaaattgagttgtccaatgagagggtaacaaggtcatctccaaggtccccttcagctctaatattctgggggaaatgtgggtggtcagcctaagggaaggaagggcataagcagccctggaagagcagtgacaggtatagggcagtggggggaaggtgtggagaaacttctaggttcctcaggtgacagtactgttgaacaactggaaagaccactaggcagatgatgtatgaaaacacaaaaggctcttcccttcccatggtaattattgtattggctttcagtagaatcaggggcattacttctgctggacacacagtggttgcgacataggatggaatggatgcaataatgagtgctaggttggcccttgaaataagtgacttaattcctccaccgtttacaagtatcgaaattacttgttaggtatttaggatgctttaaagtatgactccccctttttcctactgaagtaaagaactggcagaaatatttctggtgggatggaatgtggtctgggcagggaacactgcaggctccatgtcgctgcacctccagaatagggaacaaattcagaaaaggccacactctgagagcaaatgtagccatggccaagttaaaaaggaaggagaaggctgcagatcaaactactgagctgtcccattaccacgcggaggagataacatgagagcatgaaatgttgccctctctaagccgcaacttaattgaatgccattctgtctccgagcatggaagggcctctcagatagaagattaacagaattccaaggaggagccattttccagggattttccagcacggtccacaggggaggaggtcagagattcaacattagttttagaaatcagtacttcatgatctgataagaaatgacagagttgcattgctggcctgtaaactcagcaaacacatacaaacaggcctgtggtatgcaggtcacagtggcaacaccctgggaagtcccatcccagtcaatctaagcctcgttgtatagtaattccaaatcaacagaaatgaaactgtctcaccttcagtcttctctgccacccagtcatttatatgcttcctgcactcttcagtgtcttcagcaaaggacaactcctccagctctgcctgatagaacttctgacagtattctttaaagtcctgcaaacacaaaacactgagtttagtctcatttggtaaaggttcccacaaatgcaaaagcattttgttttctcttactgcatttctacaataagtgcttggacaggtcacactccacttcaaggtgcatgcgtgcaattaacaaaaatgtatgggccacctattcttgcctggtactgggcaaggtgcttggcttgaggaggtccacagcctgagaagaggcagctgagacacccagacacttacaggacatcatgagctgccactatccacaaaacatcacaggagcatccttgtcaaacgctctccactgactttaatattgcttccctcactaggcagtctaacacacttttactcattttgcatcaccagcaccaactacagtattgtggttttggttaaacgagaggctttataagggaactagattgttaagaaatcgtgttgaactgcaaagaaggaaatagcattttagatctgggtcatatggttggttggaagaatacagagcaaggaaggccccatgggaatggagcgtgataggcctggatcatagggtgtgtgtgcaactgggttcatacgtgtgcatgcgtgtgaacatctgtgtgcatgagtgcacatgtgtacatgtgagtgcttgggcacctgcatgtgtgtggattgcatgcatgtttgtgtgtgtgtgtacatgtgtatgtgtgtttgtgtgtatcagggaggggattaagggaaacagtggcaggggaagagccttgaaatgtaagttggggtccaatcagaaaagtcctgtacccaacacattaaaacccagataacttacctcacaaaaacctggctttggatataataggcctggaaactggctcccagcctctgtccattccctgttctcaaatattttaatttcagccaaagaaagaggatatagagggaaggaggagccaagccaccaggaaaagtagaggttggctccctgtgtcccaagtattacaacagtagtagatcaggctgatgttcaaaaatgaaatggattcatcaggagattcctgggatccagagtcaccttctgaggtagataaacccagagcaggcaaattccgggaacagcacccagcagctaccaggacagggatgtatttgagccttagaggagtcctctcagaacctccagccaataggatggccaagcttgctgcaatcagttcactaattttaagacaacatcaccctgcatatcaggtggcatgatttttgaacctcactgattgcattatgccaggctggactagggaaccttggaattatatgctgaagtaaatagaaactgcttagatttaaaacctcaattttatgaacagattttcattttagaaagacgattgtgtcagttgtaggtgcaattcagaagagggcgggcaaatttaagagaatttgagaggctacctcagcaggaggcagccgaataaccacacttgggactggggaggagctctgtacagcgtgttgaatgatgtggtcactcagggacagggaattaagggggagaagcagggctgcgtgcaggagaattgacatattgggattgataacagggatgggtgtatcagtaacaatttacaaaggacgtaattcagaagtcatcataaaatctaaaattgtaatttcagaagtgtaattccaagcacgttttaagtcagtacagttatactttattatctgctcttctgtagtctaacttactttcaatttctttgtcatcaatatgtgaatactacttactggaaggaaatcacacgtcttttctccaaagagtctgttggcagttctaagcaagtactgagtgccagttctgttaacttcactgagaagtgactggaaacctcggtgaatatctccgtctttgtataaacaaagtgcctgggaacagaaaccaacactgacttttaacgctgccattatactctcccaaagagctttctccaaacaaacctctgtactaaaaaaaccacacaggatcctactttaaaaaggtttggcctaattgtctagcacagggtttctcaacgtgggcaatgcagcattgacatttggggccaggacacttccttgtctcatataagcattgtaggatttttgcagcacttctggcttccatccactagatgtcagtagcacctctcagttatgggatctaaaactccaggtcttcgcaagggtccccaggaaggaaaaattcacccaccgacccctacctcccacccaccaggtgagaaccactagtctagcaaatttattcatgccagcaaggcagtgtgcgataattcagggggaaggcagaacagaaaaaagtaagtaactgggatgaattacaactcgggtaatacaaacaaaagcagcaataaaactaaaaatgtttcatctgcaattttacagaagttctccagaccatattataaattaaatgtcaagtcagagtgagaattaaggttattcctatatctctcgatcacccccagtccaaagcctcactcctccaatcagttgaaagaaacaaccacagaaggatgtcaactggggtccctgggtgcattttaggagactgtctatacactcctgatattggtggcaaaatgatatccatttggcaattatctgggacatcttcaccaatagtcagagaactgttcctaaagcattatcccacacctcgtttgtagataaggtcatacatcaccattttgaatcaaacagattatatattcctcaaatcactccagaagatataagtccacacttgtgttacattcctcttctaggatgcataatcatgcactaaaataacccacttaacaaaaagttaatctacgcacccacatgtctattaaaaatataatgttagcaaaagtttaaaatgtaacaaaattggaatattttgctttcttgtgggacagaattatactttatcctacttatcaaagatgtgaatttgccagactgtattgatgatctgatctagagaaaataactctatggcttgacttctcccataaacatcagaggagatgatctgtccaacacatttcacctttctacccctttctgctcttctccactgccgagctgctcctccttctacctccagctccacaggcctcagctagagcaaaagtacagaaagacagctgtatggaagcacaagggaaacacactgttctccttcctttgtgacaagcacacatacctgggacatctgggctgcagtgcttccctttgcccccatgaagaccatggccagggcagaggagatgctcatgggagagaagaatacgtttcttgagttgtcctcttcccccaatattttaaataagctgatggcaaaagtgccatttgcttcacagaggtcatccatcagagaaggtctgcatcaaaggcacagcagggaaaagcacataagccaatgactccggtagccacgcagtcattcatcccccagtcagtggacgctgaggtggtaggtggaagaaccaccctccaaagacttccactcctaattcccaaaacctgtgactacatgatctgccctggcaataaggactttgcaaatggtattcagttaagtattttgagatctgagattatcctgagttatcccagtgggcttaatggaatcacgagcgtccttaaaagagagaggcaggaggggcagagtcagagacagagacatgaggacggaagcagaggtcagagggataggggccactgatgaaggaatgcaacctcctcgagaagccagaaaaggcaaggaacagattttcttctagagactccagaaaataacattgctatgctgtaccattttggacttaggacttccagaaccataagataataaatgtgtgtcatttttcagccactaaatttgtggtaaagtgttatcacaccaataggaaacaaaaacactggtttattcataaaatagccaatctcagggcctatgacttgtatccctcttagcaacaggggcacagacaccctgcaacacctgcttacttcctcccttggtatagcagcagccaaaaggtctttgtgcggacgggtgtggtgcctcacacctgtaatcccagcactttggaaagccaaggcaggcagatcacttgaggtcagcagttcgagaccagcctggccaacaaggtgaaaccccatctctattgaaaattcaaaaattatccaggcatggtggcgggcacctgtaatcccagctacttggaaggctgaggcaggagaatcacttgagccccaggaggtggaggctgcagagagccgagatcacgccactgcactccagcctgggcaacagagtgagactccatctcaggaaaaaaaaaaaaagatcttggtgccaggcactgttttcagcactttccattatcacccaataacacattgaggtcagtatagttattgccccactttagagatgagaagactgagtgaggtttagtaactcattccagaaccctcaggtagtcataaacgcctggcacccacagtttgattccagagcctgaattcttaatcattacacaaaatccccctattgctgctaattatctttcagtttaatgtctccaatataatctataaagaatgttctaaaacttatatatgtaattctttatcaataaaaactgtatttctgtcatcacttcctgtgaaaacagctagatacatttcaccaaatttggagggtttgtttgagataggctggcctgaaatatgaggctactggaaagacaaaaaaaaaaaaaattactttttaaagtgactccaaaaagatgccccattttccccaagagttgctgaaactgggttacggagttgcaaagaaagtccataggcatggatataaaatatgaagccctaatcttgtcatcgagggagcacttttaattagatgcattgatttttggatcctcctggctaagagggtcttaacattcccctcagcttgaattaactttagacaacctaaacttgactaaataaacaaactaaacaggcttcctcctgcctctaggccctttcttcctttttcttaaagcattttatttagagaacttgtcattgtaaattctttatcagcccctttgagatgtaaatcttttaaaaaagcttcttgctagttttacatcccaggacagtctctcaaagacctggaagccatccttttgaaatgtaatcatcaagaaagatagcatccctatcaaccagttctgtggaaggtaggagcctaatctaggggggcaccttgcttaaagtgctaaaaagctattacagtttactattattataattttagtgcattattataagtcctacattgttgtttatatctattaaaccctataaaaacgtgggctatttcataaaatattgcagttcaccaactcatctcaatttccctcagcctttttgttcttacaattgagaacatttgggaaaataaaaggacaaatcattatctataatctcatgatcctacagatattttttctattatattatcctttttttcataaacatgtataattttaactgaaaaatgtagtcaaacaagaaaaatatgctccagtttggaaaattagcaaatgagcaaaatgaaaagtcctggaaactctatctagagatataaattatcgtgcatgtgttcatttctataaacagatggagcctcatccagtttcccagcggcctacaaagtgcctgttgcagcagctctgaaactgtatgcccttagtgggggtcttggacaggtcttctgggtctcttagagcacaggaggaaaggggaattgagtggctgagacaaggaagtggggaacaactgtattaaccaaagtatcttcactgttggatgttttgcttcctcctaagatatttcgcttcctcctaagatatttttcaacagaagggtgtctcttacttaaacaaaaaagtgagcttatatatttttttaaagtgtaaaggcaacactctaaatatagaacaacagtctaaagcagtgcaaattctttaaagtttaaaataaatgttagtgcagttcagaaaaaaacaagtagggttagggttatggttagggttcagaaaactaagtaaaataaatgtgaaccaagaaaacacaatcatgacataatttgcacaacagtactctatacttaagaggttgtttattatggaacaaaatattcctgtttctcactatcctcacatcataagacctctccagagcttatactagggtctgcagatgttggaaggcagagtagtatgtgcaggcaagtcccggggtcctccacaacctcatctttcactcgaggacatttcagagactgggaggcaggaaggaatattcagacttcatagaactgggccaaccagaaggtggcaccaaagaaacttaaagatggagagaattgggtaaactgggtaagacagagacacaaataaatttggatttgatccaaccttagcataagagctttggactcagggtgaagcaagcatgaacttaggttggggtgtgtgtgtgtgtgtgtgtgtgtgtgtaggtgtgtttaatcatcctgtctgaactattgaatcaatagtttcaatagttctgaatgaactgaactattgcatcaatctcatctttacattttaactgctcttagttgcaaactgctacttctgatttcttccatctttcctcttccttcatctcatgtccccacttccattccttttcaacactcaggtgctggtcagtatgtggtatttcccatcaatatctaccctgcagccacaatacatgtgcacacactcacacacaagtgcacccccttggcctctgctcaggtcagaagtcaggcaggggagaaaaccaggtaacagaatgactcttccccctgtcttcctggtgttccaatctcatttggactttatagcttttgaaagggttggaactttaaacaaaatggactttacccaatatttctaaaaagcaattaattttaatacaaaataatactttaggtgccaatgccacaattagattgtgttataaaaacacaggaatcagaagtttttatttccagccaaccaaataaattggcattaaactagtttttccttttcaggcaaacaaacaaagacagaggcaattaaatactgtccacaggactcacagaaaaagtgagagtcagcctcaaaagcaagccttacgtttccaagtatatgatattgtcctcctcctgggagccccaaatacctacgaactacctcctccactcccccatccccatacaacatccaaaagtgagtttattcaaagagcatttatgtagaatgttcactacctgttttgggaggagaaaatgaaaaataaatgcgttaatcactgcccagaagcttacaccctaaagaagacacacatatggaaatgcgtagtttcaagccagtcaggtgctctataacaggataattttgaaagtgctatggaaatcctcagaaaaaaacaagtaaataaataaataataagaagaaaacctctacagaatcctggaaagactttccagagaagctggctttaaaagctgggcaggccaaggtaatttatagattcaatgccatccccatcaagctaccaatgactttcttcacagaattggaaaaaactactttaaagttcatatggaaccaaaaaagagcccacattgccaagacaatcctaagcaaaaagaacaaagctggaggcatcacattacctgacttcaaactacactacaaggcttcagtaaccaaaacagcatggtactggtaccaaaacagagatatagaccaatggagcagaacagaggcctcagaaataataccacacatctacaaccatctgatctttgacaaaccggacaaaaacaagaaatggagaacggattccctatttaataaatggtgctgggaaaactggcatatgtagaaagctgaaactggatcccttccttacaccttatacaaaaattaattcaagatggattgaagacttaaatgttagacctaaaaccataaaaaccctagaagaaaacctaggcaataccattcaggacatagccatgggcaaggacttcatgactaaaacaccaaaagcaatggcaacaaaagccaaagtagacaaatgggatctaattaaactaaagagcttctgcacagcaaaagaaactaccatcagagtaaacaggcaacctacagaatgggagaaaatttttgcaatctacccatctgacaaagggctaatatccagaatctacaaagaactcaaacaaatttacaagaaaaaaacaaacaaccccatcaaaaagtgggcaatggatatgaacagacacttctcaaaggaagacatttatgcaggcaacagacacgtgaaaaaacgttcatcatcactggtcatcagagaaatgcaaatcaaaaccacaatgagataccatctcacaccagttagaatggtgatcattaaaaagtcaggaaacaacaggtgctggagaggatgtggagaaaaaggaacacttttaaactcttggtaggactgtaaactagttcaaccattatggaagacagtgtggcgattcctcaaggatctagaactagaaataccatttgacccagccaccccattactgggtatacacccaaaggattataaatcatgctactacaaagacacatgcacatgtatgtttattgcggcactattcacaatagcaaagacttggaaccaacccaaatgtccatcaatgatagactggattaagaaaatgtggcatatatacaccatggaatactatgcagccataaaaaaacgatgagttcatgtcctttgtagggacatggatgaagctggaaaccatcattctcagcaaactaccacaaagacagaaacccaaacaccacatgttctcactcataggtgggaactgaacaatgaaaacacttggacatagggtggggaaaatcacacaccggggcctgtggggtgggcgggggggatgtgagaggaatagcattaggagaaatacctaatgtaaatgactagttgatgggtgcggcaaaccaacatggctcatgtatacctatgtatcaaacttgcatattgtgcacttttaccctagagcttaaagtataataataaaaaagaaacaacccaaatgtccatcaaaaacagaatggataaatggattgtagtatattagtataatggaatacagaacaataaaaactgaaagaaccatatctataagttctttagtttttgacatggacaattcccacaaataatgtattaaaggattaagccacagagtaatacagtattattccatatacgtaaagttcaaaaccaggctaaactaaatcattctggatgcatgtgtagacagtaaaactatgtcgttattatcatgaaactcaggaaggtggtgatacagaggggagggaagcggatgtgatgagggaggagggtgtgatcagtagggaaacacggcctccagcgatgacaatattccagttattaaactgggtgatgggttgtactgctgttctttataattcaatacacattttaaatgttcttttgtatttattcaataggtaatgaaacttattaaaaccacatatcaaaaaaaaaaagaaaaaagaaaagccgggcaggcattccgtaggtggtgaaatgggggatgcaaataataggctgaggaattagcaggtgccaagaatgtggcaggaagacaaagggcatggtttgtgtggtgcccctggggtgggtttctatggtttcctgtgagccacgtggtagtgagataggacggacccttatgccacatttaggagtttgaacttgagattatggtctggaaagccatgcatggtttttaaacaaacgtctcatatgagtgcattggggctttagaaagataacccggctcagtatttggtatgaaaagaggctggggagctggcgcgtaggccaccaccatgggccagggactgctgaaagcagagggattgtctgcaatagagaagtgggggctggaattgccaggcagtgactgctcacatagggacagagcacaggagggcccagtcccgaggcgcctagctgttataaattcatactcagggtgggtcgtgaaccactggaattttaaatgaggagtcaggctggaatggggaatatgacttgttccatttgagatgcgtggaacacaccataccaggtgaacctgtaacaaagacagccaaggatgtttgtaaacagttagggacggggatgagtagactgtctagagccagagacctggagccatcagcatcccgattccgagaggttccatgagaatggatgaaactacacagaacatctagtggaggcacaagggaagaaatccacctatttctgtggaatttccaatatctggaggtagtaaaaggaagagacagaggagggcgaggggagagcgcgcgcgcgaaacggagactgagacccagccatcagggtgtgctgggagaactggggagacgagtctgcccctgcgagtggacccagggaagagggcgcggtctacgcagagcccgaggcagggaagcgggaggggcccggagagcagccctgggtgaggccctttggaatggagaggtctcaggagtgaagcagcagccagaggactgagagtgtctggaaagtgaggaattggttaaagtaacgccggtggctgcaacagagaggggcgtaggtgaagaagcacccccaaccccatcccatttgagaggaagcagacaatgaagaagagaatatgaaagatacagaaagatgaaggcaggggtggaaagaacggaggggggggacaggaagggaaggagaaggtgcaaccaataggctgtaagcacaggggcaagcttgggcgccaggggagcagggactcggcgcctccccgatctgccccaggcgccacggaaattccaggccccgccacccgcactcccggagcctcctccgcagcggcggctcctcccactcgccctcccggggctcccgcccaagcttccggcgcttccaaactcactgctcgcgtcaccccccgctcccatccggcagcaaatcacagcgcagcaaatcgcaagcctagaaacagacgctccctctccaggggcagcttcctattcagggcgggtctgggcgcacctgttctcgggacccggccctcggccttcgccgggtctcgcggtggggcttgggaccccggctggtgccagggcgacccccacagccccgcgactctctttcccggccgcctcccgccgtcattctcctagttcctgcccgcctttgcctcgctctgattcctgctctggccttcctcccttcccctctctcacgcactccagggaagaggtggcctccgcgggaacctgggcacctccgtgcctgccccgcccggtacctggcagaggcccccacctcctgctgctgctgctgctgccgccgccgccgccgccgctgctgctttgactattcctcctttgtagatgcttcacgtccgccttgattgagggcaaggcttccggcagatgactcagaaccgttcgattgctctggcttgtgcaatccctgattctttgggcctgtgtggatgtctgtgggcaccggagagagggccagtgcctggtccttggagggtggtttttataaaaagtgtacacattggctattgtccttttttcaccgttgattcacgtttgactccactgtagttagacgtccacccaaacattctctgaaattgctcctgataaggatactaatcattttctaattgccttggccaaaaaatacatcccaggtcttatttcattttgtttgttggcgacatttagccctttcttctttgaccttcccaggatttccagagcacccttcccttctttcctgactcctttgcttggtatcctgattcctttcctaccattctaagccctcttcttttcattataatatgatcttcttgtatgacctccactatatccataggttcatgtttcaacttttaacttatttcttcaaaatctctaacctgtctcgagccgtagatgttacctgtgtactactggacatctctcagaaacctgggtgtgtccctccgcagttcttttaactaaatgatcaacagtcctgttacttctatcacttttgtgttcctcctgtccatactctgtcacctctatcatgtctcatgtctctcctaaacaattaattttcctacctacagttctgatccatgccaacctattgcctactctgaagttcaggtaaacattccaattctaattattttatacctcagtttaaaatcctttataacatcctcttatccaaaaagataaattagaaactattgagcaaggagtgtaagcctcttcacgtcttgatttctcccttcttttctttagagccatagcaaattatttacattccttcatatcctctgttcatatacatggggttcctggcatgcaagcttgtctttcaaaattcagttcaaataatgtctcctgcaggaagctctcacagagctctccagtttgggaattctgcttttaaagatgcacattcccactctgcaccccaaaccaattaagtaaagatctcttggaggagggaacccagcagcagtattttgtaaaactcgctcagtgatttccctgtggaatcctaaaagtaactgttcccaagaactctcctcaataagtcttggtgcaggattcacccccatcttttctccatgactttgaaacccctctctctagtggctacagactcacatttgaactgtgcccaaacttaccggctctgccagttgaaaatgccaaggtcacttccccattgtgcatcaatttctccttgttggtcagaactagatccagactttgggccccactttgtccatccactccttttcctctttgaccagcaaaacagattaaaagatgaaaaatgcttgttttcctccgtgaaacttccagcaaatgcctggtggtttgaagtcctttacaaaggatataatttggcccctgggagtttggttgtaaacacagatatcccatgtaggaaacacagtgcgtgcaggtgcaaagaggcacaagatagcaccatgaaaaccatgacgtatccaggcctgcagtggtacagatagtgtggatgaggcactgcaagacaacgtctgatgtgtccacagggccatgtcgtgaaggccatgtggacgctgttggtggtgtttcttcgttcccttgtggactatggggaaacacaaaaggattttaagctggggagaaacataatcatatttgtatttacgaaggtccctctggctgcaacatggagtatattgtaggagggtaccattgcctgcagtgaggcgttaaggacgcagcatggatgagagatgaggaaagctgaaatgaaacagaggcaggagaaatggagaggaggacatggccttgaggaatttccaggacataaatttggcatcacttcttggaccatgagaggaagagggagcaggaagaatcaaagatgaatccaagaatgaacaaatctaatttttgaaataatgcaatgaaaagtagaactggtctgttttcctaggtaaggaatttgtctgcaagtcgatattcacgaggattaattgtatttattttctgagtatggacaatttaaatgctgggccctgaagggaaacccatctctcagaataactaaagcccaatttcagagtgagcctgaacaggctccccactccaccctggcccactggggacacaacaggtaactgataaggttccagagtatataattcagtgcctcagaaactgtcctcagcctcatcttctgtcttcttgcctcatacctaacttcacatctcttcagtcagtactagggagtatctgctggttctcaactcccgactcaaccttgacctagttttgtctgactccctcggctcctgactttgtggacctcaggaagggcacactcctaggctagtctcaggttgagctaccaccgtctccaaattagacttccacttgtcaagaaggaagtagagtccaggcataattggagcttcctggggtgattcgaggtctctgtactccttgctgtaaaatgcacaaatactccaccttaaagaatctcacggggcagggcacagtggctcatgcctgtaatcccagcacttttggaggccgaggcgggcggatcacctgaggtcaggagttccagaccagcctggccaacgtggtgaaaccccgtctctgctaaaaatacaaaaatgagccaggcgtggtggcaggtgcctgtaaccccagctactagggggcactgaagcaggagaactgcttgaacctcgggaggttgcagtgagttgagattgtgccactgcactccagcctgggcaacagagcgagactgtctcaaacaaacaaacaaaaaacaaagagtctcaccggagttccaaaatatccttcagtgactgaccatatactctagcaaaagttacttcatatatgcccggccacagctctcaatctcccatgagattcccagcaatcttccctggttttgagaaagtgtaaaaaaacacaaaaagcaaaaacaaaacacacttgcttcagctaattaatgctagagaaacaaacatagcatcataaaatgtaagcatttgaaagaaccatattgttgacgctgttccttatattttgcaggagaggatggtgaaatagaggtagtttgtgtgatttgcccagagtcccatgaagaattcacagaagaattgagactacagtcagtatggccacattcccagagaactgctttttctgccataccctgtggtaataatattgtcacaagacaattggacagtgtttcacatattcagtgatggaagaagagtttaattgaaataaataaatgtcaggaaaaattaattcgattgtgataacacttaaggaatggagtgttcactctctaggagaagttgggctaaatgatgaaatggagtgtccgtgtatgtgatctgtggtgctgttttgctgtagttaattcctatttttaacttgaaatgtgttctgactgcgttagtgccaatttttttgagaccggtgtacctatcctatcaggtacattcgatttacacaagtacattctcttttcagttctcccaccacttctctttattcaaacaattaaaaccttcttgaatattatttggttagtcctttgagttaatgaatcaattttacaggatctgcttctgtcatttttcattaacattccccagacaaatttgtactgcttactaacagcacaatcctaaattcaaaagattactgagttataaagtggatgtctggacaatccaaaacataatggctgtggtttgggtgtggatttttgtctccacgaaaacccacactgcgattcagtccctgctgtgccagtgttccacttagggagaggtgtctgcgtcatgtgggtggatcccttgtgaaagccttggtcctgttctcctggtagtcagtgaggtacaattagctctcatggaatggattaattcctccaagagtgggttgttataaagtaaggtttctccccatgtttggtccctcttcgcatgtgcctgcatcccctttgacctttccctcattgaacctgcaggttcaatgaaggttcactgcaacctccgcctcctgaggttcaatgtcttttgacatagcacaaaagccctctccagaagcctggggatgccagtgccatacctcttgtatattctgcagaatatgagccaaataagccttctttctttataaactacccagcctcaggtattcctatatagcaacacaaaacagactaagacaatgcttatatgaaaatgtctagattgttaccaagacaaatgtaagcaatgaaacatacatgttgtagagcctctgctgatcaagggtatggagaacccagtggaccttaatgtgaaaagcattacattaatatgcctggtattgtatttcaaggaacttataaaagaaaaaaaggctcctgatatggcattgcctggaaactgccattagtccctcattagtgaatcgtcagtctctccattaatttgaagggggttaaggggaagtattaagtgacaaagaatttggagggaattagaaagatctctttggtttctaaacgtatcttttcgtgctttgattgaatgtgttgcagataggaaagttgaataggagatccctgatagcctctcagattttagcttatcaatagagttaccattgattgttgaaacagtgtgagttcctgccacttgttctagtgtcctgtctggttcagcattaatctcagaattttcagtttttaatggagtagtttttctaatagctgcacaccctcaagccagaatgtgtttgctagatccacactgtggacttcagtctcctgccacactctttcataatagtgggagagatgcttattcagttacatcaggcagtgttcactttgcaaacaaactacatcagagatttgaagagaagagaattaatacaaagaattattaagtaatagattttttaaaaatactgtaatggcagcagatgtagaaggtacaaaagagaaattgtcacttcatgtcttggggtaagtggaaaaaaaaagaactaaggacttaggaaaagacataggtcatagactgtattatgtcctcacaaaatttttatgtaaaagccctaacccctaagatgatagtttctggagatagggcctttgggagttgattggggtaaaataaagtcaggagggttgggccctcttgaagaaattagtggcattaaaagaagaagagataaattgctctccctgccatgtgaacatagctaaaaggtggccttctgcaaaccaggaatagagccctcttcagaaaccaaatctcctggcagcctgatcttggacttcccagcctctaaaactctgagaaatgtcagttgcttaaactgcccagtctatggtattttatttttattttttgaatcagtgtctcactctgttgcccaagctgaagtgcagtggcatgatcttggttcactgcagcctctgcctcctgggttcaagcgattctcctggctcagcctcctgagtagctgggactataggcaggtgccaccatgcccagttaatttttttgtttttgttttcagttttgtattttcagtagagatggggtttcaccatgttggccaggatggtctctaactcctgacctccggtgatctgcccaccttggcttcccaaagtgctgggattataggcatgagccaccacacccagcctatggtattttattatacccacctgagcaaactaacatgactgaatgtcccaaggctgatattcagagctctttgaaggtgtggctgccacaggaacatgcagtctgcagtggctatgaaacttgtcagatggccaaagctggcttacgagaagccatatgtccttgctggagggtgtgggcaggctggggctggaccatagaagcaaatgcctggtagggatatagttgccagaggttgcatcttgaccaaatgcagaaatggccattgtgtggagaaagcattccctgagaatgtggctacagatgaccaagtgagctgctgggtgcctacactgaataatcctaagaagatcagaatccagaaagtgcatttctacttctatcttcttacagtgtctctccagctccctctattggcaaagctgaacactgggccagtcagtcaagttcttgttacaggatccaggtttactatcacaaagtccacaaaggtagatttagagctgatagacaataaattgatatctggcacagcaatgaatggagttctcaggcaagcctgtcattaaatctggatgataactactgaatctgtctcttttcctgatcctctccagaatccttcatctcaaggacaatatctaaggaattcactggtaaaacattttatgcttggtcacaagcagctagagaaagcaaactacttccggcctggtgcggtggctcatgtctgtaatctcagcactttgggaggccgaggtggtggatcacgaggacaggagttcaagaccagcctggccaacatggtgaaaccccgtctctactaaaaatacaaaaattggtcgggaggctggggcaggagacatgcttgaacccaggaggtggaggttgcaatgagctgagattgcaccactgcactccagcctgggtgacagagcgatactctgtcttggaaggaaggaagcaaggaaagaaggaaggaaggaagcaaggaaggaaggaaggaagcaaggaaggaaggaaggaaggaagcaaggaaggaagcaatgaaggaaggaagggagagagagagaaagaaagaagagagagagaagaaagaaaggaaagaaagaaagaaagaaagaaagaaagaaagaaagaaagaaagaaagaaagaaagaaagagaaagaaaagaaagaaagaaagaaaactacatctgaagtcaacactagtattggtgggagaggaattttatgctgcattccccaacagccactagatacgccaatcaggtgtgcatggtccatgctatgatctgtgagtatcagatcaacgtcagcttgttcagcaagccctcaggactggaaacctccctcaagtcctctgccccctccatctccttcaacacagaagcttcagctcaggaaaggaaaggccacctttcacatgcagaagcgcatcagcttctggaggggtacttcatgaaggtactgcctccatcttctcagtctccaaaccatcccttgctttggacctctccttttcaactgtggccctgcagccctctttgagaagagggatgcaagagccttagagttcccacttattgaagcataggaggagtgaaggctctcaggacctccattcaggactcctggagggggtagcaatgcttgcagagttcatagcgcccagatgcacctgaccttagctccatatcccaggatgctcatccctggatccaggatgatgtgcttttggtgccagaagtttctgagttccacttttcagatgggataacacagccaaagaaatcagacttcatcttaaagggagacaggtttctggtccctactcttcctgcactgtgctgacagctgcccttctggtaggagctgatataggtaagtttcacaataacaagaggaagagattactcatggcttttctggatgtaccaggcttgtttgagtaattttccagtgtttggcattaccagacattggatccacattctacctcgttgctagctacaagaacttgttcaagctacttaacttctgagtctcagattcctcatgggtttattatcactaaggattaaataatctatgcaaaatgtccaatgcaaagaaggtattcagtaaacatatctgaggaggtcagatcaactttttttcctagaattcaattctaaatggaactcactttgtggatacttacaagaaacactacatagtgtaagggaaatgtcttagtgcaatttcacaaaacacatggaattattatttgggaggctggacctttcacggaattgtagctgttttgattgtctcatattcaccttgttggtgaaggagggatctcccccattaaatagtaggagataactagacacatggcatgacacactggacagatgaaattggcggcagtttattagtcacaactgctcacagggagggaggtcaccacatgccatgcggggtcacaggagagttgcatttgggaatagagtgaaccagtaggggctgtggaaggcaggctttgcagtaacaagaggaagaggcgattcctggctcctccagatgtgacaggcttgtttgaataattttccaggctggagggaagtgagccacgttgagacccagggagggtacagctgactgcctggtaagggaacttagaagggggaagagccttcctgctcagtgggtgaatagggtgcacatatctgacaagagcaggggacttcatggccaggcctctgaggccctgagaagcccatagatgtcaaggcagcacatgagctttcaggctttacaagacagaaataaaatagtgaatgataactcttcaaagagaatatgacaaacaaaatttcacaagaccctctaggaacaatatgcatattgttatctctatgttttatgatttacatggctaaagtctgattatattatatttctataacagataaaaccattatcattcattttcataattcatattgaaatgatttgattatcctcattccaggaaagaaaaaaaaaaaacaccctcctgcttggatacttagaggttactccatttacactccccactcccacctcttactccacttgactggactatccacaaggcagtgcaaagaggtgtgtgtttccctgaagaagagggtgccaccctgttgctcctggctgtaacactcagaggctctctctgcagtggaagaatcatatgggctgattattccccacctttcagacttctttccaagtccaagtgttgggcagaggaggaagaggaatgagagtagcaaacccggcaacctctgatactccctctttcctctctcagcccacatataatgtggatgtctccttgtaatcttcagtctcattctctccctgaagctaagtagctggctttggtacaaagggaaaagttggccagttatggaaagaaaatatgggttgtgtcagtgaagttctgagggctcagggtctttctggctgcaagggggttgatggaatgtctttttagctgtgccctagaaaacaatataactctccctgttcaatctaaatcatcagtgtgttcccttatcactgatcacagcagttgcatttcaagcctgtaaaccatacagtatggcaataccagtgagggagacagctagaaatttttaaattcaaattttaacgctatttgtgaaaagaaaaactacctaatattaacaagcaaaaaaacctggaagagattatttcatctagagagacatcacagatacttgataagggaaaagctaaagctagagaaaaggtctacgagttggcaatatttccaaagaaaggttttaatgtttgcatagttgtttagagaaaaaaggactcctttttatatttgacagttttgtaaagaactgactataaaactgtttaaaatattatcgaaatagaataacaatatattttcacctttagttttatcagcaacccaggagtttacacgtgttgtggacttctctgtatcattcacaaagtctagctgttttatcgttgcttggtagaatttgccacaggaatctgtaaaaccctaggaaaaaccaaaaacataatttcacagctacagaaacaactaatatcactggcttacttcttcattgtgccacatcagaaaatcaataattttatgatttaccatatataatttgcaaataaagatgaggaattaacctaaagtccaaattcttttcagctattcctaatcttagcttataaactaagcaacgcggtgggctgcatatcaagtgattttcatcaactgctggatccttcatgagagtggcagtacacccatcaatgagataatttaaaacactgagaaatctccaagaagacatagaaatccttaataggtaagtacctaatagtggaccatcaagacacataaggcaacaactaatagaactgaaaggagaaatagatgaatccactattataactggagacttcaatatccctctatcaggaatgaacagatccagcaagcagaaaattagtaaagaacatagttgaactcaaaaacaacatcaagtaactagatataattgatatctattgactacttcatccaacaagagtagaatctatattcttctcaagcttacatgaaacatttaccaaaagagaccacattctgggccacaaagcacaacttagcatatttaaaataatagaaatcatgcaacgtctgctctcagacaataacgaattaaactagaaatcaataagagaaagatggctgaaaattccaaaagtactcggagattcaataacacacgaatcacagaagaaatctcaagtgatattaaaaacagtttgaactaaatgaaagtgaaaacacactgacaacactaaatgctggtgaaaacgtggagcaacaggaactctcattcattgctggtgggaatacaatattgtatagtcactttggaagacagtgtggaagttttttataaaactaaaaatatgatccagcaatcatgacccttaccatttacccaaaggagctgataaaacctgcacacaaatattcataacagctttattcctaaccggccaaggtagaaacaagaaagctgtcccccatgactgtactaggggatgttaatcacaggagggactatgcatgtaccttttgctcaatttttctgtgaacctaaaattgctttagaaaataaattctttttttttcttttttacaaatggtgactatagaagctgtcactcctgtaaattattaaatttgttgagtgaggattaacacagcaagtgctatatttgcctctacattttagaacaacagactataaatatgagtctgttgttccataaattacatgggaaacatatacaatcctagaccagcagtgtccatcggaactttctgggatgatgaaaatgttgtaaatctgcaatgtccagagcggtgccacataggctattgagcacttgaaatatattaatagctagttggactgaggaactgaataattaatttcatggaaaccttatactcatgtacataatgcatagcattatgggtgtttcaaactatgatcaactttccaaaaaagttcaaactatatttagttaatatattctgtagaattataatgatcttaacagaaacctacactatgccacataaaagcagaacatttggtggaaattaaatgagctgaaacaactaatttatgaaccatgcattatagaattgtttcccaatctttctctcaaactgctcacagctaaatatttaatttccctttccataattttgcttttactttggaattttatacttctgtatcagtatggcatggagaggaaatgtgactgtagttaggaacacaagttctgcaacttactagttgtgtgaccttcaaatatttgcttcctctgtttatgcctccatgttctctcctgtagaacaaggaagcagtaatagctacacctcacaagataactgtgaagtcctgatgaaatatcaccttttcatgtaacgtttttgaattctcagtaccacgtaactgatcgatgtacactatttcacttaatcctcccataattcatgcttttccaaaattgctgcctctgcctatttttcagtggtttggaaattatttaaatctctcagtgtaagaaaataatctgaaggcaaactgatgaatcacatattgatgacttcttgcacgcctaggtgatctttccatctgttaaacgtgtttttaaataactgtctgctattttaagttactatttgacaaagtgtggcaaatcaaacaaaaagaacatgtaattatatattgctattggtatgggattcttgaaaataagaataaataataaattaatctgaaatggtttacaaatatttctcatctttttaatttatattccaatatcaaaataacattttaaatatcaatgcttttctttcaaaatatccaccacgaattgatatttggttgttaacattttggttgtctttatatagaaaactagtaagctagtaaaaagatggcaataaactaaatagtgctaagactagtaaaataagtgaagttgtaaaatcacaatatcatatacaaatcttataaatatttcacttttatgtcttaccaccttcactttataccaatctgtttgcaaacaattaatttagagtcgaaagcaagtttgaaaaaatacatatcacagtaagagagatatcctctacaaaaataattcttataagtcaataagaaaaagacatacaaccccatagaaaaatgtgtaagaggatatgtaaataggcaattcacagaagagatagaaattctcaatacacataaaagtgtttaatcccatttataatcaaaagtttttattttttttttttgttgcttctcttgttttgtattaacccttccctatcgcatatgttgtaaatattccatctcagattcttttttaaaaactttattttaggttcacggatacctaagcaagtttgttatataggcaaatttcatgtttcaggggtttagtatacagattatttcatcaaccatgtaataagcctagtacccgataggtagttgttcaatcctcaccctccccacacccttctccctcaagtaagcccccatatctgttattcctttccttgtgaccatatgtactggatgtttacctccaacttataagtgagaacatgtggtatttggtttcctactcccgtattagtttgcttatgattaatagcctccagttccatctctgtggctgcaaaggacatgatctcattctgttttatggctgcatcgtatttcgtggtgtatatgtaccacatttttttaatccagtctactattgatgggcatttaggttgattccatgtttttgccattgtgaatagtgctgcaatgaacatatgtgtctttatggtagaatgatttctattccttcaggtatgtactcaataatgggattgctgggtcaaatggcaattctcttttaattttcttgagaaatcaccaaagtcctttccacagtggctgaactaatttacattcccaccagccgcatggaagtgttctcttttctgtgcagcttcaccagcatctgttttttgacattttaataacagccattctgaatggtgtgagatggtatctcatcatggttttgatttgaatttctctaatgattagtgatgttggcaatgttttcctaggcttgttggccacgcatatgtcatcttttgaaaagtgtctgtccatgtcctttggccactttttaatggagttgtgttgtttggtttttgcttgtaaatttaagtttgttttttttttaatagattctggatattagacctctatcagaggcatagtttgcaaatattttctccatttctataggttgtctgtttacactgttgatagtttcttttgccctgcagaacttttcagtttacttatgtcccatatctcaatttttgtttccgttgcaattgcttttggcatctttgtcataaaatatttgccaaatcctgtgtccagaatgttatttcctaggagggcgtagagcaatcttacttctcccaggaaccacaactgtggcctctactggggctgtggctttggtgctggtctgctacagggcccaaggcttgtagaggtccccctggactccagaattacccctggaaaacgtccaggtggcactctgccccagtctaaaagcacagtggggaaatgcggtggggacaggaggattctcccattcccaatcttgcataggtccctgtggacagtgtgaatcccactaggaactctcattcactcatccttccccattttgtaggggtcctcctgaccgcactgatcccagacaggctggtgcccagattcattcctgtctgctctgtgtgttcccctgctgcctagatggatctcgacatggtttctcagatgatcggctttcagggtcagtgttcactaccccttttgtttcctctcggtgacgccagcatcaggagctccttttagtcggccatcttggccccacccttccaaaagttttaaaggttaacattatgcagttctgctgatggtgggtggaactgaggatcctataaataccccctgcagaatgtaaatggctacaaaactgtggagagcaatttggcaatatttatgaaaacctcaaagcttcattcccttcctcccttctacaaatccattttagatagtaactcatacacatatgcacaaaactgtgtaaagaaatactcctgtctgtaatgggccagagtaaataagcttaaatactcgtcaatgcaagaaaggtctattataccatggtacagccatacttaggaataccagacaagggttaaaaagagtaatacagatccacatatactgagatggaaggaatttaggacatatttttaaggaaaagctaacaaagtataaaacaatatgcatagtacaataacattacataggagaaaaactcatgaagcaaagtttttatatgtgaaggaggaaacatcaaatctatgaaagctctaacgttaaaaaggggagttgtggcataagaaagaagctgatgacatctcttggtttatatgtgttgtttgtgtttttaaataacaagaaattacttcggtacttttcttttcaaaaactagaaaagtcatgaaagcattgtcagcgaatgcaaccagtgtaattagccaaaattagttttaaaagattttcattgctgaaacatctagtcaaactatttaatgcatacattatagtgtcatcatgcatagaaaattaaaaatatctgcaaagcaattacgagaaacagtcattttcaccacatctttgttttggctgattcattttcaactgtgttacgctctgtgtcagttcttttatgaagattatctcattaaagcttcacaaaattctgaacggtatttgctatatagcctgtgctacagatgatcaaactgaatcacaagatgcgtgtgagattcgtgcgtggagttctgagttcaggaccacctgcaggaattcatagcccatattcattctcctacttcccaccacattgctatctttattttcctaataaacaagtatgacttactgtgaggaaatcataagacttttctccaaagagcccgttggcagttctaagcacatattcagtgtcagttctgttaattgcaacaagaagtgactgaaaacctcgatgaatatctccatcttcacctccgattttactaaaacaaagtgcctgaaaataaaataataataaacatacacattctcagtattctgtgacttcaaaatataggcacaacagtaaactcagcaatatgagatttacagatgcttactacagcttaaaagatgatcaatctaataaaagatagagagtgtctcaaaagcaaccctgtctgcctacctatgcctcttctcaggcctccctataagcacatcctaaaagaaacgaatgttatctaagtattggtataagtggatatgccttttgcaaaccctaacaaaatgactcggatttcttgtgctatttatgatacttggtctaaaaacgcagctgccaaccatgtgtcctgtctctgcatacacatggtgctcctgacactaaaagatggagtctctctccccttcttcagaatctgggctggccatgtgacttgctctaaacagcagaatgcagaactggctctgttccaggcttagcccttaaaaagtctggcagcttccactttccctctcttgtaacccagctaccgtggtcgagaagcccaagctatcctgaggaaagaccacataaaaggaggaatggaaagacttgcttttagggaactgagggcccatccactagccagaaccaaggcccagacatgggagtggtatcttcttggaccttctagcccagttgcctgcagaagcacccacatgagtgaccccaggtcaccaagaaggtcaggccttgaaaacatgctccatcctgccaattctccatccacatcctgtgagactaccagagttgtctgttaaaggaggaatttcttcaagacttgaaatgcagaagtccagctcaactgataaattgatagcacttattgttgtcttgaagtggctttacgtacctgagacatctgagctgcagtgtttccctttgcccccatgaaaaccatggccaaggctgatgatatgctcatggggaaaaaaataagttgtttgagttgttttcccctagctttttcaaaaggtttaatgcaaatgtgccatttgcttctgatagagcatccatgacggtgagcctgaaacaatggatttttaaacaatattacattaatataggcttacatgtgtatttacttttcactctatttattactagttaaatggatacttgttttcattgaacaaataggtacacacacaaaaactacactagggcttttcttacaagatgtcatttaatccatacaagctgatgagataggtgctgtgagtgaatgtctgcactttacaagcaaagaaattgagccttcactgtttaagtctgttgtgcctgagatcactcaactacaaacagaaaagtaggcattagaaaccaggtctttgtggttccagggctcctcttaatcaccatggtataaggacttataacaaaagcagcagcagaaagcaagaaagttacttgcttaaaacatgaaatttgtaagaagaaaaactattgatttccaatactggtaaaataggtattccctcctatattccatatgtgattatgtaaattgggacaccaatattttcagaatgtaacttagaattatgtttttataaatggaggaaggaacacagggatggtggaagggtaggaaaaagaaaatgccatgaaaatatttatacctttaggatgaaaaattccatttcaaagaatttcactaaagataaagctcacaagtactgataaaaacagtatgcagaaagatgactactgaagcactttatatgctacagagaaaatatttaaatacccagcaacagagaaccagttagattctgtcaaatccatgattgaatataaatggagatattaaaaattacagtcatgccatgagaaaatacaatattaagtataaattaagaataaagttacatgtaagtataattcatagataaaaagactacagggaaacactgaacatcacatatttctggataatgagtaattctaattttatttatatcctttgctattttccctacaatgtgcctatatttgtttcagtgtcatttggtcattgtgcttttatgaaattaatggacttgcatgtagttgaaaagtgagataacactaggcgagatcttttttttttattattatactttaagttttagggtacatgtgcacattgtgcaggttagttatgtatacatgtgccatgctggtgcactgcacccactaactcgtcatctagcattaggtatatctcccaatgctatccctcccccctccccccaccccaccacagtccccagagtgtgatattccccttcctgtgtccatgtgatctcattgttcaattcccacctatgagtgagaatatgcggtgtttggttttttgttcttgcgatagtttactgagaatgatgatttccaatttcatccatgtccctacaaaggacatgaactcatcattttttatggctgcatagtattccatggtgttacacaaatcaaccagtgtcttcccactactcccaactccccagaaacaatcaaacctcagtgcaccctttctcttggtatttactttcatatttagaaataattgtgtatatgactatttttaaccccacaatttttgacattataaattgccttcctaatttagggctgatgattaattttcttttatcacttaccgtacttcacttcctctctccaagtgtttgccttattgcaactaaatagtattcgctgctaagccaattaatcaataatggatgcattgctttttttacacgtctgtttttccttatgtcactattgccttcccctcttttcttgttttcactttttaaaaatctatggcaaattcccctcccaccaaatattctacagatcactcaagctctttcagtaatttcttcgtatgctcaaccacatatgagtaatctatcactcccatttctcatctcccacagtttcccccctgctggtgagtgcaggtttatttagtctgtaagattttcattcccgaagcagaattttcataaacagagaccaattttttctattattataccttatttagtcaatgatcaattaatttcttgttaatttggtaaacaaattgttttccttaaactttaaagcacactgctaacacagcaaatacgttattttaaacatttaagcattgatgataaagcttaatgtatcagatgttttaaagatgtaatcactttaaagaacaaacttacaaaattaacaactatatcattaatataagcatttgtacaataataaactgaataaatcaacacttataaatctgttaaactagattctcctaagactttcaaacatgttttaaaatacaactgtacaacagtgattataaaacttatttctagcaccccactagtaatactgtgggttttatacactttatcttcactctatctttttatatttcctagggtgatggtaatgcttattttaatacttttctggggttaaattaataacagaccatctcctattcacaaaaacaataggatggggctgccagattgatggttgggacatgggctctgactgacctgacactgctacttttcaggtcatactgctttttaaagccaaacaaatgtaaggaccattttgcagtttggtcctacattgggattttgcaaagacaactattaaatttgcttttttcttttttttgccaaatgcctttccttgaaatgaaatactatttgaggttctcgtgcatctgatttgcgtaacacagagttctatccacaagttggctttcaaagtctcctggtttaatatttaggccttcaggttggtttttgtaaccactgtgcctgtcaacctaggtgcctatagtcactattcctacacagcagtcgtatatgaatcatcacacagcatttcttgtaaacattattgaaattatttcctgcctcctttaaactcatcttacatttttttttttttttttttttttttttgagagagagtctcgctttgtccctcaggctggagtgcagtggtgcaatcttggctcactgcaacctctgcctcccaggttcaagcgattcttctgcctcagcctcctgagtagctgggactacaggcccatgtcacgacacccggctaatttttgtatttttagtagagactgggtttcaccatattggccaggctgggtctcgaactcctgacctcgtgatccgcccccctcagcctcccaaagtgctgggattacaagcgtgagccaccgcgcctggcccattttacttgttttatctgaatttactaaaggactctttcttggctttgttgtaacatgtatttcaattaacacatactggtttttgttcttgagcaaattctcacaacttgtctactgggaatctctgtaaattggttactttctttttacactatcccaacacttttgctttctggcaacagtatcgtcctcattttgatttttttccagtccaaaaacatactatcaacgcctctcctggtttcttttaaaggaggaggaatgtcaagtacaaattctgaatactgagagcatacgagtttcggtaccgacaaataaatactgctgctgttgtttttcctggaggtcccaggcctcacagccccttccagcgcccaacataaacttcaccagggtgggaggcctcattgtacctacttccagctcctgcaggatgagtggagcccaggcaggactttcaccccccagctccgcgcccattcgccctggcctcccctccccgcgcctggacccatagggcacacagccaagtgcagccaagtccctgcccttagggagcccccgccagagaccgagtaaagaaggacagccccagggaaaggtctggagcaacaggtgcgctgagcacttcactgggcttccctttcgtaggttcccgcccttttcctgggctcaagagcaggtaacgagaatccagcgtgtgggaaccactcccaaagtcaaacacctaccccagttctccgcgcagctgcccaggagcgaagtgagaccgggtcggagaggcgggaatagagatgtctgagcggcgccgggtgttagttcccagtgaatggggaggcgggcggagtcgggcccagccaggcaccacctagcgggcgcctgggcaggagggcggggcgggtcccgcaggccgcggggatctcctgatgtgcaggaagtggccaatctctgtgcaggaaaaagccccaactgtccgggagttttcagtcaagaagcgataatgcgctttggctatttggggtttttcgtggttccatacaaattatagggtttttttttttttctatttctgtgaagaatgacattggtattttgacagggattgcattcaatctgtaaattgtttgggtagcattgtacttttaacattaattattccaacctatgagcatgaatatgtgtccattttctgtgtgtgttcttcacgtttttctcatcagtgttttatagttgtccttgtagaagtctttcacttttttaattaaattgattactaggtattttatacatcatatttcttatagctattgtaagtgggatttctttcttgatttctttttcagactgttcactgtagatgtatagacatcttacggatattttatatccattttgtattctgcaactttactgattcatttattagttctagtagtttttgaggggagtcttttggtttttccgattataagattataccattggtgagcaaagctaattttacatctttctttccagtttgggtgtcctttgtttctttttcttgcctaattgccccggccaggacttccagtcagatactgaataaagggggtgaaactgggcatccttgtcttgttgcagatcttataggaaaggttttcaatttttccctgttcagtatgatattagcagtgggtttttcattttggtttttattgttttgaggtatattccttctatacccaatgtgttaaggatttttatcataaagagatgtaggattttactgaatacattttcggcatctattgaaatgatcgtgcagtttttgttgttggttcttttaatgcaatgtatcgcatttattgatttgtgtatattgaatcatacttgcatacccgggtgaatctcacttgatcgtggtgagtgatctgtttaatgtgttgttgaattggagttgctagcaattttttgaggattttttctgttttttacctgttgatatgacggattcctttgattttcaaatgttgaattagtcttgtacacctgcaatcaatcttacttagtcacaatgtataatccaactattttacacatagtttgatttcatttgtgaatatgttgttcaggattttgcatctatactaatgaaagatattggtctttaagtttcttttcttctaatgtctttgtctggttttgttatcagagtaattccagccctatagaatgaattagaaagtagttcttctgcttctatcttctgaaagagatttagagaattggagtaaacgttttcttaatttgtttttagaatttaccagtgagcccatctgggtctggtaattattttagatggctattaattattgattaatttttttagtagatataggcctattcaaatgttctatttcctgttgtgtgacttttggcggattgtgtctttcaagaaattgatccacgtcatgtagtttatcaaatttgtgggcacagagctgtcctgagtattcctttatgattttttactgtacatgggatttttagcatttcaccactttcatttctgatattagtaatttaggcttttctttttgcttagcctgggtacagtctcatcaattcattggtcttttaaaagaactagcttttgttttcattgattttctctctggatttcattcatttctgcccccatttttattatttattttgttttgcttagttgggtttaatttactcttctttttctgatattctaaagtagaagctgtggtgattgattttagagtttttaaaacaaagtttctacttttttaacgtatccattcaatgccatgaatttttctggagaggtgactcacattgtccagtaagccaagaaatgttaagtgtctaaatagtaaactctaaccttgggtgagtaaaatatgtaaactagtggattaaatcatctgtgattgaatccattacttaactcactacattagttaagcaaatatttaccttcactccttcatctttaacctcttcacttttttgaagtcttatctcataattatactcaatatcttatatatttttaaaaacacactttctttacttttgtatatgattttacttacataaatttcaagaaaatgcaaactgatctatagtgacatacggtagatcaatagttgcatggaacatggaactgaggagagggcagagagaggtgggtgagaggcagtataaaggagaaggggatactttggtgtgatggatatgttcactatctagtttgtttttacaggtgtgtacgtgtatatatacatatatccaaatgtatcagattatatattttatttttcattaattaattttttattactgttaattattatactttaagttctgtggtacatgtgcagaacgtgcaggtttgttacgtaggtatacacatgccatggtggtttactgtacccatcaatctgccatctacattaggtatttctcctaatgctaccctcccctagaccccacccccatgacaggccccagtgtgtgatgttcccctccctgtgtccatgtgttctcattgttcaactcccacttatgagtgagaacatgcggtgtttggttttctgttcttgtgttagtttcccgagaaagatggtttccagcttcatccatgtccctgcaaaggacatgaactgcatagtattccatggtgtatatgtgccacattttctttatccagtctatcattgatgggcatttgagttggttccaagtctttgctattgtgaacagtgccacaataatcatacgtgtgcttgtgtctttatagtagaatgatttataatcctttgggtatatacccagtaatgggattgctggatcatatggtatttctggttccagatccttgagaaattaccacaccgtcttccacaatggttgaactaatttacactcccaccaacagtgtaaaagtattcctatttctccacatcctctctagcacctgttgtttcctgactttttaatgatcgtcattccaactggcgtgagatggtatctcattgtggttttgatttgcatttctctaatgaccagcgatgatgagcttttttcatatgtttgtcggccgcataaatgtcttcttttaagaagtgtctgttcatatccttcgcccactttttttatggtgttgtttgtttttttcttgtaaatttgtttaagttcttcttacagtctggatattagccctttgtcagatggatagattgccaaaatttttctcccattctgtaggttgcctgttcattctgatgatagtttcttttgctgtgctgaagctggatcccatttgtcaattttggcttttgttgccattgcttttggtgttttattcatgacgtctttgcccatgcctatgtcctgaatggtattgcctaggttttcttctaggattttcatggttttaggtcttacatttaatactttattacatcttgagttaatttttgtataagatgtaaggaaggggtccagttgcggttttctgcatatggctagccagttttcccaacaccatttattaaatagggaatccttcccccattgcttgtttttgtcgggtttgtcaaaaatcagatggttatagatgtgtggtgttatttctgaggcttctgttctgttccattggtctatatacctgttttggcaccagtaccatgctgttttggttactgtagtcttgtagtatagtttgagtcaggtagcgtaatgcctccagctttgttctttttgcttaggattctcttggctatgtggggtcttttttggttccatatgaaatttaaagtagttttttttttctaattatgtgaagaaagtcgctggtagcttgataggggtagcattgaatctataaattactctgggcagaatggccactttcaccatattgattcttcctatccataagcatggaatgtttttccatttgtttgtgtcctctcttatttccttgagcagtggtttgtagttctccttgaagaggtccttcacattccttgtaagttgtattcctaggtattttattctctttatagcaattgtgaatgggagttcactcatgatttggctccctgtttgtctattattggtgtataggaatgcttgtgatttttgcacattgattttgtatcctgagactttgctgaagttgcttatcagcttaaggagatttggggctgagacaatggggttttctaaatatacaatcatgtcatctgcaaacagggacaatttgacttcctctcatcctatttgaatatgctttatttctttctcttgcctgattgccctggccagaacttccaataatatgttgaataggagtggtgagagagggcgtctttatcttgtgccggttttcaaagggaatgcatccagcttttgcccattcagtatgatattggctgtgggtttgtcataaatagctcttattattttgagatacattccatcaatacctagtttattgggagtcttcattttttataccttggaaataaaactctgccttttcatttttccattctccatcaagcttctttataaaaagtgtatacacagagtgttgtctttttttcacgtttgattctctcctgaccccactgcagttagaagtccaccccaacattctctgaaattcctcctgataaggtcaccactccttttttaactgccatggccaaaagatatgccccaggtcttatctcattttgtttgttggcgacttttagccctttcgtcttcggccttccaaggatgtccagagcccccttcctttcttgctcaactcctttgcttggtatccagattcctttcctccgattctaggccctctacttttcattataatatgatcttgtcatgatcttatcatatgaccttaactgtatccataggttcaagtttcaaattttacttatttcatccatatcgctaacctggctcttgagctgtagacgttacctgtgtacaactggacatctctcagaaacctgggtgcatcccttcccagttctcttcactaaatgatcaacagctctgccatttctatcaccttcatgctgcttcaatccgctctttctcaccttcatcacatctcatttctctcctaaacaattaatttccttacagttctcctgatcaatgccaacttattctctcctctgaagtccatataaacattcctggactttttttggttggtaggctgttaattatagcctcaatttcagagcctgttattggtctattcagggattcgtcttcttcctggtttagtcttgggagggtgtatgtgtccaggaatgtatccatttcttctagattttctagtttatttgcgtagaggtgtttataatattctcttatggtggtttgtatttctgtgggatcagcggtgatatcccctctatcattttttattgcatctatttgattcttctctcttttcttctttatttgtcttgctagtggactatcagttttgttgatcttttcaaaaaaccagctcctggattcatttattttttgaagggttttttgtgtctctatttccttcagttctgctctgatcttagttatttcttgccttctgctagcttttgaatgtgtttgctcttgcttctctagttcttttaattgtgattttagggtgtcaattttagatctttcctgctttctcttgtgggcatttagtgctataaatttccctctacacactgctttaaatgtgtgccagagattctggtatgttgtgtctttgctctcattggtttgaaagaacctatttatttctgctttcatttcattatgcacccagatgtcattcaggagcaggttgttctgtttccatgtagttgagcagttttgagtgagtttcttaatcctgagttctagtttgattgcactgtggtctgagagacagtttgttataatttccgttcttttacatttgctgaggagtgctttatttccaactatgtggtcaattttggaataattgtgatgtggtgctgagaagaatgtatattctgttgatttggggtggagagttctgtagatgtgtattaggtctgcttggtgcagagctgagttcaattcctggatatccttgttaactttctgtctcgttgatctgtctaatattgacagtggggtgttaaatctctcattattattgtgtgggagtctaagtctctttgtaggtctctaaggacttgctttatgaatctggttgctcctgtattgggtgcatatatatttaggataggtagctcctcttgttgaattgatccctttaccattatgtaatggccttctttgtctcttctgatctttgttggtataaagtctcttttatcagagactaggattgcaaccccttctttttttgttgttttgcatttgcttggtagatcttcctccatccctttattttgagcctatgtgtgtctctgcacatgagatggctcttctgaatatagcacactgatgggtcttcactctttatacaatttgctagtctgtgtcttttaattggagcatttagcccatttacatttaaggttaatactgttatgtgttaatttgatcctgtcattataatattagctggttattttgctcgttagttgatgcagttccttcctagcatcgatggtctttacaatttggcatgtttttgcagtggctggtactggttgttcctttccatgtttagtgcttccttcaggagctcttgtaaggcaggcctggtggtgacaaaatcgctcagcatttgcttgtctgtaaaggattttatttctcctgcacttatgaagcttagtttggctggatatgaaattctgggttgaaaattattttctttaagaatgttgactattggcccctactctcttctggcttgtagagtttctgccaagagatccactgttagtctgatgggcttccctttgtgggtaacccaacctttctctctggctgcccttaacatgttttccttcatttctactttggtgaatctgacaattatgtgtcttggagttgctcttctcgaggagtatatttgtggcattctctgtatttcctgaatttgattgttggcctgccttactaggttggggaagttctcctggataatatcctgcagagtgttttccaacttggttccattttccctgtcactttcaggtacaccaatcagatgtagatttggtcttttcacatagtcctatatttcttggaggctttgtttgttttttcttactgtttttttctctaaacttctcttttcgcttcatttcattcattcgatcttcaatcacttataccctttcttccagttgatcaaatcagcatgtctcatgtgcatgtgtcatgtagttctcgtgccatgattttcagctccattaggtcatttaaggttttctctacgctgtttattctagttagccattcgtctaatcttttttcaaggtttttagcttctttgcgatgggtttgaacatcctcctttagctcggagaagtttgttattaccgatcatctgaagccttcttctctcaacttatcaaagtcattctccgtccagctttgttcccttgctggcgaggagctgcattcctttggaggaggagaggcactctaatttttagaattttcagcttttctgctctggtttctccctatctttgtggttttagctacctttggtctttgatgatggtgacgtgcaggtggggttttggtatagatgtcctttctctttgttagttttccttctaacagtgaagaccctcagctgcaggtctgttggagtttgctggaggtccactccagaccctgtttgcctgggtaccaccagcagaggctgccaaacagcaaatattgcagaatggcaaatgctgctgcctgatcttgcctctgaaagctttatctcagacgggcaccaggccgtatgaggtgtcagttggcctctactgggaggtgcctcccagttaggctatttgggggtcaggaatccacttgaggaggcagtctgtccgttctcagatctcaaactccgtgttgggagaaccactactctcttcaaagctgtcagacagggatgtttaagtctgcagaagtttctgctgcctttggtcatctatgccctgcccctagaggtggagtctacagagacagtcagggctccttgagctgtggtgggctcttcccagtttgagcctcctggtggctttgtttacctactcaagcctcagcaatggcgggcacccctcccccagcctcgctaccaccttgcagttcgatttcagactgctgtgctagcagcgagcgagcctccgtgggcgtgggaccctcccagccagtcgcgggatataatctcctagtgtgccatttgctaagaccattggaaaagtgcagtattaggatgggagtgacccgattttccaggtgccgtctgtcacggcttcccttcgctaggaaagggaattccctgaccaattgcgcttcccgggtgaggtgatgccccaccctgcttcagcttacactcagtgggctgcaccaactgtcctgcacccactgtccaacaagcctcagtgagataaacccggaacctcagttgaaaatgcagaaatcacccatcttctgcgttgctcacactaggagctgtagactggagctgttcctattggaccatcttggaacctgatctctcctaagttttgtatttttagtagagacaggatttcaccatgttggccaggatagtcccagtctcttgaccttatgatccaccggcctccgcctcccaaagtgctaggattacaggcgtgagccaccacgcccggccatatcttcaaatatcttagtggagggtcaagcactgaggctggaacatatttttccggcactcagcaacccgcgcctcagcttacagactggtaacaaccaagtggtgaagaagattccttatctctagtggcttccattccctgaactgaaataatagatctggaattgaagaaaattcacagtccatcaatataaatgcaaattgcttttttctcccccttactggaatttcatggaaaaattctgaataaatttacctctcttgttcccacagttaagaatcaaaataattcatttttgagggaaaaggactcagcgatgcaatgaggccatgctctcctgctccactctagatttctattgtactgttttaaaaagagaaaaagtgattgtattttttaaaccacagaccatcttttacatttagatgcttagatattttctaaatccttcccacatcaaatcttttcatcccttgaaatcccttcttcactaaataacagatttgggaaacaatataaacaaaccatttactagttgtccataacttcatttattagacttttattgtactgagcactgcaacaggccataaggaagttaagaaacaaaactaaataaaatatgtaccttctttttctctgtatctcagatgaatgggaaagacaggttgtatttacaatagagtggacagcggctttaatggagcaggtacacagcagtatgagaagagaatgatggaaacagtagctctggggactcgggatgaatccacagtagcatcagttgagctgagtcttataggatgagtgactttacagatggagagtggagaaagaatatgttaggcagaggggacatatttctaaaatgtggttgtgaaaaggtttgtagcaggtgaagaatgggtatattttgagggtggaatagaaggtgtatactggggagtggcacatggtgacattgaatgaggttaaacccagtcagtaggtcaagcctaaactgggaaagattctgtctgccattctaagaaatttggggcttattctttgagcaatggaaaaccattatagaattaaaaccagagaagtggcatgatagcatgatttattgatttctctctctctctctctctctctctctctgtctgatttactttaatgtcaatacttaggatgcaggaggagaccagaacacaagtattttgggcctaagcccagacagtagcaatgctgatgccaagaactcattagaaggcagaaatcacaggagctaatgaccaagttgattttgggggagagggcaaggaaaagagcagattactatatgacttagttttttaacatacataattggataaatgatgaccttattaacagagagagagaacatgggggaaaggagtagcaagtaaaaatgcctggagacagaagtaaagactaaggtatggtgtagaggcaccaagtgaagtttcaggagacacaaattggcccaggcagcttgcatataaataagacattggtctgatgaaagaagcctagatgatgatgccaatatttatttatttagagataaggtctttactctgtcttccaggctggagtgcagtggcacaatcatggctcactgcagctttggcctcctaggcctaagtgatcctccctcagcctcccaagtagctgggatcacagacatgcaccaccatgcccggctaatttttttttctcttttgtagaaacagggtctcactatgttgcccaggctgatctcgaactcctgagctcaagtgaccctctcacctttgcctcccaaagcgctgtgattacagaaatgagccaccatgcccagcaatgccaacatttaagatatggcttcagaaagaggacatagcaaaaaggatgtaaaaaaggagaagatacagaaaaagctggacaaagaaaaagaaggaggtgaatagtatcagcagctgctactagaggaaaaggaacattaagattgaagagagcccacttctcagtattctagtcactacttaccttagtgagagtggttgggataaaaatcaaattgaattggattgagatataaaagggaaaagtgaagacagccctcttcgactactctttcaagaaacctagccatgaggaactagagagagcatgctgtctacagatagagggaagctggaacaaggatagtatggtaaggacagatggcatttaccatgtcttttgccttctgaatagtgacaatcagaaacagcaggtaaaatactaggaaattagggaaatgtctttgatttaatgaaaaacctaaactaaaaaatgtatggaagagctgtttaaaaaggaggatggacaggacctggggattgttgggtaggagggtgaggaaggagaaagctggcatgatttgcagacttactgacttggacaagccagtgaatggcagtgacaataagcaagattggggaaagaggaaactgaatgaatttggaagagaaaatgaatttcgtttttgacatgctaaggttatatggaaagttcaggggaagtttgggaggcaaaggtgggaaggtcacttgagctcaggagttcgagaccagcctgggcaacataatgagaccctttctctacaaaaaaagaaaaacttagtggtcctcaacaaatcgatatagttgaagtcacgggaatggagtgattttccagagggtgtgtgaagagcaccagagccctaagaacgccaaaatataagcggtcaatagaggaaaaaagagccggaaaaagacacttggagaggcaagagaggcacagaaagaactagataagagagttactgaaaacaacaaataaaaggaggaggacttttgaggaggaagttgagcagtttggggagatgaccaggttcatatgggatcacagagtgcgtggccatagttaagtggcagtaaaagtgcctagattcgagcgggtgtagaatcttgtgggttgggggttagccgggcatctaggaggaactgaaagctgcagatgtgtggacagatgatggtgcaggtaccgatgaccaggaagctgagccagatgctgaaatccggagatgtcgggaaaagagcagaggctcccgtgtggtctaggagctgagtcagacggagaaatacagggctcggagtccctgaaagcaacaggtaacttcaggccctaaaattggggtagccccggcctcgaggtgatccctgcccgagcctagcgccggttctgggccagggcgcgtggggtgtaggagcgatgggcgatgctacccgtagctccgtggagagatggggccggtgaggcatccctagggccaccccccccaactccggggggagttccttcctccgggctctgagcaccagctgatggcgccctcctgagcccctgcctccccacaccgtgttcttcccttccgcgctggccttgcgggaactcgcccgcccctgctagggctccgccccacgggcgggcgtgggtttcactcggaagctctttcaaggattctgcgccgcttgcattctgcggggctggactgtgtccgcggaagctcccccatcccctcagccaaccccgtgctcccctcgagggctcctgggaggactgggctggcagcgggggtgggggtcgcaggatgccacgggtaggaggtcatggggggaggatcaccttgatgtccacttcggggagagaagggtcaagggacacctcatacaccctaaggagaccatctattagttcccgccatagcgccccgtttatttccgtcacctcaccgatcaccagctgtaattagaattaggtgttgttgaggctgtggttcgctctccacgtccactagaattagtcaccaggaggtggcagccgaggcacaccgcgttcccccgccctggaccagtgttagaggcgacgcgctcaggccttctgtgcggacccggaaggaaggtcaggtcccggggcccgaaaggaagaggcagcagccgaggatgggcgcccaggaggaggaacgcgcgctgcattgcgctcgccggggctaaggctactgggggatgttggtattgagtatttcagccatccataatgactatttttaataaaacttttattttggaataattttagattggcagaagaggtgcagagataatacagctaattctcatatactccagtacagtttccccaagtgataacatcttacatgaccacggtacatttgtcaaaactagggcaccaacattgagagattgctactagctaaacttcaaatttcatttggatttcactagttttttaagctaatgtcaatataagtattttttaaatagcagaaaaagggggtttagttttacaatcataatttatgagccaccaaaatgatttttttgagacagggtcttgctctgttgcccaggttggagtgcactggtgtgatcttggctcactgcagtctcaacctcctgggctcaagcaatcctcctacctcagcctcctgagtagctgggaacacaggtgtgcaccaccatgccgggctaaatttttgatcaaaacgatattcttgagaaacttcgtatttatcaatatcatatcacctttataaagattttttttcatttaatgatgtatatgaaatatatgataacaagtatcatggatattgcaagtgatcaagaattgtaaggcattcagttgaatagcatagtacttctgaaatgtaaatgaaagtcacattagacaaaatgttaacgtgttaaaaataaaaatcgtcattctctccagctgtacagatatgacaggatcattagaatgcattatttcaagatctatttacttatattcaaatgtgaaaaaggtttagactacaaactcatttttgtgaaaattgtgcttttccatctcatggtacatttttcacactgtaagatggtcttgtttgttgagagatatgcaggatttagggggagcataatcttccataaaaaagaatggtgttggttttattgtgcctgatgaagaagaggaatgggtgatttgcattgaattcaatggatgggactctaattcgtatatctatctcactcccactgccagctgcagcttcagtaccttgttcatttatttccacaaaagccttatggaaaacattggacaaaaataggtttcttgctgaagacattcctgagaaatcagctttgctttggctgaaggcatcactcatccccatactgctcagggttgacttgagatcataactgtcttccagcttgaacttgggaaggtgtagctgcacttcatacaactccatcatgtctgcactggtccactcattcagcttctcataggtgatggccttttccagctgcccatttggaaccaagaaagaaagaatcagtaattccaaagtggaatgaaaaacagtagtagagagagcaccctaggaattacataggtgaattatgatcactcccgttactcagggtgacaacacacacacatctggaaatcctttggttgtataatgttgaccttcaaaatcttttttctgcagattaattgctatccggatataggagaattagagtttaattcaaaatggaagtacttctgctatagtatgcaagacatcagctatacactcaaatacataaatataaatatacatttagatgttagctatacaaaagcatatactctagttcaagttgtaagccaaggcacaaaaaatcttgtttatcgacatgggagacagctctttgaaaacaaaatttggaataaaataacacgtctatactctaatcaaaacaacgtgagattaataattatagactaagtcaaaaaagaaagaaaagataagaaaatcctccaaatctcaaaagtaattattttacagtgagtttaaggatgctttccctcttctttatattttaaatttttaaaatgataaatatgtgtgtacatactatatatgtatgtatatatgtatacacaaaaatacacatgtatatatgtgtatgtttgattttttaaatgacatgtttgtttcaatgcacatgcatcttctagacggaagttttggctgtcaatggcatacctgtaaaccacttacattaataacatgacagtatcttcagctcaggaaattgcacttctaggtttttatcttacaagggcttgactctgatggttatttgtccacactggttttattttcactatcctctccagtttcacaaattttatgacataatttggaaatattttctacatactagtaggatcgtggaaacttataagtattaagagtctgacacatgtgaaaaattagacactgtgcaaagaaagacaggccatccgttgcatgagactcatgttcagtttaggcatgcaaattgctcagaagcatggtttgacaaaagttgtgtccagattaatagaaatttagctggtaaacagcttaaataattcttgaagacagtattgatagagactttttctcctgaaggttgcttacttgataaaaatgttcaacaaataagttttggaagacagcttgatgtctgcgtacgtgtgtgtatactaatgcaaatggttaaaatattaatttcaaattacataggtcctaccagagggaaacctagactgattgaaagatggttatgatcaatccacacagttctgtgactgacaaggtatatttgtatctgttgtacatgtggctatgtgtgtgatgaccccaggctggagctgtctatacattgcctaacctttctgaggaacagtaatattaattttatagagtgttgtaaagtttaaataatattgtgaaggtgaagtgcctagaactcaaggtacattggtgacctttctctataggaattaaataatagggagaacattcttcctgaggacaattgggagaaaccttggccaagcagttcttttcttagtaaacactggaaacaccctcacatcagacacactgatgttatttacctgttccagcccattaatgtcttctggcagtagtataagcaggctgaggtcacggcttttgtagtagagttgaaggcccactgcttttggcttttctatgtgaaaaatgtgaagctttttcttcataaacatcatttgcactggtttgcttgtagtctaaaaaaaacaaaaaaacaaaaaaacaggtaacaaggtcattgaagaagtattccaccaactccttaattctctaccatcattggctctcagttggtgaaactgctgattctctgctaaatgctgaataaagttatccagaccttcctttatggaaatatctgttccactgaaaagtatcagcaatagcagatatgtgagtttctgtttctaacacaactgtggtaacctctataaatcacagcaacatgtattaaatcaaactcttaatatatgatcttgtgtaaattatggaaatgccaataatccaaaaactaaggcatattttacatgaaaatatttgagaaagtattttagtattataaaaattgacattcactaagagaatgcaaagacaagcaatagatgagcagaaaatatttgcaaaacatatgagacaataataaaaactcaacaaataataaaaaactcaacaataataaaacaaacaagccagtttttttaaatgggcaaaagatctaagaaacacctcacaaagaagacatacagcacatgaaaaatgctcaatcatatgtcactaggagattgcaaattacacacggctactagaatggctaaaatatgaaacaccaaatgctgatgacgatatgaagcaacaggcactgtcatttactgctggtgggaacgcacagtggcacagccactttggaaaacagttgggcagtttcttactaagttaacctagtcttaccatacgatgcagcaattttgttcctaggtatttactcaaatgtgttaaaaacagtgtccacagaaaaacttgcacacagtgtagtatttatagaaaatttatttattattgccaaaaaattggaaggaactaagatgtgcttcaataggtgaatggataacaaactgtggtacaattgtaccctaaaatattattcagcaataaaacaaaaagatctatgaagccctgaaaagacatggaggaacattaaattcatagtgaatggagccagcctcaaaaggctacgtgctgtatgattacaactatgtgacattccagaaaagccaaaactgtacaaacaaaaaaagatcagtggctgtcaggaatttagggggaatggatgaacaggtgaagcacagggcacttttagggcagtgaaactattcggaatgccattgtaatggtggctacgtgatattgaacattttcaaaatccatagaacagtacgacatcaagagtggacccaaatgtaaactatagattttagttaataatgatgtatcaatattagatcatcaattataacaaatatgccacactaatgtaagatgttaataataagggaaactgtgtgtgtgttgaaggggggacatatgagagccctatacttcgtgcacaattttttctgtaaacctaaaaatgctctaaaaatcaagcttattttaaaataagaccaattgccaaatgaaagcaaaacaaacacccctgcaacttagatgttgtagacatatgttacttttccattctatgtcgaactgtccatcacaaagccccaccgtatgctaaggtggtggccatacgacaagggctgtttactgcgagtactctagcatactgtggttgtttccagagagagcccatgaatggccccaaccagagccctcctagaactaatggcaaacactttctctgtctactagagttgtgaagcttgaagactgaatatgaggcaaccatattccctgatcattattagagaatgagagtagcaactagagagaaaaagagaaagagacagcatgagaatgaactgggattatgtgttgagactctaaataccccaatcactcactcctggacttcccagttttttgtcacctcagctaattccaatacaattcccattttgccagagctagtttggaatacttttctatcacttgcaactgaaaatttccagtgaatatataaacccaggtaactatccagctcacttctcgttaacctactcgattatgtgttcagtttcttccccatcctcaccccactgattgagaaatacctattgcacacattcattacagccatagttccacagcagcactgtttgtggcctatccctagactaaacaactgtaacaaaccttggtcaactagttcctttcctaggaatttggaatgggattttccaactggtctctagaatggcgaaggtcctgtgagtagctatgttctgtcattgtgaactgggaagcagagaaagttagaccacaacctgagagaagaatgatggtagaaacagagttgaaaaaatagaggaaaaaatcctgacaacatttaagtccttgtttccaatcaggtcccaaggcccagccacactccagcccattcacgagagtcaaatctataaccttactttagtctccgtttttattgcttttacttatgtaagggtaagaatttggtagaagttagtactggtttagcagggcaggtacagaacttgcagtgaggactcttaggttcttgttctggactgcttctaactcaggcatgcctccagtcacttggccacactatatttctcacctaggaaataaatatgaaaactaccctacccagctcacatgggtggcaaggactgagtgattgtatattcagagaaaaacaaggacaggggaatttcaatgaattccccatggaactgaaggcttttagcccagccatcacttccctggtaagagccatagttcactagaacatacaatctaaaagaagagtgaatttataagagaatcacttgtacaatgccatgtgaaaatccaaactgatctttaaaaatttcctacctcgtttattctaaaaggcttttctgtggtgttttgcactaagaattgatgttcccagattcctttaaagtatagggcgttcaccagaatcatcctggttgtggaatccacagagtcatcaggcaggagattctggattttacctgtaagaacaaaaggcaggttgcaaatgacttgtagaaatatgtatattttaactaaagacaaccaggcttctattttttctggaactataaaagttttctttaaaagtcctggtttttgattttctggaaatggacaagtactattcctgtccagaatagctgctgtgaataaatagagaaaccctgaagaaataatatgctggactgtcagtaagcattattccacataaagtacaaatttgcattttaattataaaaacttcccctgtaatataagttgatttgtaaccctatttctttttttttttctttttttttttttttttttttgcactttagctaaagattttttatttccagaaaattacaactacttgactaacaactctttgcacctactgatttaggttggacaacttgtgaaacatagtggtaaaaaaggaagttacttctctacttcaaggtttccaagtttgtggtagaaatctaaagaacatttctcttagcactacctctgggcaagacacttgtgctaaaatgtccttctttttctctgtacctttatgtcattgttaatatactctattcatcatttcccctcctctactcttagagtataaatcaagatctgtaaaattgctcaataacaggacttaactgccctggaggaagaagggatttttctttcctttttgaaattcagagggggaaatggtggcatttaaactcctgccaaaaattttaaatttctaacaaaaaaataaaataaatactcttgaaaattaattcatatgaacctttattttccttcaaaatttactggctaaaatgatggaaaaaatatatacatatatggagattaagagaaggtggaaagctgtaattagatagggtttgagttttggcatttcgtatttgtagctgttggaaagactttctgtaggactctctggtatttggtatccaagagatactatggttcatatgttcatccataatcatactaaaaagagccaagtattgtcacaaaagcaggtttagctgggtttctcaccaaattgatttacatgtgaacatggaaagttcacataggccaccgaaattgaaatctccttaagctgataagcaacttcagcaaagtctcaggatacaaaatcaatgtacaaaaatcacaagcattcttatacaccaacaacagacagagagccaaatcatgagtgaactcccattcacaattgcttcaaagagaataaaatacctaggaatccaacttacaagggatgtcaaggacctcttcaaggagaactacaaaccactgctcaaggaaataaaagaggatacaaacaaatggaagaacattccatgttcatgggtaggaagaatcaatatcgtgaaaacggccatactgcccaaggtcatttacagattcaatgtacccctatttcgaggggactgaaatacttccttctttttcacttctgtttgtagccacaaaccgtgagctgctctgattaacatctgaaaggctgtgccaatgttttcctgttgcccaaaatattgctgtgtgcaattgcttctatcagattgttatagttacaacttaaactgatatttggctatcaaccccgaatacattctctccaaactctctaataaagcaccttgaacgatttataaaaatacgtcaaatatatatctctcgaatcaggtattataggtttgctctagaggcacatgtttgataactgaggtagataaaactggattgagaagaacctcttggccaacccagcttcactctgcaacagacactactcagaataaagggctgatcttttaaactgccaaaatacagacatgggaaagctgttaaaattataagagacagcttgtggcacttaatttaaaaaataattttccaggaaaatattaatagaatctcaagaaatagtagaaaatctgaaaaaaaaaattaaacatgattgaaattgtacaactctcaagtgattacctcccgtggaaacagcatcaaccccagacatgtagacaggtaaattcttttcaaccttcaagtaaatgataaatctctacactattcacacagtttcaaagcacaaaatagaaaattctctgattacttttatcaagctggcatatacctagtataaaaatgacaagaaagcacaaaattcccatccgtaaacccatttctcttgtgaatataaacaaataaaccctaaattacattttagtaaataaatctaattaatatacattaggaatgcaagaaacatatattaggaaatctattaatgtagcgtgttgctgatatggcctgaatatttgtgttcctccaaaattcatatgttggaacctaatgttcagtgtgatagaattaacaggtggggcctttgggaaaggattagatcaggagggctctgccctcatgaataggattagtgtccttataaagggattgagggagcctgtttgccccctccactatgcgatgacaatgagaaggtgtcactttggaattatagagcaagccctagccacacgctgaatctcctggcaccttgattgcggacttcccagcttccagaactgtgagcaataagtttctattatttataaattacccagtctaagatgttttgttatagcagctgtaatggactgagacaattgctttgatgactgataggagaaaaaaatcataacatcatctgcaaaaatacttttgatcttcaaccttcatttctaattttaaaatgatgactttgtaaattaaaatgaagaatacttctataatattaaaaataatatttttcttcaacaaacccataataatgctaacccagaaacattagtggtatttatggcctgctatgactaccgttatttaacatttttctcaaaatactaagtaaagcaaaaaaaattagatgagatattatataaaaggaagagacaaaattattatttgcaaataataaggaaagcaaggagaatcaactaaaaaacaatgaaaactaataagaagttcttttaggtgctgttacactattacgctaaaaattatcttcttatcttctaacaataaacaattagaaaatgatataattacatcctagtgatacaccacatttataataattatgcttataaaatatcgttaaatgataacatcttatatacaccactgatccaaaccaggaagttaacattaatagaatattattgacaggtcatatttaaatttcatcagttgttttaataaagtcctttttttctgggttagaaatcaatacaagaccttgcattgcatttagttgtaatttcttcttattcacctttaatctggaacagtttctcactcattcattgtctttcatgaccttgacacctttaagaaagacttcccatttgttttgtagaattctcctgaattcccagtttaggtttgtctgatgtttaatcaagataatattcaagttatgcatttttgtgagagtatcaccgtggtgggttgaattgtgtcccccaaaaacacgtgtcccacttctaacccgtagtacctgtgaatgtgaccttatttagaaatacagtctctgcagatgaaataaagttaagatgatgtcatattggattaagatgagacccaatccaatgactagtctctctgtaaggaggggggaatttggacataggcacacagagaggagagtgccatgagaagatgcagagacaaaagagagatacagataacaagaggaagccatgtaccaatgcaggcaaagattggagtgatgtatcaaccagcaaatgagtgccaaggattgtcaacaaccaccagaagctatgagaagctcatgcaagtttcttctctagagccttcagagagagcacagccttgctgacagcctgattttgacacctagcccccaaaactgtgagagaatacatttttgttgtttttaaccactctgtttaccttaatgtgttatgacagccctaggaaactaatacaaccaccaaagtcatgctgtgttcttctcaatgaatcatgtaaggaagtgcatgatgcccatatgtcctattactggtgctattaatttgaacactgatttttccaatgtaaaaggactggctggtttttttattttttggtatcatgtgggaagatactttgagagcgtgtaatatactgtttcccatcaggcttttgtccactaattttagtatccatttataattcctatctaaaacaattactactgtggttactattgcagatggtggttttctatttccatcattcttctgtattcattaactagaattctactgtgagaaagagccttcccttcttcatttatttggatcttcaattatttgtttatatcagtatagactcatgggtatttgctttattctatgggttataatccattactatcattatttattttgttgcacaaattgctccagattttgccattggaagcctcccctgttggctcctgtgtccttctgacatattcccaagtattttgagcatttccttacattctgtcaccacaaaaagtccttatcttgtacttcgcttgtcccagctctgttaccaccattttgtccaggagctctgctttctttttttgaaagaaggagtttagaaatcaagatcttggtgtcagatatgctgattactattgggatgtcattgcttccagatcctctcagcaaatggggctaggaaatgtatgcattatacacacatctgtatctatttctacatttatctattgaattatctttctgcatatatattgaagactagaaggtcatattgatactttcacttccaatctaataacacagggttcattgtagcctttgtctttgccttatttgtaatttctttcctggctctcatgatttccaatatatttccttatttgcttaatcctagaacaaacataaagtagtttcaggattgcaaatccattcttctattaaaaacaaatttaaaaactagggtacaatatttgtgtactttttttcagccttaaaatatatagtcgaagtatacttactttttttaacctttgaatttatagtcaaaaatgctgttttacaaggttacttaatgttttagaccatatgtttgtttaaaagaatgtatctgaaacagtgctacaagccaagatgccattaaaggactggaaaatttgaagaatcccgaaagagaaaatacagatggattagactgatagaggaaaccacagcatacacacacaaaaggcatcacagatgacttcttgggaggttggggtgttgctcagggagttagaaactcaaaggccatgcattcatggagcttgttgggggcagggaacatcaatgagattaatgaaataattccatttattgcagtggctcctaactttgactgcaaattagaatctcctaaagacttaaaaatactgatgcttgaacctcactccaagatatctgaattagttcatctggagttaggcttagtaatcagtaaattcaacttaatatgttgccacgattgagagtcactggtttacagaaattgtgtttattgactttctatacctccctgggtaccagcaatgggctacaaatacattagcttactgtctagagtaatgagtgaggacaccgtatgtcacagtggagttagaacagtgttcccaaggtactaggaattcccaagaaaaactggaagcttcccatgcccagaaggcaagttataaacacagcacatcaatgtcagcccaacagtgaccatagacaagttattgtgatctgtattataaactaagactggtaaactggggttgccaaacaaaaagacacacagaaaaccaaaaggattaaacaggtaagaaaatccaccctatgagagagaagcaccaacatcaacatgcactaacaggaagcagaattaatacagcaaaaagacttaacaataagcaaaatttacatattcagataaagctgactgatctcactaatgaaaaagaaataaccaataaccataagtcttgaagagaaaagaaataattgtcaaagcagaacataaataactgtggaaataaaactcttgaatagattgaatagttgaatgaacacggctggagcaaaatcatgagctggaaaaggataacagaagggcaaagcaatgaaaagtaaatataaatggaaaatcaaagcagataggatagattgtgaaatttcaaatttgtttattggaagtactgaaggcaagaagggacagaatgaaaagaagaaaatcatgaaataaataatatacacaattttgtttacctaaaggcctttatatttagatttaaaataattgccagcaaagttataaagtaaataaataaaaataaaaagacccctgcctagaacataatgttgaaacctcagaacaccaaaaaagaaagaaaagtattctaaaatcttctggggaaggttcagggtgcaggtttgttgtttttattaatgatgctggatgctactatctaaaggaaaaatgaagaccttggagaagacctgttgaaagctcaaggaaagaagtatctttgagatggttcaggtagaacctagtcatgccaaacaccctagcaactttcccttaaggacatatggaactctgcctcgacaaagtctaagttcctaaagaactaattaattccttattttatttcatgttgaatgcttccaaatagcagatatttgtgtccaaaatagaaaatacagaaggattacagctttctccctctctagacaattctgcttctgaggaatagagtgaggagggtcagctggaggaatgaaataatgggatgtggcaaacaaggctcagtaaaagattcgaactgtgtccaccatgagcttattttttggtgcctctcatttgaccagtggtatgataacagagaattttttaaagtgtggatgtcccatactgtggccatatagtactataatctgcagtctgccctttcttccataaagtaaaagattatttgctcagttcttcttcagaaatgaaacttctagacttatgctgtctaatatggtggccactagattcatgtggctacttaaatttaaattaattgaaatgaaataagttgaaaattcagttcctcagtcacattagccacatttcaaatcacctgtgttcacatgggaatagaagctactccactggacagtgcagatttatggcacgtctccatcatcataggaagatcttctggacagctctgtaattcagacacctggattttgttatgtaccttcagtgcatcagtctaagctggggaagcaaggcaaggaccaacccctaagagccatgcttatgtggcaccagacaacagctgtcttcttggctctaaaaggctttgctaggatgcaaagctagcagattagttgccccagccttccaatctgtaggatccctacctgatttccaaacaacataacagtcaccaggctaggaatctaaaacataacattttgcccaactccagcaccattcttactcctactcctgctatgttgaatttgtgtttaggtcataacatacttttgcctgtacaatcaaatgtggtctcacacaagctacaaaagtgcaagtacctagtaactgctagctaatctataattctaaacaaaacaaacttattttcagatgaatgcaaatcaggatggaagttaaatatcatagtcactcttgcttcaccacggcttttgagtcatgggctcaatgtaaaaggaactgagcacttagggctgccaatgttatcaattcattccaataaacattaatgttctgaacaactgctatgagcaggcctgttatgtgtgatttaaagctgaaggcagagttatcgcacatagagtccttgagtctagcaggggacatagatgtataagcagttagttttgataacagtgtggtaagtgtcaaagtaaaggtttgcataaatgtctgggagcaaagaggaggatgtttagcctgaatgaagccctgagaagttatttgggggcaaaggaggggttgggggtggcagggcagggaacggtatttcagatagaagaaatggcatgagtacagggccattaagtagcacagggagttcgggacattctatgacatgtagaatccctgaaatataaattttaatccagggagtgacaagagtttaagtggagagacaagcaaggctccacagtcatattggcccttatatgtctggttaagggctttgtgttgatcccataggtcatggggcagcatgaatcagttttgagtagagaagtgacatggcctatctggtctaatttgtattctaggcaggtaatttgggtggtaaatggaggttagatttgaagggaacaagattagaggcaaattattgcatttgtgcagataaaagatagcgaagaaagaaaggctgtggaggaaaggaattgaaaggaaaagtgaactctgagaaacatttaagaggaagcaaaggaaagggtagaatttagagtatgtctcagattactgttctgggcaagtgtgtgattggtgtacccattcaccaagatggagaatacatgaggaagacaaggtctgggaaaaccattctgaatgtgatttcaggtatggtgaatttgaaatgcatgggacccttggcaaatatgtctagcaggtgtttgttatgagccctgaagttcagaggagaagacagcaaaacattccatgtgcttagacacaaagcaaaggcatcatcattaatattcattaatgacaaatgatgaattaatcttcattaagtcatgaatgattaattaatcttcattaatgatgatgcctttgtatttcttcataagatataatataatatcaattgaaatgttgatttaaaaggcagcatggtgtgtagaaatcagttcagactttggaactaagctgacttatattcgaaatcctattgctgaagctttaaactgtgtaatcatagataatttctctaaaatctcagcttccttatttctaaaataaggataacaataaaagactgttaaattgcctgggagaatccttctactacttccttttagaaataaaaccctgaaatttaggtgacaaatggcaacccagctgaaaactccattttgcaggtctttcttctagcaagtatggtgactggaattgactggaatgtggttggtcactcttgcctggaattaggggctttttgatcctaaaactaggaaagtcacaggcaaaccagaatgcactggtcaccctagcagaaagtgtgctatggactaagtttagaccagtgggagatgagtggaattaacatgtgaaacttccagggcatccccttaaagacaaattagcttttgctgaaatttcttttccccacttcctgctgctactacaactggctattgtaagaaccatcttgcacctagagatggaagccatgtattgaggatggcagtgctgaacaccagcctggatctctggatggccttgggaggcaaaatgctctctctaagccaggatgcctaactacctttgaactattatttgagagaaagacatacttgtgtcttattaagccactatattttgggtctcttctttagagtagtttaatccatactctaactgatacttgaacttactttgaaaagtatttaagattctatagcacatttaatgccacatttctgttattagtagatatttttccttaaaaaccatgcatgtggcagtattttcaatttcctttttgcatttttctgctttttacccttgggctcttctttggaattagtgggagatgataattttcttgtctatttggtaaagtgatggcctgacttacttgttttagataagacttattaggaaggcagtgaatctaaaaattacgtacatgaatagaaataaatgttctgaatatttacaatgtgtccacacttctatttaaggagtatacatacttacatatgacacaaactaaaataaatacaaggatgtttgggaaaagcacgctgccaaaccccttggtgaaagcttaccctcggtctgtctttcaacccaagagttgatgtcctttctgatttgatcagaagcttccacaaagttaacaggctgaggttctgcaccaaaatatgttttcatgtcttctaaatatttctgaaagacatagataaaaatccccttttgtagagtttgttcaaaattgagaaatcaaatctatttagattatcaaaagaactaggtaggcgggcaattgcacactcccacaccatcactccatcctctctttcctgactcccagttttggtcaaaaactaaacacctcactcttcaaacttagcagggcactggctagctatgcagagctccagctgtggaaggaagtacggatgcccacatttttggggcttccacatcctcttcctttctatttttatccatgccttgaaatgaggctagaatggaaagggactacaaataacaagagtgaccccagtggccaagaggtactgggcattgtacttgactttttatggtaggtatgaaaccaaagtgaggcttgtgatactggtagcttccttctgtggattctaaacaacaagaatcttgtggaggacccttgaagaaaatgatatcctgccatgaaaatggatttctaagcagagttgaatgttatattaggcattctgaggcaaaaaaaaaaaaaatgaggccttcacctcctcctggtcctctgatgttcacccagtggcagtcatcacatgtaggctatgactgagtttgctgttatggaggcatctgccttgggaacacaatttattcttcaagacattgtagtattccttcaatttgcttactttatgcagggcttgtaggagaaaaagtgatgagttaattctgttgccaggaagtggcttgaagattatgaagtctaaattctgtggaatttaaaatctaataaaaggaaaacttaaatatttttgagggaccactgcctgtcttaattcagggcttgtggacatataaaaatatgtttgtctctatctctgtgtccctcttgtttctaagtgcccactgagtcaatcatgtctcctacttctgcatcaaacgagagtccatgagcctacaagaaaggaaactgtctttaagattccatttggatgattttatgtctttagtaataacaaagctcaattcctgaactccccagaattcactagatttgaacagagatagcattgcttgaaatcacaaggtagctttattttatcttattgctattcttctaccctatctaatttccttgctctttgcccactggacacacgggtcctcctgcccaaagagatgttggacaatgaaggaaattattgacctgggttgtcatgtgaccctcttaacagaggacagagtcatcatggtctgcattggtggggtttcaacccttagtaaagaggagaggttgatattatttcatgtttcatgttcccaaagtctgtaaagtcttgatgtgaaaactcctcacttctctcatcccaaggtcagttttgctctcaagtagacagtctccagccaagtctccatatgtatttgtaggccattctcaggggctacctctttattctagacagaaatatctgattcctagatctgatttgagacctgttattggtttcctttaccactagacactcattggttggtctaattgattgtttgcagggaagtactgaccacatgaagattgtccctggatccctaaagaaatatacagagtttactgattggtctcacattgctctttcttttccattagcttaaaataagacatttgcacttacattgtgaaatgcatacgttttctctccatatatcgcattggctgttttaagtaagtagtcatcgttgggcttgaggatttctgagataagtgtttggaaatcagagtgtatttcttccgagttgctcaagttgaattcctttaaaagagaaggaactagtcaaataaaatgttgaacaagagttacgtacatgttagcaaactaaagctaaataaaattatttggggaattgcaaacaaaagttgaaaatgttcctaaagtggataaaaatgttttctttagtgttagtgcaaaagaatgaaatgttattgaggccataattttcttcacatttagtttgcaaaagcaggcaaggaaaagtttgaaactagagaaatttttagagagattaaaagggcgtggggacaagagatctttcaaaaggtgtagatattttgtattccaaaaaaagtttctgtaactttaatatatgtttctaagaaaaattacctgctaaaagttgcttcagttatcactaaaatacatgtgctaaaagtactttttccttaattatgaaatatatattaaaggataagatataccattttcctttttttttcactttcagggtcacatttgactccctggtctctgttaaattgaagcacctgtaatttcaataaaaatacaaatgaatagactgcagagaagtaattatataaatcatacatcatctgtaaatacaagtcagtcattcctataattgcagaacatataattagttaaacaaaggggctctgacttgacttaatgtgtgtaataaaataagtcaagaactgctcttaaataaattttctaaagtcgaatttaagttgctagtctctttattttcctatgttttgtaactagaatgatgttaggaaaaccttgaggtaaaattctcttattttccgtatttcctttttactcatggttgttttaagaagaatgctaggtgtccaactgagaccatcatttagacagtttaggtaaaagccataattatgtggaaaattaaggaattttagttgttttttttctagttaaatgctcagttcctattgtctaccaccacttgacatatttaggcgcaatgtttgtgtttaccccagcatttcactggaaagttaacaggaaagatgtagatactcttctttccttggaattgtacaacaatagaacagaaggcaatacttttatctgccaagtcctggacccggctgacaataagaaatacggtatactgcttcaacggttgcattgacccatgttaacagaacaagggccacctgccatacataaaggggtgcacagttaattcatgaggcccatttgcaactaaaagtgttaacatccgaaacaaggacaagcaacacccagcaatatatttatatgaaatagattcacattggaatccacttaagattgtctatcacattaatgattaagacttttgagctaatgaacctgtcattaacaagttgtgaacaaggattttgttgttgctgttgttgtttttaaaccacctgatatcttgtgaactacaagagtaaggatgttatcccaagtccagactaatatagcatgtgttctgcccacttccctcagaattaccaaccgtgttcatatccatactttctgaggaagtcaccttcttgggtgaaaattctgtcatttggccttttggttttgcaagatttagggccgagtttactccttctgtttttttttttttttatttaaaatcacatttatatatacatattgttgcatatatatatgtattcatatatatttttacacacacatatggctattttgctatctatgactacatatatatatttacatacgcacaatgttttgaggatgcttattgcaaaaatagttctgtatataatagtattatattgttattgtaatgatcttaatgggctccttagattcattcagataaccagggactaaacaaagagagaatcaaacaaagcatgagaaacagaattcttttatttttcttttttgtgtcttcaagaaaatccttatgtgattataaagatgctgaataattctattatttaaagtttgaccaattactttttgaaataatatcatcaaaataaaattggttttggtgttgaacaaaaattgataaccttatcctttctccattttattttgctaaaggacacaggttctcaaatggttttcctagcaacagtttgcaaattgattctctttaccaagatttgttttatggtatagaatgttttaatgtatgtcttgttttagagaatgttattgtgaaatgtaaattattgagttgtcaagagaaaactgaagttaaaaggcaatgaaagcacttagcttacaagaaacagaagatagttgaccttttccactcacctgggccatttgggctgcagtggtacctttggcgcccaaatacactatggtcaaggaagttgagatgctccaggaagaaaagaagatatttttaccctgagcagattcagctagctttttgctcaactccagggcaaactggttgattgatgttgctagagagtccattgaggaaacctaagaaaaataaagcaaaaattagagagcatattaatatttatttccacttcacctttgaaaaaatatgtattcatagttgcatacatatccatatacattgtcatatgagctgaatagactttagcaaaattctagtccaataatagtattttatatattaaaacactgaagtttagagaatttacgggaattgcctcaaattttagtgctaatgtgcattagagaataaatggatgctggtcagttaaaagctgagtcccataacatagtttttgccactctcttgacatttttcacaccaccagttttgtttccatgtgtttgtatctagtttttccaactccagcataagcttattgagagcaaaatattctttttatgctgcttcgtgcacccaacagcacctagctaaggattttgcacaaaatgggtgcttgtaaatatttattactcatttacgatattcacggtttgggctgtaactaccatttttaatttatgcaaacatggaatcatgcacaactaattggaaaatgcttgtagagaaattgagaagttaagactgtttgtctttattctcactcatttgagaccctctgaaactagtcttctggaacaagagtactcagttttcctcatcttttttgatattatactcaacatctaaatgtcatctctaatggaagcaaccaaacagctggccagcctaccacacctgcatgtacctggcctgaaaatgacaggtgccccaatccacagcaaatgtgcaccaccaccactccatcacaaacactcacagcttggaccactgaggcaactgcagacattattgatgaagactacaactgaggaaactgcatggatattatgctaccaagtccaccctaaaccaaagccaatataccatacccaagcaataccataatattttaatttaaaagccttttaaaactaggtttctatagttaattccatctattttgattttatggcaaaataatttctcgaataagttaaacgatttaaccaagtgctaagggaatctgttactgctagaccagccttacaagaaatccttaaggaagttttaaaaatggaaacaaaagaatgatacctgttacttaagtacgtagcacacacactttataaagcaaccacacaatagaaaccacaaagcaaccagctaataacttcgcaataggatcaacacctcacatatcaatattaaccttgaatgtaaatgggctaaacaccatatttaaaaagcacagagtggcaagttggattaaaaaacaagacccattaatgtgctgtctttaagagacccatttcacatgtaataacacccataggcttaaactaagtggttggaggaagggctatcacacaaattgaaaacaaaatagagcagtgggtgctattcttatatcagatacaacaggctttaagccaacaaaagtaaaaagtgacaaagaagggtattatataatggcaaaaggttcaattcaacaagaagacttaactatactaaacgtatatgcacccaacattggagcactcagattcataagacaagtactcttcaacctacaaaaagacttagacagacacacaacaatagtgggggactttaacaccccattgacagcattagacagatcattgagggagaaaactaacaaagaaaaacttgaccaattgaacttattaataaacatctacagaatactccagccatcaaccacaaaatatacattcttctcatctgcacgcggaatatactccaagattgattacatgctcagccataaagcaagtctcaacaaatttttaaaaactgaaattgtaccaactatactctcagaccacagtgcaataaaaatagaaatcaatgccaagaagatttctcaaaaccacagaattacttggaaattaaacaacgtgctactgaatgactttttggtaaacaatgaaattaccgcagaaatcaacgtctttgaaatagacacagagacataacataccaaagtctctaagatgcagcgaaaagcagtgttaggaggaaagtttatagcactaaatgcctaccttaaaaagttagaaatatttcatattaactatcgaacatcagacctagacgaactagagaaacaagaacaaactaatgctaaaacttccaaaagaaaaaaacataagtaaaatcagcaaaactgaatgaaatcgagacccaaaaatccatacaaagaatcaacaaaaccaaaagttggttttttgaaagtataaacatgattgatagactgcaggctagattaacaaagaaaaaaagaggagatataaataagcacaatcagaaataacaaaagtgacattacaactgatcctggagaaatacaaatgatcctcagagactactatgactacctctatgcacacaaactagataatctagagaaaatgaataaattcctgaaaacacacaacctctcaagattaaattaggaaaatattgagaccatgaacagatcaatatcatgtttcaaaattgaatcaattataaaatcctaccaaccaaaaaacgccctggaacaagtagattcacagctgaattctaccagatgtacaaagacaagctggcatcaatgctgatgaaataatttcaaaaaattaaagaggagggactcctctctaactcattctacaaagccagcatcatcctgataccaaaacctggaaaaaacacaatagaagaagaaacctataggcaaatatctctgatgagcatagatgcaaaaatgctcaataaaatcctagcaagctgaatccagcagcatatcaaaaagttaatttaccaccatcaagtaggctttgttcctggagtactatattggttcaacatacagaagtcagtaaatgtgatttaccacacaattaaaaacaaaaaacatctgatcatctcaatagaggtggagaaagctttcaataaaatccaatatccattcatgataaaaaccctcaacagactaggtgttgaaggaatatacctcaaaataataagagctatctgtaacaaacccacagccaatatcatactgaataggcaaaagctggaagcattccccttgagaactggaacaagacaagaatgcctactctttaccactcctattcaacatagtactagaagtcctggccagagcaatgagacaagagaaagacataaaaggcacccaaataggaaaagaagaagtcaaactatctctcttcattgactatataattctatacttacaaaaccctaaagactccaccaaaagcctcctggaaccgaaaaatggcttcagtaaagcttcagaacacaaaatcgacgtacaaaaatcagtaccatttctatataccaataacattcaaacagggagccaactcaagactacaattccatttatgatagccaaaaaataaaataaaataaaatacccaggaatacacctgatgaagaaattgaaagatctctacaaagtgaggtaaaaaaaaacactgctaaaaatcataaataatacaaacaaatggaaaaacattccatgctcatggacaggaaacatcagtattgttaaaatggccatactgccgaaggcaaacctcggatggaatgctatttccatcaaactaccaacgtcatttttcacagaattggaaaaactattctaaatttcatatggaacaagaaaagagccccaatatccaaagcaaccctaagcaaaaagaacaaacctggaagcatcactttacccaacttcaaactacactataagcctgcagtaaccaaaacagcctggtgctggcacaaaaacagacacataggccaatggaacagaatagagaacttagaaataaagctgcacacctacagacatctaacctttaccaaagtaaacaaaaacaagcaattgggaaaggactctattcaataaatgatgctgggatagctggttaaccatatccagaaaaatgaaagtagaccctacttttcaacatgtacaaaaattaactaaagatagactaaagacttgaatgtaagatctcagactatcagaatcctagaaaaaaaaaaatccaggaaacgccattctggatatcagtgttgggggaagtattcatgactaagtcttcaaaagcaattgcaacaaaaaacaaagtgagacctaattaaactgaagagcttctgcacagcaaaagaaactatcaacagagtaaacagacaactacagaatggaagaaaatatttgcaaactattccactgacaaaagtttaatatccattatgtacaaggaacttaattcaacaagcaaaaaacaaccccattaaaaaatgccaaaggacatgaacagacacttctcaaaagaagacatacaagtgggcaacaaacatgaaaaaatgctcaacatcattaatcattagagaaatgcaagtccaaaccataatgaatactaccttgtatatgtcagaatggctattattaagaagtcaaaaaacaagagatgctgatgacgctgtggagaaaagggtacactgttgatggaaatgtaaattagatcagctactatggaaagcagattggagatttcccaaggaagttagaactacccttcaacccagcaatcccattactgggtatacattcaaaagaaaataaatcattctaccaaagagacacacacacacacacacacaagtgttcatcacagcactattcacaatagcaaagacatgaaatcaacctaggtgcctatcagtgttggattggataaggaatacctggtacatacacaccatggaatactatgcaaccgaaagaaggaattaaatcatgtctcttgctgcaacatggatgcactagaggctgttatcctaagcaaattatgacaggaacaaaataccacatgttctcatttataagtgggacataagcattgggtactcatagacttaaagatggcaaaaatagacactggggactactagaatggggaggcagagaatggagaaaagattgaaaaactaactattgggtactatgctcactacctaggtgatggactcaatcaaacctcaacctcagcatcacacattatacccttctaacaaacctgcatatgtaccctctgagtcaaaagtaaaaactgaaattatttaaaaaataaaataattcaaattcttatacctaggaaatctcattaatgccaaagttgtgtagagttaggtcttgcatgaacgtcacctggccttctgagaaacctagaatgagagctttggaaaacgtcaggaattctctatactcaaataacttaatcatgagagaggcatgtgtagacaaagttttggtttcatcataacttggagttaatagtgatactgtgggaatcccccctagccataaacttacacttaaccaaacagcttccccacctctaaatcatgccctaggtgtggctcctgtctactccaaaagcttatgataatcccaggctgacattatttgattaaatagaaaacaaaaaagactgtagatctgtagtaattctacactctcataaaatattgaatatttgcctgtgactttcttcctcaacagatcaaatgccatcttcaaaataactctgtatttcctttaaggctcttactcattcaaataacattttgttacctgcatacagtatatatcaagctttgtatttttaaaactcatgaattaaatttacacggaaaatgtttggtttttggccaaaccacactcttcatgaaatggggagaaagtaccatttcagaggctggggtttttctacctcagtgtatggttattccagtgtttatttacttgtaacatgttcctgaaaacaataattctctattataaaagctaatttctcatgtaagcttaatatttagttgtactcatcttcattaattcccttattaatatcattatttgtatatcagatgttacccagaacatacaagagaaacaattctttattttctttttttaaagaaatctaatgaagtactaagccatccattagtgatatttaacccaacaatgaaaaatactttctgaagggtgtagaattgtgaggtgaaattttacaggaaataattattccatacaaatgttggacaattatattttcagagaagactgcaaatgtgtaggtcagaagtctaccaggttattttcaaaattaattgctcttttgcagttttcaagagctataaactgatcatctatgagcttaacctgtagacaggctcagaggagaagccaaagagaattaagaaagcaagaaaaggatagtgtcattcatcaaaatgtctcatcatagacatttcatcatcgcatgcccctagcacatcagtgttggaaagacccttagaatctccctaacccagctcccccattttgtctgctaagaaactgaagctgagagaggttgattgagcaaaggatgcacagcagagtcaaaactgcaactgtgttatcttgactgtgaatacagttg

SEQ ID NO: 4 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 5 - DNA, 17 bp, Homo sapiens
gaccttcatcaagagcg

SEQ ID NO: 6 - DNA, 17 bp, Homo sapiens
taaataatcccctgtgg